(19) United States
(12) Patent Application Publication
(10) Pub. No.: US 2025/0129346 A1
(43) Pub. Date: Apr. 24, 2025

(54) ENGINEERING CELLS FOR CELL-BASED THERAPIES, AND ASSOCIATED COMPOSITIONS AND METHODS

(71) Applicant: Sana Biotechnology, Inc., Seattle, WA (US)

(72) Inventors: William Dowdle, South San Francisco, CA (US); Edward Rebar, San Francisco, CA (US)

(21) Appl. No.: 18/682,796

(22) PCT Filed: Aug. 11, 2022
(86) PCT No.: PCT/US2022/074846

§ 371 Date: Feb. 9, 2024

Related U.S. Application Data
(60) Provisional application No. 63/232,142, filed on Aug. 11, 2021.

(51) Int. Cl. C12N 9/22; C12N 5/07; C12N 5/10; C12N 15/11; C12N 15/90

(52) U.S. Cl. CPC C12N 9/22; C12N 5/06; C12N 5/10; C12N 15/111; C12N 15/902; C12N 2310/20; C12N 2510/00

(57) ABSTRACT

Provided are methods for genetically modifying cells to knock out or modify one or more genes associated with blood type, e.g., ABO, FUT1, RHD. Also provided are cells and compositions derived therefrom, as well as methods of using the same to treat various human diseases.

### CROSS-REFERENCE TO RELATED APPLICATIONS

This application claims the benefit of U.S. Provisional Patent Application No. 63/232,142, filed on Aug. 11, 2021, the contents of which are incorporated by reference in their entirety.

### SUMMARY

An emerging cell therapy approach called adoptive cell transfer (ACT) is rapidly changing the scene of human disease treatment. ACT involves collecting cells from a patient (autologous) or healthy donors (allogeneic), engineering these cells (e.g., through gene, mRNA, or protein modifications), and transferring the cells into the patient to fight diseases. One of the complications of allogeneic therapies is that it requires blood type matching between a donor and a recipient. Blood types are determined by the presence or absence of certain antigens on the surface of red blood cells (RBCs) and many other cell types in the body. Since the presence of these antigens can trigger a recipient's immune system to attack the infused cells, safe and effective allogeneic therapies depend on blood type matching.

As of 2019, a total of 41 human blood group systems are recognized by the International Society of Blood Transfusion (ISBT). The two most commonly referenced blood group systems are ABO and Rh. Four major blood types are determined by the presence or absence of the A and B antigens (A, B, AB, and O). In addition to the A and B antigens, the Rh factor can be either present (+) or absent (−), creating the eight most common blood types (A+, A−, B+, B−, AB+, AB−, O+, and O−). Several of the blood type-determining antigens are controlled by a single gene.

The presence of the blood type-determining antigens is usually associated with the absence of antibodies against those antigens in the subject's plasma thereby preventing a potential agglutination reaction. Thus, for allogeneic therapies, blood type compatibility is important to avoid graft rejection and other negative immune reactions. Because type O− individuals do not have A, B, or Rh antigens on the surface of their cells, they are usually referred to as universal donors for any recipient having any blood type.

The present technology provides methods for genetically engineering cells to knock out, knock down or otherwise alter one or more genes associated with blood type, e.g., ABO, FUT1, RHD, to improve the efficacy and safety of allogeneic cell therapies. Also provided herein are cells and compositions derived therefrom, as well as methods of using the same to treat various human diseases.

In some aspects, methods are provided for genetically modifying one or more genes associated with blood type in a cell, the methods comprising introducing into the cell a site-directed nuclease or a nucleotide sequence encoding a site-directed nuclease, wherein the one or more genes associated with blood type are selected from the group consisting of ABO, FUT1, and RHD. In some embodiments, the methods further comprise introducing to the cell a guide RNA (gRNA) targeting the ABO, FUT1, or RHD locus.

In some aspects, gRNAs are provided for use in genetically modifying one or more genes associated with blood type in a cell, wherein the one or more genes associated with blood type is selected from the group consisting of ABO, FUT1, and RHD.

In some embodiments, the site-directed nuclease is selected from the group consisting of a zinc finger nuclease (ZFN), a transcription activator-like effector nuclease (TALEN), a meganuclease, a CRISPR-associated transposase, and a CRISPR/Cas nuclease.

In some embodiments, the site-directed nuclease is a CRISPR/Cas nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

In some embodiments, the gRNA comprises a CRISPR RNA (crRNA) and optionally a transactivating CRISPR RNA (tracrRNA). In some embodiments, the gRNA comprises a crRNA and a tracrRNA as two separate molecules. In some embodiments, the gRNA comprises a crRNA and a tracrRNA as a single guide RNA (sgRNA). In some embodiments, the sgRNA comprises a complementary region, a crRNA repeat region, a tetraloop, and a tracrRNA.

In some embodiments, the crRNA repeat region comprises, consists of, or consists essentially of a nucleotide sequence set forth in SEQ ID NO:5, SEQ ID NO:9, SEQ ID NO:13, or SEQ ID NO:18. In some embodiments, the tetraloop comprises, consists of, or consists essentially of a nucleotide sequence set forth in SEQ ID NO:6 or SEQ ID NO:17. In some embodiments, the tracrRNA comprises, consists of, or consists essentially of a nucleotide sequence set forth in SEQ ID NO:7, SEQ ID NO:11, SEQ ID NO:15, or SEQ ID NO:16.

In some embodiments, the crRNA comprises a complementary region specific to a region of the ABO locus, including, for example, a coding sequence (CDS), an exon, an intron, a sequence spanning a portion of an exon and a portion of an adjacent intron, or a regulatory region. In some embodiments, the complementary region comprises, consists of, or consists essentially of a nucleotide sequence complementary to a nucleotide sequence set forth in any of SEQ ID NOs: 20-203.

In some embodiments, the crRNA comprises a complementary region specific to a region of the FUT1 locus, including, for example, a CDS, an exon, an intron, a sequence spanning a portion of an exon and a portion of an adjacent intron, or a regulatory region. In some embodiments, the complementary region comprises, consists of, or consists essentially of a nucleotide sequence complementary to a nucleotide sequence set forth in any of SEQ ID NOs: 204-420.

In some embodiments, the crRNA comprises a complementary region specific to a region of the RHD locus, including, for example, a CDS, an exon, an intron, a sequence spanning a portion of an exon and a portion of an adjacent intron, or a regulatory region. In some embodiments, the complementary region comprises, consists of, or consists essentially of a nucleotide sequence complementary to a nucleotide sequence set forth in any of SEQ ID NOs: 421-580.

In some embodiments, the genetic modification occurs via non-homologous end-joining (NHEJ). In some embodiments, the genetic modification occurs via homology-directed repair (HDR). In some embodiments, the genetic modifications include both HDR- and NHEJ-induced modifications.

In some embodiments, provided are compositions comprising the gRNA according to various embodiments of the present technology. In some embodiments, the compositions further comprise a site-directed nuclease or a nucleotide sequence encoding a site-directed nuclease protein as described herein.

In some embodiments, the compositions comprising the gRNA according to various embodiments of the present technology are formulated for delivery into a cell. In some embodiments, the cell further comprises a site-directed nuclease or a nucleotide sequence encoding a site-directed nuclease protein as described herein.

In some aspects, provided are methods of identifying a new genomic locus for genetically modifying one or more genes associated with blood type in a cell, the methods comprising (a) locating a genomic locus based on a known gRNA; and (b) scanning a region of about 500 to 4000 bp on either side of the genomic locus for a PAM sequence, wherein the one or more genes associated with blood type is selected from the group consisting of ABO, FUT1, and RHD. In some embodiments, the known gRNA targets the ABO, FUT1, or RHD locus. In some embodiments, the gRNA comprises a complementary region comprising, consisting of, or consisting essentially of a nucleotide sequence complementary to a nucleotide sequence set forth in any of SEQ ID NOs: 20-580.

In some aspects, provided are cells having one or more genes associated with blood type genetically modified according to various embodiments of the present technology. In some embodiments, the cell is an autologous cell. In some embodiments, the cell is an allogeneic cell. In some embodiments, the cell is a pluripotent stem cell, an embryonic stem cell (ESC) or an induced pluripotent stem cell (iPSC). In some embodiments, the cell is differentiated from a pluripotent stem cell (e.g., an ESC or an iPSC). In some embodiments, the cell is the cell is a primary cell. In some embodiments, the cell is a blood cell, e.g., a red blood cell, a platelet cell, a mast cell, a basophil, an eosinophil, a neutrophil, a monocyte, a natural killer (NK) cell, a natural killer T (NKT) cell, a macrophage, a T cell, a B cell, or a plasma cell. In some embodiments, the cell is a T cell, an NK cell, or an NKT cell. In some embodiments, the cell is a cardiomyocyte. In some embodiments, the cell is a retinal pigment epithelial cell (RPE). In some embodiments, the cell is an endothelial cell. In some embodiments, the cell is a β islet cell. In some embodiments, the cell is a glial progenitor cell (GPC).

In some embodiments, the cell is modified to have reduced expression of one or more MHC I molecules and/or one or more MHC II molecules, optionally, wherein the one or more MHC I molecules are selected from the group consisting of HLA-A, HLA-B, HLA-C, and optionally, wherein the one or more MHC II molecules are selected from the group consisting of HLA-DR, HLA-DQ, HLA-DP, HLA-DM, and HLA-DO. In some embodiments, the modification is by modulation of the B2M, TAP1, CIITA, MIC-A, and/or MIC-B loci. In some embodiments, the modulation of the B2M, TAP1, CIITA, MIC-A, and/or MIC-B loci comprises B2M, TAP1, CIITA, MIC-A, and/or MIC-B knockout. In some embodiments, the modulation of the B2M, TAP1, CIITA, MIC-A, and/or MIC-B loci comprises knock-in of a transgene at the B2M, TAP1, CIITA, MIC-A, and/or MIC-B loci.

In some embodiments, the transgene encodes one or more tolerogenic factors selected from the group consisting of A20/TNFAIP3, CD16, CD16 Fc receptor, CD24, CD35, CD39, CD46, CD47, CD52, CD55, CD59, CD200, CCL22, CTLA4-Ig, C1 inhibitor, complement receptor (CR1), DUX4, FASL, H2-M3, IDO1, IL15-RF, HLA-C, HLA-E, HLA-E heavy chain, HLA-G, IL-10, IL-35, MANF, PD-1, PD-L1, SERPINB9, CCL21, and MFGE8. In some embodiments, the one or more tolerogenic factors comprise CD47, for example, human CD47. In some embodiments, the human CD47 comprises an amino acid sequence that is at least 80% identical to the amino acid sequence set forth in any one of SEQ ID NOs: 583-588. In some embodiments, the human CD47 comprises an amino acid sequence that is at least 80% identical to the amino acid sequence set forth in SEQ ID NO:584. In some embodiments, the one or more tolerogenic factors comprise HLA-E. In some embodiments, the one or more tolerogenic factors comprise CD24. In some embodiments, the one or more tolerogenic factors comprise PD-L1. In some embodiments, the one or more tolerogenic factors comprise CD24, CD47, and PD-L1. In some embodiments, the one or more tolerogenic factors comprise CD46. In some embodiments, the one or more tolerogenic factors comprise CD55. In some embodiments, the one or more tolerogenic factors comprise CD59. In some embodiments, the one or more tolerogenic factors comprise C1 inhibitor. In some embodiments, the one or more tolerogenic factors comprise CD46, CD55, CD59, and C1 inhibitor. In some embodiments, the one or more tolerogenic factors comprise HLA-E, CD24, CD47, PD-L1, CD46, CD55, CD59, and C1 inhibitor.

In some embodiments, the cell is modified to have reduced expression of one or more MHC I molecules and/or one or more MHC II molecules; increased expression of CD47, and optionally CD24 and PD-L1; and increased expression of CD46, CD55, CD59, and CR1.

In some embodiments, the cell is modified to have reduced expression of one or more MHC I molecules; reduced expression of TXNIP; increased expression of PD-L1 and HLA-E; and optionally increased expression of A20/TNFAIP3, and/or MANE.

In some embodiments, the cell is modified to have increased expression of CCL21, PD-L1, FASL, SERPINB9, HLA-G, CD47, CD200, and MFGE8.

In some aspects, provided are pharmaceutical compositions comprising cells having one or more genes associated with blood type genetically modified according to various embodiments of the present technology.

In some aspects, provided are methods of treating a disease in a subject in need thereof, the methods comprising administering the subject cells having one or more genes associated with blood type genetically modified according to various embodiments of the present technology, or pharmaceutical compositions comprising the same.

In some embodiments, the disease is cancer, e.g., a hematologic malignancy. In some embodiments, the hematologic malignancy is selected from the group consisting of myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), B cell acute lymphoid leukemia (B-ALL), T cell acute lymphoid leukemia (T-ALL), T cell lymphoma, and B cell lymphoma.

In some embodiments, the disease is an autoimmune disease, e.g., lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

In some embodiments, the disease is diabetes mellitus, e.g., Type I diabetes, Type II diabetes, prediabetes, and gestational diabetes.

In some embodiments, the disease is a neurological disease, e.g., catalepsy, epilepsy, encephalitis, meningitis, migraine, Huntington's, Alzheimer's, Parkinson's, Pelizaeus-Merzbacher disease, and multiple sclerosis.

In some embodiments, the disease is a cardiac disease, e.g., pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, cardiomegaly, mitral insufficiency, and autoimmune endocarditis.

### DETAILED DESCRIPTION

The present technology provides methods for engineering cells, including through genetic editing, to alter the expression of one or more genes associated with blood type, e.g., ABO, FUT1, and/or RHD. Also provided are site-directed nucleases and guide RNAs, as well as compositions and vectors thereof, for use in these methods. Moreover, the present technology provides genetically modified cells and cell populations generated using these gene editing methods as well as methods of using these cells and cell populations to treat various human diseases.

While the present disclosure is capable of being embodied in various forms, the description below of several embodiments is made with the understanding that the present disclosure is to be considered as an exemplification of the invention and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios, such as about 2, about 3, and about 4, and sub-ranges, such as about 10 to about 50, about 20 to about 100, and so forth. It also is to be understood, although not always explicitly stated, that the reagents described herein are merely exemplary and that equivalents of such are known in the art.

To the extent any materials incorporated by reference herein conflict with the present disclosure, the present disclosure controls.

### Definitions

The term “about,” as used herein when referring to a measurable value, such as an amount or concentration and the like, is meant to encompass variations of 20%, 10%, 5%, 1%, 0.5%, or even 0.1% of the specified amount.

The term “antibody” is used to denote, in addition to natural antibodies, genetically engineered or otherwise modified forms of immunoglobulins or portions thereof, including chimeric antibodies, human antibodies, humanized antibodies, or synthetic antibodies. The antibodies may be monoclonal or polyclonal antibodies. In those embodiments wherein an antibody is an immunogenically active portion of an immunoglobulin molecule, the antibody may include, but is not limited to, a single chain variable fragment antibody (scFv), disulfide linked Fv, single domain antibody (sdAb), VHH antibody, antigen-binding fragment (Fab), Fab′, F(ab′)2 fragment, or diabody. An scFv antibody is derived from an antibody by linking the variable regions of the heavy (VH) and light (VL) chains of the immunoglobulin with a short linker peptide. Similarly, a disulfide linked Fv antibody can be generated by linking the VH and VL using an interdomain disulfide bond. On the other hand, sdAbs consist of only the variable region from either the heavy or light chain and usually are the smallest antigen-binding fragments of antibodies. A VHH antibody is the antigen binding fragment of heavy chain only. A diabody is a dimer of scFv fragment that consists of the VH and VL regions noncovalent connected by a small peptide linker or covalently linked to each other. The antibodies disclosed herein, including those that comprise an immunogenically active portion of an immunoglobulin molecule, retain the ability to bind a specific antigen.

The term “antigen” refers to an immunogenic molecule that provokes an immune response. This immune response may involve antibody production, activation of specific immunologically competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can also be produced by cells that have been modified or genetically engineered to express an antigen.

The term “autoimmune disease,” “autoimmune disorder,” “inflammatory disease,” or “inflammatory disorder” refers to any disease or disorder in which the subject mounts an immune response against its own tissues and/or cells. Autoimmune disorders can affect almost every organ system in the subject (e.g., human), including, but not limited to, diseases of the nervous, gastrointestinal, and endocrine systems, as well as skin and other connective tissues, eyes, blood and blood vessels. Examples of autoimmune diseases include, but are not limited to Hashimoto's thyroiditis, systemic lupus erythematosus, Sjögren's syndrome, Graves' disease, scleroderma, rheumatoid arthritis, multiple sclerosis, myasthenia gravis and diabetes.

The term “codon-optimized” or “codon optimization” when referring to a nucleotide sequence is based on the discovery that the frequency of occurrence of synonymous codons (i.e., codons that code for the same amino acid) in coding nucleotide is biased in different species. Such codon degeneracy allows an identical polypeptide to be encoded by a variety of nucleotide sequences. Codon optimization refers to the process of substituting certain codons in a coding nucleotide sequence with synonymous codons based on the host cell's preference without changing the resulting polypeptide sequence. A variety of codon optimization methods are known in the art, and include, for example, methods disclosed in at least U.S. Pat. Nos. 5,786,464 and 6,114,148.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of introducing a specific nucleic acid sequence into a cell or into another nucleic acid sequence, or as a means of transporting another nucleic acid molecule. Vectors may be, for example, plasmids, cosmids, viruses, an RNA vector, or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

The term “expression” refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “hypoimmunogenicity,” “hypoimmunogeneic,” “hypoimmunogenic,” “hypoimmunity,” or “hypoimmune” is used interchangeably to describe a cell being less prone to immune rejection by a subject into which such cell is transplanted. For example, relative to an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cell is transplanted. In some examples described herein, genome editing technologies are used to modulate the expression of MHC I and MHC II genes, and thus, to generate a hypoimmunogenic cell. In other examples described herein, a tolerogenic factor is introduced into a cell and when expressed can modulate or affect the ability of the cell to be recognized by host immune system and thus confer hypoimmunogenicity. Hypoimmunogenicity of a cell can be determined by evaluating the cell's ability to elicit adaptive and innate immune responses. Such immune response can be measured using assays recognized by those skilled in the art, for example, by measuring the effect of a hypoimmunogenic cell on T cell proliferation, T cell activation, T cell killing, NK cell proliferation, NK cell activation, and macrophage activity. Hypoimmunogenic cells may undergo decreased killing by T cells and/or NK cells upon administration to a subject or show decreased macrophage engulfment compared to an unmodified or wildtype cell. In some cases, a hypoimmunogenic cell elicits a reduced or diminished immune response in a recipient subject compared to a corresponding unmodified wild-type cell. In some cases, a hypoimmunogenic cell is nonimmunogenic or fails to elicit an immune response in a recipient subject. Detailed descriptions of hypoimmunogenic cells, methods of producing the same, and methods of using the same are found in WO2016183041 filed May 9, 2015; WO2018132783 filed Jan. 14, 2018; WO2018176390 filed Mar. 20, 2018; WO2020018615 filed Jul. 17, 2019; WO2020018620 filed Jul. 17, 2019; WO2021022223 filed Jul. 31, 2020; WO2021022223 filed Jul. 31, 2020; WO2021041316 filed Aug. 24, 2020; WO2021222285 filed Apr. 27, 2021, 2020; and WO2021222285 filed Apr. 27, 2021, the disclosures including the examples, sequence listings and figures are incorporated herein by reference in their entirety.

The term “nucleic acid” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA), which includes cDNA, genomic DNA, and synthetic DNA, either of which may be single- or double-stranded. A nucleic acid molecule encoding an amino acid sequence includes all nucleotide sequences that encode the same amino acid sequence.

The term “subject” refers to a mammalian subject, preferably a human. A “subject in need thereof” may refer to a subject who has been diagnosed with a disease or is at an elevated risk of developing a disease. The phrases “subject” and “patient” are used interchangeably herein.

A “therapeutically effective amount” as used herein is an amount that produces a desired effect in a subject for treating a disease. In certain embodiments, the therapeutically effective amount is an amount that yields maximum therapeutic effect. In other embodiments, the therapeutically effective amount yields a therapeutic effect that is less than the maximum therapeutic effect. For example, a therapeutically effective amount may be an amount that produces a therapeutic effect while avoiding one or more side effects associated with a dosage that yields maximum therapeutic effect. A therapeutically effective amount for a particular composition will vary based on a variety of factors, including, but not limited, to the characteristics of the therapeutic composition (e.g., activity, pharmacokinetics, pharmacodynamics, and bioavailability), the physiological condition of the subject (e.g., age, body weight, sex, disease type and stage, medical history, general physical condition, responsiveness to a given dosage, and other present medications), the nature of any pharmaceutically acceptable carriers, excipients, and preservatives in the composition, and the route of administration. One skilled in the clinical and pharmacological arts will be able to determine a therapeutically effective amount through routine experimentation, namely by monitoring a subject's response to administration of the therapeutic composition and adjusting the dosage accordingly. For additional guidance, see, e.g., Remington: The Science and Practice of Pharmacy, 22^{nd} Edition, Pharmaceutical Press, London, 2012, and Goodman & Gilman's The Pharmacological Basis of Therapeutics, 12^{th} Edition, McGraw-Hill, New York, NY, 2011, the entire disclosures of which are incorporated by reference herein.

The term “tolerogenic factor” as used herein includes hypoimmunity factors, complement inhibitors, and other factors that modulate or affect the ability of a cell to be recognized by the immune system of a host or recipient subject upon administration, transplantation, or engraftment.

The terms “treat,” “treating,” and “treatment” as used herein with regard to a disease refers to alleviating one or more symptoms of the disease partially or entirely; preventing the disease; decreasing the likelihood of occurrence or recurrence of the disease; slowing the progression or development of the disease; eliminating, reducing, or slowing the development of one or more symptoms associated with the disease; or increasing progression-free or overall survival of the disease. For example, “treating” may refer to preventing or slowing the existing disease from progressing and/or slowing the development of certain symptoms of the disease. In some embodiments, the term “treat,” “treating,” or “treatment” means that the subject has a lesser degree of the disease comparing to a subject without being administered with the treatment. In some embodiments, the term “treat,” “treating,” or “treatment” means that one or more symptoms of the disease are alleviated in a subject receiving the treatment as disclosed and described herein comparing to a subject who does not receive such treatment.

A “vector” refers to a DNA construct containing a nucleic acid molecule that is operably linked to a suitable control sequence capable of effecting the expression of the nucleic acid molecule in a suitable host. Such control sequences may include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself.

### Methods of Genetically Engineering Cells

Provided herein in certain embodiments are methods of genetically engineering a cell or population of cells to knock out, knock down, or otherwise alter the expression of one or more genes associated with blood type, including but not limited to ABO, FUT1, and RHD. As used herein, “knock out” includes deleting all or a portion of the target nucleotide sequence in a way that interferes with the function of the target gene. For example, a knock out can be achieved by altering a target nucleotide sequence by inducing an indel in a functional domain of the target nucleotide sequence (e.g., a DNA binding domain) or where base editing and prime editing can be used to change single nucleic acid bases to an alternate base in order to alter the genome sequence. “Knock down” refers to genetic modifications that result in reduced expression of the edited gene. As used herein, “indel” refers to a mutation resulting from an insertion, deletion, or a combination thereof, of nucleotide bases in the genome. Thus, an indel typically inserts or deletes nucleotides from a sequence. As will be appreciated by those skilled in the art, an indel in a coding region of a genomic sequence will result in a frameshift mutation, unless the length of the indel is a multiple of three. A gene editing system, e.g., the CRISPR/Cas system, of the present disclosure can be used to induce an indel of any length in a target polynucleotide sequence.

In certain embodiments, the methods provided herein utilize gene editing. Gene editing is a type of genetic engineering in which a nucleotide sequence may be inserted, deleted, modified, or replaced in the genome of a living organism. Current gene editing techniques generally utilize the innate mechanism for cells to repair double-strand breaks (DSBs) in DNA.

Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knockouts, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. The methods provided herein may utilize HDR-mediated repair, NHEJ-mediated repair, or a combination thereof.

### A. Gene Editing Systems

In some embodiments, the methods provided herein for genetically modifying a cell or population of cells to knock out, knock down, or otherwise modify one or more genes utilize a site-directed nuclease, including, for example, prime editing, zinc finger nucleases (ZFNs), transcription activator-like effector nucleases (TALENs), meganucleases, transposases, and clustered regularly interspaced short palindromic repeat (CRISPR)/Cas systems.

### 1. Prime and PASTE Editing

Prime editing is a versatile and precise genome editing method that directly writes new genetic information into a specified DNA site using a catalytically impaired Cas9 endonuclease fused to an engineered reverse transcriptase, programmed with a prime editing guide RNA (pegRNA) that both specifies the target site and encodes the desired edit. See, e.g., Anzalone et al., Nature, 576:149-157 (2019); WO2021072328; WO2022067130, all of which are incorporated herein by reference in their entirety. Cas9 and a reverse transcriptase can also be used to insert an integrase site into the genome for insertion of a nucleic acid of interest in a process called Programmable Addition via Site-specific Targeting Elements (PASTE) editing. See, e.g., loannidi et al., bioRxiv 2021.11.01.466786; doi.org/10.1101/2021.11.01.466786, all of which are incorporated herein by reference in their entirety.

### 2. ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

### 3. TALENs

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

### 4. Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

### 5. Transposases

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

### 6. CRISPR/Cas

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, Ill, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096):816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complexes, including in certain embodiments via a single gRNA. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 1 below.

TABLE 1
Exemplary Cas nuclease variants and their PAM sequences

| CRISPR Nuclease | Source Organism | PAM Sequence (5′→3′) |
|---|---|---|
| SpCas9 | Streptococcus pyogenes | ngg or nag |
| SaCas9 | Staphylococcus aureus | ngrrt or ngrrn |
| NmeCas9 | Neisseria meningitidis | nnnngatt |
| CjCas9 | Campylobacter jejuni | nnnnryac |
| StCas9 | Streptococcus thermophilus | nnagaaw |
| TdCas9 | Treponema denticola | naaaac |
| LbCas12a (Cpf1) | Lachnospiraceae bacterium | tttv |
| AsCas12a (Cpf1) | Acidaminococcus sp. | tttv |
| AacCas12b | Alicyclobacillus acidiphilus | ttn |
| BhCas12b v4 | Bacillus hisashii | attn, tttn, or gttn |

r = a or g;
y = c or t;
w = a or t;
v = a or c or g;
n = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example, the Cas nuclease may have one or more mutations that alter its PAM specificity.

### B. Genomic Loci for Gene Editing

In some embodiments of the methods provided herein, the genomic locus for site-directed knockout, knockdown, or other modification is a gene associated with blood type. In some embodiments, the one or more genes associated with blood types are selected from the group consisting of ABO, FUT1, and RHD. In some embodiments, two or more locations in a gene are modified. In some embodiments, two or more genes are modified.

The specific site for editing within a gene may be located within any suitable region of the gene, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the gene editing occurs in one allele of the specific genomic locus. In some embodiments, the gene editing occurs in both alleles of the specific genomic locus.

### 1. ABO

The ABO gene encodes blood group ABO system transferase, which is an enzyme with glycosyltransferase activity, and determines the ABO blood type of an individual by modifying the oligosaccharides on cell surface glycoproteins. The ABO gene locus encodes three alleles. The A allele produces α-1,3-N-acetylgalactosamine transferase (A-transferase), which catalyzes the transfer of GaINAc residues from the UDP-GaINAc donor nucleotide to the Gal residues of the acceptor H antigen, converting the H antigen into A antigen in A and AB individuals. The B allele encodes α-1,3-galactosyl transferase (B-transferase), which catalyzes the transfer of Gal residues from the UDP-Gal donor nucleotide to the Gal residues of the acceptor H antigen, converting the H antigen into B antigen in B and AB individuals. The O allele lacks both enzymatic activities because of a frame shift near the N-terminus resulting in translation of an almost entirely different protein. Thus, neither A nor B antigen is found in O individuals.

The human ABO gene resides on chromosome 9 at the band 9q34.2 (chromosome 9: 133,233,278-133,276,024, reverse strand). The ABO genomic sequence as set forth in Ensembl ID ENSG00000175164.16 is set forth in SEQ ID NO:1.

### 2. FUT1

The FUT1 gene encodes galactoside 2-alpha-L-fucosyltransferase 1, which is involved in the synthesis of the H antigen (determinant of blood type O).

The human FUT1 gene resides on chromosome 19 at the band 19q13.33 (chromosome 19:48,748,011-48,755,390, reverse strand). The FUT1 genomic sequence as set forth in Ensembl ID: ENSG00000174951.12 is set forth in SEQ ID NO:2.

### 3. RHD

The Rh system is the second most important blood-group system with currently 50 antigens, of which the D antigen is the most significant Rh antigen for its likelihood to provoke an immune system response. The Rh D antigen is encoded by the RHD gene. Other Rh antigen encoding genes include RHCE, RhAG, RhBG, and RhCG.

The human RHD gene resides on chromosome 1 at the band 1p36.11 (chromosome 1: 25,272,393-25,330,445, forward strand). The RHD genomic sequence as set forth in Ensembl ID: ENSG00000187010.21 is set forth in SEQ ID NO:3.

In some embodiments, the modifications to the specific genomic loci may prevent expression of the genes entirely (i.e., knockout). In some embodiments, the modifications to the specific genomic loci may result in reduced expression of the genes (i.e., knockdown). In certain of these embodiments, gene knockout or knockdown may be achieved by any of the site-directed nuclease-based gene editing systems described, including, for example, the CRISPR/Cas system. In some embodiments, the gene knockout or knockdown occurs through insertion-deletion (indel) mutations at the target loci (e.g., through the NHEJ pathway) including, for example, frameshift-inducing indels or indels in a protein-coding region that result in loss-of-function mutations. In some embodiments, the gene knockout or knockdown occurs through deletions of the genes or portions thereof through either the NHEJ or HDR pathway, although HDR is a preferred pathway for introducing specific deletions. In some embodiments, the gene knockout or knockdown occurs through introduction of silencing or loss-of-function mutations at the target loci through the HDR pathway.

In some embodiments, the modifications to the specific genomic loci may involve insertion of exogenous genes to be expressed in place of the genes being edited (i.e., knock-in). In certain of these embodiments, gene knock-in may be achieved by the introduction of a site-directed nuclease and a transgene to be inserted through homologous recombination. The transgene can be flanked by homology arms (e.g., left homology arm (LHA) and right homology arm (RHA), respectively) and delivered to the cell for insertion into specified loci by HDR-based approaches as described. The homology arms are specifically designed for the target loci to serve as a template for HDR. The length of each homology arm is generally dependent on the size of the transgene being inserted, with larger insertions requiring longer homology arms. Any of the gene editing systems described, including, for example, the CRISPR/Cas system, may be employed for gene knock-in. In addition to expression of transgenes, gene knock-in may result in lost or reduced expression of the original genes at the target loci.

In some embodiments, the gene editing occurs at one or more genomic loci associated with blood types including, for example, ABO, FUT1, and RHD, and results in reduced or no expression of one or more of these genes. By modulating (e.g., reducing or deleting) expression of the ABO, FUT1, and/or RHD gene, the blood type of the cells may be modified. For example, the blood type of the cell may be changed from A, B, or AB to O by knocking out the A and/or B alleles of the ABO gene. For another example, the blood type of the cell may be changed from Rh+ to Rh− by knocking out the RHD gene.

C. Guide RNAs (gRNAs) for Gene Editing

In some embodiments, gRNAs are provided for use in targeted gene editing as described herein, especially in association with the CRISPR/Cas system. The gRNAs comprise a crRNA sequence, which in turn comprises a complementary region (also called a spacer) that recognizes and binds a complementary target DNA of interest. The length of the spacer or complementary region is generally between 15 and 30 nucleotides, usually about 20 nucleotides in length, although will vary based on the requirements of the specific CRISPR/Cas system. In certain embodiments, the spacer or complementary region is fully complementary to the target DNA sequence. In other embodiments, the spacer is partially complementary to the target DNA sequence, for example at least 80%, 85%, 90%, 95%, 98%, or 99% complementary.

In certain embodiments, the gRNAs provided herein further comprise a tracrRNA sequence, which comprises a scaffold region for binding to a nuclease. The length and/or sequence of the tracrRNA may vary depending on the specific nuclease being used for editing. In certain embodiments, nuclease binding by the gRNA does not require a tracrRNA sequence. In those embodiments where the gRNA comprises a tracrRNA, the crRNA sequence may further comprise a repeat region for hybridization with complementary sequences of the tracrRNA.

In some embodiments, the gRNAs provided herein comprise two or more gRNA molecules, for example a crRNA and a tracrRNA as two separate molecules. In other embodiments, the gRNAs are single guide RNAs (sgRNAs), including sgRNAs comprising a crRNA and a tracrRNA on a single RNA molecule. In certain of these embodiments, the crRNA and tracrRNA are linked by an intervening tetraloop.

In some embodiments, one gRNA can be used in association with a site-directed nuclease for targeted editing of a gene locus of interest. In other embodiments, two or more gRNAs targeting the same gene locus of interest can be used in association with a site-directed nuclease.

In some embodiments, exemplary gRNAs (e.g., sgRNAs) for use with various common Cas nucleases that require both a crRNA and tracrRNA, including Cas9 and Cas12b (C2c1), are provided in Table 2. See, e.g., Jinek et al., Science (2012) 337 (6096):816-821; Dang et al., Genome Biology (2015) 16:280; Ran et al., Nature (2015) 520:186-191; Strecker et al., Nature Comm. (2019) 10:212. For each exemplary gRNA, sequences for different portions of the gRNA, including the complementary region or spacer, crRNA repeat region, tetraloop, and tracrRNA, are shown. In some embodiments, the gRNA comprises all or a portion of the nucleotide sequences set forth in SEQ ID NOs: 4-7. In some embodiments, the gRNA comprises all or a portion of the nucleotide sequences set forth in SEQ ID NOs: 8-11. In some embodiments, the gRNA comprises all or a portion of the nucleotide sequences set forth in SEQ ID NOs: 12-15. In some embodiments, the gRNA comprises all or a portion of the nucleotide sequences set forth in SEQ ID NOs: 16-19.

In some embodiments, the gRNA comprises a crRNA repeat region comprising, consisting of, or consisting essentially of the nucleotide sequence set forth in SEQ ID NO:5, SEQ ID NO:9, SEQ ID NO:13, or SEQ ID NO:18. In some embodiments, the gRNA comprises a tetraloop comprising, consisting of, or consisting essentially of the nucleotide sequence set forth in SEQ ID NO:6 or SEQ ID NO:17. In some embodiments, the gRNA comprises a tracrRNA comprising, consisting of, or consisting essentially of the nucleotide sequence set forth in SEQ ID NO:7, SEQ ID NO:11, SEQ ID NO:15, or SEQ ID NO:16.

TABLE 2
Exemplary gRNA structure and sequence for CRISPR/Cas

| SEQ ID NO: | Sequence (5′→3′) | Description |
|---|---|---|
| 4 | nnnnnnnnnnnnnnnnnnnn | Exemplary spCas9 1 |
| | | Complementary region |
| | | (spacer) |
| 5 | guuuuagagcua | Exemplary spCas9 1 |
| | | crRNA repeat region |
| 6 | gaaa | Exemplary spCas9 1 |
| | | tetraloop |
| 7 | uagcaaguuaaaauaaggcuaguccguuaucaacuug | Exemplary spCas9 1 |
| | aaaaaguggcaccgagucggugcuuuuuu | tracrRNA |
| 8 | nnnnnnnnnnnnnnnnnnnn | Exemplary spCas9 2 |
| | | Complementary region |
| | | (spacer) |
| 9 | guuusagagcuaugcug | Exemplary spCas9 2 |
| | | crRNA repeat region |
| 10 | gaaa | Exemplary spCas9 2 |
| | | tetraloop |
| 11 | cagcauagcaaguusaaauaaggcuaguccguuauca | Exemplary spCas9 2 |
| | acuugaaaaaguggcaccgagucggugcuuuuuu | tracrRNA |
| 12 | nnnnnnnnnnnnnnnnnnnn | Exemplary saCas9 |
| | | Complementary region |
| | | (spacer) |
| 13 | guuuuaguacucug | Exemplary saCas9 |
| | | crRNA repeat region |
| 14 | gaaa | Exemplary saCas9 |
| | | tetraloop |
| 15 | cagaaucuacuaaaacaaggcaaaaugccguguuuau | Exemplary saCas9 |
| | cucgucaacuuguuggcgagauuuuuu | tracrRNA |
| 16 | gucgucuauaggacggcgaggacaacgggaagugcca | Exemplary AkCas12b |
| | augugcucuuuccaagagcaaacaccccguuggcuuca | tracrRNA |
| | agaugaccgcucg | |
| 17 | aaaa | Exemplary AkCas12b |
| | | tetraloop |
| 18 | cgagcggucugagaaguggcacu | Exemplary AkCas12b |
| | | crRNA repeat region |
| 19 | nnnnnnnnnnnnnnnnnnnn | Exemplary AkCas12b |
| | | Complementary region |
| | | (spacer) |

S = c or g; n = any base

In some embodiments, the gRNA comprises a complementary region specific to a blood type gene locus, for example, the ABO locus, the FUT1 locus, or the RHD locus. The complementary region may bind a target sequence in any region of the blood type gene locus, including for example, a CDS, an exon, an intron, a sequence spanning a portion of an exon and a portion of an adjacent intron, or a regulatory region (e.g., promoter, enhancer). Where the target sequence is a CDS, exon, intron, or sequence spanning portions of an exon and intron, the CDS, exon, intron, or exon/intron boundary may be defined according to any splice variant of the target gene. In some embodiments, the genomic locus targeted by the gRNA is located within 4000 bp, within 3500 bp, within 3000 bp, within 2500 bp, within 2000 bp, within 1500 bp, within 1000 bp, or within 500 bp of any of the loci or regions thereof as described.

In some embodiments, the gRNA used herein for targeted gene editing comprises a complementary region that recognizes a target genomic sequence of the ABO gene locus. In certain of these embodiments, the target sequence is located in a CDS, an exon, an intron, a sequence spanning a portion of an exon and a portion of an adjacent intron, or regulatory regions of the ABO gene. In certain embodiments, the gRNA comprises a complementary region that recognizes a target genomic sequence located entirely within an exon of the ABO gene, for example an exon as identified in Ensembl ENSG00000175164.4, ENST00000611156.4, or ENST00000538324.2, or in NCBI NC_000009.11. In certain embodiments, the gRNA comprises a complementary region that recognizes a 15-30 nucleotide target sequence located within nucleotides 1-63, 806-863, 13857-13926, 14651-14707, 16159-16206, 17893-17983, 18483-18616, 19669-26168, or 42416-42747 of SEQ ID NO:1. In certain embodiments, the gRNA comprises a complementary region that recognizes a 15-30 nucleotide target sequence located within nucleotides 824-863, 13857-13926, 14651-14707, 16159-16206, 17893-17928, 18483-18616, or 19669-20849 of SEQ ID NO:1. In certain embodiments, the gRNA comprises a complementary region that recognizes a 15-30 nucleotide target sequence located within nucleotides 810-863, 13857-13926, 14651-14707, 16159-16206, 17893-17928, 18483-18501, 18504-18616, or 19669-20849 of SEQ ID NO:1. In certain embodiments, the gRNA comprises a complementary region that recognizes a 15-30 nucleotide target sequence located within nucleotides 810-863, 13857-13926, 14651-14707, 16159-16206, 17893-17928, 18483-18501, 18504-18616, 19669-20350, or 20355-20423 of SEQ ID NO:1.

Exemplary target genomic sequences, the strand in which they are located, their associated PAM sequences, and cut sites of gRNAs targeting the ABO gene are provided in Table 4. In some embodiments, the gRNA targeting the ABO gene comprises a complementary region comprising, consisting of, or consisting essentially of a nucleotide sequence complementary to a nucleotide sequence set forth in any of SEQ ID NOs: 20-203. In some embodiments, the gRNA targeting the ABO gene comprises a complementary region comprising, consisting of, or consisting essentially of a nucleotide sequence complementary to the reverse complement of any of SEQ ID NOs: 20-203.

In some embodiments, the gRNA used herein for targeted gene editing comprises a complementary region that recognizes a target genomic sequence of the FUT1 gene locus. In certain of these embodiments, the target sequence is located in a CDS, an exon, an intron, a sequence spanning a portion of an exon and a portion of an adjacent intron, or regulatory region of the FUT1 gene. In certain embodiments, the gRNA comprises a complementary region that recognizes a target genomic sequence located entirely within an exon of the FUT1 gene, for example an exon as identified in Ensembl ENSG00000174951.12 or ENST00000645652.2, or in NCBI NG_007510. In certain embodiments, the gRNA comprises a complementary region that recognizes a 15-30 nucleotide target sequence located within nucleotides 1-101, 1731-2066, 2269-2901, or 4108-7380 of SEQ ID NO:2. In certain embodiments, the gRNA comprises a complementary region that recognizes a 15-30 nucleotide target sequence located within nucleotides 2750-2901 or 4108-7380 of SEQ ID NO:2. In certain embodiments, the gRNA comprises a complementary region that recognizes a 15-30 nucleotide target sequence located within nucleotides 33-101, 1731-1743, 1828-2066, 2269-2901, or 4108-7380 of SEQ ID NO:2. In certain embodiments, the gRNA comprises a complementary region that recognizes a 15-30 nucleotide target sequence located within nucleotides 33-101, 1731-1743, 1828-2066, 2269-2499, or 4108-7380 of SEQ ID NO:2. In certain embodiments, the gRNA comprises a complementary region that recognizes a 15-30 nucleotide target sequence located within nucleotides 33-101, 1731-1743, 1828-2066, 2666-2901, or 4108-7380 of SEQ ID NO:2. In certain embodiments, the gRNA comprises a complementary region that recognizes a 15-30 nucleotide target sequence located within nucleotides 33-101, 1731-1743, 1828-2066, 2750-2901, or 4108-7380 of SEQ ID NO:2.

Exemplary target genomic sequences, the strand in which they are located, their associated PAM sequences, and cut sites of gRNAs targeting the FUT1 gene are provided in Table 4. In some embodiments, the gRNA targeting the FUT1 gene comprises a complementary region comprising, consisting of, or consisting essentially of a nucleotide sequence complementary to a nucleotide sequence set forth in any of SEQ ID NOs: 204-420. In some embodiments, the gRNA targeting the FUT1 gene comprises a complementary region comprising, consisting of, or consisting essentially of a nucleotide sequence complementary to the reverse complement of any of SEQ ID NOs: 204-420.

In some embodiments, the gRNA used herein for targeted gene editing comprises a complementary region that recognizes a target genomic sequence of the RHD gene locus. In certain of these embodiments, the target sequence is located in a CDS, an exon, an intron, a sequence spanning a portion of an exon and a portion of an adjacent intron, or regulatory regions of the RHD gene. In certain embodiments, the gRNA comprises a complementary region that recognizes a target genomic sequence located entirely within an exon of the RHD gene, for example an exon as identified in Ensembl ENSG00000187010.21, ENST00000328664.9, ENST00000622561.4, ENST00000423810.6, ENST00000342055.9, ENST00000568195.5, ENST00000357542.8, ENST00000417538.6, ENST00000454452.6, or ENST00000648012.1, or in NCBI NG_007494.1. In certain embodiments, the gRNA comprises a complementary region that recognizes a 15-30 nucleotide target sequence located within nucleotides 1-303, 12181-12367, 18249-18399, 28554-28701, 29128-29294, 30930-31067, 34204-34550, 35255-35424, 44608-44687, 49497-49570, or 56506-58053 of SEQ ID NO:3. In certain embodiments, the gRNA comprises a complementary region that recognizes a 15-30 nucleotide target sequence located within nucleotides 117-303, 12181-12367, 18249-18399, 28554-28701, 29128-29294, 30930-31067, 34204-34337, 44608-44687, 49497-49570, or 56506-58053 of SEQ ID NO:3. In certain embodiments, the gRNA comprises a complementary region that recognizes a 15-30 nucleotide target sequence located within nucleotides 94-303, 12181-12367, 18249-18399, 28554-28701, 29128-29294, 30930-31067, 34204-34337, 35255-35424, 44608-44687, 49497-49570, or 56506-58053 of SEQ ID NO:3. In certain embodiments, the gRNA comprises a complementary region that recognizes a 15-30 nucleotide target sequence located within nucleotides 156-303, 12181-12367, 18249-18399, 28554-28701, 29128-29294, 30930-31067, 34204-34337, 35255-35424, 44608-44687, or 56506-56819 of SEQ ID NO:3. In certain embodiments, the gRNA comprises a complementary region that recognizes a 15-30 nucleotide target sequence located within nucleotides 156-303, 12181-12367, 18249-18399, 28554-28701, 29128-29294, 30930-31067, 34204-34337, 35255-35424, or 56506-56819 of SEQ ID NO:3. In certain embodiments, the gRNA comprises a complementary region that recognizes a 15-30 nucleotide target sequence located within nucleotides 156-303, 12181-12367, 18249-18399, 28554-28701, 29128-29294, 30930-31067, 34204-34337, 44608-44687, or 56506-56819 of SEQ ID NO:3. In certain embodiments, the gRNA comprises a complementary region that recognizes a 15-30 nucleotide target sequence located within nucleotides 156-303, 12181-12367, 18249-18399, 28554-28701, 29128-29294, 30930-31067, 34204-34337, or 56506-56819 of SEQ ID NO:3. In certain embodiments, the gRNA comprises a complementary region that recognizes a 15-30 nucleotide target sequence located within nucleotides 156-303, 12181-12367, 18249-18399, 28554-28701, 29128-29294, 30930-31067, 44608-44687, or 56506-56819 of SEQ ID NO:3. In certain embodiments, the gRNA comprises a complementary region that recognizes a 15-30 nucleotide target sequence located within nucleotides 106-303, 12181-12367, 18249-18399, 28554-28701, 29128-29294, 30930-31067, or 56506-56819 of SEQ ID NO:3. In certain embodiments, the gRNA comprises a complementary region that recognizes a 15-30 nucleotide target sequence located within nucleotides 47-303, 12181-12367, 18249-18399, 28554-28701, 29128-29294, 30930-31067, 44608-44687, 49497-49570, or 56506-56769 of SEQ ID NO:3. In certain embodiments, the gRNA comprises a complementary region that recognizes a 15-30 nucleotide target sequence located within nucleotides 117-303, 12181-12367, 18249-18399, 28554-28701, 29128-29294, 30930-31067, 34204-34337, 44608-44687, 49497-49570, or 56506-58053 of SEQ ID NO:3.

Exemplary target genomic sequences, the strand in which they are located, their associated PAM sequences, and cut sites of gRNAs targeting the RHD gene are provided in Table 4. In some embodiments, the gRNA targeting the RHD gene comprises a complementary region comprising, consisting of, or consisting essentially of a nucleotide sequence complementary to a nucleotide sequence set forth in any of SEQ ID NOs: 421-580. In some embodiments, the gRNA targeting the RHD gene comprises a complementary region comprising, consisting of, or consisting essentially of a nucleotide sequence complementary to the reverse complement of any of SEQ ID NOs: 421-580.

In some embodiments, provided are methods of identifying new loci and/or gRNA sequences for use in the gene editing systems as described. For example, for CRISPR/Cas systems, when an existing gRNA for a particular locus (e.g., any of the exemplary gRNAs provided) is known, an “inch worming” approach can be used to identify additional loci by scanning the flanking regions on either side of the known locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in association with any of the gene editing system described. In certain embodiments, the new gRNAs identified using this approach can target a genomic locus within 4000 bp, within 3500 bp, within 3000 bp, within 2500 bp, within 2000 bp, within 1500 bp, within 1000 bp, within 500 bp, within 400 bp, within 300 bp, within 200 bp, within 100 bp, or within 50 bp of any of the genomic cut sites provided in Table 4. In certain embodiments, the gRNA is configured to produce a cut site at a position within 5, 10, 15, 20, 30, 40, or 50 nucleotides of any of the genomic cut sites provided in Table 4.

In some embodiments, the activity, stability, and/or other characteristics of gRNAs can be altered through the incorporation of chemical and/or sequential modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not being bound by a particular theory, it is believed that certain modified gRNAs described herein can exhibit a reduced innate immune response when introduced into a population of cells, particularly the cells of the present technology. As used herein, the term “innate immune response” includes a cellular response to exogenous nucleic acids, including single stranded nucleic acids, generally of viral or bacterial origin, which involves the induction of cytokine expression and release, particularly the interferons, and cell death. Other common chemical modifications of gRNAs to improve stabilities, increase nuclease resistance, and/or reduce immune response include 2′-O-methyl modification, 2′-fluoro modification, 2′-O-methyl phosphorothioate linkage modification, and 2′-O-methyl 3′ thioPACE modification.

One common 3′ end modification is the addition of a poly A tract comprising one or more (and typically 5-200) adenine (A) residues. The poly A tract can be contained in the nucleic acid sequence encoding the gRNA or can be added to the gRNA during chemical synthesis, or following in vitro transcription using a polyadenosine polymerase (e.g., E. coli Poly(A)Polymerase). In vivo, poly-A tracts can be added to sequences transcribed from DNA vectors through the use of polyadenylation signals. Other suitable gRNA modifications include, without limitations, those described in U.S. Patent Application No. US 2017/0073674 A1 and International Publication No. WO 2017/165862 A1, the entire contents of each of which are incorporated by reference herein.

D. Delivery of Gene Editing Systems into a Cell

In some embodiments, provided are compositions comprising one or more components of a gene editing system described herein, including one or more gRNAs, a site-directed nuclease (e.g., a Cas nuclease) or a nucleotide sequence encoding a site-directed nuclease protein, and optionally a transgene for targeted insertion. In some embodiments, the compositions are formulated for delivery into a cell.

In some embodiments, components of a gene editing system provided herein, including one or more gRNAs, a site-directed nuclease (e.g., a Cas nuclease) or a nucleotide sequence encoding a site-directed nuclease protein, and optionally a transgene for targeted insertion, may be delivered into a cell in the form of a vector. The delivery vector can be any type of vector suitable for introduction of nucleotide sequences into a cell, including, for example, plasmids, adenoviral vectors, adeno-associated viral (AAV) vectors, retroviral vectors, lentiviral vectors, phages, and HDR-based donor vectors. The different components may be introduced into a cell together or separately, and may be delivered in a single vector or multiple vectors.

In some embodiments, the vector may be introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods.

In some embodiments, the present technology provides compositions comprising a vector according to various embodiments disclosed herein. In some embodiments, the compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising cells as disclosed herein further comprise a suitable infusion media.

In some embodiments, provided are cells or compositions thereof comprising one or more components of a gene editing system described herein, including one or more gRNAs, a site-directed nuclease (e.g., a Cas nuclease) or a nucleotide sequence encoding a site-directed nuclease protein, and optionally a transgene for targeted insertion.

### E. Additional Genetic Modifications for Hypoimmunity

In some embodiments, in addition to the gene editing methods as described to knock out, knock down, or otherwise alter the expression of one or more genes associated with blood type (e.g., ABO, FUT1, and RHD) in a cell, the cell, for example, in cases of allogeneic cells, may have additional genetic modifications, to further reduce potential graft-versus-host risks after infusion into the recipient or risks of being eliminated by the recipient's innate immune system. These additional modifications may include, for example, reducing or eliminating the expression of major histocompatibility complex (MHC) class I and/or MHC class II (MHC I and/or MHC II) genes, which encode cell surface molecules specialized to present antigenic peptides to immune cells. Reduced expression of MHC I and/or MHC II molecules in allogeneic cells may prevent recognition of these cells by the immune cells of the recipient and thus rejection of the graft. The step of modifying (e.g., reducing or eliminating) MHC I and/or MHC II molecules may occur before, at the same time as, or after the step of modifying the expression of one or more blood type genes. The MHC in humans is called human leukocyte antigen (HLA). Class I HLA (HLA I) corresponding to MHC I include the HLA-A, HLA-B, and HLA-C genes, and Class II HLA (HLA I) corresponding to MHC II include the HLA-DR, HLA-DQ, HLA-DP, HLA-DM, and HLA-DO genes.

In some embodiments, the additional modifications to a cell to reduce the immunogenicity of the cell comprise genetically modifying the cell to reduce the expression of one or more immune factors, including, for example, class II transactivator (CIITA), P2 microglobulin (B2M), NLRC5, CTLA-4, PD-1, HLA-A, HLA-BM, HLA-C, RFX-ANK, NFY-A, RFX5, RFX-AP, NFY-B, NFY-C, IRF1, MIC-A, MIC-B, TXNIP, CD142, CD38, PCDH11Y, NLGN4Y, and TAP1.

In some embodiments, the cell may be modified to have reduced expression of MHC I genes by targeting and modulating one or more of the HLA loci individually, such as HLA-A, HLA-B, and/or HLA-C, or collectively with HLA-Razor. In some embodiments, the modulation occurs through insertion-deletion (indel) modifications of one of more of the HLA loci, including HLA-A, HLA-B, and/or HLA-C, for example, by using the CRISPR/Cas system as described. By modulating (e.g., reducing or deleting) expression of any of the HLA genes, the cell can be rendered hypoimmunogenic and have a reduced ability to induce an immune response in a recipient subject. In some embodiments, reduced expression of any of the HLA loci reduces or eliminates expression of one or more of the HLA-A, HLA-B, and HLA-C genes. In some embodiments, the cell has HLA-A, HLA-B, and/or HLA-C knockout. In some embodiments, the genetic modification targeting any of the HLA loci comprises inserting an exogenous nucleic acid or transgene encoding a polypeptide (e.g., a tolerogenic factor) as described herein at the HLA locus. In certain of these embodiments, insertion of the transgene into any of the HLA loci results in HLA-A, HLA-B, and/or HLA-C knockout.

In some embodiments, the cell may be modified to have reduced expression of MHC I genes by targeting and modulating the B2M locus. The B2M gene encodes a component of MHC I molecules. In some embodiments, the modulation occurs through insertion-deletion (indel) modifications or targeted mutations of the B2M locus, for example, by using the CRISPR/Cas system as described. By modulating (e.g., reducing or deleting) expression of B2M, surface trafficking of MHC I molecules is blocked, and the cell is thus rendered hypoimmunogenic. In some embodiments, the allogeneic cell modified to have reduced expression of MHC I genes has a reduced ability to induce an immune response in a recipient subject. In some embodiments, reduced expression of B2M reduces or eliminates expression of one or more of the HLA-A, HLA-B, and HLA-C genes. In some embodiments, the cell has B2M knockout. In some embodiments, the genetic modification targeting the B2M locus comprises inserting an exogenous nucleic acid or transgene encoding a polypeptide (e.g., a tolerogenic factor) as described herein at the B2M locus. In certain of these embodiments, insertion of the transgene into the B2M locus results in B2M knockout.

In some embodiments, the cell may be modified to have reduced expression of MHC I genes by targeting and modulating the TAP1 locus. TAP1 encoded by the TAP1 gene assembles with TAP2 encoded by the TAP2 gene to form the transporter associated with antigen processing (TAP) complex, which is found in the endoplasmic reticulum (ER) and transports peptides of foreign origin into the ER to be attached to MHC class I proteins for presentation on the cell surface to the immune system. In some embodiments, the modulation occurs through insertion-deletion (indel) modifications of the TAP1 locus, for example, by using the CRISPR/Cas system as described. By modulating (e.g., reducing or deleting) expression of TAP1, surface trafficking of MHC I molecules is blocked, and the cell is thus rendered hypoimmunogenic. In some embodiments, reduced expression of TAP1 reduces or eliminates expression of one or more of the HLA-A, HLA-B, and HLA-C genes. In some embodiments, the cell has TAP1 knockout. In some embodiments, the genetic modification targeting the TAP1 locus comprises inserting an exogenous nucleic acid or transgene encoding a polypeptide (e.g., a tolerogenic factor) as disclosed herein at the TAP1 locus. In certain of these embodiments, insertion of the transgene into the TAP1 locus results in TAP1 knockout.

In some embodiments, the cell may be modified to have reduced expression of MHC II genes by overexpression of CD74.

In some embodiments, the cell may be modified to have reduced expression of MHC II genes by targeting and modulating the CIITA locus. CIITA is a member of the nucleotide binding domain (NBD) leucine-rich repeat (LRR) family of proteins and regulates the transcription of MHC II by associating with the MHC enhanceosome. In some embodiments, the modulation occurs through insertion-deletion (indel) modifications of the CIITA locus, for example, by using the CRISPR/Cas system as described. In some embodiments, reduced expression of CIITA reduces or eliminates expression of one or more of the HLA-DR, HLA-DQ, HLA-DP, HLA-DM, and HLA-DO genes. In some embodiments, the cell has CIITA knockout. In some embodiments, the genetic modification targeting the CIITA locus comprises inserting an exogenous nucleic acid or transgene encoding a polypeptide (e.g., a tolerogenic factor) as disclosed herein at the CIITA locus. In certain of these embodiments, insertion of the transgene into the CIITA locus results in CIITA knockout.

In certain embodiments, the cell comprises a modification, such as a genetic modification, targeting the MIC-A gene. MIC-A is a protein having known isoforms and variants (see, e.g., UniProt Q29983, accessed Jul. 18, 2022); all such forms of MIC-A are encompassed by the disclosure provided herein. In some embodiments, the genetic modification targeting the MIC-A gene is by using a targeted nuclease system that comprises a Cas protein or a polynucleotide encoding a Cas protein, and at least one guide ribonucleic acid sequence for specifically targeting the MIC-A gene. In some embodiments, the genetic modification occurs using a CRISPR/Cas system as described. For example, in some embodiments, a gRNA with a targeting sequence GATGACCCTGGCTCATATCA (SEQ ID NO:581) can be used. In some embodiments, methods of gene editing with a CRISPR/Cas system and gRNA targeting MIC-A, such as with a targeting sequence GATGACCCTGGCTCATATCA (SEQ ID NO:581), knocks out all alleles of MIC-A in a cell. In some embodiments, the cell has MIC-A knockout. In some embodiments, the genetic modification targeting the MIC-A locus comprises inserting an exogenous nucleic acid or transgene encoding a polypeptide (e.g., a tolerogenic factor) as disclosed herein at the MIC-A locus. In certain of these embodiments, insertion of the transgene into the MIC-A locus results in MIC-A knockout.

In certain embodiments, the cell comprises a modification, such as a genetic modification, targeting the MIC-B gene. MIC-B is a protein having known isoforms and variants (see, e.g., UniProt Q29980, accessed Jul. 18, 2022); all such forms of MIC-B are encompassed by the disclosure provided herein. In some embodiments, the genetic modification targeting the MIC-B gene is by using a targeted nuclease system that comprises a Cas protein or a polynucleotide encoding a Cas protein, and at least one guide ribonucleic acid sequence for specifically targeting the MIC-B gene. In some embodiments, the genetic modification occurs using a CRISPR/Cas system as described. For example, in some embodiments, a gRNA with a targeting sequence GTTTCTGCCTGTCATAGCGC (SEQ ID NO:582) can be used. In some embodiments, methods of gene editing with a CRISPR/Cas system and gRNA targeting MIC-B, such as with a targeting sequence GTTTCTGCCTGTCATAGCGC (SEQ ID NO:582) knocks out all alleles of MIC-B in a cell. In some embodiments, the cell has MIC-B knockout. In some embodiments, the genetic modification targeting the MIC-B locus comprises inserting an exogenous nucleic acid or transgene encoding a polypeptide (e.g., a tolerogenic factor) as disclosed herein at the MIC-B locus. In certain of these embodiments, insertion of the transgene into the MIC-B locus results in MIC-B knockout.

In some embodiments, the cell has genetic modifications at the B2M, TAP1, CIITA, MIC-A, and/or MIC-B loci, have B2M, TAP1, CIITA, MIC-A, and/or MIC-B knockout, or have CD74 overexpression. The B2M, TAP1, CIITA, MIC-A, and/or MIC-B knockout can occur at one allele, or both alleles, of the respective gene locus. In some embodiments, the B2M, TAP1, CIITA, MIC-A, and/or MIC-B loci are modified so that the cell has reduced or no expression of B2M, TAP1, CIITA, MIC-A, and/or MIC-B, respectively. In these embodiments, the cell has reduced expression of MHC I and/or MHC II genes (HLA I and/or HLA II in humans) as a result of B2M, TAP1, CIITA, MIC-A, and/or MIC-B deletion or knockout, or overexpression of CD74.

In some embodiments, the transgene for targeted insertion (i.e., knock-in) at a genomic locus for genetic modification as described (e.g., the ABO, FUT1, RHD, B2M, TAP1, CIITA, MIC-A, and/or MIC-B loci) may encode a tolerogenic factor that can improve the hypoimmunogenicity of the resulting cells so that they will not be subject to immune rejection when transplanted into a recipient and thus increasing the effectiveness of cell-based therapies. Examples of a tolerogenic factor include, but are not limited to, A20/TNFAIP3, CD16, CD16 Fc receptor, CD24, CD35, CD39, CD46, CD47, CD52, CD55, CD59, CD200, CCL22, CTLA4-Ig, C1 inhibitor, complement receptor (CR1), DUX4, FASL, H2-M3, IDO1, IL15-RF, HLA-C, HLA-E, HLA-E heavy chain, HLA-G, IL-10, IL-35, MANF, PD-1, PD-L1, SERPINB9, CCL21, MFGE8, and truncations, modifications, or fusions of any of the above.

In some embodiments, the tolerogenic factor is CD47, which is a leukocyte surface antigen and has a role in cell adhesion and modulation of integrins. It is expressed on the surface of a cell (e.g., a T cell) and signals to circulating macrophages not to phagocytize the cell. Overexpression of CD47 thus can reduce the immunogenicity of the cell when grafted and improve immune protection in allogeneic recipients.

CD47 is a transmembrane protein that, in humans, is encoded by the CD47 gene. It is a member of the immunoglobulin (Ig) superfamily. CD47 has a molecular weight of about −50 kDa. It is glycosylated and ubiquitously expressed by virtually all cells in the human body. It has a single IgV-like domain at its N-terminus, a highly hydrophobic stretch with five membrane-spanning segments, and an alternatively spliced cytoplasmic tail at its C-terminus. In addition, it has two extracellular regions and two intracellular regions between neighboring membrane-spanning segments. A signal peptide, when it exists on a CD47 isoform, is located at the N-terminus of the IgV-like domain.

CD47 is involved in a range of cellular processes, including apoptosis, proliferation, adhesion, and migration. CD47 interacts with multiple extracellular ligands, such as TSP-1, integrins, other CD47 proteins, and SIRPa. The CD47/SIRPa interaction regulates a multitude of intercellular interactions in many body systems, such as the immune system where it regulates lymphocyte homeostasis, dendritic cell (DC) maturation and activation, proper localization of certain DC subsets in secondary lymphoid organs, and cellular transmigration. CD47 on cells, including on donor cells in the context of transplantation or cell therapy applications, can function as a “marker of self” and regulate phagocytosis by binding to SIRPα on the surface of circulating immune cells to deliver an inhibitory “don't kill me” signal. CD47-SIRPα binding results in phosphorylation of immunoreceptor tyrosine-based inhibition motifs (ITIMs) on SIRPα, which triggers recruitment of the SHP1 and SHP2 Src homology phosphatases. These phosphatases, in turn, inhibit accumulation of myosin II at the phagocytic synapse, preventing phagocytosis (Fujioka et al., Mol. Cell. Biol., 16:6887-6899 (1996)). Phagocytosis of target cells by macrophages is ultimately regulated by a balance of activating signals (e.g., FcγR, CRT, LRP-1) and inhibitory signals (e.g., SIRPα-CD47). Elevated expression of CD47 can help the cell evade immune surveillance, subsequent destruction, and innate immune cell killing. Thus, CD47 can be used as a tolerogenic factor to induce immune tolerance, for example, when there is pathological or undesirable activation of an otherwise normal immune response. This can occur, for example, when a patient develops an immune reaction to donor antigens after receiving an allogeneic transplantation or an allogeneic cell therapy, or when the body responds inappropriately to self-antigens implicated in autoimmune diseases.

The human CD47 gene has six naturally occurring transcripts, five of which each encode a protein isoform of CD47 (Ensembl, Gene: CD47, ENSG00000196776). The six transcripts are named CD47-201, CD47-202, CD47-203, CD47-204, CD47-205, and CD47-206. The coding DNA sequence (CDS) of the six transcripts are as set forth in SEQ ID NOs: 589-594, respectively. The amino acid sequences of the five protein isoforms are as set forth in SEQ ID NOs: 583-588 respectively (see Table 3).

Transcript CD47-201 (SEQ ID NO:589) encodes isoform CD47-201 (SEQ ID NO:583), which has 305 amino acids. Isoform CD47-201 has a C-terminal truncation of 18 amino acids from isoform CD47-202. All splice junctions of the CD47-201 transcript are supported by at least one non-suspect mRNA.

Transcript CD47-202 (SEQ ID NO:590) encodes isoform CD47-202 (SEQ ID NO:584), which has 323 amino acids. CD47-202 is the longest transcript of the human CD47 gene. It is designated as the representative transcript in the Ensembl database. In identifying the representative transcript, Ensembl aims to identity the transcript that, on balance, has the highest coverage of conserved exons, highest expression, longest coding sequence and is represented in other key resources, such as NCBI and UniProt. All splice junctions of the CD47-202 transcript are supported by at least one non-suspect mRNA. Amino acids 1-18 are the signal peptide. The amino acid sequence of CD47-202 without the signal peptide is set forth in SEQ ID NO:585.

Transcript CD47-203 (SEQ ID NO:591) encodes isoform CD47-203 (SEQ ID NO:586), which has 86 amino acids. The only support for the transcript model is from a single expressed sequence tag (EST).

Transcript CD47-204 (SEQ ID NO:592) does not encode any protein. All splice junctions of this transcript are supported by at least one non-suspect mRNA

Transcript CD47-205 (SEQ ID NO:593) encodes isoform CD47-205 (SEQ ID NO:587), which has 109 amino acids. Isoform 205 comprises 3 transmembrane domains and a truncated intracellular domain from isoform CD47-202. The best supporting mRNA for the transcript model is flagged as suspect or the support is from multiple ESTs.

Transcript CD47-206 (SEQ ID NO:594) encodes isoform CD47-206 (SEQ ID NO:588), which has 183 amino acids. Isoform 206 comprises a truncated extracellular domain and 5 transmembrane domains from isoform CD47-202.

In some embodiments, the transgene for targeted insertion (i.e., knock-in) at a genomic locus for genetic modification as described (e.g., the B2M, TAP1, CIITA, MIC-A, and/or MIC-B loci) may encode CD47, for example, human CD47. In certain of these embodiments, the human CD47 comprises or consists of an amino acid sequence set forth in any one of SEQ ID NOs: 583-588, or is at least 80% identical (e.g., at least 80%, at least 85%, at least 90%, at least 95%, at least 96%, at least 97%, at least 98%, at least 99%, or 100% identical) to the amino acid sequence set forth in any one of SEQ ID NOs: 583-588. In some embodiments, the human CD47 comprises or consists of an amino acid sequence set forth in SEQ ID NO:584 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:584. In some embodiments, the nucleotide sequence encoding CD47 corresponds to an mRNA sequence of human CD47. In some embodiments, the nucleotide sequence encoding CD47 is at least 80% identical (e.g., at least 80%, at least 85%, at least 90%, at least 95%, at least 96%, at least 97%, at least 98%, at least 99%, or 100% identical) to the nucleotide sequence set forth in any one of SEQ ID NOs: 589-594. In some embodiments, the nucleotide sequence encoding CD47 is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:590.

In some embodiments, the nucleotide sequence encoding CD47 is codon-optimized for expression in a mammalian cell, for example, a human cell. In some embodiments, the codon-optimized nucleotide sequence encoding CD47 is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:595.

TABLE 3
Exemplary sequences of CD47

| SEQ ID NO: | Sequence | Description |
|---|---|---|
| 583 | MWPLVAALLLGSACCGSAQLLFNKTKSVEFTFCNDTV | CD47-201 |
| | VIPCFVTNMEAQNTTEVYVKWKFKGRDIYTFDGALNK | amino acid |
| | STVPTDFSSAKIEVSQLLKGDASLKMDKSDAVSHTGN | sequence |
| | YTCEVTELTREGETIIELKYRVVSWFSPNENILIVIFPIFA | |
| | ILLFWGQFGIKTLKYRSGGMDEKTIALLVAGLVITVIVIV | |
| | GAILFVPGEYSLKNATGLGLIVTSTGILILLHYYVFSTAI | |
| | GLTSFVIAILVIQVIAYILAVVGLSLCIAACIPMHGPLLISG | |
| | LSILALAQLLGLVYMKFVASNQKTIQPPRNN | |
| 584 | MWPLVAALLLGSACCGSAQLLFNKTKSVEFTFCNDTV | CD47-202 |
| | VIPCFVTNMEAQNTTEVYVKWKFKGRDIYTFDGALNK | amino acid |
| | STVPTDFSSAKIEVSQLLKGDASLKMDKSDAVSHTGN | sequence |
| | YTCEVTELTREGETIIELKYRVVSWFSPNENILIVIFPIFA | |
| | ILLFWGQFGIKTLKYRSGGMDEKTIALLVAGLVITVIVIV | |
| | GAILFVPGEYSLKNATGLGLIVTSTGILILLHYYVFSTAI | |
| | GLTSFVIAILVIQVIAYILAVVGLSLCIAACIPMHGPLLISG | |
| | LSILALAQLLGLVYMKFVASNQKTIQPPRKAVEEPLNA | |
| | FKESKGMMNDE | |
| 585 | QLLFNKTKSVEFTFCNDTVVIPCFVTNMEAQNTTEVYV | CD47-202 |
| | KWKFKGRDIYTFDGALNKSTVPTDFSSAKIEVSQLLKG | amino acid |
| | DASLKMDKSDAVSHTGNYTCEVTELTREGETIIELKYR | sequence |
| | VVSWFSPNENILIVIFPIFAILLFWGQFGIKTLKYRSGG | without |
| | MDEKTIALLVAGLVITVIVIVGAILFVPGEYSLKNATGLG | signal |
| | LIVTSTGILILLHYYVFSTAIGLTSFVIAILVIQVIAYILAVV | peptide |
| | GLSLCIAACIPMHGPLLISGLSILALAQLLGLVYMKFVA | |
| | SNQKTIQPPRKAVEEPLNAFKESKGMMNDE | |
| 586 | XWTESLYCGVYTNAWPSSDFRFEYLSSSTITWTSLYE | CD47-203 |
| | ICGVQRDTYSQLKGKKRQASNQKTIQPPRKAVEEPLN | amino acid |
| | AFKESKGMMNDE | sequence |
| 587 | XKNATGLGLIVTSTGILILLHYYVFSTAIGLTSFVIAILVIQ | CD47-205 |
| | VIAYILAVVGLSLCIAACIPMHGPLLISGLSILALAQLLGL | amino acid |
| | VYMKFVASNQKTIQPPRKAVEEPLNE | sequence |
| 588 | MEAQNTTEVYVKWKFKGRDIYTFDGALNKSTVPTDFS | CD47-206 |
| | SAKIEVSQLLKGDASLKMDKSDAVSHTGNYTCEVTEL | amino acid |
| | TREGETIIELKYRVVSWFSPNENILIVIFPIFAILLFWGQF | sequence |
| | GIKTLKYRSGGMDEKTIALLVAGLVITVIVIVGAILFVPG | |
| | EYSLKNATGLGLIVTSTGILILLHYYVF | |
| 589 | atgtggcccctggtagcggcgctgttgctgggctcggcgtgctgcggatcag | CD47-201 |
| | ctcagctactatttaataaaacaaaatctgtagaattcacgttttgtaatgaca | CDS |
| | ctgtcgtcattccatgctttgttactaatatggaggcacaaaacactactgaag | nucleotide |
| | tatacgtaaagtggaaatttaaaggaagagatatttacacctttgatggagct | sequence |
| | ctaaacaagtccactgtccccactgactttagtagtgcaaaaattgaagtctc | |
| | acaattactaaaaggagatgcctctttgaagatggataagagtgatgctgtct | |
| | cacacacaggaaactacacttgtgaagtaacagaattaaccagagaagg | |
| | tgaaacgatcatcgagctaaaatatcgtgttgtttcatggttttctccaaatgaa | |
| | aatattcttattgttattttcccaatttttgctatactcctgttctggggacagtttggt | |
| | attaaaacacttaaatatagatccggtggtatggatgagaaaacaattgcttt | |
| | acttgttgctggactagtgatcactgtcattgtcattgttggagccattcttttcgtc | |
| | ccaggtgaatattcattaaagaatgctactggccttggtttaattgtgacttcta | |
| | cagggatattaatattacttcactactatgtgtttagtacagcgattggattaac | |
| | ctccttcgtcattgccatattggttattcaggtgatagcctatatcctcgctgtggt | |
| | tggactgagtctctgtattgcggcgtgtataccaatgcatggccctcttctgattt | |
| | caggtttgagtatcttagctctagcacaattacttggactagtttatatgaaattt | |
| | gtggcttccaatcagaagactatacaacctcctaggaataactga | |
| 590 | atgtggcccctggtagcggcgctgttgctgggctcggcgtgctgcggatcag | CD47-202 |
| | ctcagctactatttaataaaacaaaatctgtagaattcacgttttgtaatgaca | CDS |
| | ctgtcgtcattccatgctttgttactaatatggaggcacaaaacactactgaag | nucleotide |
| | tatacgtaaagtggaaatttaaaggaagagatatttacacctttgatggagct | sequence |
| | ctaaacaagtccactgtccccactgactttagtagtgcaaaaattgaagtctc | |
| | acaattactaaaaggagatgcctctttgaagatggataagagtgatgctgtct | |
| | cacacacaggaaactacacttgtgaagtaacagaattaaccagagaagg | |
| | tgaaacgatcatcgagctaaaatatcgtgttgtttcatggttttctccaaatgaa | |
| | aatattcttattgttattttcccaatttttgctatactcctgttctggggacagtttggt | |
| | attaaaacacttaaatatagatccggtggtatggatgagaaaacaattgcttt | |
| | acttgttgctggactagtgatcactgtcattgtcattgttggagccattcttttcgtc | |
| | ccaggtgaatattcattaaagaatgctactggccttggtttaattgtgacttcta | |
| | cagggatattaatattacttcactactatgtgtttagtacagcgattggattaac | |
| | ctccttcgtcattgccatattggttattcaggtgatagcctatatcctcgctgtggt | |
| | tggactgagtctctgtattgcggcgtgtataccaatgcatggccctcttctgattt | |
| | caggtttgagtatcttagctctagcacaattacttggactagtttatatgaaattt | |
| | gtggcttccaatcagaagactatacaacctcctaggaaagctgtagaggaa | |
| | ccccttaatgcattcaaagaatcaaaaggaatgatgaatgatgaataa | |
| 591 | tggactgagtctctgtattgcggcgtgtataccaatgcatggccctcttctgattt | CD47-203 |
| | caggtttgagtatcttagctctagcacaattacttggactagtttatatgaaattt | CDS |
| | gtggggttcagagggacacctacagtcagttgaaaggcaagaagagaca | nucleotide |
| | agcttccaatcagaagactatacaacctcctaggaaagctgtagaggaac | sequence |
| | cccttaatgcattcaaagaatcaaaaggaatgatgaatgatgaataa | |
| 592 | agatattttcttgttcaatttaaggagaggtaaatttggtatcaatagaaaaaat | CD47-204 |
| | gtttctgaaaaatttaaaccctggaaatgtatttatggcatggagtcagatgttt | CDS |
| | cagggagagaagaacaaatcaagaagcattgcaagtatgctcatatgga | nucleotide |
| | atgcttaaggcttgtggttaaaaaatatatatatatggctgtcaatgtcttaggct | sequence |
| | catggtagcagcagaaatcgtaataattcttttgtcacatgggttatatccatat | |
| | tggagagaattaactcaggtgaaattaacttgtacactgtttggttttataatatt | |
| | tagagggatcacaactgactgatgtccctttgaagtaccattcttcataaatctt | |
| | tttttttcagaatgggccagccaactgtgacatcccttggatcggagatttaga | |
| | actagaaagtattctttctacattattagggaagaaaaggagttacttggcggt | |
| | tagcaatattctattttgttttgttttgtttttagagacagggtctcattatgttgacca | |
| | ggctggcctcgagctcctgggctcaagcaatgctcccacctcagcctccca | |
| | agtagctgggactacaggcatgtgccactacacctggcagtgtttattctgat | |
| | aaatacatttatgagctcaaaaatgtaactctaaaaccttatctctgaacttcc | |
| | atattaccatcagaaatttagatagttgtttagttctctttttctttgtagaacatag | |
| | atataaggcatggtttcattgaagtcagttgtatatacatgtaactatcctgatgt | |
| | tcccaaataaagctctgtatttctgcttagtttattggggaggctgctaaatgta | |
| | gtgcatcccaacccattttaccctgttctactttaaaaagaggttggcttcttgttt | |
| | ggatacaaggaccaagtcactcccccaggttcctccacagtaagggaggc | |
| | ctatttaaagccgcccatggcactaacagaaactggactcctatgagctca | |
| | gatacataactgggcctcacagggggggacagtatgtagtctaggaattg | |
| | gaaggatccattccatatcaaagaactgaagcatcgtgttgccctctcagca | |
| | gcaagagtaaggtgatgcccctgtcagttatagttcctgagttcctctgtctttg | |
| | attctttgcctattagccagctagctcaccctcttgtttatgccactgttttttatcct | |
| | attcatgccttctcacagacaacttttcttacctacagctttggactcatccttgtc | |
| | tcctttctgtttctttttcactttcccttcccatcaccaactttctgggtttttttctgtttct | |
| | tcttagagtccagtggcagggagaaacttgtcagtccagtctgttgccatttttc | |
| | ctgtttgagaaagactcaccagcttttggctggctcacagattggctttccttgg | |
| | gtcaggacccacccttttccctgccagctttggaagcttgacagaattcgagt | |
| | gtgcagtggtggtaaataaatagtaaggaacacagagcagtcctggaggc | |
| | gtgcctccatctgctgatgagaaaatccagtgctgtcatccagcccaggtcc | |
| | cagcggaatgggcctctctgttcagtaggatccccctcctgctgagtggttcat | |
| | ggcatgtttctgttcaacgcttttccatctgtaggattcttattctgtatttatttgttttt | |
| | ttgggtttttttattttttgagatggagtctcgctctgtcgcccaggctggagtgca | |
| | gtggcacgaccccagctcgctgcagcctctgcctcccaggacgagggag | |
| | atcctcccacctcagccttccacgtagctgggactacaggcatgcaccaca | |
| | ggcatgcaccaccacgccagctaatttttgtatttttggtagagacagggttgc | |
| | atcatgttgcccaggctggtcttgaatgcctgagctcaagcaatctatttgcctt | |
| | ggcctcccaaagtgctgggattacaggcatgagccaccacggccagcctt | |
| | ctcatttgttttttttataaggaagctatctcttcttccctccccaactagggtattct | |
| | ttttccctttcgtcactttgctcatgtactgtattccttcaacttcattaatgaatccat | |
| | ttggaagcagtgaaaaaggcaactcagaaagctaagaagaaatagata | |
| | gaggaatactcagagctatctgagtattttctttagtttgttagctctttggagcttt | |
| | gaaactggaaagacccagggagtgatgtggagaaagagactgagcttgt | |
| | aagacacaggagcagtgagctaagggagatggagtagtggggacaaat | |
| | tctggcacattctgtctacactctgggtagatagaggagggaggatggagc | |
| | acccatggtgggggtatgttggtgacagcattttcccaccagccagtgtaac | |
| | aagtggctgatttgggggaaagatggcataaacaaatgagagaatgtgttt | |
| | actatttgatgtagatgggttatttgcttcatttttcaaatcagtgtatataatcaag | |
| | aatattcagcatgtttgaatagactgtcagagctggaactctttcattaacatct | |
| | ctggcacctttagttttagccctgaacattttatcttaaaattaaacattaccaaa | |
| | tgccttagtttatttcatttattaaatttatattcttatttgttatttatatcagcttccaat | |
| | cagaagactatacaacctcctaggaataactgaagtgaagtgatggactcc | |
| | gatttggagagtagtaagacgtgaaaggaatacacttgtgtttaagcaccat | |
| | ggccttgatgattcactgttggggagaagaaacaagaaaagtaactggttgt | |
| | cacctatgagacccttacgtgattgttagttaagtttttattcaaagcagctgta | |
| | atttagttaataaaataattatgatctatgttgtttgcccaattgagatccagtttttt | |
| | gttgttatttttaatcaattaggggcaatagtagaatggacaatttccaagaat | |
| | gatgcctttcaggtcctagggcctctggcctctaggtaaccagtttaaattggtt | |
| | cagggtgataactacttagcactgccctggtgattacccagagatatctatga | |
| | aaaccagtggcttccatcaaacctttgccaactcaggttcacagcagctttgg | |
| | gcagttatggcagtatggcattagctgagaggtgtctgccacttctgggtcaa | |
| | tggaataataaattaagtacaggcaggaatttggttgggagcatcttgtatga | |
| | tctccgtatgatgtgatattgatggagatagtggtcctcattcttgggggttgcc | |
| | attcccacattcccccttcaacaaacagtgtaacaggtccttcccagatttag | |
| | ggtacttttattgatggatatgttttccttttattcacataaccccttgaaaccctgt | |
| | cttgtcctcctgttacttgcttctgctgtacaagatgtagcaccttttctcctctttga | |
| | acatggtctagtgacacggtagcaccagttgcaggaaggagccagacttgt | |
| | tctcagagcactgtgttcacacttttcagcaaaaatagctatggttgtaacatat | |
| | gtattcccttcctctgatttgaaggcaaaaatctacagtgtttcttcacttcttttct | |
| | gatctggggcatgaaaaaagcaagattgaaatttgaactatgagtctcctgc | |
| | atggcaacaaaatgtgtgtcaccatcaggccaacaggccagcccttgaat | |
| | ggggatttattactgttgtatctatgttgcatgataaacattcatcaccttcctcct | |
| | gtagtcctgcctcgtactccccttcccctatgattgaaaagtaaacaaaaccc | |
| | acatttcctatcctggttagaagaaaattaatgttctgacagttgtgatcgcctg | |
| | gagtacttttagacttttagcattcgttttttacctgtttgtggatgtgtgtttgtatgtg | |
| | catacgtatgagataggcacatgcatcttctgtatggacaaaggtggggtac | |
| | ctacaggagagcaaaggttaattttgtgcttttagtaaaaacatttaaatacaa | |
| | agttctttattgggtggaattatatttgatgcaaatatttgatcacttaaaactttta | |
| | aaacttctaggtaatttgccacgctttttgactgctcaccaataccctgtaaaa | |
| | atacgtaattcttcctgtttgtgtaataagatattcatatttgtagttgcattaataat | |
| | agttatttcttagtccatcagatgttcccgtgtgcctcttttatgccaaattgattgt | |
| | catatttcatgttgggaccaagtagtttgcccatggcaaacctaaatttatgac | |
| | ctgctgaggcctctcagaaaactgagcatactagcaagacagctcttcttga | |
| | aaaaaaaaatatgtatacacaaatatatacgtatatctatatatacgtatgtat | |
| | atacacacatgtatattcttccttgattgtgtagctgtccaaaataataacatat | |
| | atagagggagctgtattcctttatacaaatctgatggctcctgcagcactttttc | |
| | cttctgaaaatatttacattttgctaacctagtttgttactttaaaaatcagttttgat | |
| | gaaaggagggaaaagcagatggacttgaaaaagatccaagctcctatta | |
| | gaaaaggtatgaaaatctttatagtaaaattttttataaactaaagttgtaccttt | |
| | taatatgtagtaaactctcatttatttggggttcgctcttggatctcatccatccatt | |
| | gtgttctctttaatgctgcctgccttttgaggcattcactgccctagacaatgcca | |
| | ccagagatagtgggggaaatgccagatgaaaccaactcttgctctcactag | |
| | ttgtcagcttctctggataagtgaccacagaagcaggagtcctcctgcttggg | |
| | catcattgggccagttccttctctttaaatcagatttgtaatggctcccaaattcc | |
| | atcacatcacatttaaattgcagacagtgttttgcacatcatgtatctgttttgtcc | |
| | cataatatgctttttactccctgatcccagtttctgctgttgactcttccattcagtttt | |
| | atttattgtgtgttctcacagtgacaccatttgtccttttctgcaacaacctttcca | |
| | gctacttttgccaaattctatttgtcttctccttcaaaacattctcctttgcagttcct | |
| | cttcatctgtgtagctgctcttttgtctcttaacttaccattcctatagtactttatgc | |
| | atctctgcttagttctattagttttttggccttgctcttctccttgattttaaaattccttc | |
| | tatagctagagcttttctttctttcattctctcttcctgcagtgttttgcatacatcag | |
| | aagctaggtacataagttaaatgattgagagttggctgtatttagatttatcact | |
| | ttttaatagggtgagcttgagagttttctttctttctgttttttttttttgtttttttttttttttt | |
| | ttttttttttttttttgactaatttcacatgctctaaaaaccttcaaaggtgattatttttctc | |
| | ctggaaactccaggtccattctgtttaaatccctaagaatgtcagaattaaaat | |
| | aacagggctatcccgtaattggaaatatttcttttttcaggatgctatagtcaatt | |
| | tagtaagtgaccaccaaattgttatttgcactaacaaagctcaaaacacgat | |
| | aagtttactcctccatctcagtaataaaaattaagctgtaatcaaccttctaggt | |
| | ttctcttgtcttaaaatgggtattcaaaaatggggatctgtggtgtatgtatgga | |
| | aacacatactccttaatttacctgttgttggaaactggagaaatgattgtcggg | |
| | caaccgtttattttttattgtattttatttggttgagggatttttttataaacagttttact | |
| | tgtgtcatattttaaaattactaactgccatcacctgctggggtcctttgttaggtc | |
| | attttcagtgactaatagggataatccaggtaactttgaagagatgagcagtg | |
| | agtgaccaggcagtttttctgcctttagctttgacagttcttaattaagatcattg | |
| | aagaccagctttctcataaatttctctttttgaaaaaaagaaagcatttgtacta | |
| | agctcctctgtaagacaacatcttaaatcttaaaagtgttgttatcatgactggt | |
| | gagagaagaaaacattttgtttttattaaatggagcattatttacaaaaagcc | |
| | attgttgagaattagatcccacatcgtataaatatctattaaccattctaaataa | |
| | agagaactccagtgttgctatgtgcaagatcctctcttggagcttttttgcatag | |
| | caattaaaggtgtgctatttgtcagtagccatttttttgcagtgatttgaagacca | |
| | aagttgttttacagctgtgttaccgttaaaggtttttttttttatatgtattaaatcaat | |
| | ttatcactgtttaaagctttgaatatctgcaatctttgccaaggtacttttttatttaa | |
| | aaaaaaacataactttgtaaatattaccctgtaatattatatatacttaataaaa | |
| | cattttaagctat | |
| 593 | aagaatgctactggccttggtttaattgtgacttctacagggatattaatattact | CD47-205 |
| | tcactactatgtgtttagtacagcgattggattaacctccttcgtcattgccatatt | CDS |
| | ggttattcaggtgatagcctatatcctcgctgtggttggactgagtctctgtattg | nucleotide |
| | cggcgtgtataccaatgcatggccctcttctgatttcaggtttgagtatcttagct | sequence |
| | ctagcacaattacttggactagtttatatgaaatttgtggcttccaatcagaag | |
| | actatacaacctcctaggaaagctgtagaggaaccccttaatgaataa | |
| 594 | atggaggcacaaaacactactgaagtatacgtaaagtggaaatttaaagg | CD47-206 |
| | aagagatatttacacctttgatggagctctaaacaagtccactgtccccactg | CDS |
| | actttagtagtgcaaaaattgaagtctcacaattactaaaaggagatgcctct | nucleotide |
| | ttgaagatggataagagtgatgctgtctcacacacaggaaactacacttgtg | sequence |
| | aagtaacagaattaaccagagaaggtgaaacgatcatcgagctaaaatat | |
| | cgtgttgtttcatggttttctccaaatgaaaatattcttattgttattttcccaatttttg | |
| | ctatactcctgttctggggacagtttggtattaaaacacttaaatatagatccg | |
| | gtggtatggatgagaaaacaattgctttacttgttgctggactagtgatcactgt | |
| | cattgtcattgttggagccattcttttcgtcccaggtgaatattcattaaagaatg | |
| | ctactggccttggtttaattgtgacttctacagggatattaatattacttcactact | |
| | atgtgttt | |
| 595 | atgtggcccctggtcgccgccctgttgctgggctcggcatgctgcggatcag | Codon- |
| | ctcagctactgtttaataaaacaaaatctgtagaattcacgttttgtaacgaca | optimized |
| | ctgtcgtgatcccatgctttgttactaatatggaggcacaaaacaccactgaa | nucleotide |
| | gtgtacgtgaagtggaaattcaaaggcagagacatttacacctttgacggc | sequence |
| | gccctcaacaagtccaccgtgcccactgactttagtagcgcaaaaattgag | encoding |
| | gtcagccaattactaaaaggagatgcctctttgaagatggacaagagcgat | CD47 |
| | gctgtcagccacacagggaactacacttgtgaagtaacagagttaacccg | |
| | cgaaggtgaaacgatcatcgagctgaagtatcgagtggtgtcctggttttctc | |
| | cgaacgagaatatccttatcgtaattttcccaattttcgctatcctcctgttctgg | |
| | ggccagtttggtatcaagacactcaaatatcggtccggtgggatggatgag | |
| | aagacaattgccctgcttgttgctggactcgtgatcaccgtcatcgtgattgttg | |
| | gggccatccttttcgtcccaggggagtacagcctgaagaatgctacgggcc | |
| | tgggattaattgtgacctctacagggatactcatcctgcttcactactatgtgttc | |
| | agtaccgcgattggactgacctccttcgtcattgccatattggtgattcaggtg | |
| | atagcctacatcctcgccgtggttggcctgagtctctgtatcgcggcgtgcata | |
| | cccatgcatggccctcttctgatttcagggttgagtatcctcgcactagcacag | |
| | ttgctgggactggtttatatgaaatttgtggcctccaaccagaagactataca | |
| | gcctcctaggaaggctgtagaggagcccctgaatgcattcaaggaatcaa | |
| | aaggcatgatgaatgatgaa | |

### Cells, Cell Populations, and Compositions Thereof

Provided herein in some aspects are cells that have been genetically edited according to various embodiments disclosed herein. In some embodiments, the cells have genetic modifications at one or more genomic loci associated with blood type, including, for example, the ABO, FUT1, and/or RHD loci. In certain of these embodiments, the cells have modified expression (e.g., reduced or no expression) of one or more of these genes. As a result of the modified expression of the ABO, FUT1, and/or RHD gene, the cells may have modified blood type.

In some embodiments, the cell is an autologous cell, i.e., obtained from the subject who will receive the cell after genetic modification. In some embodiments, the cell is an allogeneic cell, i.e., obtained from someone other than the subject who will receive the cell after genetic modification.

In some embodiments, the cell is a mesenchymal stem cell or a hematopoietic stem cell. In some embodiments, the cell is a hematopoietic cell or a blood cell, for example, a red blood cell (erythrocyte), a platelet cell (thrombocyte), a mast cell, a basophil, an eosinophil, a neutrophil, a monocyte, a natural killer (NK) cell, a natural killer T (NKT) cell, a macrophage, a lymphocyte (e.g., a T cell, a B cell), or a plasma cell.

In some embodiments, the cell is a T cell, for example, a naïve T cell, a helper T cell (CD4+), a cytotoxic T cell (CD8+), a regulatory T cell (Treg), a central memory T cell (T_{CM}), an effector memory T cell (T_{EM}), a stem cell memory T cell (T_{SCM}), or any combination thereof. More specifically, the T cell can be naïve (not exposed to antigen; increased expression of CD62L, CCR7, CD28, CD3, CD127, and CD45RA, and decreased expression of CD45RO as compared to T_{CM}), memory T cells (antigen-experienced and long-lived), or effector cells (antigen-experienced, cytotoxic). Memory T cells can be further divided into subsets of T_{CM} (increased expression of CD62L, CCR7, CD28, CD127, CD45RO, and CD95, and decreased expression of CD54RA as compared to naïve T cells) and T_{EM} (decreased expression of CD62L, CCR7, CD28, CD45RA, and increased expression of CD127 as compared to naïve T cells or T_{CM}). Effector T cells refer to antigen-experienced CD8+ cytotoxic T cells that has decreased expression of CD62L, CCR7, CD28, and are positive for granzyme and perforin as compared to T_{CM}. Helper T cells are CD4+ cells that influence the activity of other immune cells by releasing cytokines. CD4+ T cells can activate or suppress an adaptive immune response, and which of those two functions is induced will depend on the presence of other cells and signals. T cells can be collected using known techniques, and the various subpopulations or combinations thereof can be enriched or depleted by known techniques, such as by affinity binding to antibodies, flow cytometry, or immunomagnetic selection. In some embodiments, the T cell can be a primary T cell obtained from a number of sources, including peripheral blood mononuclear cells, bone marrow, lymph node tissue, cord blood, thymus tissue, tissue from a site of infection, ascites, pleural effusion, spleen tissue, and tumors. In other embodiments, the T cell can be derived or differentiated from embryonic stem cells (ESCs) or induced pluripotent cells (iPSCs). In some embodiments, T cells may be modified to delete T cell receptors, e.g., by deletion of the TRAC or TRB locus, and may also be modified to express a chimeric antigen receptor.

In some embodiments, the cell is an NK cell. NK cells (also defined as large granular lymphocytes) represent a cell lineage differentiated from the common lymphoid progenitor (which also gives rise to B lymphocytes and T lymphocytes). Unlike T-cells, NK cells do not naturally express CD3 at the plasma membrane. Importantly, NK cells do not express a TCR and typically also lack other antigen-specific cell surface receptors. NK cells' cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and they balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins. On contact with a target cell, NK cells release proteins such as perforin, and enzymes such as proteases (granzymes). Perforin can form pores in the cell membrane of a target cell, inducing apoptosis or cell lysis. In some embodiments, the NK cells can be primary NK cells obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the NK cells can be derived or differentiated from ESCs or iPSCs. There are a number of techniques that can be used to generate NK cells from pluripotent stem cells (e.g., iPSCs). See, for example, Zhu et al., Methods Mol Biol. 2019; 2048:107-119; Knorr et al., Stem Cells Transl Med. 2013 2(4):274-83. doi: 10.5966/sctm.2012-0084; Zeng et al., Stem Cell Reports. 2017 Dec. 12; 9(6):1796-1812; Ni et al., Methods Mol Biol. 2013; 1029:33-41; Bernareggi et al., Exp Hematol. 2019 71:13-23; Shankar et al., Stem Cell Res Ther. 2020; 11(1):234, all of which are incorporated herein by reference in their entirety and specifically for the methodologies and reagents for differentiation. Differentiation can be assayed as is known in the art, generally by evaluating the presence of NK cell associated and/or specific markers, including, but not limited to, CD56, KIRs, CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the cell is an NKT cell. NKT cells are a heterogeneous group of T cells that share properties of both T cells and NK cells. Many of these cells recognize the non-polymorphic CD1d molecule, an antigen-presenting molecule that binds self and foreign lipids and glycolipids. They constitute only approximately 1% of all peripheral blood T cells. In some embodiments, the NKT cells can be primary NKT cells obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the NKT cells can be derived or differentiated from ESCs or iPSCs.

In some embodiments, the cell is a pancreatic islet cell, including, for example, a β cell (also referred to as beta cell or β islet cell). Exemplary pancreatic islet cell types include, but are not limited to, pancreatic islet progenitor cells, immature pancreatic islet cells, mature pancreatic islet cells, and the like. In some embodiments, the β islet cells can be primary β islet cells. In some embodiments, the β islet cells can be derived or differentiated from ESCs or iPSCs. Useful method for differentiating pluripotent stem cells into pancreatic islet cells are disclosed, for example, in U.S. Pat. Nos. 9,683,215; 9,157,062; and 8,927,280. In some embodiments, the β islet cells genetically engineered by the methods as disclosed herein secretes insulin. In some embodiments, a β islet cell exhibits at least two characteristics of an endogenous pancreatic islet cell, for example, but not limited to, secretion of insulin in response to an increase in glucose, and expression of beta cell markers. In some embodiments, the β islet cells disclosed herein are administered to a subject to treat diabetes. Exemplary p cell markers or p cell progenitor markers include, but are not limited to, c-peptide, Pdxl, glucose transporter 2 (Glut2), HNF6, VEGF, glucokinase (GCK), prohormone convertase (PC 1/3), Cdcpl, NeuroD, Ngn3, Nkx2.2, Nkx6.1, Nkx6.2, Pax4, Pax6, Ptfla, Isll, Sox9, Soxl7, and FoxA2. In some embodiments, the PSCs are differentiated into beta-like cells or islet organoids for transplantation to address type I diabetes mellitus (T1DM). Cell systems are a promising way to address T1DM, see, e.g., Ellis et al., Nat Rev Gastroenterol Hepatol. 2017 October; 14(10):612-628, incorporated herein by reference. Additionally, Pagliuca et al. (Cell, 2014, 159(2):428-39) reports on the successful differentiation of β cells from hiPSCs, the contents incorporated herein by reference in its entirety and in particular for the methods and reagents disclosed there for the large-scale production of functional human β cells from human pluripotent stem cells). Furthermore, Vegas et al. shows the production of human β cells from human pluripotent stem cells followed by encapsulation to avoid immune rejection by the recipient; Vegas et al., Nat Med, 2016, 22(3):306-11, incorporated herein by reference in its entirety and in particular for the methods and reagents disclosed there for the large-scale production of functional human B cells from human pluripotent stem cells. Additional disclosure of pancreatic islet cells including pancreatic β islet cells for use in the present technology are found in WO2020/018615, the disclosure is herein incorporated by reference in its entirety.

In some embodiments, the cell is a pluripotent stem cell, for example, an ESC or iPSC. In some embodiments, the cell is a cell differentiated from pluripotent stem cells, e.g., ESCs or iPSCs. ESCs and iPSCs have the ability to differentiated into any cell type of the body, including, for example, neurons, astrocytes, oligodendrocytes, retinal epithelial cells, epidermal cells, hair cells, keratinocytes, hepatocytes, pancreatic β islet cells, intestinal epithelial cells, lung alveolar cells, hematopoietic cells, endothelial cells, cardiomyocytes, smooth muscle cells, skeletal muscle cells, renal cells, adipocytes, chondrocytes, thyroid cell, NK cells, NKT cells, macrophages, T cells, B cells, and osteocytes.

In some embodiments, the cell is a primary cell, including, for example, neurons, astrocytes, oligodendrocytes, retinal epithelial cells, epidermal cells, hair cells, keratinocytes, hepatocytes, pancreatic β islet cells, intestinal epithelial cells, lung alveolar cells, hematopoietic cells, mesenchymal stem cells, hematopoietic stem cells, endothelial cells, cardiomyocytes, smooth muscle cells, skeletal muscle cells, renal cells, adipocytes, chondrocytes, thyroid cells, NK cells, NKT cells, macrophages, T cells, B cells, and osteocytes. In some embodiments, the cell is a cardiomyocyte, a retinal pigment epithelial cell (RPE), an endothelial cell, a β islet cell, or a glial progenitor cell (GPC).

In some aspects, the present technology provides pharmaceutical compositions comprising a cell according to various embodiments disclosed herein.

In some embodiments, the compositions can have various formulations, for example, injectable formulations, lyophilized formulations, liquid formulations, oral formulations, etc., depending on the suitable routes of administration.

In some embodiments, the compositions can be co-formulated in the same dosage unit or can be individually formulated in separate dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e., 1 to 10, 1 to 8, 1 to 6, 1 to 4, or 1 to 2) of times per day, or as many times as needed to elicit a therapeutic response.

In some embodiments, a single dosage unit includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells.

### Methods of Treatment

In some aspects, provided are methods for treating and/or preventing a disease in a subject in need thereof. The method entails obtaining a cell from the subject (autologous) or from a donor (allogeneic); genetically modifying the cell, including targeted gene editing at one or more genomic loci associated with blood types, according to various embodiments disclosed herein; and administering to the subject a therapeutically effective amount of the genetically modified cell or a pharmaceutical composition containing the same.

In some embodiments, the disease is cancer. In some embodiments, the cancer is a hematologic malignancy. Non-limiting examples of hematologic malignancies include myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), B cell acute lymphoid leukemia (B-ALL), T cell acute lymphoid leukemia (T-ALL), T cell lymphoma, and B cell lymphoma.

In some embodiments, the disease is an autoimmune disease, including, for example, lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

In some embodiments, the disease is diabetes mellitus, including, for example, Type I diabetes, Type II diabetes, prediabetes, and gestational diabetes.

In some embodiments, the disease is a neurological disease, including, for example, catalepsy, epilepsy, encephalitis, meningitis, migraine, Huntington's, Alzheimer's, Parkinson's, Pelizaeus-Merzbacher disease, and multiple sclerosis.

In some embodiments, the disease is a cardiac disease or disorder, i.e., a condition and/or disorder relating to the heart, including the valves, endothelium, infarcted zones, or other components or structures of the heart. Cardiac diseases or disorders include, for example, pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, cardiomegaly, mitral insufficiency, and autoimmune endocarditis.

In some embodiments, the genetically modified cell or pharmaceutical composition containing the same according to the present technology may be administered in a manner appropriate to the disease, condition, or disorder to be treated as determined by persons skilled in the medical art. In any of the above embodiments, the genetically modified cell may be administered intravenously, intraperitoneally, intratumorally, into the bone marrow, into a lymph node, or into the cerebrospinal fluid, so as to encounter the target antigen or cells. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In some embodiments, the amount of genetically modified cells of the present technology in a pharmaceutical composition is typically greater than 10² cells, for example, about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰ cells, or more.

In some embodiments, the methods comprise administering to the subject the genetically modified cells or pharmaceutical composition containing the same once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the genetically modified cells or pharmaceutical composition containing the same can be administered every day, every other day, every third day, weekly, biweekly (i.e., every other week), every third week, monthly, every other month, or every third month.

In some embodiments, the genetically modified cells or pharmaceutical composition containing the same may be administered over a pre-determined time period. Alternatively, the genetically modified cell or pharmaceutical composition containing the same may be administered until a particular therapeutic benchmark is reached. In some embodiments, the methods provided herein include a step of evaluating one or more therapeutic benchmarks in a biological sample, such as, but not limited to, the level of a disease related biomarker, to determine whether to continue administration of the genetically modified cell or pharmaceutical composition containing the same.

In some embodiments, the methods further comprise administering the subject a pharmaceutically effective amount of one or more additional therapeutic agents to obtain improved or synergistic therapeutic effects. In some embodiments, the one or more additional therapeutic agents are selected from the group consisting of an immunotherapy agent, a chemotherapy agent, and a biologic agent. In some embodiments, the subject was administered the one or more additional therapeutic agents before administration of the genetically modified cells or pharmaceutical composition containing the same. In some embodiments, the subject is co-administered the one or more additional therapeutic agents and the genetically modified cells or pharmaceutical composition containing the same. In some embodiments, the subject was administered the one or more additional therapeutic agents after administration of the genetically modified cells or pharmaceutical composition containing the same.

As one of ordinary skill in the art would understand, the one or more additional therapeutic agents and the genetically modified cells or pharmaceutical composition containing the same can be administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known components and functions have not been shown or described in detail to avoid unnecessarily obscuring the description of the embodiments of the technology. Where the context permits, singular or plural terms may also include the plural or singular term, respectively. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

TABLE 4
Exemplary ABO, FUT1, and RHD target sequences and cut sites for
targeted gene editing

| SEQ | Target sequence | Target | | | Genomic |
|---|---|---|---|---|---|
| ID NO: | (5′→3′) | Gene | Strand | PAM | Cut Site |
| 20 | ggccagcgtccgcaacacct | ABO | −1 | cgg | 133,275,179 |
| 21 | ggccgaggtgttgcggacgc | ABO | 1 | tgg | 133,275,170 |
| 22 | ggtcgaagtgcgtggcattt | ABO | −1 | tgg | 133,262,157 |
| 23 | ggatcataggtcgaagtgcg | ABO | −1 | tgg | 133,262,149 |
| 24 | attattaggaaaaggatcat | ABO | −1 | agg | 133,262,136 |
| 25 | agacaagcattattaggaaa | ABO | −1 | agg | 133,262,128 |
| 26 | agaccaagacaagcattatt | ABO | −1 | agg | 133,262,122 |
| 27 | tttcctaataatgcttgtct | ABO | 1 | tgg | 133,262,114 |
| 28 | atgcttgtcttggtcttgtt | ABO | 1 | tgg | 133,262,104 |
| 29 | gcattagacttctggggctt | ABO | −1 | agg | 133,261,359 |
| 30 | ttcctggcattagacttctg | ABO | −1 | ggg | 133,261,353 |
| 31 | cttcctggcattagacttct | ABO | −1 | ggg | 133,261,352 |
| 32 | gcttcctggcattagacttc | ABO | −1 | tgg | 133,261,351 |
| 33 | agccccagaagtctaatgcc | ABO | 1 | agg | 133,261,344 |
| 34 | aacccccgttccaggcttcc | ABO | −1 | tgg | 133,261,337 |
| 35 | aagtctaatgccaggaagcc | ABO | 1 | tgg | 133,261,336 |
| 36 | taatgccaggaagcctggaa | ABO | 1 | cgg | 133,261,331 |
| 37 | aatgccaggaagcctggaac | ABO | 1 | ggg | 133,261,330 |
| 38 | atgccaggaagcctggaacg | ABO | 1 | ggg | 133,261,329 |
| 39 | tgccaggaagcctggaacgg | ABO | 1 | ggg | 133,261,328 |
| 40 | gagacgcgctgcagatggtc | ABO | −1 | agg | 133,259,844 |
| 41 | gcaacgagacgcgctgcaga | ABO | −1 | tgg | 133,259,839 |
| 42 | gtgtcagcacctttggctgg | ABO | −1 | ggg | 133,258,117 |
| 43 | ggtgtcagcacctttggctg | ABO | −1 | ggg | 133,258,116 |
| 44 | gatggtctacccccagccaa | ABO | 1 | agg | 133,258,115 |
| 45 | cggtgtcagcacctttggct | ABO | −1 | ggg | 133,258,115 |
| 46 | acggtgtcagcacctttggc | ABO | −1 | tgg | 133,258,114 |
| 47 | gacaatgggagccagccaag | ABO | −1 | ggg | 133,257,514 |
| 48 | agacaatgggagccagccaa | ABO | −1 | ggg | 133,257,513 |
| 49 | cagacaatgggagccagcca | ABO | −1 | agg | 133,257,512 |
| 50 | cttggctggctcccattgtc | ABO | 1 | tgg | 133,257,500 |
| 51 | aatgtgccctcccagacaat | ABO | −1 | ggg | 133,257,500 |
| 52 | ttggctggctcccattgtct | ABO | 1 | ggg | 133,257,499 |
| 53 | gaatgtgccctcccagacaa | ABO | −1 | tgg | 133,257,499 |
| 54 | gctggctcccattgtctggg | ABO | 1 | agg | 133,257,496 |
| 55 | ctggctcccattgtctggga | ABO | 1 | ggg | 133,257,495 |
| 56 | ggagcctgaactgctcgttg | ABO | −1 | agg | 133,257,465 |
| 57 | acatcctcaacgagcagttc | ABO | 1 | agg | 133,257,458 |
| 58 | ttaacccaatggtggtgttc | ABO | −1 | tgg | 133,257,444 |
| 59 | aggctccagaacaccaccat | ABO | 1 | tgg | 133,257,438 |
| 60 | ggctccagaacaccaccatt | ABO | 1 | ggg | 133,257,437 |
| 61 | aaacacagttaacccaatgg | ABO | −1 | tgg | 133,257,436 |
| 62 | ggcaaacacagttaacccaa | ABO | −1 | tgg | 133,257,433 |
| 63 | ccgtctccaggaacagcttc | ABO | −1 | agg | 133,256,338 |
| 64 | ggctttcctgaagctgttcc | ABO | 1 | tgg | 133,256,333 |
| 65 | cctgaagctgttcctggaga | ABO | 1 | cgg | 133,256,327 |
| 66 | agtgcttctccgccgtctcc | ABO | −1 | agg | 133,256,326 |
| 67 | gaagctgttcctggagacgg | ABO | 1 | cgg | 133,256,324 |
| 68 | gacggcggagaagcacttca | ABO | 1 | tgg | 133,256,309 |
| 69 | ggcggagaagcacttcatgg | ABO | 1 | tgg | 133,256,306 |
| 70 | gcggagaagcacttcatggt | ABO | 1 | ggg | 133,256,305 |
| 71 | agacatagtagtggacacgg | ABO | −1 | tgg | 133,256,293 |
| 72 | tgaagacatagtagtggaca | ABO | −1 | cgg | 133,256,290 |
| 73 | ggtcggtgaagacatagtag | ABO | −1 | tgg | 133,256,284 |
| 74 | ctatgtcttcaccgaccagc | ABO | 1 | cgg | 133,256,267 |
| 75 | gggcaccgcggccggctggt | ABO | −1 | cgg | 133,256,267 |
| 76 | cgcggggcaccgcggccggc | ABO | −1 | tgg | 133,256,263 |
| 77 | cttcaccgaccagccggccg | ABO | 1 | cgg | 133,256,261 |
| 78 | gtcacgcggggcaccgcggc | ABO | −1 | cgg | 133,256,259 |
| 79 | cagcgtcacgcggggcaccg | ABO | −1 | cgg | 133,256,255 |
| 80 | ccggtccccagcgtcacgcg | ABO | −1 | ggg | 133,256,247 |
| 81 | accggtccccagcgtcacgc | ABO | −1 | ggg | 133,256,246 |
| 82 | gaccggtccccagcgtcacg | ABO | −1 | cgg | 133,256,245 |
| 83 | cgcggtgccccgcgtgacgc | ABO | 1 | tgg | 133,256,243 |
| 84 | gcggtgccccgcgtgacgct | ABO | 1 | ggg | 133,256,242 |
| 85 | cggtgccccgcgtgacgctg | ABO | 1 | ggg | 133,256,241 |
| 86 | ccccgcgtgacgctggggac | ABO | 1 | cgg | 133,256,236 |
| 87 | gcgtgacgctggggaccggt | ABO | 1 | cgg | 133,256,232 |
| 88 | cagcactgacagctgccgac | ABO | −1 | cgg | 133,256,228 |
| 89 | cggtcggcagctgtcagtgc | ABO | 1 | tgg | 133,256,216 |
| 90 | tcggcagctgtcagtgctgg | ABO | 1 | agg | 133,256,213 |
| 91 | cacgtcctgccagcgcttgt | ABO | −1 | agg | 133,256,195 |
| 92 | aggtgcgcgcctacaagcgc | ABO | 1 | tgg | 133,256,193 |
| 93 | gcgcgcctacaagcgctggc | ABO | 1 | agg | 133,256,189 |
| 94 | gatcatctccatgcggcgca | ABO | −1 | tgg | 133,256,171 |
| 95 | ggacgtgtccatgcgccgca | ABO | 1 | tgg | 133,256,168 |
| 96 | agtcactgatcatctccatg | ABO | −1 | cgg | 133,256,164 |
| 97 | tgatcagtgacttctgcgag | ABO | 1 | cgg | 133,256,142 |
| 98 | cgagcggcgcttcctcagcg | ABO | 1 | agg | 133,256,126 |
| 99 | ccaggtaatccacctcgctg | ABO | −1 | agg | 133,256,125 |
| 100 | gcggcgcttcctcagcgagg | ABO | 1 | tgg | 133,256,123 |
| 101 | cctcagcgaggtggattacc | ABO | 1 | tgg | 133,256,114 |
| 102 | tgtccacgtccacgcacacc | ABO | −1 | agg | 133,256,107 |
| 103 | ggtggattacctggtgtgcg | ABO | 1 | tgg | 133,256,105 |
| 104 | ttacctggtgtgcgtggacg | ABO | 1 | tgg | 133,256,099 |
| 105 | ggtgtgcgtggacgtggaca | ABO | 1 | tgg | 133,256,093 |
| 106 | tctccacgcccacgtggtcg | ABO | −1 | cgg | 133,256,077 |
| 107 | catggagttccgcgaccacg | ABO | 1 | tgg | 133,256,075 |
| 108 | atggagttccgcgaccacgt | ABO | 1 | ggg | 133,256,074 |
| 109 | tcaggatctccacgcccacg | ABO | −1 | tgg | 133,256,071 |
| 110 | gttccgcgaccacgtgggcg | ABO | 1 | tgg | 133,256,069 |
| 111 | gggtgccgaacagcggagtc | ABO | −1 | agg | 133,256,053 |
| 112 | gagatcctgactccgctgtt | ABO | 1 | cgg | 133,256,047 |
| 113 | gggtgcagggtgccgaacag | ABO | −1 | cgg | 133,256,046 |
| 114 | tccgtagaagccggggtgca | ABO | −1 | ggg | 133,256,033 |
| 115 | ctgttcggcaccctgcaccc | ABO | 1 | cgg | 133,256,032 |
| 116 | ttccgtagaagccggggtgc | ABO | −1 | agg | 133,256,032 |
| 117 | ggctgcttccgtagaagccg | ABO | −1 | ggg | 133,256,026 |
| 118 | cggctgcttccgtagaagcc | ABO | −1 | ggg | 133,256,025 |
| 119 | ccggctgcttccgtagaagc | ABO | −1 | cgg | 133,256,024 |
| 120 | accctgcaccccggcttcta | ABO | 1 | cgg | 133,256,023 |
| 121 | ccggcttctacggaagcagc | ABO | 1 | cgg | 133,256,013 |
| 122 | cggcttctacggaagcagcc | ABO | 1 | ggg | 133,256,012 |
| 123 | cttctacggaagcagccggg | ABO | 1 | agg | 133,256,009 |
| 124 | gctcgtaggtgaaggcctcc | ABO | −1 | cgg | 133,256,005 |
| 125 | gggccggcgctcgtaggtga | ABO | −1 | agg | 133,255,997 |
| 126 | ggactggggccggcgctcgt | ABO | −1 | agg | 133,255,991 |
| 127 | aggccttcacctacgagcgc | ABO | 1 | cgg | 133,255,989 |
| 128 | tgtaggcctgggactggggc | ABO | −1 | cgg | 133,255,981 |
| 129 | gggatgtaggcctgggactg | ABO | −1 | ggg | 133,255,977 |
| 130 | cgagcgccggccccagtccc | ABO | 1 | agg | 133,255,976 |
| 131 | ggggatgtaggcctgggact | ABO | −1 | ggg | 133,255,976 |
| 132 | tggggatgtaggcctgggac | ABO | −1 | tgg | 133,255,975 |
| 133 | gtccttggggatgtaggcct | ABO | −1 | ggg | 133,255,970 |
| 134 | cgtccttggggatgtaggcc | ABO | −1 | tgg | 133,255,969 |
| 135 | gccctcgtccttggggatgt | ABO | −1 | agg | 133,255,964 |
| 136 | gtcccaggcctacatcccca | ABO | 1 | agg | 133,255,961 |
| 137 | agaaatcgccctcgtccttg | ABO | −1 | ggg | 133,255,957 |
| 138 | tagaaatcgccctcgtcctt | ABO | −1 | ggg | 133,255,956 |
| 139 | ggcctacatccccaaggacg | ABO | 1 | agg | 133,255,955 |
| 140 | gtagaaatcgccctcgtcct | ABO | −1 | tgg | 133,255,955 |
| 141 | gcctacatccccaaggacga | ABO | 1 | ggg | 133,255,954 |
| 142 | cgagggcgatttctactacc | ABO | 1 | tgg | 133,255,937 |
| 143 | gagggcgatttctactacct | ABO | 1 | ggg | 133,255,936 |
| 144 | agggcgatttctactacctg | ABO | 1 | ggg | 133,255,935 |
| 145 | gggcgatttctactacctgg | ABO | 1 | ggg | 133,255,934 |
| 146 | ggcgatttctactacctggg | ABO | 1 | ggg | 133,255,933 |
| 147 | gcgatttctactacctgggg | ABO | 1 | ggg | 133,255,932 |
| 148 | accccccgaagaaccccccc | ABO | −1 | agg | 133,255,930 |
| 149 | tactacctgggggggttctt | ABO | 1 | cgg | 133,255,924 |
| 150 | actacctgggggggttcttc | ABO | 1 | ggg | 133,255,923 |
| 151 | ctacctgggggggttcttcg | ABO | 1 | ggg | 133,255,922 |
| 152 | tacctgggggggttcttcgg | ABO | 1 | ggg | 133,255,921 |
| 153 | acctgggggggttcttcggg | ABO | 1 | ggg | 133,255,920 |
| 154 | gggggggttcttcggggggt | ABO | 1 | cgg | 133,255,916 |
| 155 | cttcggggggtcggtgcaag | ABO | 1 | agg | 133,255,907 |
| 156 | ggtcggtgcaagaggtgcag | ABO | 1 | cgg | 133,255,899 |
| 157 | aagaggtgcagcggctcacc | ABO | 1 | agg | 133,255,890 |
| 158 | agaggtgcagcggctcacca | ABO | 1 | ggg | 133,255,889 |
| 159 | catggcctggtggcaggccc | ABO | −1 | tgg | 133,255,883 |
| 160 | gctcaccagggcctgccacc | ABO | 1 | agg | 133,255,877 |
| 161 | gaccatcatggcctggtggc | ABO | −1 | agg | 133,255,877 |
| 162 | ggtcgaccatcatggcctgg | ABO | −1 | tgg | 133,255,873 |
| 163 | cctggtcgaccatcatggcc | ABO | −1 | tgg | 133,255,870 |
| 164 | ggcctgccaccaggccatga | ABO | 1 | tgg | 133,255,868 |
| 165 | gttggcctggtcgaccatca | ABO | −1 | tgg | 133,255,865 |
| 166 | ccaggccatgatggtcgacc | ABO | 1 | agg | 133,255,859 |
| 167 | atgatggtcgaccaggccaa | ABO | 1 | cgg | 133,255,852 |
| 168 | cggcctcgatgccgttggcc | ABO | −1 | tgg | 133,255,852 |
| 169 | ccacacggcctcgatgccgt | ABO | −1 | tgg | 133,255,847 |
| 170 | cgaccaggccaacggcatcg | ABO | 1 | agg | 133,255,844 |
| 171 | ccaacggcatcgaggccgtg | ABO | 1 | tgg | 133,255,836 |
| 172 | gtggctctcgtcgtgccaca | ABO | −1 | cgg | 133,255,832 |
| 173 | gcagcaggtacttgttcagg | ABO | −1 | tgg | 133,255,813 |
| 174 | ggcgcagcaggtacttgttc | ABO | −1 | agg | 133,255,810 |
| 175 | tggtgggtttgtggcgcagc | ABO | −1 | agg | 133,255,798 |
| 176 | agagcaccttggtgggtttg | ABO | −1 | tgg | 133,255,789 |
| 177 | gctgcgccacaaacccacca | ABO | 1 | agg | 133,255,784 |
| 178 | tcgggggagagcaccttggt | ABO | −1 | ggg | 133,255,782 |
| 179 | ctcgggggagagcaccttgg | ABO | −1 | tgg | 133,255,781 |
| 180 | gtactcgggggagagcacct | ABO | −1 | tgg | 133,255,778 |
| 181 | ctggtcccacaagtactcgg | ABO | −1 | ggg | 133,255,766 |
| 182 | gctggtcccacaagtactcg | ABO | −1 | ggg | 133,255,765 |
| 183 | tgctggtcccacaagtactc | ABO | −1 | ggg | 133,255,764 |
| 184 | ctgctggtcccacaagtact | ABO | −1 | cgg | 133,255,763 |
| 185 | tgctctcccccgagtacttg | ABO | 1 | tgg | 133,255,761 |
| 186 | gctctcccccgagtacttgt | ABO | 1 | ggg | 133,255,760 |
| 187 | cgggccagcccagcagctgc | ABO | −1 | tgg | 133,255,747 |
| 188 | cttgtgggaccagcagctgc | ABO | 1 | tgg | 133,255,745 |
| 189 | ttgtgggaccagcagctgct | ABO | 1 | ggg | 133,255,744 |
| 190 | gggaccagcagctgctgggc | ABO | 1 | tgg | 133,255,740 |
| 191 | ctcagcttcctcaggacggc | ABO | −1 | ggg | 133,255,728 |
| 192 | cctcagcttcctcaggacgg | ABO | −1 | cgg | 133,255,727 |
| 193 | tgggctggcccgccgtcctg | ABO | 1 | agg | 133,255,725 |
| 194 | gaacctcagcttcctcagga | ABO | −1 | cgg | 133,255,724 |
| 195 | cagtgaacctcagcttcctc | ABO | −1 | agg | 133,255,720 |
| 196 | ccgccgtcctgaggaagctg | ABO | 1 | agg | 133,255,716 |
| 197 | gaggaagctgaggttcactg | ABO | 1 | cgg | 133,255,706 |
| 198 | cggaccgcctggtggttctt | ABO | −1 | ggg | 133,255,692 |
| 199 | ccggaccgcctggtggttct | ABO | −1 | tgg | 133,255,691 |
| 200 | tgcggtgcccaagaaccacc | ABO | 1 | agg | 133,255,688 |
| 201 | ggtgcccaagaaccaccagg | ABO | 1 | cgg | 133,255,685 |
| 202 | acgggttccggaccgcctgg | ABO | −1 | tgg | 133,255,684 |
| 203 | ccaagaaccaccaggcggtc | ABO | 1 | cgg | 133,255,680 |
| 204 | ggcagagctgacgatggctc | FUT1 | −1 | cgg | 48,748,518 |
| 205 | aggccaggcagagctgacga | FUT1 | −1 | tgg | 48,748,512 |
| 206 | gagccatcgtcagctctgcc | FUT1 | 1 | tgg | 48,748,504 |
| 207 | cacagactagcaggaaggcc | FUT1 | −1 | agg | 48,748,497 |
| 208 | gaggacacagactagcagga | FUT1 | −1 | agg | 48,748,492 |
| 209 | cagagaggacacagactagc | FUT1 | −1 | agg | 48,748,488 |
| 210 | ggaggaagaagattacagag | FUT1 | −1 | agg | 48,748,473 |
| 211 | agctgtcttgatggatatgg | FUT1 | −1 | agg | 48,748,455 |
| 212 | gaaagctgtcttgatggata | FUT1 | −1 | tgg | 48,748,452 |
| 213 | catgtggaaagctgtcttga | FUT1 | −1 | tgg | 48,748,446 |
| 214 | catcaagacagctttccaca | FUT1 | 1 | tgg | 48,748,434 |
| 215 | atcgacaggcctaggccatg | FUT1 | −1 | tgg | 48,748,430 |
| 216 | gacagctttccacatggcct | FUT1 | 1 | agg | 48,748,428 |
| 217 | gacacaggatcgacaggcct | FUT1 | −1 | agg | 48,748,422 |
| 218 | ggtctggacacaggatcgac | FUT1 | −1 | agg | 48,748,416 |
| 219 | ccaggcggcggtctggacac | FUT1 | −1 | agg | 48,748,407 |
| 220 | ggtgtcaccaggcggcggtc | FUT1 | −1 | tgg | 48,748,400 |
| 221 | cctgtgtccagaccgccgcc | FUT1 | 1 | tgg | 48,748,396 |
| 222 | ctgggggtgtcaccaggcgg | FUT1 | −1 | cgg | 48,748,395 |
| 223 | ccactgggggtgtcaccagg | FUT1 | −1 | cgg | 48,748,392 |
| 224 | tggccactgggggtgtcacc | FUT1 | −1 | agg | 48,748,389 |
| 225 | ccgcctggtgacacccccag | FUT1 | 1 | tgg | 48,748,381 |
| 226 | aggcagaagatggccactgg | FUT1 | −1 | ggg | 48,748,379 |
| 227 | caggcagaagatggccactg | FUT1 | −1 | ggg | 48,748,378 |
| 228 | gcaggcagaagatggccact | FUT1 | −1 | ggg | 48,748,377 |
| 229 | ggcaggcagaagatggccac | FUT1 | −1 | tgg | 48,748,376 |
| 230 | agtacccggcaggcagaaga | FUT1 | −1 | tgg | 48,748,369 |
| 231 | agtggccatcttctgcctgc | FUT1 | 1 | cgg | 48,748,363 |
| 232 | gtggccatcttctgcctgcc | FUT1 | 1 | ggg | 48,748,362 |
| 233 | ggcccatcgcagtacccggc | FUT1 | −1 | agg | 48,748,359 |
| 234 | ttggggcccatcgcagtacc | FUT1 | −1 | cgg | 48,748,355 |
| 235 | ctgcctgccgggtactgcga | FUT1 | 1 | tgg | 48,748,351 |
| 236 | tgcctgccgggtactgcgat | FUT1 | 1 | ggg | 48,748,350 |
| 237 | gacaggaagaggaggcgttg | FUT1 | −1 | ggg | 48,748,338 |
| 238 | ggacaggaagaggaggcgtt | FUT1 | −1 | ggg | 48,748,337 |
| 239 | gggacaggaagaggaggcgt | FUT1 | −1 | tgg | 48,748,336 |
| 240 | gtgctggggacaggaagagg | FUT1 | −1 | agg | 48,748,330 |
| 241 | agggtgctggggacaggaag | FUT1 | −1 | agg | 48,748,327 |
| 242 | ggaagcagggtgctggggac | FUT1 | −1 | agg | 48,748,321 |
| 243 | gagagggaagcagggtgctg | FUT1 | −1 | ggg | 48,748,316 |
| 244 | ggagagggaagcagggtgct | FUT1 | −1 | ggg | 48,748,315 |
| 245 | cggagagggaagcagggtgc | FUT1 | −1 | tgg | 48,748,314 |
| 246 | aggtgccggagagggaagca | FUT1 | −1 | ggg | 48,748,308 |
| 247 | caggtgccggagagggaagc | FUT1 | −1 | agg | 48,748,307 |
| 248 | cagcaccctgcttccctctc | FUT1 | 1 | cgg | 48,748,302 |
| 249 | gacagtccaggtgccggaga | FUT1 | −1 | ggg | 48,748,300 |
| 250 | agacagtccaggtgccggag | FUT1 | −1 | agg | 48,748,299 |
| 251 | ctgcttccctctccggcacc | FUT1 | 1 | tgg | 48,748,295 |
| 252 | ggggtagacagtccaggtgc | FUT1 | −1 | cgg | 48,748,294 |
| 253 | gccattggggtagacagtcc | FUT1 | −1 | agg | 48,748,288 |
| 254 | acctggactgtctaccccaa | FUT1 | 1 | tgg | 48,748,278 |
| 255 | gattaccaaaccggccattg | FUT1 | −1 | ggg | 48,748,275 |
| 256 | ggactgtctaccccaatggc | FUT1 | 1 | cgg | 48,748,274 |
| 257 | tgattaccaaaccggccatt | FUT1 | −1 | ggg | 48,748,274 |
| 258 | ctgattaccaaaccggccat | FUT1 | −1 | tgg | 48,748,273 |
| 259 | gtctaccccaatggccggtt | FUT1 | 1 | tgg | 48,748,269 |
| 260 | gtcccatctgattaccaaac | FUT1 | −1 | cgg | 48,748,266 |
| 261 | tggccggtttggtaatcaga | FUT1 | 1 | tgg | 48,748,258 |
| 262 | ggccggtttggtaatcagat | FUT1 | 1 | ggg | 48,748,257 |
| 263 | gggacagtatgccacgctgc | FUT1 | 1 | tgg | 48,748,237 |
| 264 | ctgggccagagccagcagcg | FUT1 | −1 | tgg | 48,748,237 |
| 265 | gtatgccacgctgctggctc | FUT1 | 1 | tgg | 48,748,231 |
| 266 | ggcccggcggccgttgagct | FUT1 | −1 | ggg | 48,748,219 |
| 267 | ctggctctggcccagctcaa | FUT1 | 1 | cgg | 48,748,218 |
| 268 | aggcccggcggccgttgagc | FUT1 | −1 | tgg | 48,748,218 |
| 269 | tggcccagctcaacggccgc | FUT1 | 1 | cgg | 48,748,211 |
| 270 | ggcccagctcaacggccgcc | FUT1 | 1 | ggg | 48,748,210 |
| 271 | caggcaggataaaggcccgg | FUT1 | −1 | cgg | 48,748,206 |
| 272 | tggcaggcaggataaaggcc | FUT1 | −1 | cgg | 48,748,203 |
| 273 | atgcatggcaggcaggataa | FUT1 | −1 | agg | 48,748,198 |
| 274 | gggcggcatgcatggcaggc | FUT1 | −1 | agg | 48,748,191 |
| 275 | gccagggcggcatgcatggc | FUT1 | −1 | agg | 48,748,187 |
| 276 | cggggccagggcggcatgca | FUT1 | −1 | tgg | 48,748,183 |
| 277 | gcctgccatgcatgccgccc | FUT1 | 1 | tgg | 48,748,177 |
| 278 | gcggaataccggggccaggg | FUT1 | −1 | cgg | 48,748,174 |
| 279 | catgcatgccgccctggccc | FUT1 | 1 | cgg | 48,748,171 |
| 280 | gatgcggaataccggggcca | FUT1 | −1 | ggg | 48,748,171 |
| 281 | tgatgcggaataccggggcc | FUT1 | −1 | agg | 48,748,170 |
| 282 | cagggtgatgcggaataccg | FUT1 | −1 | ggg | 48,748,165 |
| 283 | gcagggtgatgcggaatacc | FUT1 | −1 | ggg | 48,748,164 |
| 284 | ggcagggtgatgcggaatac | FUT1 | −1 | cgg | 48,748,163 |
| 285 | ccagcacgggcagggtgatg | FUT1 | −1 | cgg | 48,748,155 |
| 286 | ttctggggccagcacgggca | FUT1 | −1 | ggg | 48,748,147 |
| 287 | cttctggggccagcacgggc | FUT1 | −1 | agg | 48,748,146 |
| 288 | ccgcatcaccctgcccgtgc | FUT1 | 1 | tgg | 48,748,144 |
| 289 | tccacttctggggccagcac | FUT1 | −1 | ggg | 48,748,142 |
| 290 | gtccacttctggggccagca | FUT1 | −1 | cgg | 48,748,141 |
| 291 | gcccgtgctggccccagaag | FUT1 | 1 | tgg | 48,748,132 |
| 292 | cgtgcggctgtccacttctg | FUT1 | −1 | ggg | 48,748,132 |
| 293 | gcgtgcggctgtccacttct | FUT1 | −1 | ggg | 48,748,131 |
| 294 | ggcgtgcggctgtccacttc | FUT1 | −1 | tgg | 48,748,130 |
| 295 | gcagctcccgccacggcgtg | FUT1 | −1 | cgg | 48,748,116 |
| 296 | aagtggacagccgcacgccg | FUT1 | 1 | tgg | 48,748,115 |
| 297 | tggacagccgcacgccgtgg | FUT1 | 1 | cgg | 48,748,112 |
| 298 | ggacagccgcacgccgtggc | FUT1 | 1 | ggg | 48,748,111 |
| 299 | tgaagctgcagctcccgcca | FUT1 | −1 | cgg | 48,748,109 |
| 300 | gggagctgcagcttcacgac | FUT1 | 1 | tgg | 48,748,091 |
| 301 | gcagcttcacgactggatgt | FUT1 | 1 | cgg | 48,748,084 |
| 302 | gcttcacgactggatgtcgg | FUT1 | 1 | agg | 48,748,081 |
| 303 | ctggatgtcggaggagtacg | FUT1 | 1 | cgg | 48,748,072 |
| 304 | aagccagagagcttcaggaa | FUT1 | −1 | agg | 48,748,049 |
| 305 | aggggaagccagagagcttc | FUT1 | −1 | agg | 48,748,044 |
| 306 | gatcctttcctgaagctctc | FUT1 | 1 | tgg | 48,748,041 |
| 307 | ggaagaaagtccaagagcag | FUT1 | −1 | ggg | 48,748,026 |
| 308 | tctctggcttcccctgctct | FUT1 | 1 | tgg | 48,748,025 |
| 309 | tggaagaaagtccaagagca | FUT1 | −1 | ggg | 48,748,025 |
| 310 | gtggaagaaagtccaagagc | FUT1 | −1 | agg | 48,748,024 |
| 311 | ggatctgttcccggagatgg | FUT1 | −1 | tgg | 48,748,005 |
| 312 | ggactttcttccaccatctc | FUT1 | 1 | cgg | 48,748,004 |
| 313 | gactttcttccaccatctcc | FUT1 | 1 | ggg | 48,748,003 |
| 314 | tgcggatctgttcccggaga | FUT1 | −1 | tgg | 48,748,002 |
| 315 | actctctgcggatctgttcc | FUT1 | −1 | cgg | 48,747,996 |
| 316 | cgtgcagggtgaactctctg | FUT1 | −1 | cgg | 48,747,984 |
| 317 | ttcccgaaggtggtcgtgca | FUT1 | −1 | ggg | 48,747,970 |
| 318 | cttcccgaaggtggtcgtgc | FUT1 | −1 | agg | 48,747,969 |
| 319 | tcaccctgcacgaccacctt | FUT1 | 1 | cgg | 48,747,962 |
| 320 | caccctgcacgaccaccttc | FUT1 | 1 | ggg | 48,747,961 |
| 321 | tctgcgcctcttcccgaagg | FUT1 | −1 | tgg | 48,747,960 |
| 322 | cactctgcgcctcttcccga | FUT1 | −1 | agg | 48,747,957 |
| 323 | gcacgaccaccttcgggaag | FUT1 | 1 | agg | 48,747,955 |
| 324 | ggaagaggcgcagagtgtgc | FUT1 | 1 | tgg | 48,747,940 |
| 325 | gaagaggcgcagagtgtgct | FUT1 | 1 | ggg | 48,747,939 |
| 326 | tgtgctgggtcagctccgcc | FUT1 | 1 | tgg | 48,747,925 |
| 327 | gtgctgggtcagctccgcct | FUT1 | 1 | ggg | 48,747,924 |
| 328 | ggtcccctgtgcggcccagg | FUT1 | −1 | cgg | 48,747,921 |
| 329 | ggcggtcccctgtgcggccc | FUT1 | −1 | agg | 48,747,918 |
| 330 | cagctccgcctgggccgcac | FUT1 | 1 | agg | 48,747,915 |
| 331 | agctccgcctgggccgcaca | FUT1 | 1 | ggg | 48,747,914 |
| 332 | gctccgcctgggccgcacag | FUT1 | 1 | ggg | 48,747,913 |
| 333 | tgcgcgggcggtcccctgtg | FUT1 | −1 | cgg | 48,747,912 |
| 334 | cgccgacaaaggtgcgcggg | FUT1 | −1 | cgg | 48,747,900 |
| 335 | ggacgccgacaaaggtgcgc | FUT1 | −1 | ggg | 48,747,897 |
| 336 | tggacgccgacaaaggtgcg | FUT1 | −1 | cgg | 48,747,896 |
| 337 | gaccgcccgcgcacctttgt | FUT1 | 1 | cgg | 48,747,891 |
| 338 | gcgcacgtggacgccgacaa | FUT1 | −1 | agg | 48,747,889 |
| 339 | gatagtccccacggcgcacg | FUT1 | −1 | tgg | 48,747,876 |
| 340 | gtcggcgtccacgtgcgccg | FUT1 | 1 | tgg | 48,747,873 |
| 341 | tcggcgtccacgtgcgccgt | FUT1 | 1 | ggg | 48,747,872 |
| 342 | cggcgtccacgtgcgccgtg | FUT1 | 1 | ggg | 48,747,871 |
| 343 | taacctgcagatagtcccca | FUT1 | −1 | cgg | 48,747,867 |
| 344 | gcgccgtggggactatctgc | FUT1 | 1 | agg | 48,747,859 |
| 345 | tgcaggttatgcctcagcgc | FUT1 | 1 | tgg | 48,747,842 |
| 346 | accacacccttccagcgctg | FUT1 | −1 | agg | 48,747,842 |
| 347 | ggttatgcctcagcgctgga | FUT1 | 1 | agg | 48,747,838 |
| 348 | gttatgcctcagcgctggaa | FUT1 | 1 | ggg | 48,747,837 |
| 349 | gcctcagcgctggaagggtg | FUT1 | 1 | tgg | 48,747,832 |
| 350 | tcagcgctggaagggtgtgg | FUT1 | 1 | tgg | 48,747,829 |
| 351 | cagcgctggaagggtgtggt | FUT1 | 1 | ggg | 48,747,828 |
| 352 | tgggcgacagcgcctacctc | FUT1 | 1 | cgg | 48,747,809 |
| 353 | gtccatggcctgccggaggt | FUT1 | −1 | agg | 48,747,808 |
| 354 | cgacagcgcctacctccggc | FUT1 | 1 | agg | 48,747,805 |
| 355 | accagtccatggcctgccgg | FUT1 | −1 | agg | 48,747,804 |
| 356 | ggaaccagtccatggcctgc | FUT1 | −1 | cgg | 48,747,801 |
| 357 | cgcctacctccggcaggcca | FUT1 | 1 | tgg | 48,747,799 |
| 358 | acctccggcaggccatggac | FUT1 | 1 | tgg | 48,747,794 |
| 359 | ccgtgcccggaaccagtcca | FUT1 | −1 | tgg | 48,747,793 |
| 360 | ggcaggccatggactggttc | FUT1 | 1 | cgg | 48,747,788 |
| 361 | gcaggccatggactggttcc | FUT1 | 1 | ggg | 48,747,787 |
| 362 | ccatggactggttccgggca | FUT1 | 1 | cgg | 48,747,782 |
| 363 | cgggggcttcgtgccgtgcc | FUT1 | −1 | cgg | 48,747,780 |
| 364 | gctggtgaccacgaaaacgg | FUT1 | −1 | ggg | 48,747,763 |
| 365 | tgctggtgaccacgaaaacg | FUT1 | −1 | ggg | 48,747,762 |
| 366 | ttgctggtgaccacgaaaac | FUT1 | −1 | ggg | 48,747,761 |
| 367 | gcacgaagcccccgttttcg | FUT1 | 1 | tgg | 48,747,760 |
| 368 | gttgctggtgaccacgaaaa | FUT1 | −1 | cgg | 48,747,760 |
| 369 | gttttcgtggtcaccagcaa | FUT1 | 1 | cgg | 48,747,747 |
| 370 | acaccactccatgccgttgc | FUT1 | −1 | tgg | 48,747,745 |
| 371 | cgtggtcaccagcaacggca | FUT1 | 1 | tgg | 48,747,742 |
| 372 | tcaccagcaacggcatggag | FUT1 | 1 | tgg | 48,747,737 |
| 373 | agaaaacatcgacacctccc | FUT1 | 1 | agg | 48,747,709 |
| 374 | gaaaacatcgacacctccca | FUT1 | 1 | ggg | 48,747,708 |
| 375 | aaacgtcacatcgccctggg | FUT1 | −1 | agg | 48,747,706 |
| 376 | agcaaacgtcacatcgccct | FUT1 | −1 | ggg | 48,747,703 |
| 377 | cagcaaacgtcacatcgccc | FUT1 | −1 | tgg | 48,747,702 |
| 378 | cagggcgatgtgacgtttgc | FUT1 | 1 | tgg | 48,747,690 |
| 379 | gatgtgacgtttgctggcga | FUT1 | 1 | tgg | 48,747,684 |
| 380 | gacgtttgctggcgatggac | FUT1 | 1 | agg | 48,747,679 |
| 381 | gtttgctggcgatggacagg | FUT1 | 1 | agg | 48,747,676 |
| 382 | atggacaggaggctacaccg | FUT1 | 1 | tgg | 48,747,665 |
| 383 | agcagggcaaagtotttcca | FUT1 | −1 | cgg | 48,747,659 |
| 384 | gtggttgcactgtgtgagca | FUT1 | −1 | ggg | 48,747,643 |
| 385 | tgtggttgcactgtgtgagc | FUT1 | −1 | agg | 48,747,642 |
| 386 | tgccaatggtcataatggtg | FUT1 | −1 | tgg | 48,747,624 |
| 387 | gaaggtgccaatggtcataa | FUT1 | −1 | tgg | 48,747,619 |
| 388 | aaccacaccattatgaccat | FUT1 | 1 | tgg | 48,747,615 |
| 389 | ccagaagccgaaggtgccaa | FUT1 | −1 | tgg | 48,747,610 |
| 390 | attatgaccattggcacctt | FUT1 | 1 | cgg | 48,747,606 |
| 391 | gtaggcagcccagaagccga | FUT1 | −1 | agg | 48,747,601 |
| 392 | ccattggcaccttcggcttc | FUT1 | 1 | tgg | 48,747,599 |
| 393 | cattggcaccttcggcttct | FUT1 | 1 | ggg | 48,747,598 |
| 394 | cggcttctgggctgcctacc | FUT1 | 1 | tgg | 48,747,586 |
| 395 | agtgtctccgccagccaggt | FUT1 | −1 | agg | 48,747,583 |
| 396 | ttctgggctgcctacctggc | FUT1 | 1 | tgg | 48,747,582 |
| 397 | tgggctgcctacctggctgg | FUT1 | 1 | cgg | 48,747,579 |
| 398 | agacagtgtctccgccagcc | FUT1 | −1 | agg | 48,747,579 |
| 399 | tggcggagacactgtctacc | FUT1 | 1 | tgg | 48,747,562 |
| 400 | ctggcagggtgaagttggcc | FUT1 | −1 | agg | 48,747,555 |
| 401 | agagtctggcagggtgaagt | FUT1 | −1 | tgg | 48,747,550 |
| 402 | caggaactcagagtctggca | FUT1 | −1 | ggg | 48,747,541 |
| 403 | tcaggaactcagagtctggc | FUT1 | −1 | agg | 48,747,540 |
| 404 | atcttcaggaactcagagtc | FUT1 | −1 | tgg | 48,747,536 |
| 405 | cctccggcttaaagatottc | FUT1 | −1 | agg | 48,747,522 |
| 406 | gttcctgaagatctttaagc | FUT1 | 1 | cgg | 48,747,514 |
| 407 | cctgaagatctttaagccgg | FUT1 | 1 | agg | 48,747,511 |
| 408 | gaagatctttaagccggagg | FUT1 | 1 | cgg | 48,747,508 |
| 409 | tcgggcaggaaggccgcctc | FUT1 | −1 | cgg | 48,747,506 |
| 410 | gcccacccactcgggcagga | FUT1 | −1 | agg | 48,747,496 |
| 411 | taatgcccacccactcgggc | FUT1 | −1 | agg | 48,747,492 |
| 412 | aggcggccttcctgcccgag | FUT1 | 1 | tgg | 48,747,491 |
| 413 | ggcggccttcctgcccgagt | FUT1 | 1 | ggg | 48,747,490 |
| 414 | gcattaatgcccacccactc | FUT1 | −1 | ggg | 48,747,488 |
| 415 | ggccttcctgcccgagtggg | FUT1 | 1 | tgg | 48,747,487 |
| 416 | tgcattaatgcccacccact | FUT1 | −1 | cgg | 48,747,487 |
| 417 | gccttcctgcccgagtgggt | FUT1 | 1 | ggg | 48,747,486 |
| 418 | atgcagacttgtctccactc | FUT1 | 1 | tgg | 48,747,458 |
| 419 | ggcttagccaatgtccagag | FUT1 | −1 | tgg | 48,747,455 |
| 420 | cttgtctccactctggacat | FUT1 | 1 | tgg | 48,747,451 |
| 421 | ggcagcgccggacagaccgc | RHD | −1 | ggg | 25,272,568 |
| 422 | aggcagcgccggacagaccg | RHD | −1 | cgg | 25,272,569 |
| 423 | ctaagtacccgcggtctgtc | RHD | 1 | cgg | 25,272,572 |
| 424 | cccagaggggcaggcagcgc | RHD | −1 | cgg | 25,272,580 |
| 425 | gtgttagggcccagaggggc | RHD | −1 | agg | 25,272,589 |
| 426 | tccggcgctgcctgcccctc | RHD | 1 | tgg | 25,272,590 |
| 427 | ccggcgctgcctgcccctct | RHD | 1 | ggg | 25,272,591 |
| 428 | tccagtgttagggcccagag | RHD | −1 | ggg | 25,272,593 |
| 429 | ttccagtgttagggcccaga | RHD | −1 | ggg | 25,272,594 |
| 430 | cttccagtgttagggcccag | RHD | −1 | agg | 25,272,595 |
| 431 | gcccctctgggccctaacac | RHD | 1 | tgg | 25,272,603 |
| 432 | gagagctgcttccagtgtta | RHD | −1 | ggg | 25,272,603 |
| 433 | tgagagctgcttccagtgtt | RHD | −1 | agg | 25,272,604 |
| 434 | agtgggtaaaaaaatagaag | RHD | −1 | agg | 25,272,631 |
| 435 | ctctaaggaagcgtcatagt | RHD | −1 | ggg | 25,272,648 |
| 436 | cctctaaggaagcgtcatag | RHD | −1 | tgg | 25,272,649 |
| 437 | ccactatgacgcttccttag | RHD | 1 | agg | 25,272,660 |
| 438 | gagccccttttgatcctcta | RHD | −1 | agg | 25,272,663 |
| 439 | cgcttccttagaggatcaaa | RHD | 1 | agg | 25,272,669 |
| 440 | gcttccttagaggatcaaaa | RHD | 1 | ggg | 25,272,670 |
| 441 | cttccttagaggatcaaaag | RHD | 1 | ggg | 25,272,671 |
| 442 | agaggatcaaaaggggctcg | RHD | 1 | tgg | 25,272,678 |
| 443 | ccgccatcacggtcagatct | RHD | −1 | tgg | 25,284,583 |
| 444 | tggccaagatctgaccgtga | RHD | 1 | tgg | 25,284,591 |
| 445 | ccaagatctgaccgtgatgg | RHD | 1 | cgg | 25,284,594 |
| 446 | caagccaatggccgccatca | RHD | −1 | cgg | 25,284,594 |
| 447 | ctgaccgtgatggcggccat | RHD | 1 | tgg | 25,284,601 |
| 448 | cgtgatggcggccattggct | RHD | 1 | tgg | 25,284,606 |
| 449 | ggtgaggaagcccaagccaa | RHD | −1 | tgg | 25,284,606 |
| 450 | gtgatggcggccattggctt | RHD | 1 | ggg | 25,284,607 |
| 451 | gtctccggaaactcgaggtg | RHD | −1 | agg | 25,284,622 |
| 452 | gctgtgtctccggaaactcg | RHD | −1 | agg | 25,284,627 |
| 453 | gcttcctcacctcgagtttc | RHD | 1 | cgg | 25,284,629 |
| 454 | cactgctccagctgtgtctc | RHD | −1 | cgg | 25,284,637 |
| 455 | cgagtttccggagacacagc | RHD | 1 | tgg | 25,284,641 |
| 456 | gagacacagctggagcagtg | RHD | 1 | tgg | 25,284,651 |
| 457 | cgccagcatgaagaggttga | RHD | −1 | agg | 25,284,663 |
| 458 | caccaagcgccagcatgaag | RHD | −1 | agg | 25,284,670 |
| 459 | ggccttcaacctcttcatgc | RHD | 1 | tgg | 25,284,672 |
| 460 | aacctcttcatgctggcgct | RHD | 1 | tgg | 25,284,679 |
| 461 | tgctggcgcttggtgtgcag | RHD | 1 | tgg | 25,284,689 |
| 462 | gctggcgcttggtgtgcagt | RHD | 1 | ggg | 25,284,690 |
| 463 | tgtgcagtgggcaatcctgc | RHD | 1 | tgg | 25,284,702 |
| 464 | cagtgggcaatcctgctgga | RHD | 1 | cgg | 25,284,706 |
| 465 | ggctcaggaagccgtccagc | RHD | −1 | agg | 25,284,706 |
| 466 | tcccagaagggaactggctc | RHD | −1 | agg | 25,284,721 |
| 467 | ccaccttcccagaagggaac | RHD | −1 | tgg | 25,284,727 |
| 468 | ttcctgagccagttcccttc | RHD | 1 | tgg | 25,284,730 |
| 469 | tcctgagccagttcccttct | RHD | 1 | ggg | 25,284,731 |
| 470 | tgatgaccaccttcccagaa | RHD | −1 | ggg | 25,284,733 |
| 471 | gtgatgaccaccttcccaga | RHD | −1 | agg | 25,284,734 |
| 472 | gagccagttcccttctggga | RHD | 1 | agg | 25,284,735 |
| 473 | ccagttcccttctgggaagg | RHD | 1 | tgg | 25,284,738 |
| 474 | caccgacaaagcactcatgg | RHD | −1 | tgg | 25,290,658 |
| 475 | cagcaccgacaaagcactca | RHD | −1 | tgg | 25,290,661 |
| 476 | ggccaccatgagtgctttgt | RHD | 1 | cgg | 25,290,667 |
| 477 | tttgtcggtgctgatctcag | RHD | 1 | tgg | 25,290,682 |
| 478 | gatctcagtggatgctgtct | RHD | 1 | tgg | 25,290,694 |
| 479 | atctcagtggatgctgtctt | RHD | 1 | ggg | 25,290,695 |
| 480 | tctcagtggatgctgtcttg | RHD | 1 | ggg | 25,290,696 |
| 481 | agtggatgctgtcttgggga | RHD | 1 | agg | 25,290,700 |
| 482 | tgtcttggggaaggtcaact | RHD | 1 | tgg | 25,290,709 |
| 483 | gaaggtcaacttggcgcagt | RHD | 1 | tgg | 25,290,718 |
| 484 | ggtcaacttggcgcagttgg | RHD | 1 | tgg | 25,290,721 |
| 485 | cttggcgcagttggtggtga | RHD | 1 | tgg | 25,290,727 |
| 486 | gcagttggtggtgatggtgc | RHD | 1 | tgg | 25,290,733 |
| 487 | gttggtggtgatggtgctgg | RHD | 1 | tgg | 25,290,736 |
| 488 | ggtggtgatggtgctggtgg | RHD | 1 | agg | 25,290,739 |
| 489 | ctggtggaggtgacagcttt | RHD | 1 | agg | 25,290,752 |
| 490 | tgacagctttaggcaacctg | RHD | 1 | agg | 25,290,762 |
| 491 | agctttaggcaacctgagga | RHD | 1 | tgg | 25,290,766 |
| 492 | tattactgatgaccatcctc | RHD | −1 | agg | 25,290,767 |
| 493 | agatgtgcatcatgttcatg | RHD | −1 | tgg | 25,300,960 |
| 494 | tacgtgttcgcagcctattt | RHD | 1 | tgg | 25,300,993 |
| 495 | acgtgttcgcagcctatttt | RHD | 1 | ggg | 25,300,994 |
| 496 | ggccacagacagcccaaaat | RHD | −1 | agg | 25,300,995 |
| 497 | agcctattttgggctgtctg | RHD | 1 | tgg | 25,301,004 |
| 498 | attttgggctgtctgtggcc | RHD | 1 | tgg | 25,301,009 |
| 499 | tagaggctttggcaggcacc | RHD | −1 | agg | 25,301,016 |
| 500 | cctcgggtagaggctttggc | RHD | −1 | agg | 25,301,023 |
| 501 | gttccctcgggtagaggctt | RHD | −1 | tgg | 25,301,027 |
| 502 | tcctccgttccctcgggtag | RHD | −1 | agg | 25,301,033 |
| 503 | cctgccaaagcctctacccg | RHD | 1 | agg | 25,301,034 |
| 504 | ctgccaaagcctctacccga | RHD | 1 | ggg | 25,301,035 |
| 505 | tctttatcctccgttccctc | RHD | −1 | ggg | 25,301,039 |
| 506 | aaagcctctacccgagggaa | RHD | 1 | cgg | 25,301,040 |
| 507 | atctttatcctccgttccct | RHD | −1 | cgg | 25,301,040 |
| 508 | gcctctacccgagggaacgg | RHD | 1 | agg | 25,301,043 |
| 509 | acccagtttgtctgccatgc | RHD | 1 | tgg | 25,301,088 |
| 510 | ccagaacatccacaagaaga | RHD | −1 | ggg | 25,301,529 |
| 511 | gccagaacatccacaagaag | RHD | −1 | agg | 25,301,530 |
| 512 | ccctcttcttgtggatgttc | RHD | 1 | tgg | 25,301,540 |
| 513 | agcagagcagagttgaaact | RHD | −1 | tgg | 25,301,552 |
| 514 | tgctgagaagtccaatcgaa | RHD | 1 | agg | 25,301,582 |
| 515 | acggcattcttcctttcgat | RHD | −1 | tgg | 25,301,582 |
| 516 | agcatagtaggtgttgaaca | RHD | −1 | cgg | 25,301,601 |
| 517 | gctgactgctacagcatagt | RHD | −1 | agg | 25,301,613 |
| 518 | ctatgctgtagcagtcagcg | RHD | 1 | tgg | 25,301,628 |
| 519 | agcgtggtgacagccatctc | RHD | 1 | agg | 25,301,644 |
| 520 | gcgtggtgacagccatctca | RHD | 1 | ggg | 25,301,645 |
| 521 | agccaaggatgaccctgaga | RHD | −1 | tgg | 25,301,646 |
| 522 | agccatctcagggtcatcct | RHD | 1 | tgg | 25,301,655 |
| 523 | cttcccttgggggtgagcca | RHD | −1 | agg | 25,301,661 |
| 524 | tcatccttggctcaccccca | RHD | 1 | agg | 25,301,668 |
| 525 | catccttggctcacccccaa | RHD | 1 | ggg | 25,301,669 |
| 526 | ttatgtgcacagtgcggtgt | RHD | 1 | tgg | 25,303,341 |
| 527 | gtgcacagtgcggtgttggc | RHD | 1 | agg | 25,303,345 |
| 528 | cacagtgcggtgttggcagg | RHD | 1 | agg | 25,303,348 |
| 529 | tgcggtgttggcaggaggcg | RHD | 1 | tgg | 25,303,353 |
| 530 | gttggcaggaggcgtggctg | RHD | 1 | tgg | 25,303,359 |
| 531 | ttggcaggaggcgtggctgt | RHD | 1 | ggg | 25,303,360 |
| 532 | agaagggatcaggtgacacg | RHD | −1 | agg | 25,303,374 |
| 533 | caagccacggagaagggatc | RHD | −1 | agg | 25,303,384 |
| 534 | ccatggcaagccacggagaa | RHD | −1 | ggg | 25,303,390 |
| 535 | gtcacctgatcccttctccg | RHD | 1 | tgg | 25,303,391 |
| 536 | accatggcaagccacggaga | RHD | −1 | agg | 25,303,391 |
| 537 | cccagcaccatggcaagcca | RHD | −1 | cgg | 25,303,397 |
| 538 | cccttctccgtggcttgcca | RHD | 1 | tgg | 25,303,401 |
| 539 | tccgtggcttgccatggtgc | RHD | 1 | tgg | 25,303,407 |
| 540 | agccacaagacccagcacca | RHD | −1 | tgg | 25,303,407 |
| 541 | ccgtggcttgccatggtgct | RHD | 1 | ggg | 25,303,408 |
| 542 | tgccatggtgctgggtcttg | RHD | 1 | tgg | 25,303,416 |
| 543 | atggtgctgggtcttgtggc | RHD | 1 | tgg | 25,303,420 |
| 544 | tggtgctgggtcttgtggct | RHD | 1 | ggg | 25,303,421 |
| 545 | gtggctgggctgatctccgt | RHD | 1 | cgg | 25,303,435 |
| 546 | tggctgggctgatctccgtc | RHD | 1 | ggg | 25,303,436 |
| 547 | ggctgggctgatctccgtcg | RHD | 1 | ggg | 25,303,437 |
| 548 | gctgggctgatctccgtcgg | RHD | 1 | ggg | 25,303,438 |
| 549 | caggtacttggctcccccga | RHD | −1 | cgg | 25,303,440 |
| 550 | gggtgttgtaaccgagtgct | RHD | 1 | ggg | 25,306,613 |
| 551 | tgtggggaatccccagcact | RHD | −1 | cgg | 25,306,613 |
| 552 | ggtgttgtaaccgagtgctg | RHD | 1 | ggg | 25,306,614 |
| 553 | tagcccatgatggagctgtg | RHD | −1 | ggg | 25,306,629 |
| 554 | gtagcccatgatggagctgt | RHD | −1 | ggg | 25,306,630 |
| 555 | tgtagcccatgatggagctg | RHD | −1 | tgg | 25,306,631 |
| 556 | gattccccacagctccatca | RHD | 1 | tgg | 25,306,636 |
| 557 | attccccacagctccatcat | RHD | 1 | ggg | 25,306,637 |
| 558 | gctgaagttgtagcccatga | RHD | −1 | tgg | 25,306,639 |
| 559 | gggctacaacttcagcttgc | RHD | 1 | tgg | 25,306,657 |
| 560 | ggctacaacttcagcttgct | RHD | 1 | ggg | 25,306,658 |
| 561 | ttcagcttgctgggtctgct | RHD | 1 | tgg | 25,306,667 |
| 562 | gatcatctacattgtgctgc | RHD | 1 | tgg | 25,306,693 |
| 563 | ctgctggtgcttgataccgt | RHD | 1 | cgg | 25,306,709 |
| 564 | gtgcttgataccgtcggagc | RHD | 1 | cgg | 25,306,715 |
| 565 | gataccgtcggagccggcaa | RHD | 1 | tgg | 25,306,721 |
| 566 | ccccaatgctgaggaggacc | RHD | −1 | tgg | 25,317,015 |
| 567 | tgagttccccaatgctgagg | RHD | −1 | agg | 25,317,021 |
| 568 | ttccaggtcctcctcagcat | RHD | 1 | tgg | 25,317,024 |
| 569 | agctgagttccccaatgctg | RHD | −1 | agg | 25,317,024 |
| 570 | tccaggtcctcctcagcatt | RHD | 1 | ggg | 25,317,025 |
| 571 | ccaggtcctcctcagcattg | RHD | 1 | ggg | 25,317,026 |
| 572 | cagcattggggaactcagct | RHD | 1 | tgg | 25,317,038 |
| 573 | agacatgagagctatcacga | RHD | −1 | tgg | 25,317,050 |
| 574 | atcgtgatagctctcatgtc | RHD | 1 | tgg | 25,317,063 |
| 575 | ctttccatattttaagattt | RHD | −1 | agg | 25,321,902 |
| 576 | tgctcctaaatcttaaaata | RHD | 1 | tgg | 25,321,909 |
| 577 | aatatggaaagcacctcatg | RHD | 1 | agg | 25,321,925 |
| 578 | tcaaaatatttagcctcatg | RHD | −1 | agg | 25,321,927 |
| 579 | attttgatgaccaagttttc | RHD | 1 | tgg | 25,321,954 |
| 580 | taaaatccaacagccaaatg | RHD | −1 | agg | 25,328,907 |

In the ″Strand″ column, “1” denotes the forward strand; “−1” denotes the reverse strand.

SEQ ID NO: 1 (ABO):
agtgtaaactcctctgagagatatcaggaaaagcaggaagaagcctctgggacccttcgggaggtaactcctcttcg
cagcggggcgcgctctcccagtccctgcagccgccgccgccctctcctgagcttcctcgagcggacgccaggcaag
ggcgggggtcgtagcggggcggagcggggctttgtccacggaccgcgcgaagaggcctcagggcccggcgcgg
gcgccggagggggactcgctcgcagggggaacgcgaaggttcctcagtctgcgggacgcagagctccgtggggc
cgcgagccggggccggggaagcgactctgcctagggggacgtcgcgggcgcggggcacagggtcctgcgggg
ctggagggctacaggctgcggcgcgcgcgagccggaaggccggggatcgtgggttctggggccgcagcttcacg
ggttcgtctcccccgcctccccgggggagcaggatgtcagggggtcgcccccgcccgggagacagggtgtcaagg
ggcccccggggacggggcttcaggggcacccggagccgctcggccccagggcgggatgcggggacagggccc
caaggtaccagggccacgaggggcgcgcgggtcccttggggatgcgcgcgaggaggcgccgtcccttcctagca
ggggtccctggggacccgcggccgcctcccgcgcccctctgtcccctcccgtgttcggcctcgggaagtcggggcg
gcgggcggcgcgggccgggagggggcgcctcgggctcaccccgccccagggccgccgggcggaaggcggag
gccgagaccagacgcggagccatggccgaggtgttgcggacgctggccggtgagtgcaggcctcggccccgggt
gcccgcgagggagccgctaccgcagggaatgcggggtgcacccgacagccgggccggggtgggggcgctcag
ggctgcgaggcttcgggccggccgccgccccagcctccgagaccctgcgtcctggggagccggcgggcaggtgg
gctcggccgcgctgtgggtgcctgggacccgcagggaggatgggcgcggtggcgcggcctggcggggggctcgt
ctccggggtccccgggtcctggtgagagcggggtccctcgacgccgtggcggtctccagcctctcctcgcccctccac
gctccccgccttccatgagctgctattttcagcacctaccgcccgaccctggactaggacaaggctctgggctgccctg
cccgccccccagcccttccctcgggcacggcggccaggcgcccgggttgaccgggaacagcctccataccccaa
acgcggaggcgcctcgggaaggcgaggtgggcaagttcaatgccaagcgtgacgggggaactgtgccccgggc
cctcaggtgatataggagttaagaagaaattattgaggcaaccagatgcggtgactcaggcctgtaaccccagcact
ttgggaggccgagggtggatcacctgtccttaattttcttggcgccagaagatgaattgagtatttacccagacaacaa
cgtcgcttcagagggagggatgcagaacgcagggccacggggtgcaggctgcaggccagtgaaccccaacgcc
aaaggccagggagagccgggtggggtacccagagccagcacacagccctttaatttagaggagtgctgtgtacac
atctggggagagatgttttactttgatttggaatcaggtggcggataaggcatactgaggcctgacttggtgagggctcc
tgccccggaggtgcagccctggaggagcgggaggcagaggagtggaaaattcatgaagaaaaccgggtatggt
gtaggtcgaggccctgccctcagtaatgctcaccatttgtcagtgtttactgtgaggcagcactgtgttcagtatcgctga
gttctcaggaaggaacggtaaatacttcccgggtcattctttcacccacgggaaaacaggtttggagagatctgggac
agtgctctggtcctaggcaggaagggctgagtggggcctgggactcaggtctgactgcaaacacctgcctcctccct
gtgctgccagcgccttccgggttcttccctgtccctcctttgtggtctttgttttcccttttttgtcttaatatgtttcaacggatgtat
acaataaaccgcacatgaaaggtacagctggatacatcttgacccagtcaagatgatgaacacagctgccacccc
aggagtctgtcctgccccacgggttatgctgtcttagttggtctcatgtcagggagccttggaggaccagggtcgggca
gttggtctctatactcctggggttctggcactggctctggcccatgaccgcacccaagacaaacgtcttgaagactaag
aggttaggtctttgagaaacctggcaaatgagtgcccattctcaggttacccacattctgcatgttgatttagtcatccaac
caatgttggttgaacactgatgagaacaagcaggcctgtgctagaaggtgcctgcagccaggagctggtgagctggt
gtccttagggacaccaatggcgagggacccagtgtgtggaaatctgggggacaagcatgccagggagaagagct
cacatggggaaggcccggctccacaaatcagtcaggcttgttggggcgggcaggagagcagggtagtggagtca
gagggagtgatcccccgaaaggcaggaagaggacatgagagagccttggagatgacggtgaggatgtggttggt
gggcggtgagctgggtgtcatgtgctggctccttagagaatgctcagctccttcacacccatcataatccctggaggac
tgagaccacgtgcaggagttttggaagctggcagtgcacccagtcccggctctcctccattctggtgggtctcaccaga
gattggccaagaagagatcaaactgttcctggaccaaactgagggtggggctgctatctctcgcggcccaataacga
gatgcagatgaactggggagaaagagagtttttatttctgtaaccagttacaaggagaagacctggaaattatctcca
gaccaactcaaaattacaaagttttccagagcttatataccttctaagctatatgtctatgtgtaagtgtgcattcatctcaa
gacataagtaattgacttatgttaatctataactaaggtctgagtcctgaagaccttcctctggatcctcagtaaatttactt
aatctaaaacccttatcttgtctcctaaatcatgggggtttgggaagttccttcagacccccagtaaacttatttgtggagtc
ctggggaatttcttcagatccccaataaaacttgtttaatcctaaatgggtcctgttaagaattccttcgttattttgtcatgctt
gaaggcccaggaaaggtctaggcaaaactcttggtgggattttgttatattccagcctttttataagggcactggcttttaa
tatttaatttaaccactcagtcagtactgaaacagttgttagggaggcctgcgttagtgagacctgacctgccacaacac
atcttactcggaatgctgcccataacttcaaaaaatcagctttgacggagccctactgaacacacctagcatctctcttc
cttcagcttagggtcaaggggctggggttgatggcaccattgaaagaaacagctttattgccgtgtcattgatatgccat
aaaattcacctgtttcaaatgaattattttcagttagtttacagagttgtgaaataaattttataacttttccatccccagccca
ccaaaactccctggaactcctccgcagtcattccccattcccacctggcctcagacaatcactttctgtctctccagtcttg
ccttttctggacagttcctatgaatggagtcctgtgttacatggtcttttgcatctgacttccttcacttagaataatgattccga
gattcatgtatgttgtagtatgtatcagtatttaattcctttttattactgaataatccattgtacagatagaccacattttgtttat
ccattcatcagctgaaggacatttcggctgtttctgcttttttagctattttaaactgcacgcagcactgctatgaacatttgtg
tacaagactttgtgtgaacatgttttcatttctcttgggttgatacccagcagaggaattgctgggtcatacagaaagtctg
atttaacattttaagaaactagcaaactgttttccaaagtgactgccccattttacattcccatcagcagtgtataacggttc
taattttcttttcctttttcttttcttgagacaaggttttgctctgtcacccaggctggagtgcagtggcatgatcttggctcactg
cagcctcaatctcttgggctcaattggtcctcccacctcagcctcctgagtagctgggactacaggtgagtaccaccac
atccagctaatttttgtatttttggtagagatggggttttgccatgttgcccaggctgttctcaaactcctgggctcaagtgat
ctgcccacctcggcctccccaagtcctgggattataggtgtgagccgctgcgcctggctgagggttccactgtctgtac
gtctgcagcaatacataccattcttgtgggtaaaaggtggtatctcattatagttttgatttgcatttccccaaggtcaaatg
atggcaagtggcttttcttgtgctttttagccatttgtatatgtttttggtgaaatgcctattgaaatgttttgcctttttaaaaattg
agttgtctcctttgttcagttttgagagttctttacatacacagtatcatatatatatatatatatatatatatatatatatatatata
cacaccagatatatgatttgcaaatattttcaaccatcaatagtttaacacttttttattggtattttgaagtagaaaagtttta
cattttgatccattccaatttattaactttttttttattgtgtatctggtatcatatctaagaaatcttaatccagtgtcacaaaga
tttaatcttatattttcttctaaccgctttctagtttatgtgtaagaatctgtccattttacctaagttgcataatttgttggcaaaca
gttgtacatagtatttccttgaaatccttttaatctctgtaagattggaattgctgtctcctcttttagtcctgattttagttatttgtgt
tctctctctctctggtcaatgtagctaaagctttgtcaattctcttgatcttttcagagaactgccatttgatatttttttactttatctt
tttctttctgttctctagttcattggtttccactcttctcactttgggtttaattcgttctgtcttttcttattgtagttacttacaatggaa
gcttacacacttgatttaagatttttttttctaatgtagacatttacagctataaatttcccttgaaacacagctttagttatatct
cataaattttggtatgttgtgtttacattttcattcagctcagtgtatcttttgattttcttctttgacccattgcttatttagaattatgt
tgtttaatttctatgtatttataggtttcccaaatttcttttgttaatttctgatttcattcccctgtggctagagaagaaactctgtg
gatatcagtcctttcgaatttctcagaattgttttatggtccagcatatgattgaccttggacactgttccatgtgcacttgag
gagaacgtgacttcggctcttgccaggtggagtgttctagagatgtcagttggtgtccagtgatgtcaagtcatttgtatct
actgattttctttctaattattctatccattactgagagtggggtaggattttggtgtccaattattgtttttttgtttttttttttttgtttgtt
tttttttttttttgaggtggagtctcactttgtcacccaggctggagtgcagtggtatgatcacagctcactgcagcccccac
ctcccaaggcttaggtcacctcagcctcctgagtagctgcaactacaggcaagtaccatcatgcctagctgatttttgca
ttttttatagacagggttttgccatgttacttaggctggtcacgaactcctgggctcaagtgacccacccacctcggcctcc
caaagtgctgggattacaagcgtgagtcaccatgcctggcccaaccattactggtgaattgactgtttcacccttcaatt
ctgtgagtttttgcttcatgtattgctgcgggataattaaggaatcagagagaccgatggggttgaggaggaattatttaa
ttatttaggtgcaccgacccaatcagattaacatccaaaggactgggccccaaacaaagagtcaagctaccttttaag
cattttgtggggtggggggagatttgtgcagggggaagagtattacagaagcaagaaacaaagacagttattcagtt
aagacatgcattacattatttcttacttttcaaggaacaacacgttttatgactcaagattatctgtttagtgaccttgcagct
gcacagctagagaaacagagtcttcgcaatgcctgggaaagggagagataaggctcactagccacagaaaaac
agccagttaatttttaaaggactccagccctttctcttcctcaaggggaattggttttttacatacaactgagtttttgcttaca
cagtttttaatttcttttaattcctgttctagtattttggggctaggttgtcaggtatgtatatatttctgtttgttatattttcgtgatgta
ttcactttatatcatggcagaatgtttctctttagtaagatttttgatcttaaaaaagttggccagatggggggctcatgcct
gtgatcccagcactttgggaggccaaggcaggtggatcacctgaggtcaggagttcaagactagcctggcgaacat
ggtgaaaccccgtctctactgaaaatacaaaaaaattagccagctgtgatggtgtgcgcctgtaatccctgctacttgg
gaggctgaggcacgagaattgcttgaacccgggaggctgaggttacagtcagccaagatcgtgccactgcactcca
acctgggtgatagagtgagactctgtctcaaaaaaacaaaaaaattatttagtctgacgttagcatttctcctcaagctc
ccttacggttgtttgcatagcaaatctactatccttttgctttgacctatttgtatctttgtttctaaagtatgtatgtctctcacag
gcagcatatagtcaaagcttaaaaaaaaaatccagtctaacgatctctgccttttgattggcatgttcattctattcccattc
aatgttattattgctgtggttggatttccatctatcagttcacaatttgttttctatgttttatgcctttttgttcttctgttcatcttttact
accttcttttgtattaagtattttctagtgtagcatttaaattcccttttttttttaagtgtatattttaaagttattttgttagtgtttgct
ccagggattacaatatgcattttaatttatcaggatctacttcagattaatactaattttagtaaaatacaggaacttgactc
cagtataactccatttcctccctccttgcttgtggtttgtagtattattgtcgtatatgttcatctatatatgttataaactcaaca
acatggtgttataattattgtttcacacaatcttatttcttttcaattcagtaagacaagtaaggagaaaaacacttttcaagt
cttttatattacactgtatatttatcactgactttactcttgatttcttcctgtatattcaagctattgtctggtgacctttccttgctcc
agtatatataataacttcattgcctccttgctcctttatgctattattgtcatatatattatatatgtttatgctgtgagcccatcag
ctaagtcagcttagcaaggtctcaagatacaaagtcaatgaataaatcaggctgggtgtggtggctcacacctgcaat
cccagcactttgggaggccaagataggtagatcacttgaggccaggagtttgagagcagcctggcccacatggcaa
gactccatctttacaaaaatacaaaaaaaaaaaaaaagttgagtgtggtggtgcgcctgtaatcccacctacttggga
ggctgaggcatgagaattgcttgaacccaggagggagaggttgcagtgactggagatcacaccactgcactccag
actgggcaacagggcaagacacagtctaaaaaaaaaaaaaaaagaaaaaagaaaaacaagaataaatcaatt
atttttctataatacttgccacaatcaattgaaccatgaaaaattttaaataccatttacagtagcatcataaaacatgaaa
tatttagagaataatttaccaaattaggagaaatgtctatacaattaaaactgcaatataaaccaggcaccatggctca
tgcctgtaatcccagcactttgggaggtcaaggtggccagatcacttgagcccagaagtttgagaccagcctgggtga
catagtgagaccctgtctctacaaaaaaaaaaaaaaattagctgggcatggtggcatgcacctgtagcccctgctact
caggaggctgaggtgagaggatgacttgagcccagtaggcagagattgcagtgcattccagcctgggctacacag
caagaccctgacaaaaaaaaaaaaaaaaagaaaaagaaggtcacaaaaaaacagaaaactgcaaaatattg
atgacaaaaattaaagaagacataaataaatggagaaatataccatgctcatggattggaagacttacattgctaaa
ctgtcacttctccccagatggatctacagagtccacataatctcacttaaaactccagaagaaatttttgtaaagttgaca
gctgattctaaaattttacatagtaatcagaataggttgatattaggatagacagatagttcaatggaatagaatgcaga
gtttagaaatagacctacacagaaatagtccattgattttttaagagttgctttttgataagggtgctaaggtaatgtgata
gagaaaggaaagtattttcaattcaaatggctgaaatgactggatatccattgagggaagaaagggactttagcctttc
acacaatacacaaaaattatggaattctgaagaaaataagagaaaatgttcatgaacttggggtaggcaaaaattta
atagatgaggcaaaaaaaaaaaaaggcccaaaccataaaaatggtttcatttttataaggataaattagagtttataa
aaattaacacttcccttcaaaagaaaaattaaggaaaaatgaataaataagccaccgactgaaagaaaatatttgttt
tctaagaagtattttgtttttcattttatgggttcatagtaggtgtatatatttatggggtccctgagatattgtggttcagtcatac
aatggaaaattcacatcatggagaactggtatccatcctcttgagcaattatcctttgtgttacaaacaatccaactatac
tcttttagttatttttattctttttttttttttttttgagatggagtctcgctctgtcacccaggctggagtgtggtggcgccatctcaa
ctcactgcaacctccgcctcccaggtttaagtgattctcctgcctcagtttcctgagtagctgggactacaggcacccac
caccacgcccggctagtttttgtatttttaaactttttttttctttttttttttcttctttttttttttttgagatggagtctcgctctgtcgcc
cgggctggagtgcagtggtgcaatctcggctcactgcaacctccgtctcccaggttcaggagattctcctgcctcagcc
tcccaagtagctgggattacaggcatgcgccaccacacccagctaattttttgtatttttagtagaggcaggatttcaccg
tgttggtcaggctagactcgaatgcctgacctcgtgatccacccacctcggcctcccaaagtgctggggttacgggcgt
gagccaccacgcctggccttagtttttgtatttttagtagagatgaggtttcaccatgttggccaggctggtcttgaactcct
gatctcaggtgatccacctgcctcggcctcccaaagtgctgagattacaggcatgagccactgtgcccgacctctctttt
agttatttttaaatgtgcaattaaattattattgactatagtctccctgttatgctatcaaatactgggtcttattcattctttctattt
atttgtacacattaaccatccctacataccctctcacccctgccactacccttccaagtctctgcgaaccatccttctattct
ctatctccatgagtttaattgtttcgatgttttgatactacagataagtgagaacatgcagtgtctgtctctctgtgcctggcg
agaaaatatttgtaatatttgtatttggcaaaggacttgtatccaaaatatagaaataaattctgctcaataataaaaaga
gaaacaacttaatgagacccagtgtctacaaaaagtagaataattagccgggcatgttggtgcatgtctgtagtccca
cctaatcaggaggctgagggggaaagatcacttaagctcaggagttcgaggttgcagcgagctgtgatcgtgccact
gcactccagcctgggtgacagagtaagatcctgtctaaaaaaaaaaaaatagagggccaggtgaggtggcttatg
cctgtaatcccagcacattgggaggctgaggcaggtggattgcttaagcccaggagttcaagaccagcctgggcaa
catggtgaaaccccatctctaccaaaaccacaaaaaatcagctagatgtggtgcatgcctatagtctcagctactcaa
aaggctgaggtgggaggatcacctgagcccaggaagtcgaggctgtagtgagccatgatcatgccaccacactcc
agcctgggcaactggagtgggactgtgccccccaaaaatatatataagtaaataaataaataaataaataataaaat
gagctaaagatctggacaggcttcataagaagcaagcaaatggccaataaatacatgaaaagatgatttacctcctt
agtcattggacacacttagataccactcctcatccactagcatggctaaaggataaaagagtgaccatcaagtgttgg
caaggactgtggttctcgtacattcctggcgggaatggaaaattcagtcaccactttggaacacagtttcttacaaagtt
aaacatacacttaccatatgacccatcttttccattccttggtatttactcaagggaaatgaaaacacaggtccacaaat
acctgcacatgagtgtttacagaagctttgttgctagtggccccgaacagtgaaaaaaccacagtgtctgtcaatagg
agacagaataaatgaattgtggcggattaacaatggcgtgcttctcagaaatgaaaaagaacgaacgattgataca
cgtgacaatataggtgaatcttcaaaacagcatgcggtgtgatgcggccagacataaaagagcatgtatcagagga
ttccgtgtatgggaaacgccagggaatcagatccatggttgtcagggtcaggagggaccttactggggtgattacatgt
gtacatacattgtgtgaaactcatcaaaaccgtatacctaaagtgggcacattttattggatgtaagttatccctcaaatta
aattaattttcaaagttttaaaaaatggaaccatttttgccacattgaaggaaaattatttccaccaagatttccctacagc
caaacgatctaccaactacaaaaatggaaaaaataatttaggacatgtaaagttcaaatgttttgcctcccacgtttctg
tttcaagaagctattcgagataaatcgctccgtggtcacaggacttagaaaggtggaggtaaacacacacaagcatt
ataagataagaagtaacagatgaattagttgaaagggactgatttcgggggaaggaataggaactgggccaggag
atagacgcctttcaggcttgcccttgtgaaaacgatagtcccctcttacctgcgggggataggttccgagatccccagt
ggatgtctgaaaccacagatagtaccaaaccctacagagagtatgttttctcctgtacacacatacctatgatagggttt
aatttataaattaggcaccgggtgggattaacaaccataattaatagtaaaatagaacaattataacaatatactgtcc
gaggcgggtggatcacttgaagtcaggagatcgagaccagcctggccaacatggtgaaacctgtctctactaaaaa
tacaaaaaattagccaggcgtagtggtgggcgcctgtaaacccagctactcgggaggctgaggcaggagaattgct
tgaacccgggaggcggaggctgtagtgagccgaaggtgcccctttgcactccagcctgggcaacgagcaaaactc
catctcaaaaacaacaacaaaacaacaacaacaacaaaaacaatatactgtaataaaggttacgtgaatgtggtct
ctctaaaaaaaaatgtgtatatatcttagtttgtgggttttttgttgttgtagttttgtttattattaagagacagggtcttgctctgt
tacccaggctggagtgcagtggtgtgatcatggcttactgcagccttcacctcctaggctcaagtgatccttccacctca
gcctcctgggaccagaggcatgcaccactatgcccagctaattgttttttttttttttttcttgatagaaatgagttctcactatg
ttgcccaggctggtctagaactcctggcttccagcaattctgctggctcagcctcccaaagtcctgggattataggcacg
agccacgatgcctgtcctcaaaatattttactgtactgtactcacctattattggcctttggttgaccatgggtaactgaaac
tccagcaagcgaaggtgtggataagggaactactgtacgtgactttaatatacataaaatcaccttaaagacaatgat
tgtttccagaacatgagcctctgagacagcagggagatactaggggaagttggtggctcctctccctggcaggaact
ggtctgggctcctggctcagcctggccatgaggctgtcctggcctccctcggtgggttcgacagtgacctcgacgtgct
catttcagtgtggttcattccggtctcccaggggaagggggtgctgagtggaaggaggtcaatgggaagccggggtg
gctctcagagtctgcaggagcagtcgggctgatgagctgggaggagcagaccgcctccctcttctctgagtgggagg
agggccagatctggactgggtttggagatgctcaggtggggctcagagcatcacctgtggggcagagggaccatctt
ggcagatgaaggcccgtcgcagggtgtgatgcctgaattacaaggcgggacaggtaaagtggggcaggtgagag
aaggagggtgagtgatgtgatttttctactcctgttttccaggaaaaccaaaatgccacgcacttcgacctatgatcctttt
cctaataatgcttgtcttggtcttgtttgggtaagacacatttgaccatcgaggctggcctggtttggggagaagtgacca
cagcagccaatcagacccatggggcctccctgagctccccaagtatcacagttatcagggtcctaaggacagttattg
cctgcgtccagctctggcggagggtgtgcttacttgctcccttattttagcctcacctgggcaacaggctcatctcactccc
atttaaaattttcctaagtgtggagtctggggctgggagagcaagccccttgcccacaattgcgtggctgggggtgggg
aaggcaattctgggtcccaagctgttagtcgcttccagacacagaaggtcccagaaccaagagtgaagtcacctgtc
acctctactggggcatctctggacacggtctggaaacactccctgacgtggcctcagggactgcactgaccaaggca
ctggtggcggggggtgagggagctggggctctggagctccagcaggtgcccatacgtgagcaatatcccagggac
caccctcctgcccacctcccggtgtgggacgtggcgaggcgcctgagctttgctgagaacttgccctacctgcctcga
ggccttgcagcttcaccgggaactcttgtgctcacgctgctggccgcaccatgcactttttggaggaagggaccaaca
ggcagtcttcgttctgtgtcctgagtcttggcacacttcctttctgcagttacggggtcctaagccccagaagtctaatgcc
aggaagcctggaacgggggttctggtgagtgcagggaagagcaggtggagcatccatgctggccggggtgctggc
tgtgggcgggggtcccactctgggaactccccctccccttcctgggcccgctctctatgctctgcccagtgtagacatgc
tcaactctgtggccttgaagggtcacctggatacctctggagtcagccttgacctcccttctgacctccagtccccaactc
caggcttacccagagttcccatgcatggtctctgctcccccatccccacccctcctccaccagccatcctcaactctccc
tttgctctcacccctacactgggttcccaaatcctgccagccctgctcttcagcagctcgcgtgcccactggcctccctgc
cttcgacgtcctgctccagggctccggtggggctgccctcatctctgtgacagcctcagacttgtctccccatccccatc
agccctgtcccccttcttctcacagcagctggagtgatctttccagaacataaggtaggaggctctccatgatctgatcct
gccttcctgacctccccagcctcatctctccctcctctgccctcgccctctgtgctccagccaacgtggcctgtcactcgtc
cacctgccatactgtcctgactccaggcctttgcctgtgctatggcctctgttgggaccactcttgtctctcccctgctgtgtc
tgctaattcccactcgtgtcagtgatccatggctgcagaacacaccactccatccatggaaaacaatcacaatcattga
ttactatctctccctgggcttcctggggggactgggctcagctgggtggttcactttggggcctcagcagtcatgggcag
acagtggctggggtcactgcaaagacttccctgcatctctggcagttgatgctggctgtcatctgagacacctacccag
ggcctctccctggggcctgggctcctgcttagcttggttgggaggctccaagaccaacatcccaagaaagatgagac
agaagccagatcacctttttgggcctggcttcagaagtcacccagcatcacttctgctgcatttatttcttagaaacacag
aatatcagaccccatctcttgatgggagggggcctcatggtttgtagacatgttctgaaactccactctgcccggccttg
gcttcgacgtgtctggtgaatgtgtgggctgtgaggctccccgaacgtagacctcagactgcaacgctggccgttaca
gggtctggcacacgggcccacgtcaggcccatcgccacagtgatggttgttctgtgactgtttctggtggcctctgctcc
acactccaggctgacgctgtgccccttccactgggaccctcgggtggcttccatgcacttgtgccctaaatcctgctcct
agactaaacttcatctcctgtgttctcattctgcagcatggctgttagggaacctgaccatctgcagcgcgtctcgttgcca
aggtataatgtcagtgcctcccttcagtggctcccatgtcacagaattgtcctgcagccctggcacatgtgtgccatgtg
ggagctggggcaggtcctctttcctcctgtggctccgagggagggggccgctccttccccagtctctaccctgacttgg
ccctcgtcctgcagccactcagagagcacgatggagctggagcttcagttttgaccaaatgcgtgtcgccggcttttgt
gtgtgtgtgtgtgtgatggagtctcgctctgtcgcccaggctggagtgcattggcaccatctctgcccactgcaacctctg
cctcctgggctcaagtgattctcctgcctcagcctcctgagtagctgggattacaggcgcccaccaccacacctggcta
atttttgtatttttagcagagacagcgtttcaccatgttggccaggctagtcttgaactcctgacttcaaatgatccgcccgc
ctcagcctcccaaagtgctgggattacaggcatgagccccggcacctggccttcaaattggcttttaaagaagatgac
cagctggtctgttctgtctggggccttggagggctgtttcccaggatgtgggcctttatcagtgctgacctaggcaatcaa
ggccaagctggccacactgcttttgattttttttaaattgtgatgaaatacgcttaacataaaatttgccatcttagtcattttg
gagtgttcagtagctttaagtccattcacattgctgtgcagccatcaccaccatccatctccagaactcttttcatcttgcca
acctgaagctctgtccccataaaatagcaactccattttccctcccccagctcaccattccatggtctgttgttatgagtct
gctactccaactaccttttataagtggagtcaatggtacctgtgtttttatgtcgggctcaaatcactgagcgtgatgtcctc
acggtccatccaggttgtaccaagtgtcagaatttccttcctgttttctgctgactccctgcttttaaaggccatcttggacat
tcaaattgtggccattactgacagttaaaatggccccggctcatgtctcctgagcctctacaatcctttaggtggggaca
gtactgcggcccacacgtccccacaggctgcgtgtgacacctgacagtgctttgtgccactgtggagcaggtgtcact
ctcccacttctcagagaggaagccgggctctggaagccagcagcccacaaggaggcccagcaggcttcgggccc
aggtggggaggtctgcacatctcagggcctttccctgggaacaaccagggacagaggccacacacccagcctcct
ctctgcccacagtgaattgagacacaaccccttacgtccctcccagccttaagtcaagtggaaagcaatggcccctg
aggtcacttagtccctcttgccagtttgtaagtgctgaatcagagactctgagctttggccctttcccgagttcccgggatt
atgagctgacacagcccccaagcaaccacagtgattccagcctgggagtgtggccttggcgggagggtcaatgcc
cagtcatggttcagcggctgtgcacacccctgcttacctgcatcccacgctttccatgcaaactcacgtggggcgcttt
gcgcttgcaggatggtctacccccagccaaaggtgctgacaccgtggtgagtaaagttactgacactgaaactgaac
gcagctcaaggggctgttctgaaggtattagagggcggtttccttgatgtaaatctcagttggggctgcttcgttctctcct
ctcagattctcctgacatctgaattcagaggcccacatggctgtcctctttccctttgctttctctgacttgcgtctcttgtttcct
gtccctttgttctccaaagcccctgcaaaggcctgataggtacctcctacctggggaggggcagcgggggttgggtgc
tggggagggtttgttcctatctctttgccagcaaagctcagcttgctgtgtgttcccacaggtccaatgttgagggagggc
tgggaatgatttgcccggttggagtcgcatttgcctctggttggtttcccggggaagggcggctgcctctggaagggtgg
tcagaggaggcagaagctgagtggagtttccaggtgggggcggccgtgtgccagaggcgcatgtgggtggcaccc
tgccagctccatgtgaccgcacgcctctctccatgtgcagtaggaaggatgtcctcgtggtaccccttggctggctccc
attgtctgggagggcacattcaacatcgacatcctcaacgagcagttcaggctccagaacaccaccattgggttaact
gtgtttgccatcaagaagtaagtcagtgaggtggccgagggtagagacccaggcagtggcgagtgactgtggacat
tgaggtctctccttgtgttcaagacagagtggggggcggccagccttgtcctcccagagggtagatgggaaaggtca
ttcatgcagcatcttactgagctcatgtgggctcgtgggctcgtgggctcgccaggtcggtaaaacccagctccttctcc
agaggctgcgtctcacccagggatggtggcttctgctgccccctcctctctgtaactgtggccggccgtcatgctgagc
caccccctcaatacaaggctccagatgtttcctgctcactgaccagagatagcaggagggggacacctgtttgctgtc
cttggaccctagaaagaggatgctggcagagccgtggtcacttctctgtcagatgtaggtggggcaggcaaagcagt
tggccccagacaccaaaggaagtggctgacccacaaggccctgggactctgggccaggccagagagggagcta
gccaggcaaccgcagacacatacttgacttctcggcagctgtgggcagctgggccagcgacagtggcggaggcc
aggaatgacttactcttaggaataggtgcagttcaagcctggagggaggaagctctagggtgcagaggcgggtgtgt
ggaggcctcgcgtgcagcttataatgagggagcacgtggccggcctggccataagaggggcagctgcgtgggga
ggcgtggctcaggccaggctgagggggagtgagcagacgccagcctgcggcctgctaccagcctccagccacct
gccctcagccctccttagtaagagggggtgctggtggtcccccatcgctgggaagaggatgaagtgaatcgcagcc
cgaggactcgctcaggacagggcaggagaacgtggtgcatctgctgctctaagccttccaatggccgctggcgggc
gggtgcaggacgggcctcctgcagcccaggggtgcacggccggcggctcccccagcccccgtccgcctgccttgc
agatacgtggctttcctgaagctgttcctggagacggcggagaagcacttcatggtgggccaccgtgtccactactatg
tcttcaccgaccagccggccgcggtgccccgcgtgacgctggggaccggtcggcagctgtcagtgctggaggtgcg
cgcctacaagcgctggcaggacgtgtccatgcgccgcatggagatgatcagtgacttctgcgagcggcgcttcctca
gcgaggtggattacctggtgtgcgtggacgtggacatggagttccgcgaccacgtgggcgtggagatcctgactccg
ctgttcggcaccctgcaccccggcttctacggaagcagccgggaggccttcacctacgagcgccggccccagtccc
aggcctacatccccaaggacgagggcgatttctactacctgggggggttcttcggggggtcggtgcaagaggtgca
gcggctcaccagggcctgccaccaggccatgatggtcgaccaggccaacggcatcgaggccgtgtggcacgacg
agagccacctgaacaagtacctgctgcgccacaaacccaccaaggtgctctcccccgagtacttgtgggaccagc
agctgctgggctggcccgccgtcctgaggaagctgaggttcactgcggtgcccaagaaccaccaggcggtccgga
acccgtgagcggctgccaggggctctgggagggctgccggcagccccgtccccctcccgcccttggttttagcagaa
cgggtaaactctgtttcctttgtccgtcctgttgtgagtaactgaagcctaggccccgtccccacctcaaatcacacaca
ccccctccccaccacagagacaccattacatacacagacacacacagaaagacacacacagacacaaaatcac
acacacaccctccccgccacagagacaccattacatacacagacacacacagaaagacacagacacaaaatc
acacacacaccctccccgccacagagacacaccattacatacacagacacgcaatcgcagatacgcccttccgg
ccacagaaacacaccattacacacacatacacagaaagacacacacagacacacaatcacacgcagcccctcc
ccgccacagagacacaccattacatacacagacacacacagaaagacacacacagacacaaaatcacacaca
caccctccccgccacagagacacaccattacatacacagacacacacagacacacaatcacacacagcccctcc
ccgccacagagacacaccattacatacacagacacacacagacacacaatcacagataccccctcccggccata
gagacacaccgttacacacacatacacagaaagacacacacagcccctccccgccacagagacacaccattac
atacacagacacacacacacacacaatcacacacacaccccccgccacagagacacaccgttacatacacaga
catacacacacacacagacatacaccagacacgcaaagacacacagacacagatacacagatacaaagacac
agacatatagacacacagacatgcacagagacacatggagacacatgcaaaaatgcacagagaaagacatac
agaagtgtacacacagacacatagaccacacagacacacagacatgcatgcaaacacacagacatgcagacat
gcacacaaacacagactcacgcacacagacttaggcagcccaaattcagcgcctggggcataagttcctggagg
ggtggccaccttcagcccccacggtaaggtcctgaggaaccttccccttagacaagggatcatggaggaggtctcttc
cggagcctggagggaggcctcaagtggtccttccacctcggcatcccaaagtgctaggattataagcatgagccact
gcacctggccccaacatcatttattgaacagactgtcgtttccacattgtgtttcttggcatttctgtcaaaaatcagttgac
cgtaaatgcatggattcacttccgggctctctattctgtttcattggtctgtttgtttttatgccaataccatgatgttttgattatta
tagctttatagtatatcttgaagttaggtagtgtattgtctccagctttgttctttttgcttaagattgctttggctatttaggggtctt
tgtggccattactgacagttaaaatggccctggctcatgtctcctgggcctctacagtactttaggtggggaagtactgtg
gcccacatgtcccaacaggctgtgtgtggcacctgacagtgctttgtgccactgtggagcaggtgttactccctcatacg
aattttaggattattttatttatttcagtgaaaaatgtcaatgaaattttgatagggattgcattgaatctgtagattgtttggggt
tgtatggacattttaacaatattaattcttccaattcatgaacatgggatgtcttttttttggcacctgatctcagatatgggac
agctttctaattatttgtatcttcaattttttcacaaatgttttatagttttcagcatacagatctttcacttccttggttggatttattt
gcaagtattttttgtagctattgtaaatgacattgttttcttgatttctttgttagggtatagaaatgctacagattgttgtatgtta
attttatatcctgcaactttactgatttcatttattctaacatcttattggtggaatctttagggttttctataagatcatgtcatctgt
aaacagggacaatttaagttcttcctttccaatttggataccttttatttcttcctcttgcctaattattttggctgagggccaact
tgttgggtttgttgttgttggtttttgtctgtgtcttttggtgtttcgtgttgctggcttttccagcacacagtcacagatatatgagg
cagaaagaaaacccagggaactcctgtcatgtcattcctcaggtcccaaggtccctacatggtctgccttcttctctcca
ccttttagtgtttgatatacatacgtttatgtatgtagttatatatgtatgtgtaatacccagggaaatatacagagattttaact
gtccttagtgtagggcgtggggacaggtgcatctattctaccttgcttggaagtggaaaaatcttcaagaaatcaatttat
caccaactggcatgacagaactcctggtttagtgtctagctgtctccactaaaatcaatcaccatgccatcaggcactct
cttccctctggtgtgtactggtacctgaaacagcgtccagcaccctgagagcacagaacgcagcttcttatgtgcccca
cccaataatcccaaatcgagcacagacctgggagctgaagagacaagctggaggtcaggcagttctattacttaag
cctggttcttcctgactccaaaagccaggcgccaccccatgctccttcccagcagaagctggttttcaccgggtcagga
ggacaggatggctattcctgaccgttgtacagcagtgccacatctcctagtttccagaacatttaatgtgttgctgtggag
gcctggcagctctgaagtagccccttgcgcccaggacaaaccgggcctggaggggggggtggggtaagctgaag
cttccagtatcccacgtgggcattctgttgctgagggcaaccgcccctccctgcaagggaggggaaggagcaggca
ggcacacccttcctccctccacatttcactggagttgacctgtgtcctactgtggttgcacctggaccagcccagaggg
actggagtgactcttcccgcaggtagggcacagccagcctgggcacagggcctcactgcaatccctgcgccccgcc
tccctgggggatttccaggggttgtcctgctgcttggtgagctgggggtgtgggggaccacagatgaggcaggcgcct
aaattgcttgggcggagtccggctgctctctccctccaggccctttcctgtgtcctcgggcccctcgggtccctgcacca
ggccagcagcaatcgggtgtcagaggttcaagtccaggctgactcctgggacgcaccgactttgcccaggagggct
ggggagagggcagccccaaagtgtcatctttgattattctttgtccccaaagccagccttgggcatgtgcccagcaca
gggagagctgctgcctctgtgggcttgagtcatttttgcttccttgcccctctctgctctgtttggtttaaaaaacaaaaacc
aacacccatgggtgaggcgaggcgaggcgaggcttgccttgatccacccgcttcctcccctgcagcctcgggcccc
atcctcccgcgtggccgcactaggggtgctgggggcggagtggggaccagacctgagtgggttctgggttcagcttc
gcagcaggagcagctggggcatcatcagttgggcaggtgacccaggatctgagcaggaatcttccaggtggaggt
ggggccgcccctctgcttgccacagctgccttctgctctgggggcagcactggctgagaatccagtgaagtaaggcct
ttgaaatggttttgattgtaaaagtaatgtttatgtttagccattcctttttttattttttattttttgagacgggtctccctctgtcacc
caggctggagtgcagtcgtgaaatgtgggctcactgcaacctccacctcccaggttcaagtgattctcctgcctcagcc
tcctgagtagttgggattacaagtgcctgccaccacacctgactaagttttgtattttttagtagagatggagtttcaccatg
ttggccaggctggtctcaaactcatatcctcaagtgatccaccctcctcgtcctcccaaagtgctgggattttgccatgag
ccaccacgctcagccatgtttagccatttttaaaaggtgtgaacagataattaagtctattagcaacattagaaaatttaa
gttaaaatttaaatgactctagcaagaattagcctttaggtgccctgggtccccctcctcaccgccccccaccccgccg
ccttctgagaagcgttttgtaatttttcttatttataaaatgggtttgattcaaaactgacttttcctcttcacagtctcacaggtc
cttccccaggtccaagaggctcttctgtgtcctgatgacaagtagctgcctagccgtggtggcacctcctatcacatgtta
agggacccctccccagggccacacctggcagaaggtggcttatgatgttcgcagcttgaaagtagtgtaaaccaaa
gataaaattctaagcccactcccccagccatcggaatggacccctcctcttggccagggcactccaaagttaacctg
aaaaaccggttcaggctgtgaagagaaggtggagtggacatgcctcatttatgtcctcctcccttttggaattcagcaa
agctgaccagcatgaacattaacacagaccttaagtctgattagtggcatttacaatctatactctctgaagcgtgctac
ctggagtcttcctttgcatgataaaactttggtctccacaaccccttatcataacctagacactcctttctagtgataataac
tctttcaaccaattgccaataaaaaaattttgaatctacctataacctggaacctccccgctccaccttcgagttgtcctac
ctttctggacagaagcaatgtggatcttgcatgtatttgattgatgtctcatgtctccctaaaatgtatacaattaggctgtgc
ccagatcaccctgggcacatgttctcaggccctcctgaggtctctgtctcgggccattggtcactcagattcggctcaga
ataaatctcttcaaatatttttcagagtttgactctttttgtcgacaataagctcatcagattaaagctgttggaactttaaatt
acttcgagccttgaaagaatgtgattctgacaccctagtcaagcagcaagtcgctgtgaactttgcttctccaattataga
ttaacgctcttcctttcttgatttgtaaaatgttatgaaagactaaatggcaccagatgtaagaccccttccctttcttagttac
tgaccttttgttacgaattaacttctttcttttcttgcacccaactcagaccagatggtacaaaagaccccatgcctatcaa
attccataagacagtatgttaaatatacttttcccgaagggaacaggatataaccaaacaaattgctgcaactcataag
ccaatcttatgtggaaaatgtaatcatgctaaacctccttgtattttcccatttaagtgaagccctaatccctccacctcag
agcactgagcccattcctttagagtctgtgttttttgagtggtggtcgtcaaacttagagttcgaataaactctataacattg
gattttgaacctttcaattatttcaggtggcagaagaaacacacagaccccatctcttcctttggagttctgtactgctcattt
caaagggatttcatcctgccctctgtaaccacccaaggggttcctcctgctccctttctaaataaagaccatgcattcca
gtaaagaaagagtttaatagacatgaggccaaccacatggtagagagagtttgtactcaaatcatctcatcaaaggc
tcatagctaggggtttctcaaggcagtttggggggtcaggggggccaagtaacaggtactgattggctggggcaga
catgaactcatggagggtcaaagctgtcctcctgcaggctaaatcgcttcttaggggggtgtcacaggagtgaggttg
ggggttccaggtagagccatgggtatcagacctgcaaaaaacctgaaaagatatctcaaaagaccaatctacagtt
ggggaaattgtgtattttgctagagaggtctacaccttagcttctccctctcgcctcatagcttttcattaggtttacacaggc
agaagagttttgggggaggcctgctgttatttaaactataaattaaatgtctcccaaggttagctcagcccaaaagccc
aggaatcattaagggaaaggaaagatgaggggcgggttagctcagctcactcttacaatttttctaattgatagagttttt
gcaaaggcggtttcactttcagcgtggtcgttctcctaacctctcctgcttccccaggtcctcaccctaaaccagcaccc
atcctgcactcccagggtgaaccctcccttccctcctgctcacctgggcccaacccagtttccccccaggagctgcgg
aagggcccagtggtgccaccttggcctccaggcatcactctccaacttcttccctcctccctccacaggacccttgcctc
aaatgtcagtcccgctgctctctgctgcactcagcctcttcctggcaagtgtgcccctcccctcttgaagccatcaccag
cctctcgtgcccacccccatctaggccccccactcctctccagtcccggtgtcttggaagagcctctggatcacagggg
ctgggaaattcgacttgtcatccccaccaagcaaacacatgcttccatccagggcccctcagtgggtgaaggctgag
ccccagtaaaggatagatcatcactgtcagaggcgagtgaactagagcgactccatgttgaatagggactgggtaa
aatgaggctgaaccttctgcactgcattctcagggggttaggcattcttagtcacaggataagataggaggttggcagg
attagtatcacaagatacaagtcaccaagaccctgctgatcaaatgggatgcagtaaagaagccagccaaaaccc
accaaaaccaagatggcgacaaaaggaacctccagttgtcctcactgctcattatgcgctaattataatacattagctg
ctaaaagacactcccacctgtgccacaacagtttacaaatgccatggcaacgtccagaagttaccccatatggtctaa
aaaggagagggaccctcagttcagaaaaatcccctcccctttcttggaaaactcacaaacaatccacctgttgttgag
cctataatccagaaatcagtataagtatactcagctgagatccaagaaccctgtcttggggtttggactgggactcctttc
tggtaacaatactagggtatacggagcccccacatccccgggcaagtggtgcagaagccggagccatcagcagc
cgcccctggcaggctcctgtgtggactccttgctgcagggcatttggtttaccaggcgtgagagacccacacgtgaag
cgctagcccagccccatcaccgcagcacagcctcaccactgcagcccagaaaaaccactgcagcccagacaca
cgactgcagctgaggcccatcactgcagcccagacacaccactgcacccactgcagctcagcccaccactgcacc
ccagccccaccactgtagctcagcccgaccactgcagcccagcccaccattgcagcccagacacaccactgcag
cccagcccaccactgcagcccagccccaccattgcagcccaggccctgcagttgtccctactaggtctgagtggag
gaatcagagagaaaactctggaaagtgctagacacaggggaggccctcaaggaatggtggctgccactgggaac
tctgtttctgtggtgcttactggacagataggctcctttgcagatttttttattccaaaactaagctcccatcacccagtcctgt
cctccctgctggcagagcttgtccctggatccaagctgtctcctctgcaccaaatccggtggcctcaagtcccagctctg
ctttgcacaggggcttttcgaagccctgtctcccctgctgtgagatgggggtcacccagctgccccccagggggtggtg
ttgctgacggagcaggtgaagacaatgccaagagctgcgcgtggtctggctttcccaggctggatgtgcccctctgat
cctcagtctccccacatggacaatggctgatgccgaccacccacactaggtttgccccagagtcaaagtgagctcctt
gaggacagggaactgacccccacacagggccccccaggatctgcttgaggaggcaccaggcagttctcccagaa
ggcagggcgtgtcttcagagctctctgggttgagtgggcctaggccatcgctgcctctgcccaagtctttctagaggctt
cccaagtaccagagctgccctggagggccagagttaccagaaagtccaaagggagaaagtcagagagacacca
gcacacccaagaagtccagggccttggggacactaagggtgaactctgtgctgtgaagggcaggcctggcccgga
gagagggagcctgcagaggagacgggatctctcggtgctcttccgaagaagcccctggatctcccactgtgttctgct
ctcccgggcgggcaggtgtcaggatggcgcctggtacagctggttttgggcagaagccccgcccgtggcctgcacgt
tgggtgtctgcgtgttgggcccctgctctgcagcctcagcaggtactgtgacgggggatggccgggcagggacctac
ctggccagctctttgtctcagctgccttcacacggcagggtttgctttgcgcatcccatccctgtgaacatacaggtgcga
ttagtttgctggtttgtggcagggtggttttttcttcttcctaaaaatggatttagtagatttttagcttcacaggtttcatctgaac
caatgacttggaatttaagacaatggctacacgcttttgactgggtttgggatttgtgatccgaaacggttcatttgttatat
ggctagaatatgacatgacttttcatgtgttttaaaaaatataatgaggcggcggctgcgtccacgtcgaccaagactg
gagcgaagtttaaggaagggacagaatcgccggggagtgcccttgttgttgctgggggactcccagccttccctgtg
gcccgagaaggagaagcagtggctctctgaagcaggcatggagagtagaaaactggagttattagcataccctagt
acctcttacaactttcccttccatgttagcactttagtgctggcttctcagttttcttaacattgagacaataaatgtgtgttgtgt
cttgtagatggcataaagagtaaataagaagttttagagttgttctggaaaatgtcagaataaatctccacttgagttgtg
tattctgctagtccaagtggacagcaacttcctgctaccctcccttgcaaccttgcagacaagtctctcctctgaagaac
gaattagatttagctaattagaattaatccttgctttcatcgccatcatctgtaaaatactttgttgggtagaccactttatacc
tttgcaatacggtctccgtgggtaaaaaacaaatatctgtaatgcagacaggaggatgtgaaaagttctgttgcacgtg
ttttttttttttgttttttgttttttgttttttgtttttttgagacagtctcgctctgtcccccaggctggagtgcagtggcgcgatctcgg
ctcactgcaagcaagctccgcctcctgggttcacgccattctcctgcctcagcctccggagtagctgggactacggac
gcctgccaacatgcccggctaattttttgtatttttagtagagactgggtttcaccggttgcacgtatttttaattctgtggctgt
cacatgatagagaatggaattgaatggagatttcccttttgcttcttagggttggaaatattcccatgaaaatgttcaaattt
ggaatttgaaagccaccaaatgaatctttatgtataaatccttgtaaatgatagattccataggtgagacttttatgtatttc
aggtgggagcctactggcatatatttttaaatgttcatattacttagaatctccaataggaagtctttatttgaaatagttgaa
tccgtgttctagtattttcctttcagcaagatctgttaggtttttaccccttcaaaaataagttttatttcatctgcaaattgtggc
aatgttatagcgatcagaaactacgtaaggaatgttatataggcttgtcagttcccatttatcttaacaacaataaatatc
acatttcttcttttgaaaatgacacatatttaggccaggcgtggtggctcactcctgtaatcccagccctttgggaggccg
aggtgggcggatcacgaggtcaggagatccagacaatctggttaacatggtgaaaccccgtctctattataaataca
aaaaaattagcagggcatggtggcaggtgcctgtagtcccagttactcgggaggctgaggcaggagaatggcgtg
aacctgggaggtggagcttgcagtgagcggagatcgcgccactgcactccagcctgggcgacagagcaagactc
cgcctcataaaacaaaacaaaacatatttaaacacttagaaaataaagttaacacttactgaagtgccggtactaca
ctgtcctagtactaaaaggaagcaggttggaacatacatatggcctatcatttataacagaattaacttgttgaatgtctg
taaatgatttttttttgcaaaggaaaaagttgatcctggaaaagattgttgtgcatagttattagtcatttgtaacctcgcttaa
gtgtttctcagttgttcaacatagacacttttttctccttaccatgtatttttaaaaatagtctattacttgactttgaacgtaaag
ctttaatcataatttcccacgtatacatagttcatctgacggtaagctggatttgaaggtaggggtttcagcgttacttaagtt
ggtagctgagggtatcaggcatcagttcatgcaataagacaaaaaaatatatatcctttgcttgccaaggggtagagt
gatgtgcatttatctgttttctgttctgtaagtttagactttcaaaccattttgtaaaccaacccttgggaaatttgaaattacctt
ataacttaagactctgtggtctctggaatcaccctatctgtttcttttctgcgtaggtatttataacattgctgtttgactatagc
gtgcactctgaaatgttatcagtggaaatttgtttgagtttcattaatgctatttcactagttagacataattacttctaccagt
gtaaatgacactgatgcgcacagagcttccagatctttcagactcaactactaggtcaattagtttgcataataaaactt
ggcagtttctacaagcctattatgacaaaccaggagctaattctgtaatgaaaaactatccattctgaatgatagggac
gtaattatttgctgctgctgtcctttgtaaattttgaacatgacattatactctgtgcctactaaaggtatcctctggagttttttg
agaggagggaaactggaaaattaaattgtatttttgccagaagactcttacttgcatgtgtctcagggtcttcagtttttcta
gaagtttccatatccaaggttcagagttcatgtgaaatacttctttgggagaaaaaaatccttcattcctggtattcattgga
ttggaaatctgcagcaaaatgctgtttaaaattactatgtggtttttctatcttatccttagctctctggctattgaacttttttttctt
ctttgaagttagcttcaaatttgcttctatgctaaattacctgtaaatattctggataggaactatttgaaatagtatttgttaaa
agaaatgataaaatgaaaatgttcaaactacagagattttaaaatgccataacgatcttgcaagactaactttaaaata
tactataaatgattattatgattttggtggtaacgatcccccacacacaaccgctgtgaagaaatgacgccacattttccc
ctattgtaccaaaaagataaagatggtaaacattaatcaaggtattttgtattgtcaacgcgtgcatattctaaagagtta
aatgccaactcagcagcactggcttcctggctggtcaaccataggaaacctcgttcatttctcccagtgttgtgatgttca
tacttctacaatcttccctgtcatgactttaacttctacgtttcattaaccattcctgatgttagttctcagagcttcttcttttttttttt
tttttttttttgagatggagtctcactctgtcgcccaggctggagtgcagtggccccatctcggcacactgcaacctccgcc
tcccgggttcaagcgattctcctgcctcagcctcccaagtagctgggactacaggcgtgcgccaccacgcccagctat
tttttgtatttttagtagaggtgtggtttcaccctgttggacaggatggtctcaatctcttgacctcgtgatccgcccgcctcgg
cctcccaaagtgctgggattacacgtgtgagccactgccggttcttagagtttcttaaggcaaaaataaaattattcaatt
ctgaaaaaatataaataaataaataataaaaaatataacgaatctggaggactgcatggtgtcctatagcgtgacggt
taaaagcttgggctttggaaccaagtcggcctcgaaatcgcagctttgctgctgcctgggccactggaccttctgagcg
tcgttttcctcctgtgtcagctgagggtggtgacttgggctcccgctcaggggttgaaagagattctgcagcctctggagc
tgcccctcagcgcatcatcactgggtctgctgtcctcggtacctgcctgggtggaggtttctgtgacttctcaacggaagt
ggctgtctcaggcgaacgctgtaacagtgaccatcccttgaggtgcctgtttcttgctctgtttttatttaaagatctgtgga
aatgtagaatatgaaaaatgaactgcaatattcgggatgaaaatggactaggctaagtattcctgatttgaaaaaactt
ttatcttaggtttaggggatcgtgtgcgggattgttatataggtaaactccgtgtctcgggggtttggtgtacaggttatttagt
cacccaggtaataagcatagtacccgataggtatttatttttgatcctttccctactcccacgcttcactctcaagtaggcc
ccattatctgttgttcccttcttcgtgtccaagcactcctgattaatgagcctaaagaaatactgaaagaacaagcgaaa
cacatctgttctctttcctgctaccctctccgcatgtccccctctctctagaaaacaatggttttaatcttggctgcatgttaga
gtcacctagggtgcttttaaaattccctttgccaggcgccacacttgaacgttgtgattatttgcctggcccaggtgtcaat
attttttttaagagatgggggtctttctctgttgcccaggctagaatgcagtggcgtaatcatagcgcactgtagcgctgaa
ctcctgggttcaagagatcctccgacctcaacctcttgagtagctgggactactgacacgtgccaccatgcctggttaat
ttttaaaaagtgtttgtagagatggagtcttactatgttgcccaggctggtctcttaacttctaggctcaagggatctcctgc
ctcaggctcccaaagtgctgggaatataggcgtgagccactgcgcccggctggcatcagtatcttttgaagttccgatg
attctacagagcagccacagttgacaaccattgccctagaatcatcgtcttcactcacatttcatttttaaatgaccgtaat
tgaaaagacacatttggtcacgaggaataggaaatggacaactcacatctcatacaaatcacaagtacctcctggtg
gaagcagcaggcctctgggaaggttctagaaagaatgggtccctgtgtctgaagctgtgcttctgagcaactgcctgg
ctggggcgcccctgctcactgggtggtcactgaacttcacacactgctccccagctatgctttcctccttcatgcttccttct
tgcttgaccggactccaacaaggaagcctctttttcttcatgattaaaagagtaataaatggtcatgggagaaaaataa
agtcatatagaagcagacaggctgggtgtggtggctcatgcctgtaatctcagcactttgggaggccaagatgggtgg
atcactggagctcaggagtttgagaccagcctggggaacataacgaaaccccatctctacaaataatgcaaaaaa
aaaaaaattaggcatggtggcgctctcctgtagtcccaattactcgggagggtgaggtgggaggttcacttgagcctg
ggagttggaggctacaatgagctgtgatcaggccactgcattccagcctgggggacccagccaaaccatgtcccaa
aataaaataaaataaaataaaattaacataaacataaaaaaggaaggagatgagcagaaagtaaccacccacc
cccaaaagctcctctacctgggacagtaggttgggggagtgtggcaccctggagccaaaggtcccaggctcctgtcc
aactcactacaggccatctctgctctctaaccccaagcctgagtttccttgcctgtaaaatggagaggacagtagagcc
tgcccctgggcctgggaatgaggagtaaaggagtcggagcattactggcacgtggggggcagaagtgctgtgtcc
tgtttttacacacatgcgcagctgtaaaacggtttattttatttttcattttttgacagagtctcgctctgtcgcccagtgcagtg
gcgcgatctcggctcactgcaagctccgcctcccgggttcacgccattctcctgccttagcctcccgagtagctggaac
tacagacacccaccaccacgcggggctaattgtgtgtgtgtgtgtgtgtgtctgtgtgtctgtgtgtgtttagtagagacgg
gatttcaccgtgttagccaggatggtttcagtctcctgatctcatgatccacctgcctcggcctcccaaagtgctgggatta
caggcatgagccaccgcgcccggccaaaaggtttttaaaaacatgaaatgagtcattctgtgcacaccattttgttgtc
ccgactttgtgcccatagctgagtttgccagacgagggacccccagtcagcaccgtgggctcaagagagggcagcc
tcagaatctcaggtggccggactcagggctagccctggtgtcctggacagtgcccagacctgaaggccgcagggg
ccccctcccccacaccgcccagagtgaaagctccagaaacagctcagggcctgagtcgggaatccctgatcccta
aagtggagttacagaactttcagggctgtggggaggatgatgaaaggggctcggggcggcagccccgttgagtca
agggggagtgcccagctttgacattttatccacaggaggctcggaagggcaaagtcccatttcgcttccatctggctttc
ccgccgcttgctctgtgtgcctggttttgttttgtttttgttgttgttgttttgagggagtctcgccctgttgccaggctggagtgc
agtggtgctatctcggctcactgcaacctccacctcccgggttcaagcgcttctcctgcctcagcctccaatgtagctgg
gattacaggcgcccgccaccacacccagctacttttttgtgtttttagtagagacgaggtttcaccatgttggtcagggtg
atctcgaactcctgacctcaagtgatccgcccacctcagcctccccaaagtgctaggatccgtgtgcctttaactccttg
cctttaggagtcgccgtgctgtaagcagcactgccccggggtaagggcgggcgctggagactggcagtgctcacca
gcgcccccagctggtaagtatggcagtcatcgagcctggggggaacggaaagcaccactgggatttctccccgctg
agctgggcagttctctcctctgcccagggcgctgccgtgcccccagagggctttgggaaatgtgagttggctcttgtgg
ctgttcctatgcctggggaatttggaagagaccaatgtcattgggagggacagaaagtaccacgcactgaagaagtg
tgcccacttcccaaccccatgcccagggtgctcaggagaggaccactgggaagaccgcggatgaacattccagat
cccctcggggagagaaggcgctcctagccagctggtccttgagcgcacgcgcactccgtgttggagcccatttagaa
taaacattcaaaccctcagagcccactggagggctcagggcctttcaccttggctaagaaacgatccccacagcctt
gaagctgggtggaagctcaggtaggtggtcagggtccctgggtcagcctgtctgcccagctccaggcagctttcctga
tccttacaaactccgggtgcagctccacaccgccgggtcccgcccaccggctccgctccgaccacgcccactctacc
cctcaacacagccttgaccctgccccactggccctgccccaaccacgctcatcccactcctctgccccgcccttctcg
gccccgcctcccccgctcagctcagcccccactgggcctggacctccctggttcctgtctatcctcccctccaccctgcc
ctctctaaccgaatccccgccccatctagagccctaacctctagatcggccgggggttcatagatcataagccaggcc
ctgctggtgacctggaggccgcggacagccgcccttcaccctggtggggccaccctgccaggagctggcctggag
gcggggcccggggctgaatttggggtcgctgctcccactgcccacggttcttcccccaaccctgcccagctctggggg
cctttctctcatctctctcccttttgttgttgctgaaaaatgctgctgtttttctcgtggctaagttatatatgctcttaaaaagtaat
gcccggcatggaggctcacacctttaatcccaacgctttaggaggtggaggcgggaggatcgcttgagcccaggag
ttcaagaccagcctgggtaacaaagtgagaccccatctccataaacaaacacataaatagtaagaacttgataaag
gtctgagaaagaaattagagatcactgtgaatcctttccatgctgacattttggcacattttccccaggctgcgtgaggcc
caggctgaatgtgcacattggcatcttgttttctttattaacatcatttcagaagcattttctgagatgagtacaaactgaaa
acattcttctgaacacctgcatggagccctcttttcacacatgaccctgtgatgggccagctgggctttggataaaaaaa
agaaaaaaaggaaaaaatgattagaggtgatccagtagcgcgatgctcctcccatctgaggcaaaggaagaaga
aataatttaaaaatgttcccagagcaaaaacagccgttttctaaagaatggccccacaattggttcaagttgaagagc
ctccccgaggaataacagcttcgtcatccatctagttcaggcgttgtttctatagtaacctgtgacgtggtccccttcccaa
aacacaaaacaaaacaaaaaacagaaacaaaaacaatgaatccctgagaacagtgagaaagaaaaagctctt
gcttcctttggcctacggaagggtgtggctgttgagctgagccttggagttcctgggtttgccggtttccccctctggcagt
gctgggggctttgagccagcaacctcaaagaatgcccatgccagggaggccagcatgcacgcaggtattgatgttg
gctttccacgggtgcagtgagccgcataatggtttggggtgaggatttaaacgttttgagacaggaacggcttgcctctg
ttttaaagatcagctgctgccatttcaaagatacgttctgttactttgccaaggggcctcattccatgttgctgggaatcagc
agcagtgctgctctgatgtggctggtggccgtcccaagcccccatgcctccctgacctctcgctgcccagcacttcccg
ctccaggggtgctctcaccagccatgactccccctcccgatgcctacatcccccgcctgtctgctgtgccctgtccaca
gcgccctgtccacagcacagctggctcaacacatgtggcatccaagccgggcaccgtgctttgcctcacactgctga
gtccacctggccttacatgccttggctggagtccaggcgtggggcaggcttcggggatggtgtcaaggactgggcact
tcctgcctctctcctcaaggtccccagggctgcctcatcccaactgggtcccctgctgcagttctcagatgccctctgcat
gttccttctctgggctccgtggctcattccagagcaatcacagaccagagggacggcgccccattagatcaatcagac
aagccctggagtggggagtgcagtcaccatccatgagggtgcgggtgtgttggggggatgcggggaggggaatgg
atgccgcgtcagccaccaacagccctgacgggcccctgacttcctgcactaacattgtccctgtgctcacatctgccc
agtaccactggggccattctcacctacggctctaccaggtgcccaggaaatgcccccaaacgggcaggatgctcac
aaggcaaggccacctgccctgaaggacgcgggggccacagggtaatggaaagcaaagggatgggggaattatc
cccaaatcagtcacacgcctgatggcagccctgtcaaaatccctagggaggttttttgttttgttttgtttttggagggggat
gactttgtagagaaactgcattttttcttaaaaatgtaaaatgatctgcaatgcaaatataccatgttctgtttagggcaagt
agcctcttctgattttgagtctacgaataatcagaagcactgaaaattccaaagcaggagttaaagtcctcagcagcg
cttgggtgtgcggtggggtccgtgatggatgctgaaaccgtgggctccaaaggccagcacaggaaggaaggagag
ggcctcaaaggagggggcaggttggacctggccaaccccagcgtggcaccctcagcccttctcagctcccagtgcc
tcttaacattgctggcaggtgtgagcctggggtcgtgctagcctctgagtgagctcagccccttaccatttccagcctcac
cttcctctcccaagaaagtggcatgagggtaacagcaacctcatgggagtgcaaggctggcacaggctttagccgc
atctctgagccctttcaccaaaaaggactcactgaaccaaagagctgcacagaacattagcatccacccccacccc
agagagggcttcctagtgctcacctgggggagggtgtgtgtgcaggtgggaggacggagatcgtggcctccctccat
gtggctcgtggtatgaagaccccacctcagacatctcctgcctcccaggtggcagctgcctgagtggtgggtggtggc
tggaaccaggagacccagttctgcctggctgggcctcagtttcccaggaggtgacctggacagggtcacgctgcccc
atctctgtggcacctggcttgggactccctccctccccgctgctcttccttctccttactcctggggtcttccaggaaagtgc
cgatcccggagtaagatttggggggcagttcctttgctggcctcaagccccctgcgcactccttccccagcggcagag
cgagggcatccggtcctccaggagaagagctgggctaggagcttagtgtgttccccatgtcacggggcctcttccacc
ccctgactcatggaccccatgctggccacgctgcggaggcctgccaggcttccagatgccggcgactcccctcctgg
ggccagccttctggaagggggtgttgagcccatgaggagctccataagtggggaagcaagtggggacggccatgt
ccacagcatcaccttggcagctgatgggcgggaggacaggtccaggcggcctccaggcacgttcctgtttgtgtgac
attcaccgtgacatggtgcatgccgtggcacacagggtcctgtgttcagataagccagggcctcggaggaactcagg
cggaggaaagagccggaacacaaacacagcgcgacctttcccgggaagcggcccctccaggaatgcgccctgg
gcccccctgccaagccctgggcctccatggccaagtacccctcacttccaccatccgcgactcggttcactccagttg
agccccaagagcctctgctgagcccaacccggaagggcgagggagcctcggtggccagagagggccgaggcct
gttgggatgactgcactccttcaggacaggcaccctctgtgacggggacatgcagggaccactgcggctgccctggc
ccaagacgccctggaggccaggcgggcagggccccttcatccttcaggaggtgggactgggctgggttttgaagga
tggcagcgtccaggtagactgagggcacagacagggaaggccacacaggcctaaaagcacacagacttaggcg
ggcgggaaatgtgaggtcaaatcctggagccctcggagggggactcaggaaatcagatgctcagtagggggccg
agtttggagccttggccttggggcaggcgccgtgtccagcagaggggctgctgcccgccacggctggccttgcccttg
gcattggccccagggaagtgcagggcgcaggagagatgccacaaggcccttgggtgttcttcccgcagcttccttcg
gtcgcaggcggatgacgcatctttgtcactgcatccacgtaagatgctctgttagaaaaaagacaggaaggaaactg
ttccaactcgcgagtgagtcactggggaggtgggattggaagtgattttctcccctcttccagctttctaagatttataaca
cgtgtgcatcctttaaactcgtgtcaggaagcgacctcactagggcattgtgtctgagcaggaagaatctcaggactcc
ctgggtttactcatttgagcgccagcctccaggagacggggcaaagggctgctcgtcctccgtgaccaggcggcccc
tctgcctttgtcgcatgtgtggtcaacgtggtggctgaaaggctcacgtgttccttagcccgggggacctgagaaacctg
accgtcaccgtgtctctgtagcctagttggtggcccggcagtggacagaggccctgcaggccaccaaggaggacg
gaaccccagggacagaggaggagccgcagtgcagggggtccaggcccgcccacagccccaccctcgggaca
ccccaacccctgcaccctctgtgcttgccttggggcatttgcttttcaactccacagaaaatattgtgacagtaacgtggg
agcagcaagaacccaaataaaagaacgtgtcattcacgctgcctccctttgtcctgagctgcaggtaccagcacatct
gggcactttctccatggtccgctgtcctaatcctgactatttcagcacccagggagcaagccaagaacaaaaacacct
atcatccatgcccggcattagaaaatctcattgttggtgagaatgatggtttccagcttcatccgtgtccctacaaaggac
atgaactcatcgttttctatggctgcgtagtattccatggtgtatatgtgccacattttcttaatccagtctatcattgatggac
atttgggttggtttcaagtctttgctattgtgaatagtgctgcaataaacatacgtgagcatgtgtctctatgg.
SEQ ID NO: 2 (FUT1):
gaaagtccctgactggagttggcagccaagccaggccctggagtgggcacccagagggaagacaggttggctaa
tttcctggagcccctaagggtgcaagggtaggccttctgtgtctgagggaggagggctggggctctggactcctgggt
ctgagggaggaggggtggggggcctggactcctgggtctgagggaggagggtctgggcctgtactcctggatctga
gggaggaggggctggggaacttgggctcctgggtctgagggaggagggagctttggtctggactcctgggtctgag
ggagtaggggctagggatctggactcgtgggtgtgaggaaggaggggctggggtcctggactcctgggtctgagga
aggaggggcagggggcttggactcctgggtctgaggaaggaggggccgggagcctggactcctaagtctgaggg
aggagggtctgggggcctggactgctgggtgtgagcagaagggtctgggtgctgggagtcccgagcctggggaga
tgatggttaaacttctgggaatcaagtcaaactcctgagtctttgacattgatgtatcttgaatgggagggtcagtctgtgg
ggaaggattacccaggtgccgaggcaagagactgaaggcacaaactgtttcagtataataaagaaaatagttaga
ataagaatagttatcatacaaattagatatagagatgatcatggacagtatcaatcattagtgtaaacattattaatcatt
agctattacttttattctttgttgtataactaatataaccaggaaacaaccggtgggtatagggtcaggtactgaagggac
attgtgagaagtgacctagaaggcaagaggtgagccttctgtcacaccggcataagggcctcttgagggctccttggt
caagcgggaacgccagtgtctgggaaggcacccgttactcagcagaccacgaaagggaatctccttttcttggagg
agtcagggaacactctgctccaccagcttcttgtgggaggctgggtattatctaggcctgcccgcagtcatcctgctgtg
ctgtgcttcaatggtcacgctccttgtcctcttgcattttcctcccgtactcctggttcctctttgaagttcgtagtagatagcgg
tagaagaaatagtgaaagcctttttttttttttttttgaggcggagtctcgctctgtcccccaggctggagtgcagtggcgtg
atctcggctcactgcaatctccgcctcctgggttcacaccattctcctgcctcaccctcccaaatagctaggactacagg
cgccctccaccacgcgcccggataattttttgtatttttagtagagacagggtttcaccgtgttagccaggatggcctcca
cctcctgaccttgtgatccgcccgcctcagcctcccaaagtgctgggattacaggcgtgagccaccgcgcccgcccg
aaatagtgaaagtcttaaagtctttgatctttcttataagtgcagagaagaaaacgctgacatatgctgccttctctttctgc
ttcggctgcctaaaagggaagggccccctgtcccatgatcacgtgacttgcttgaccttatcagtcatttggacgactca
ccctccttatcctgcccccccttgtcttgtatacaataaatatcagcgcgcccagccattcggggccactaccggtctctg
cgtcttgatggtagtggtcccccgggcccagctgttttctctttatctctttgtcttgtgtctttatttcttacaatctctcctctcctc
acaggggaagaacacccacccgcaaagccccgtagggctggaccctacgttagcctgccctgctcggggttggcg
atgctggaggtgggccttggaccagagaaaatgctttaattaggtgacaagcgggcagaggcctttgtctctggcgcc
ggcagccacggcccccgctgacggcgtgggaaacagaccctgttccactccggtctccagccttggaatggttgcct
tcgtgcagtgcaggtctggaaagtagcagtttggcacgggaccctagaattccccaaaaggagtgactaggggctg
ggattctggaatttgagtgtggacggtgaggcggggggtgtgggagatcggagaccctggtgggcgcgggagcac
ctgcaggctggaggccctcgcgcgctccggcggcagcctggcaaacaggttctccatcccccaggaggacgcggc
agagggcggacgatcgctccactcgccgggaccaggtgcgggggccctgcccagccgctggggcgtggccagg
ctcgaagcacccaggtgtcgggggccgactctaagccctggcaccggaagagagagggcggcggattggacctc
ccggctccagcattgcaactgggcgctccgtctcctggtccacgcaatgatgctgcggctgctcagaagccaggtag
cctgccctgggtgaagccttcgcgcaggtcaatgacggggcggaggggcagggcgcggtcccctgcatccccgat
ctggggagcggtgggcccaggggccatcgccttagcccctggcgctggggctcggcgccaagtgacgggcgggg
ctccaccttccagccatccgcccggcccgggagggcggacgctgcgagactcccggccgcgccctctccttcctctc
ctccccaagccctcgctgccagtccggacaggctgcgcggaggggagggctgccgggccggatagccggacgc
ctggcgttccaggggcggccggatgtggcctgcctttgcggagggtgcgctccggccacgaaaagcggactgtgga
tctgccacctgcaagcagctcgggtaagtggggactgccccactcagttgttcctgggacccaggaacaactccttca
gaaccaggaggtgcacccccaacctcttctccaggtcttcctaaggccctaggaatctccgccacctccccagccatt
actcctccaggaaccaagatgctccttccgctcctgaccctccagcctctcttgttttacttgaactatcgtttcccatcacc
acctctgtggtggattttgcgcctcacagacaggtactcctgagaaacaggctggtggaagagtccagtatcagcgg
aacttacaggaggggagactcgagattccttcaggaaaggtgtaggaacctggaccactttcttttttttttttttttttttttttt
aagacagggtccctctctgtcgcgcaagctggagtgcagtcagcggtgctatcgcggctcattgtgagctccggggat
cctcccgccttagcatccggtgtagctgagaccacagacatgtgccaccatgccaagctaattttatttatttttttttggag
acggagtttcactcttgttgcccaggctggagtgtaatggcatgatctcagctcaccgcaactcccgccccccgggttc
aggcgattctcctgcctcagcctcccgagtggctgggattacaggcatgcgccaccatgcccggctaattttgtattttaa
gtagagacagggtttctccacgttggtcaggctggtctcgaactcccaacctcaggtgatccacccaccttggcctccc
aaagtgctggggttacaggtgtgagccaccgcgcctggcccatgccaagctaattttaaaatttttttgtaagagtgctct
gttgcccaggctgatcttgaactcctgggctcaagggatcctcccatctcagcctcccaatatgctgggattacaggtgt
gagccacagtgcccagccaaaccatggctatcttgaaaaccacttgtcttccagtccccatgccccgaaattccaag
gctctcatccctgaaacctaggactcaggctctccctacctcagccccaggagtctaaacctttaacttcctctttccctg
ggactaaggagtgctgcaccccaggcgcctcccttaccccacatccctcctcagcctcccctcctcagcctcagagc
atttgctaattcgcctttcctcccctgcagccatgtggctccggagccatcgtcagctctgcctggccttcctgctagtctgt
gtcctctctgtaatcttcttcctccatatccatcaagacagctttccacatggcctaggcctgtcgatcctgtgtccagaccg
ccgcctggtgacacccccagtggccatcttctgcctgccgggtactgcgatgggccccaacgcctcctcttcctgtccc
cagcaccctgcttccctctccggcacctggactgtctaccccaatggccggtttggtaatcagatgggacagtatgcca
cgctgctggctctggcccagctcaacggccgccgggcctttatcctgcctgccatgcatgccgccctggccccggtatt
ccgcatcaccctgcccgtgctggccccagaagtggacagccgcacgccgtggcgggagctgcagcttcacgactg
gatgtcggaggagtacgcggacttgagagatcctttcctgaagctctctggcttcccctgctcttggactttcttccaccat
ctccgggaacagatccgcagagagttcaccctgcacgaccaccttcgggaagaggcgcagagtgtgctgggtcag
ctccgcctgggccgcacaggggaccgcccgcgcacctttgtcggcgtccacgtgcgccgtggggactatctgcaggt
tatgcctcagcgctggaagggtgtggtgggcgacagcgcctacctccggcaggccatggactggttccgggcacgg
cacgaagcccccgttttcgtggtcaccagcaacggcatggagtggtgtaaagaaaacatcgacacctcccagggc
gatgtgacgtttgctggcgatggacaggaggctacaccgtggaaagactttgccctgctcacacagtgcaaccacac
cattatgaccattggcaccttcggcttctgggctgcctacctggctggcggagacactgtctacctggccaacttcaccc
tgccagactctgagttcctgaagatctttaagccggaggcggccttcctgcccgagtggggggcattaatgcagactt
gtctccactctggacattggctaagccttgagagccagggagactttctgaagtagcctgatctttctagagccagcagt
acgtggcttcagaggcctggcatcttctggagaagcttgtggtgttcctgaagcaaatgggtgcccgtatccagagtga
ttctagttgggagagttggagagaagggggacgtttctggaactgtctgaatattctagaactagcaaaacatcttttcct
gatggctggcaggcagttctagaagccacagtgcccacctgctcttcccagcccatatctacagtacttccagatggct
gcccccaggaatggggaactctccctctggtctactctagaagaggggttacttctcccctgggtcctccaaagactga
aggagcatatgattgctccagagcaagcattcaccaagtccccttctgtgtttctggagtgattctagagggagacttgtt
ctagagaggaccaggtttgatgcctgtgaagaaccctgcagggcccttatggacaggatggggttctggaaatccag
ataactaaggtgaagaatctttttagttttttttttttttttttggagacagggtctcgctctgttgcccaggctggagtgcagtg
gcgtgatcttggctcactgcaacttccgcctcctgtgttcaagcgattctcctgtctcagcctcctgagtagatgggactac
aggcacaggccattatgcctggctaatttttgtatttttagtagagacagggtttcaccatgttggccaggatggtctcgat
ctcctgaccttgtcatccacctgtcttggcctcccaaagtgctgggattactggcatgagccactgtgcccagcccggat
attttttttttaattatttatttatttatttatttattgagacggagtcttgctctgtagcccaggccagagtgcagtggcgcgatct
cagctcactgcaagctctgcctcccgggttcatgccattctgcctcagcctcctgagtagctgggactacaggcgcccg
ccaccacgcccggctaattttttttgtatttttagtagagacggggtttcatcgtgttaaccaggatggtctcgatctcctgac
ctcgtgatctgcccacctcggcctcccacagtgctgggattaccggcgtgagccaccatgcctggcccggataatttttt
ttaatttttgtagagacgaggtcttgtgatattgcccaggctgttcttcaactcctgggctcaagcagtcctcccaccttggc
ctcccagaatgctgggtttatagatgtgagccagcacaccgggccaagtgaagaatctaatgaatgtgcaacctaatt
gtagcatctaatgaatgttccaccattgctggaaaaattgagatggaaaacaaaccatctctagttggccagcgtcttg
ctctgttcacagtctctggaaaagctggggtagttggtgagcagagcgggactctgtccaacaagccccacagcccc
tcaaagacttttttttgtttgttttgagcagacaggctaaaatgtgaacgtggggtgagggatcactgccaaaatggtaca
gcttctggagcagaactttccagggatccagggacacttttttttaaagctcataaactgccaagagctccatatattgg
gtgtgagttcaggttgcctctcacaatgaaggaagttggtctttgtctgcaggtgggctgctgagggtctgggatctgttttc
tggaagtgtgcaggtataaacacaccctctgtgcttgtgacaaactggcaggtaccgtgctcattgctaaccactgtctg
tccctgaactcccagaaccactacatctggctttgggcaggtctgagataaaacgatctaaaggtaggcagaccctg
gacccagcctcagatccaggcaggagcacgaggtctggccaaggtggacggggttgtcgagatctcaggagccc
cttgctgttttttggagggtgaaagaagaaaccttaaacatagtcagctctgatcacatcccctgtctactcatccagacc
ccatgcctgtaggcttatcagggagttacagttacaattgttacagtactgttcccaactcagctgccacgggtgagaga
gcaggaggtatgaattaaaagtctacagcactaa.
SEQ ID NO: 3 (RHD):
aactccatagagaggccagcacaaccagccttgcagcctgagataaggcctttggcgggtgtctcccctatcgctcc
ctcaagccctcaagtaggtgttggagagaggggtgatgcctggtgctggtggaacccctgcacagagacggacac
aggatgagctctaagtacccgcggtctgtccggcgctgcctgcccctctgggccctaacactggaagcagctctcatt
ctcctcttctatttttttacccactatgacgcttccttagaggatcaaaaggggctcgtggcatcctatcaaggtgagagttc
attggaaaagtggtcacaggagcaaatagcaggggcaggggcgggggaggcctgtggttctccaggggcacag
atgttcctttctacaaaatcccaaggaaaaagattcccccatcttcttccgtagattgcaccgaaattcagccaacaatg
taagctttcctttagaagcagcctgggcatgccctcttctgtgaagcctgccttgatttttcagcacagtgagaggcatcct
ctttggtgttcctcaaattccctctaccaaatggtcttcataattctctgcttctctgcttccccttctctctcctcagtggcaagg
aatttttttatttttatagatttaggggatacaagtgcagctatcttatgcaagcaatttcatgttgttgggtttttggtttttgtttcct
ttttgtggcctctcgctcatttcttatttctttttgaggcagggtctcactctgttgcccaggctgaagtgcagtggcatgatcat
ggttcactgcagccttgacctcctagtctcaagcaatcttcccacctcagcctcccaagaagctgggaccacaggag
ggcaccaccatgcctggctaattttttttttttttttttttggtagagatgtgggtctccctgtgtttcccagactggtctcaaactc
ctggacacaagcgatcctccagcctcagtctcccaaagtgctggaattacaggcgtgaagcactgtgcccagctctct
tgctcatatctatactagttttcttttggaagcttcagcctgttgctaccccccacccccacccccaccgaccccagctttctt
ctcacttaggggctgggaagtctgcatgctgtctataaatccagaaccagaaggtatggctgaaggggagggtagg
atgatggttattttatattcagctaaaaatattcccagactgtgatgagacaactgtaaataagacagatgtccacaatg
gtgtgactttgcttttttaaaaatattgaaatgagtttcaggcatctcagtgggctgataggttgttgataatagacagggcc
tccttgaagaatgtccctgagacaaagttgaagcttgagcctggttgagtccttgcttgttcctaggttgatatgaacggct
agttaactggaagcaaagagaagtcatcctgggggccatggcagtgacaagtaggacttagggagggaagccctt
ataccatttaaggtgctggcccagagaggagccttcagtgacagacaaacaagagctggcacaattttaattcacttc
aatttactctaattcatttcaatccaatacaattcaatgcattccattcattcaaccatgtatgacatccaatgtgggatcca
gactcatgatgattagagctgatatttatgagcacttactatgtaccaggcactattctacatgctttacattgaaccctcac
aataacccaatgaggtgggtactattatgatcttcgtttttcatatgaggaaactaggcatatggatgttgagtaatttgcc
cacggtcgctcagctagcaatagcacagcgtatttaaatttagccaccctggatttagtttccttacacttaaccattatgc
atcatggccccattttacagtgggcttgagtctttgtcatataacccagtaggttagcagccactattccaaccctgtagat
tgactctagggtccatgttctttacccctgcaccgtgctactaacgtaggtacaaaatgtcctcagaaactcactttatac
ggaagctcagaggagggtccacaacccaggcaggggagacgatggtgtcaggggagggaggtgactgcccag
ccaggtcttgaaggctcagtaggaattacctgtgggacaaaggagggtcatccaagtgagggcacagtgggtgcca
tggcgtgcacacacaatagagcagactgagcctgggcttaacattgcattgccctggagcctaaaaggggaaaca
aagggccgggcgacgtggctcacgcctgtaatcccggcacattgggaggccaaggctggagaatcacctgaggtt
aggagttcgagaccagcctggccaacatggcaaaaccgcatctctactaaaattataaaaactggctgggtgtggtg
gcacacgtctataatccgagctacttgggaggccattacactccagcctgggcgccagagtgagacttcatctcaaa
aaaccaaacaacaaaaacaacaacaagaacaacaaaaaaacaaagaggagagcagggactgggtgtggtg
actcatgcctgtaatcccaaacactttgggagaccaaggcaggcagatcacctgaggtcaggagttcgagaccagc
ctggccaacatggtaaaaccctgtctctactaaaaatacaaaaattagccggatgtggtggcacgtgcctgtagtccc
agctgcttgggaagctgagggaggagaattgcttgaacccaggaggcagaggttgctgagctgagaacatgccact
gcactccaccctgggtgacagagtgggactctgtctgaaaaaaataatagtaataaataaaaataaagagggaag
cagcgggtggcagactcactgggctgcatacgaagtttggcttcagtctgaggtccgaatagtaaacagcagcgag
acaagtttgggtttgggtcatggaggaagccatgccagggctggtgttgggcacagggaaaggggcatggcttgag
acaccagaccagcgtggaggctgtagtgtagtattgacctgaggacttcaacattctgatggtgtacacacgattttttg
agcatgtaccatggttatatattacactttaagtattactttaagtattactacattaatatattttgtatgttacaataaatacat
acaaattaggaaaattgaaagagatcaaaatgaaatatataatattttcaaattactaatcataatggtgtcaatctcca
ggcagggtccattgctacagttgacgatagtggatgaaaattcactcctcagagtcttcttgataatttgaaattgtcttgat
tgacttgtcagatctgattagatcaacatgttttaaatctcgaatgtgactgacagcttgtacgaggagaagtttcactctg
ccttttcccttttgttcacttgactgccattatttctatgcttccaatctgtgtttttctgcacgagttggttaagccattacttcatttt
gtgaaagtttgttgagttaaacttaggtaacttaatctgtcaatccacttaattgaattcagtcctggtaaactataatagatt
attcaaacctgccaattctaaaaagacattttgagacaatcaggaaatctgaatatagcatgaatatcttacgatataca
aggattattgttaattttgttaggtatgataaaagcatggtgggttgtttttgtttttgttttttaagtctccatctgttagagaggc
acattgaaatggcatgatatctggggtttgcttttatgccagaaaaaagaaaaagtacagaaggattatagaaacaa
gattggtctcatgtgacaatcatcagagtttggagatgggcacgtagggtcatcgtgctgttctctctgttttcgtatatgcttt
aaaagttctgtaatagttaattaaaaaaaaaaaaaaacaccctggctgagcatttagggaggccaagtggggagga
tcgcttaaaccaaggagttcaagacgagcctaggaaacatagggagacccccccccatctctaaaaaaaaaaaa
aaaaaaaaaaactttaaaatttaacccagtgtggtggcacatgcctatagtcccagctactcagtaggctgaggtgag
aggcttgcttgagcctgggagcttgaggctgcagtgggacgggattgtaccacttcactccagcatgggcgacagag
caagaccctgtctcaaaaaaaataaaaatatttgaggtgaagcgaggctgtaataacaaatttaaaaatataaataa
aacataaaggctgggtgtagtggctcacgcctgtaatcccagcactttgggaggccaaagcaggcagatcacgag
gtctggagatggagaccatcctggctaacacgatgaaaccccatctctaccaaaaatacaaaaaaattagccgggt
gtggtggcgggtgcctgtagtcccagctacttgggaggctgaggcaggagaatggcgtgaacccaggaggcggag
ctttcagtgagctgagattacgccactgcactccagcctgggcaacagagcgagactccgtctaaaaaaaaatgaa
aataaaaataaatgaaacataaaaccctgccattagttgcaatatgaagaatatagagaaatgcatatcaaatccttc
tcattggaccaatattcccttagggcaccttccaaagctaggagactcaaggctgtatgacatcctgagcaagtgagg
ggtggcttctgggtgaatctgaatattaaatatttgcagaattgaaaacttcacaaagtacctttagagatagaatagcct
agatccatgtttctcaaagtgtggtccccagacctgctgcctcagcatctcctggaaatttagtagaaatgcagattctca
ggccctaggccagacctactgatcagaagctctgggcctggggcccagcagtctgtgttttcacaagccctcttggtga
ttcttctgtgcatgaaagttcgagaattcctggagctagactgattcaaatcttgcctctgtatcttagagaccttgggcag
attagtcaacctctttctgcctctgtttctacttctgtcagaggatgatagtacttgtttcattaagttgttgaaaggataaatga
attgacacacataaagagtattagcttttattatcaaaagctttttttttgagacagagttttgctcttattgcccaggggagt
gcagtggtgcgatcttggctcaccgcaacctccacctcccaggttcaagtaattctcctgcctcagcctcccgagtagct
gggattacaggcatgcgccaccacgcccggctaattttgtatttttagtagagatggggtttctccatgttggtgaggctg
gtctcgaactcccaacctcaggtgatgcacccgccttggcctcccaaagtgctgggattacaggcgtgagccaccgc
gcctggcccaaaagctttaatttcttaattttttaaataaaataaataaaactagaattgcttgttttcttccagctaccctggt
gattgtattgagcattttctggggtgtgtgttctttgctgtaatgactactggtctggatgacctgtgatgagaccagatggg
caggggcagtggaggagattctagagatatttaggagataagtcagctgtacttgatgaaaagagtggggagttaag
gctggctgcagatgtatgatttggcatagagaggtgccagttcctgagatgagagacagaaggggagggacaggtt
gtgaggatgaatgaacaatgatatgttcattctgggcttggagttaaggggcctatgatatgcttaggggaagcagag
agtatcaattacctattgctgcataacagccaccccaaacttagtggcttaaaatagtaaccttttaatttactcatgatcat
gattctgtggtgcaacaactgggctgggttcagctgggcagttcttctgttagtttcacccagggtcattcatgcatctgca
gtttggggtgggatggcctcagatgacctcattcacgtgtttggcagttggtgattcactgggggccattactgtaacaat
cgcctaccaggcagagcttccctaaggcttccaaactaggagactatcctgggtcctgtgctgtggataccactcagtc
ccccatccccaccccatattcctcaaaggcagagagaggggctactagaagacagaggagttttcccagtgacatg
taaacactccaaaccctggcaccttccacactgcagctttggtctgcccctttgggaaatctctgtttttcttcccaggctgc
tggaggggtgagagtcgccggtagagtagaggctgtgggcgaggaggtggcggcctcctgaggctgcagtggtctt
tccaggcagcagtgggagcacagggtggaggtcaaccctagagcctgggagagtgaagctgggtgtgacttcaga
gctgttggtgctgaagtttctgcaggccagaaggaggggcaagagtggggggggcgcagatccagaatcacgg
aggcagctgaccggaggaggcagctgcccaaggggatggactcagaaggccaaagtgctgttatccaaacgaa
ctctttgcaagtggtctctttgcaacaggcctgggggagagcagtcttgcctaaagtcacaccgctaatcagcggccg
gcacggggtaacagttactaacactcactacgtacccaatgctgggcgaagtgacttgcatgagccagcgagctca
atgctcatggcaatcctctgagcagctggcattgtttcatctcaattttacagctcaggaagctgggacacagaggaag
agccaggctctgaacactgacaacctgattgagagacccacactgttcatcaccgttacgctatatatgctgtatagaa
aggcaggatggcataatggttaaacctaggtaggtagggtttgaatcctcctgctaccatttactagctctgtgacttgga
ctagttatagcacctctctgtgcctccctttccccatctctaaaatggggataataaatcgtacctcctacctgaggctgttg
tgggctaagtctgtaaggcacgtagaacagtgcctggaacgtggggtactgtctatctgtgtgcctgctgttacaacaat
ggtgagtattgccttatctctcgctgctgaactaccaggttagacttctttctgcaagtcatgaggctttcataaacttttcctg
aaggctttccgtagaatgtacaattcccctctgggtccaggcatgggcgcccgggtagcacatccacttcttatcaccc
ctgaacaccttagagcccatcagcttatcaaaccagcagctgatgtgagtgcagagcagactgtgagaggtggagg
ctgataccagtgaggatgctccaagctgggacccagccctgaagcgggagcccagataatggatgggtggaaatg
ggcctggagcccaggagaagtgggaggatgagggggcagggggaggagaagcctgaaatcaaatgttatttcct
gaccagtttggggtgcatgagctctgtcaacagctcatggaaactgctgccctaatttcatcttgttggctgaggcacaat
tcctctctcagggacagtgtagagccttggggaggaaggccctgagcgcgtatacctggaatcagggaatcgggat
caggggcagcagctgtgcccaataaagcccccacccaggatcctctgacttcctcatctctttttttttttttttgagctgca
gtctcactctgtcatccaggctggagtacagtggtgcgatctcggctcactgcaacctcagccttctgggttcaagcgatt
ctcctgcctcagcctcctgagtagctgggattacaggcatgcgccaccatgccaggctaatttttgtatttttagtagaga
cggggtttcaccatgttggccaggctggtctcaaactcctgacttcaagtgatctgcccacctcagcctcccaaagtgct
aggattacagacataagccactgtgcctggccttttttttttttttttttttgtaaacagggtctccctctgtcacccaggctgct
ggagtgtagtggtgtgaccgcagctcactgcagccttaaccttctaggcacaagccatcctcctacctcaccctcctga
gtagctgggactacaggcactcgccaccacgcccaagtaattttgtattttttgtagagacaaggtcttgctatgttgccta
ggctggtcttgaactcctcagctcaagcaatcctccctccttggcctcccaaagtgctgggattgtgctgggattacaggt
gtgagccaccatacctggtctgacttcctaatctttagggccccaactctgcccttatccaggcaactctcctctccccat
cttccactaacttctttggaatattccagagctgtaaaagccttagagagtatcaagtccaactcctatgtgttacagaca
gggaaactgaggcctaaagagggtaatggacttgcctaagatcacttagtgaggtgagagaagaaagagctagag
acagcctagcctgtgcaaggacatagttccaggcattcagagctgggctctgctgccggcatgtttggggcctggtagt
tagttcactgctgaactaccaggttagattttctttctccaagttgtggagctttcataaacttttcctgaaggtcttccttacaa
tgtacaattctcctctgggcccggtcatgagcgcccctcacaggctctctctggtccccttctgtaaaatgagaggaaaa
tggaagaattgctctactcatggaatcttcaataagtctgggccctatgcatatagcattgctacaaaatggcagatgca
ctttaacaatcgtgtttaataaaaggttggatttgcatatctgaagtggggcatgcagtctccaactgaacacaagcctc
actgctcccgcatgtgcactgcaccttcatatacatatttcctgcttggctcctgagggaatttgagtaatcccaagagga
acccctgtagaaaatgtcccctggccacacacccccattcctaaggatgcaagcaggagatagaaacattccctgc
acctccctccttgttgtcagaagaagtgcaaagagttgaatccttcctaatgcccacttctcacccacgccccaaatccc
caggtcccatggaggtccttgggggcctcctatatcctggtggtgtcaggttgatttggaaatgtcagtgtcctcccttgtc
ctctctggcagaccctgggtatgtgtatgtttcaatggaagtgaatttaaatgtactttataaatcaaagactttttctgaga
ctttggagagttccagtaatgagagcttctcattgttatcaaggccagggctggagaccagtggcaggtgagttcctatt
gctgtgattgtcatgatgatgttgatgaacagtcactatttattgagcgttctccatgtgccagtcactgtactaaacattattt
cctttggatttcccagaaacctctcaggtgggtctaattacccttattcagctgataaggaaagtaagcaacttacaaga
ccacagggctatgaagtggaaacacataaattgatatttcattttatttatttatttattttgagacagagtctcactgtgtcg
cccaggctggagtgcagtggtgcggtctcagctcactgcaacctctgcctcccgggttcaagcgattctcctgcctgcct
cccgagtagctgggattacaggtgcccaccaccacatccagctaatttttttgtaattttagtagagacggggtttcacca
tgttggccaggctagtctcgaactgctgacttcatgatctgcccacctcatcctcctaaattggtatctttatatgtccaaaa
gagtcaactggtggcaatttagtgaggtttaatctaataggaaatgatagagctgggatcgaacagagccatgtgaac
tcaaaacctatgcttccccttccacctttttgaaaaacattgtctaggctgggcacgatggctcatgcctgtaatcccagc
actttgggagacggaggtgggtggattacatgaggtcaggagttcgagaccagcttggccaaaaattagccaggcg
tggtggcgcgcgcctgtggttcccactgaagcacaggaggctgaagcacaagaatcacttgaacccgggaggtgg
aggttgcagcgagccgagatcgcaccactgcactccaacctgggcaacagagagactctgtctcgaaaaaaaaa
aattgtctacatgctggttgcagaaaatttaaacactaaaactaaaaaagtaaaacacctcccaaacttagagacaat
attaatgacggaaaaaaaattcttcaagatctctctctctccagtcatttattcatgtgcgaaaacagttggtgattattgat
aaaatagcttttagagtttggagcaattatgtgcattacatataccatttgattctggcaacctaatgaaggagtatgatca
tttcccctatttaacagacaagaacaagaagagggagggcagatggtgtggtagtctaaggcacaggctccagcag
attatctaggtgtaaatcttggctgtaggccaggccctgtggctcatgtctgtaatcccatcactttgggaaaccgaggtg
ggcagatcacttgaggtcaggagttcgagaccagcttggccaacatagcgaaaccccttctctattaaaaatacaaa
aattagccgggcacggtggcaggcacctgtaatcccagctacttgggaggctgaggcaggagaatcacttgaaccc
aggaggcagaggttgcagtgagccaagatcttgccactgtactccagcctgggtgacgagtgaaactctatctcgat
attaaaaaaaaaaatcttagctctacccaccggggcaagttacgtaacgcctctgtgccttggttttcatatctgtaaaat
ggtgacagtaacagcacccacgtcaaagtgtggttgtgagaacgaaacaagatagtctatgtaaagtgattaaaac
agcgtaggcacatggtaaacgcttaggaaatgtaggctgttataaagctcagagatgttaagtaactagatcaagatc
acacagttagagggtgccagagtcctgatttgaacccaagtttgtctcgttctggagctcaagctgctaaccctttttcaa
aactggaattaaaccaaagtgctcaccctccgctttgctgggcccctccctgccctcaggtgcgtctcttccactcacct
gccacagcagcctctgctcagggtctgagaccgggaaaggtgagggctacccaggtggccctgatgttttctgccag
ccagctcaccaggtccctcgcagcaggcggcaaagggagggaggtttgctgtgaagattatgtggttcccaacaac
aagagcgctgggcctatctctgccctctcttttctgtgtgtcctgggacaagtcacttggcttctgtggcttcattttctcatgt
gcccagccagggggttggccctcatatgcaataacagcagcaatgacctttactgagtgtccatgtgcgtcaagcac
gtgtgctttacacttgttcttattattaggtttaataatagaataattgccacatttactgagcactcattatgggccaggccct
gccctaagtgcttaattagctttagctcctctaatccttatcttatccccacacggcatgttatgttatccccattattcagttga
gaacattgaggctcaaagaggcaaagtaacttgaccaaatacttgtaaacgatcttgcatgccccttccagctgccatt
tagtaagactctaatttcataccaccctaaatctcgtctgcttccccctcgtccttctcgccatctccccaccgagcagttg
gccaagatctgaccgtgatggcggccattggcttgggcttcctcacctcgagtttccggagacacagctggagcagtg
tggccttcaacctcttcatgctggcgcttggtgtgcagtgggcaatcctgctggacggcttcctgagccagttcccttctgg
gaaggtggtcatcacactgttcaggtattgggatggtggctggatcacttctgggtcatagagggaatggaccccgaa
aggacaggttccagaagatctgggatattgccccctctctgtctagcaccagtgctgtgcaatatttaggacatccttata
ctaaaagattattcattgtttaaaattcaaattaactgggcatcctgtattttactggacagccctactccgtgtatcacaag
gaatccaggcctacattcctcctgcatcctttctttcctgttattgtcgattatgattttgtaaagttacataatcaatataagttt
atggaaaacgtaagaaggaaacacgttagacagagagaaatagacatgccacacctagagagacattctattttttt
ttttttttttgagacggagtttcacttttgttgcccaggctggagtgcaatggcgctatctcggcacaccacaacctcagcct
tctgggttcaagcgattctcctgcctcagccgcctgagtagctgggattacaggcatgtgccaccgcgcctggctgatttt
gtatttttagtagagatagggtttctccgtgttggtcaggctagtctcaaactcctgacctcaggtgatccgcccgcctcgg
cctcccaaagtgctgggattacagacatgagccaccgcgtccagcctgagagacattctcttgaaaagaaaggactt
tcagccccctaatgctgctagacaataaatagccatgcctttattttcattaaattacctgtgctttgtttacatgcatttgtgtg
aaatgctaagaaccatcacaactaatgtatggtgccagaagtcagaatagttgttacctgggcaggaggtggatattg
attaggaaggaacacaaaataaccgcatggggtgcagaaaatgttctctatgttcacctgggtgatgattacacatca
agctatacacgttttaaaagggcattggcacttaataggaggaagtaggctaaattttttcctgaaacattgttttgttttgtt
caaacctctgaatccctgtgctgcccagatgatggtaaacgtcatcctaggcatcttagggacctctcaaggccattcc
agcctccccttctaagaccctgctaaacctctgggcactgctgttaaacatttctctatgagccaggaactgtgctgagc
actccacaaatattattttgtttaactcttccgggtagggatctaacctggtatacaggtaaggaagtggaagctcagag
agggcaaggcacttgcctagggccacacagctaagtggtggagatggctccaactttttattataaccttttccacatgc
tccagagtgctcagaacatgaaacacagtctagccagctcccgattggccctggagggaaaaaactttatatatttttct
tttttaaaaggtttagaggctgggcatggtggttcacacctgtaatcccagtacttttgggaaccgaggtgggcagatca
cttgagcccagaagtttaagaccagcctgactaacacagtgagatcctgtctctgcagaaaatagaaaaatcagcta
ggcgtggtggtgtgcacccacagtcccagctacttgggaggctgaggcaggaggatcacctgaacccagtgaggtt
gaggctgagtgagccatgatcgtgccacttcactccagcctggacaacagagtgagaccctgtctcaaaaaacagtt
ttaggggccgggcgcagtggttcatgcctgtaatcccagcactttgggaggccaaggcggggggatcatgaggtca
ggagatcgagaccatcctggctaactcggagaaaccctgtctctactaaaaatacaaaaaattagccgggcgtggt
ggtgggcgcctgtagtcccagccactcgggaggctgaggcaggagaatggcgtgaacccgggaggcggagtttg
cagtgaaccgagatggtgccactgcactccagcctgggtgacagagcgagactccgtctcaaaaaaaaaaaaca
aaaacagttttaggccaggcgcggtggttcatgcctgtaatcctagtactttaggaggcctagcaggtggattacctga
ggtcaggagtccgagaccaacctgagcaacatggtgaaatcctgtctctactaaaaacacaaaaattagctgggtgt
ggcggcaggcacctgtaatcccagctacttgggaggctgaggcaggcgaatcacttgaacccgggaggcggagg
ctatagtgagccgagatcgcaccattgcactgtagcctgggcgacagagtgaggctctgtctcaaaaacaaaacaa
aacaaaaacagtctatgagttaattcccaccagaattcaatacacacacgcacacatgcacgcatacacacactgt
gtccacctgggaagtgacaaagggcaccctgggggatttcaaatggtggtggccctggtttggtgttgctgccttagctt
aaggtcacaccagccttcagcctcctgccccacagtctagggctgctcccctcatctgatgtccacagggacctgtttgt
tcttgactcaatctagaaagacgagaagggagagaagtcactcgcagcctgagtgaactcccctgccccacccctg
actgcttggatccccctaggggtgacccctgctgaaactggctccttcctgaccggttcccgtcagggctgtgctgatgg
gtggtgcccaggcctgcccctggggacggggtactctcccttggcaacactccagcttgtgccacttgacttgggactg
atttggttctgttttgagtcccttcaggggaggggcctatcttattcaacgttgttgtttgttttcctcacatactgataacttagc
aaatggctattggagcaaaaatgaaaataaacggaactctgaagtgggatgttttaaaattttatttatttttttagagaca
gggtcttgctctgttgcccagtctggagtgcagtggtacaatcatagctcattgcagcctgtgcctcctgggctcaagtga
tcctcccacctcagcctcctgagttaaatttttttacaggcgcctgctaccatgccctgctaatttttgtatttttagtagacaa
ggggtttcaccaggtgggtcaggttggtctggaactcccgacctcaagtgatccacctgcctaggcctcccaaagtact
gggattacaggcgtgagccactgtgtccagcctaaaactgtttttgagacagggtctcactctgttgtccaggctggagt
gaagtggcatgttcatggctcactcagcctcaacctcactgggttcaggtgatcctcctgcctcagcctcccaagtagct
gggactgtgggtgcacaccaccacgcctagctgatttttctattttctgcagagacaggacctcactgtgttgctcaggct
ggtctcaaactcctgggctcaagtgatctgcccacctcggctctgaaaagtactggaattacagcctcctgagtagctg
agaccacaggcacacaccaccacacctagctttttttttttttgctttttgtagagatggagtctcactatgttgcccaggct
ggtctcaaactccaggccttaagcaatcctcccacctcagcctcccaaagtgcgaagattacaggtgtgagccacca
ttcctggccttaaaagtgtgatatttttaatgtattttgaaatctgcaggactctccctagaagataatagcaataaccaact
cctttattgtgcttgacgtatatcaactcactttgcccttaccgtggctccagaggcattgggtccaccttataaatggagg
caccaaggcacagagtgattaaataaattgcccaggatcacacagccagaaagtgtctgagtcaagattccagccc
aggcagcctagacctgagagcacgctcctaaccactgcacatcactgtcttagcacctcctcagcacaaactggccc
ttgaggaatgaaataccgccgccggcacacacgctcctgagttaagcctttgtcaatgaaatgaacacccacttaaa
aggaataacctgtccaggcacgatggaacattgagtaaccccttattctaaattcctggtccctgtaagactccttcccc
atgcccttgcccttttctgaccttcccctaaagtccttgaggcttaagcgggcatagtctgcagcaaacactggggaagc
tgagtccagacttcagagcacaggctttggatctaggccagctggatttgaacctcacatttgtgatcagctggcatgac
tgtttccaaaaagtccattttaatcctctacgtgaccctctgtaaaatgggatactgaatggtgagctagcacgattttaca
gagagtgaattttttttgtgtgtgtgtgaggcagtcttactctgttgcccaggctggagtgcagtggtgcagtctcggccca
ctgaaacctctgcctcccgggttcaagcgactgccatgcctcagcctcgagagtggctgggattacaagcatgcacc
accatgcccgggtaatttttgtatttttagttgagacagagtttcaccatgttggccaggccactcttgaacccctggcctc
aagtgatccacctgccttggcctcccaaagtgctgggagtacaggcatgagccactgcacccagccttatagggtta
aaatttaaaagaggtgatgctgttacaagcctgttttacaaaatgctcttataataaatcattatcatcactgttgctgtggtt
gtagcatcatcatcattaactcccagagggaggagggagtctcagagcaagctgctcaggggagactggatgtcca
tggattgtccagctcagtaccacttcctccaggaagtcctccctgataagtccagtcagcatcaccctctccttccaatga
accccactagccttgtgatatcacagatattcttagttgacaggctcatggtgtagcctgtctagatcataagtacattttttt
tttttttggatcataagtatcttcaagaccaaaataattttctactcctgagcatgctcattggtcaaaggaaggaaggaat
cataatagcgttaataaggctagcgtcttttcagaagttggttctttgtgccagtcttggtgctagacacaccgataggaa
gaatactccttcacatccccaggacaccaacatgggatacgtttgatcatcattcttaatttgcagaaggagaaatagg
ctcagtgagatgaaatagccactccagtggcaaggctgggactggaagccgggcttgtcctgattccaaatccagttt
ctttccactgccacggagacggagagaagggacagtggccccagatggggatggggtgactggatgtgggcagg
cctgcgggggaagagtgccctctgttgagcatccgaatgatggcagcagaaaagaagactgggcagaatcccagt
tatcagatcccctgagggaacagtcaccccgatcaccctcagtcagatgagtgtgtgtagatcaatgcctcatagatg
aaggcactgaggcacagagtggttaagtcatctgccagaccacatggctcagggtgcagaggccaccttaacggg
agaagagatggtcactccactctgcagcatcagcgcccaggtgggtagaaatcttgtcttctattcccacagaaagta
ggtgcccaacagtgtttgttgaaagaatgaatgaatgaatgaatgaatgaatgaatgagtgagaggcatccttccttct
cagtcgtcctggctctccctctctcccccagtattcggctggccaccatgagtgctttgtcggtgctgatctcagtggatgc
tgtcttggggaaggtcaacttggcgcagttggtggtgatggtgctggtggaggtgacagctttaggcaacctgaggatg
gtcatcagtaatatcttcaacgtgagtcatggtgctgggaggagggacctgggagaaaagggccaaaagctccattt
ggtggggtttccagggttttgaaaaataaagacaacctgtaatcccagctacttgggaggttgaggagggaagatca
cttgaggccaggagtttgagaccagcctgggcatcatagcaagatcctcatctctaaaaagtaattttttctaaattatcc
agttgtggtggcatgcacctgtagtctcagttactcaggaggctgaggtgtgagttggaaggattgtttgagcccagga
gttagggaccgagctgggcaacatagcaagacctcatctctaaataaataggtaggtggatagacagatagataga
tagacagacagacagacagacagacaggctgggtacagtggctcacacctgtaatcccagcactttgggaggcca
aggagggcagatcacctgaggtcaggagttcaagaccagcctggtcaacatgggggaacctcatctctactaaaa
atacaaaatttagctgggcatggtggcaggcgcctgtaatcccagctactcaggaggctgaggcaagagaatcgctt
gaacccgagaggtggaggttgcagtgaaccgagatcgcgccattgcactgcagcctgggggacaagagcaaga
cttcatctcaaatttaaaataaagaaaaaagaaaagaaaagattgatagatagatagatatccaaatgagtttacaa
aaatgtggtctgtgcaaatgtttaaacacaacaaaccaatgcctttaactactacagtataatcctgtaggattgtgctatt
catgatataattatggttatataaaagtaattaattctcagagcctcaccagcagtgggtccagcaagtttgtacagcca
gcatcttctttcagtcagtgcgtgtcagtaactgcatatgtcctctcattgggagagcctgtcgaaagtctaaatttgaagg
cagctgtgaaggtaaggccaatccaaatggctctcccagatcctctgctgtaaccctgaccctgagtgaggacatag
ccaaccttcccatctcataggtgagaaagctgatgcctggagaggggaagggactgcccaagatcacatagcaag
atagtggcagaacccaagcgagaacccacagttccagcctggcttagaagaaagtgcactggacttggagtcaaa
ggctggggtttgcatcccagctctgccataaatccctgtgtgactctgggcaatttaacctcttagagctttagtttcttcatc
tgtaatatgagggtagcagtactaccacatagggttttgagggagtaattgaattaatcacatgagatgatgcatgtttac
aaaaaaaagcatgaagcccctttactgtgcctcagtgtcccaaaggactttggattttactctgagaaatacagggag
aactagggagtgttgggcagaggagagccatgatctgacttatgttttaagatactctggcttctgggttcagaaaaga
ctgaaggggcaagagaggaagcaggtggagaccagagcggcagtgattgccatcatccagactcagactagga
caatagctgtgagagtgatgggaagtggttggatcctgactgtattttaatagcagaattgacaggatttgctgatagact
gcacgtgggggggagagggtcaagatgacttcaaggttctcatctggcacaactcagcggctgctggtgccatttac
tgagatggggaatgttggggtgggatagatctgggagggaaaacccagagttcagtgtcgaatgtggtagcgttagg
gttaaggttgggggagggggggtagagatgtgtatgaaacatcccagtggagacactgaatggagatgtacaagtc
tgaagcttagtggaaaggttagggctagggatataaatttgggagttgttacaatacagatggtgtttaaagccatgag
acccaaggagatcactcaggagtgaggataaagagagatgggaagaagtctgaggactgagtcctagaacaccc
tgcattttagaggggggacatgtgtaagagccagcaaaggagacagaattgtgcttggagaggcaggaggaagcc
caggagagcgtgaggtcctggaaggcaaggaaagagagggccccaggtgggctgaatgctgctgagaggtcaa
gtcggatgagggctgggaagtagccattggatttggccaggagaccttggcatgcatggttgtagaggaggatgaag
gcaacagcctggcttgactgattcaagagcaggagatgagaaagtggagacagcatgcaggggcagctctgcca
aggactttgctataaaggggaacagagaaatggaggagaagcaggagggcaataatccgatagagaggaaaa
atctgatgatacagaagagagatgaactgcaagagtcaagcctttgagttggaaagcaggagtgggattttgagcac
tgatacctttaggccgatgcagggacagttcatcttttttttttttttatacaacattttatttaaaaaaattattttcatagaatac
attttcacattagagattcccattgtgcggaaataacaatttattacttatagttttatatttgtggacagattgttttagaacaa
gtagaatacatttgagaattaaatctcagtttacaatggataatattttgatatgtctctggggaaacttgcccttaaatgga
acttctgtatcttcagaagcactccaagcgtttcttcctaggatttagaaatttataatatgagatagcagcatttcctaatttt
aaaatttccctagtatatgtaaccatcagtaggtggtatctactgactagagagggaagtttttgaaaattaaacactgtc
taattttctgcaaagtttttattcatgaattaagagtatttccctttgtccattattcccaaggcaaatatggaaatttgatcatg
tactaatcataataaagctggattctctttaagagattgagaaattaaaaggcaaaagctgatatatcatgtttagttatat
tgtgagtcttataagaagctgggaggcaaccccattaactcaccagaatacagaactcagtctcacaacttagatata
attcctctcaaaccttttcctcaaagattaaattctgaaaataatcttgtgattaagagaagaaggctgtccaccaatggg
cttatctgttatttcttccttattgtgagcttaatggcatgacaaagcagaggcaaagaggcatacatcaattcttcaaagt
aggaagtcaaaaaggtcagagcttccacagcatggcaacagctttgcagatgcccacatcgtgatagttgaaatag
caaagcccagcaaaggttaaagctgaaaatgccaaaagccctgccttggcagctttctgcgaggcatccccatgaa
cataatcagtaacaacttgtccaaggccccagtgaccatgaagagtgagggctgcagccagggaatagtccgtcgc
agagcaaggattcaaataagcagccggaagcagacccgggagcaaaacactgacaaccctctcgctagtccagt
ggagagatgcagccttggagccagaatggtggctcggtgacaagtgtatgtgctgcactccacaccattctgggata
ggtcggtcctgaagaaatgctgagatatgagcaggtctgaccactggagttcgcagcaacagagctcggcctccttg
ggcaccgcaaacggcactcagcctccagggaaccgccatctcgttcctgaggcggagagttcatcttaacgagaga
aatggcagggactgtgaataggccggcagatttggtggcgggtgccacaggttcagtctcctgcagggagaggaga
aaatgccttactaattccttgtattttctcagagaaacaagaggcaccgtcatcagcctcatgtgagggtgggaaggag
ggatggggtttgcggagagggaaagtgtggtatggtcatctgtgggagtggaagagagtgagagggctgcaggggt
gcagcgggactgcaggctggcaccagggtccctagggcttgtagttggtggaaagtgcatcagtgaccagggctgt
gtgcagctgctccaggcaggtgtggaagaagcagagttgaacttgcccagcctggagtgctgcccagagtgagccc
aaagcccaggggagaccagagatggggctgtttgcaaaggaggaagtataacagtagcccacaaaatctgagct
ggttaagaaaggagagagagtgaaaatggggagcccagcctggcagcctgggtacacatctcagctcaacccac
actagctgaatccatttgggccccttcgttgacctctctgtgcctcagtttccctatctatagaatggggataagaataagg
ctacttcctagggctgttgtgaggattgaacaagtgaccgaacacttgttcaattttgaacactgttctaaagcatttagga
cagtgcctggcatggggtaagtgttgcggcagtgctgttattttcatcatcaccattgttctcaggctgcgttgattggagct
gctgaagggaggcaatttaaggaagtgagccggacagataggaggtggtggtggttatcaggtgcgatgcttgaaa
ctgaggcttcggaggcaacagttactggtaatgacaaggtctaaggcttgacagtgggtggcagaagtgtaacgca
gggaaagagacgagcggtcaaggagccgagagggaaggagttgggtggactaagatcatttgtggaagaatgat
ggagagaaaggctgaagggcaggggctgacatcatcagtgaccaagaggcggccgggaggctgagaccacag
caagaaagggagagtgtgatggcatcttcttcaagggagctggggatgtttggggtggaaaaaagaacaatggtct
gggagggaatatgggaaatttttttttttttttttttttttttttttgagatggagtttcgctgttgtcatccaggctggattgcaatgtt
gcaatcttggctcactgcaacttctgccttccaggttcaagtgattctcctgtctcagcttcccgagtagctgagattacag
gcacacaccaccacgcctggcttacttttgtatttttagtagagacggagttttgccatgttggccaggctggtctcaaact
cctgacctcaggtgatccacccgccttggcctcccaaagtgctgggattagaggtgtgagccaccgcgcccagcctg
gaagtttgtatttattaatttttggttgtcttcatctgtgtatgtgactttaacccctaaatacttcagtgtacatttctttttttttttttct
ttgagacagagtcttgctccatcaatcacccaggctggagtgcggtggtgtgatctcggctcactgcaacctccgcctc
ctggattcaagcaattcttgtgcctcaccctcccgagtagctgggattaggggcatgccaccatgcccagttaatttttgt
atttttagtagagatggagtttcaccatattggccaggctggtcttgagctcctggcctcagttgatccacctgtctcagcct
cccaaattgctgagattacaggcgtgggccaccataaccggcctcagtgtatatttctgatgcagttgggttctgtatccc
cctccaatctcatctcgaattgtaatccccacgtgttgagggcatgacctcgtgggaggtgattggatcacaggggtggt
ttcccccatgctgttcttgtgacagtgagtgggttttcaggagagctgatggtttgaaagtgtggcacttcctctctctctttct
ctctctctctcacctgacaccacgtaagatgtgccttgcttccctttcaccttccaccatgattgtaagtttcctgaggcctcc
ccggccatgccaaactgtgagtcaattcagcctcttttgtttataaattacgcagtctcaggaagtatctttatagcagtgt
gaaaacagactaacacaatttcctaaaacaaggggacattctcttacataaccttttttcagttaacaaaaatgagaaa
ttgacattgatatattatgattaccttattctcatttcaccaattttctcaataatatcttttctagaaaaaaatatatattttttgtg
gtcgaggattacatcttgcatttagttctcatgtcttattaaattccatcaatctggagcagtttcttcatctttctttatctttcatg
accttgacatgttttgaagtttcgagccagttcttttgtagaatgtgggtttgtctgctgttcctcatgattagattgtgggtatg
catttttggtaggaattctccaagagccgtgtgtgcccttcttagtatatcatatcagaagacatgctatcaatttgccccatt
actgggtgtgttaactgtgatcattgggttaagatggtacctgccaggatcttccactgcaaagttactattttcccctttgta
attaataaacatcttgtgaggagataatttcctatagaaatcctgttgatcatccaactttcacccactgattttagtgttcatt
gattcttccctgaataaattagtactataataattgccaatggtggttttctaattccatctttccttcagtagttggcattcttct
gtaaggaaaagctttcgcttctctgttcatccactcatctatgtacttatttatatcaccatgggctcctggattccggtttaca
cacttccattttctgccttttctctctgcttaatataaggattaatgagaactccctgattcccaggaagaaaatgtcagcag
agctttcttaggcggaatgaagagaattcagtgtaagaaccataaaggtgtatctgtgtagtatggacagttttaaaaa
acaaacaaacacaaagaacctccaagggcaggaggtgctgccagactcaggagggcactagaactggctatga
gaagccactgagatcccaggtagtctgtgctctccatcttttggctcttattctctccgtacatctaacatctctgtacacca
gctttctctttagcgaaaaacgtgtcccctccacccacccatccacctccacttgttcctgcatttctatgtcccagatcctg
cagaaaacaactcttttctctcagttagtctcaattctgtagtccagggagagagaatctgatcagtcccctgggtcattttt
ccactctggtccaagcagctacagctggcatgggaaatagttcacacagtaaaaacatggctgtcaagaagagga
gtaaatttcagaggcagaacactccctgtgagcccgaacctcttcctgctttgttgcagtcttcataacgattgctttaaaa
gactgcattgatataacatcatctctcttctctgcatctttgacttgctagcttaactggtctagaggagggcttagcactgat
tttgagtattcattttcctcaaaacttcaattcagcctgggtttcttcagcaggagggcccgggggaaccagagccaggg
accagagtcatttcagtgcaccagctcaagaaatgaatattccaggccaagaatccccaagtgttcttcctgaactcct
tcctggtggagttcaaagagatgaaaaacacaagcccgcttttcagttcttatcaggaaactgcatagactttcctcttta
tgtatgactgagggctttttaccatcatttgttcccttcacaaatatttatttggtatttactatataccagggactcttgtggca
gtggaaaatacaactctcatggaacgtctgttccagaaggaaagactgccaataaacaataaaataggcaaaaga
tatagcatgttagagagtggtaagtaccacagataaaaatgaaatggagaaaagaaacacgaaaagttggggag
agaggataactgtttgagagggtggccaggggcagcttcatcttatcaagagggtgattttttgagtacagacctgaag
gtaacgagtgcacaagccatatgggtacctgagaacagcggcagaacaatggcagggtgctgggagggctgttta
ccagccacgctgtttagaattgtcagcacatggtgataaaaaaaaaaaaaaaaaaaaaaaaacaggctgggagc
agtggctcatgcctgtaatcccagcgctttgggaggccaaggcggatggatcacttgaggtcaggagttcgagacca
ggctggggaacatggtgaaaccccgtctctactaaaaatacaaaaattagccgggcacggtggtgggtgcctgtaat
cccagctacttgggaggctgaagcaggagaatcgcttgaacccaacgggtggaggttgcagtgagccaagatggc
accagtgcactctagcctggcgacagagtgagactccgtctcaaaaataaataaataaataaatacaaataaaaag
cagacagactttttagttggctttagaattcttagacaccctctacagacaaggcaccccgattgcttgcacccagggtg
gactactccctccaccctgcccttgttacaccctggctgggggtcagcatttcaggcagctgaatgacccaaagtggg
aacacgctagtgggtttgaggatgagcaagtggaggagggcaataggaggtgacgcccgagaggtcaggtgaga
gtggatcctgcagggtcgtggcaagaacctggaccttgactttgagtgacatgggagccgctggaggcttctgagca
gaggagtaacatgatctgacttgcattttattttatttatttatttgacgcagtgtcactctgtcgctgaagctggagtgcagt
ggcgacatctcagctcactatagcctccgcctcccaggttccagtgaatctcctgcatcagcctcccaggtagatagg
attacaagcaagcatcaccacgcctggctaatttttgtatttttagtagagacagggttttgccatgttggccaggctggta
tcgaactcctgacctcaggtgatccacccacctcagcctcccaaagtgctgggattacaggcaaaattagaatatatct
agaatttcctgaagaccttagtttggtattataagaagtctggttgcttcatgttgcaaaatttatatcactcatcactcccgc
agagttaaaattccgctgagaagtaggaatcagtgaggtgcgtgtccatgtgggtttttgccacacctaagtgaaccttg
gtcaaaagcatataagagctactgataggccgggtgtggtggctcatgcctgtaatctcagcactttgggagggaagg
atctcttgagcccaggagttcaagaccagcctgagcaacatagcaagattccatctttacacaaaatttaaaaattggc
caggcatggttgtacattcctgtaatcccagctactcaggaggctgaggtgggaggattgcttgagcctgggagttgga
gactacagtgagctgtggccacaccactgcactccagcttgagcaatggagcaagactctgtctcaaaaaaaaaaa
aaaaaggccaggcgcagtggctcatgcctgtaatcccagcactttgggaggccgaggcgggtggatcgcctgaggt
caggagtttgagaccagcctggcaaacacggtgaaaccccatctctactaaaaatacaaaattagcccagcgtagt
ggcgcatgcctgtaatcccagctactagggaagctgaggcaggagaatcgcgtgaacctgggaggcaaatgttcc
agtgagccgagatcgtgccattgcactccagcctgggcagagcctgctgggttgggctgggtaagctctgaacacca
gtctcatggcttcaagtcacacctcctaagtgaagctctgaactttctccaaggactatcagggcttgccccgggcaga
ggatgccgacactcactgctcttactgggttttattgcagacagactaccacatgaacatgatgcacatctacgtgttcg
cagcctattttgggctgtctgtggcctggtgcctgccaaagcctctacccgagggaacggaggataaagatcagaca
gcaacgatacccagtttgtctgccatgctgggtaaggacaaggtggggtgagtggtctcctacttgggctgagcagaa
tggctcagaaaaggctctggctgaaaaaatctccctcctttaccaagttcccctgggtgtctgaagcccttccatcatgat
tcatttctttgagtagtgtttgctaaattcatacctttgaattaagcacttcacagagcaggttcaggaggcctggggtatgc
agatttcaaccctcttggcctttgtttccttgtctgtaaaatgtggttagctggtatcagcttgagagctcggaggggagac
gtgacttccccatctaactctaagtgacaaggctgagactctccagccctaggattctcatccaaaacccctcgaggct
cagacctttggagcaggagtgtgattctggccaaccaccctctctggcccccaggcgccctcttcttgtggatgttctgg
ccaagtttcaactctgctctgctgagaagtccaatcgaaaggaagaatgccgtgttcaacacctactatgctgtagcag
tcagcgtggtgacagccatctcagggtcatccttggctcacccccaagggaagatcagcaaggtgagcagggcgct
gcccttgggcagcacttgggtctaacaggactagcacacatatttatgcccctccccaccccagggccagcgtgggtt
gggagagggcatgccgggtggtggagctgtgcctgcctctacagtggagctctaggtagaatgctgggtggtcacag
tgggcctgggactcaggagactgtccagtgatcaaaggctttctgggggtagtgattaaatccatccatgctaacatga
aacagacctcagtttgaaccccatttctgctagttgctaaagtcagtcaccatgagcgagagtcagcagcaacagact
agactagaattagccagcctctctcttccccccaacaaatttcaagaatggaaccatcagaatcagaagtagagaag
tatgtgacactagccatgtggctctggtcaagccacttcaacgttttgagtctcagtggcctcatctgtaaagtgggaatt
aagagatggtgcatgtaaagtgcttaacggggagtaaatggtaggcaaacattagctgctgctattagtaaagagag
acgatggtgtgtgtgagtcttgtgggcagagatgggtgagaggggagacaaaacaagttctcatgatgatggggga
aggggctccagctggtggtgtcggagggaagtctggacagaccagtggggggctcgggtgggaggcactgggg
gggctggagtggaaagaatgtggccacagatgacagcttcacagcagaattcagtgctaagaggaagtgagtggc
catgagttccatggtgacagaaagtctaagacacccagcaaggcaggagtgggtgtcaactcagggaagcccag
aggctaatcctaggtgagagctgagggtgtcagataagagcaaggcaaggctccggttctggagcagtgaaggac
atagcagagctatgacccaggaacaaggcccagcttattgaaactgggcccagtcacacagggggcacaggca
ccaagtagccaataataataataaaaacaataacaatgatttgtgtctactgggcatttattcatgttctatgccagacac
tgggctaagagctttatatgtggaaactcatttaatccttacaataaccttatgaagaaggtacatccaaaaccccattct
tctaggccaggtgcagtggctcacacctgtaatcccaatattttgggaggctgaggcaagaggattggttgaggccag
gagttcaagaccagcccaggcaacatagcaagaccctgtctctaaaaaaaaaacaaaaacccattcttcccgctg
cccagggacacaccactaatgagtgtgatgggtgcctaggatgctgagcacctggacttcccagctcattccctaaat
gctgcacaatcagggtaactgtgccctgagcctaagaggcagtagtgagctggcccatcatgtccactgatgaagga
cacgtagccccaacacaggggagaagtggtttcaggatcagcaaagcagggaggatgttacagggttgccttgttc
ccagcgtgctggtcacttgcagcaagatggtgttctctctctaccttgcttcctttacccacacgctatttctttgcagacttat
gtgcacagtgcggtgttggcaggaggcgtggctgtgggtacctcgtgtcacctgatcccttctccgtggcttgccatggt
gctgggtcttgtggctgggctgatctccgtcgggggagccaagtacctgccggtaagaaactagacaactaacctcct
ctgctttggctgaaggccagcaggacgctgggacctgatgggccactgtgcagtgcacagctgcattaggcaggtgt
cggcgcattctcttattggcttcaacgcctagtgagggatccatcctggctcggtggcgcatttgttaagatgctcgggag
caggtggcagaacccatttgagcttgcttgggcattggggagaatttgttatcaggctactggggtgtcacagaactca
aggacagggactggagtgttgtggggagccccgaagcccctgttttacttctttctttgcttttcctgaatatctgctttattctt
actctatagacatgcttcctcctctttcaccccacattgtggggtgtagtcttttgcttcaagaaagcagcctggtggatgg
aatctcttggccccaatcccaaattctctggagaaggggctctttggtttaacttggataatgttgtcttcagctgggggtg
ggcacatcgtgcatatgtggctgctgccggggaaccacgtggatgatgtgagaggagcagcacccagaagaggg
agtgctgggctgatggtccaggtcgtgtccacttctgattgtttaattcttcttctaagtggatggatctttctccaatactcag
caaatcctgatcgttccagaatacttcattatagccaattggttataatgtgcttctctaagagaaatatttagggacaaca
aatcttcatgggtttgaagacttgatggaggaaaaaggagtagattttcgaaggctggatttggatgaacaggggctat
tcagggagtgcattccaacctaaaattaggaaaaactggctgggcgcagtggctcacgcgctttgggaggccgagg
cgggcagatggcctgaggtcaggagttcaagaccagcctggccaacatggtgaaacccatctctactaaaagtaca
aaaattagccaggcatggtggcgggcacctgtcatcttagcgactcaggaggctgagacacgagaatcacttgaac
ctgggagacagagcttgcagtgagctgaaatcgtgccatggcactccagcctgggcgacagaacaagactctgtctt
aaaaaaaaaaaaagtggtttatatacagagtggaatattatttagccataaaaagaatgaaatcctgtcatttgcagc
aacatggatggaactggaggtaattaaaaaataaaattaaataaggaaaaacgtatcaatacttcgattaaccaaa
accagggcaaatctgattttcatctttgcaaggggaacaaatttcttttatctcctctggctttgaaaccctgaaatgaaag
gaggaagggcagaaaaaagaacacatagcaagttatcatcagtctcagcgcccatcgcattccctgagcttgtttcct
tgacttcatcactggcaggactattcaaaaatgattcgctcattcattcatatattcattcattcatcattccttcattcaacac
atacgttttaacactcatcttgcttttcaagctatagtttagtgagcgaaatggatacacacaatacagtgtgagaacagc
aagagggcacatctgagctagcctgggatgggtctggaaatgcttcctggagcagaggaaacggttgacagccaa
gtgttgacagagaagtagtattagccaggcagagacatggggaatgtattccaggcagaaggcacagtgtgtatga
aagcttattgttaagaagagtgtgtggcccaaccaggaaacagacattctaaaggcatagggtccacccaggagca
tggtggacccagatccctgaaagatgggaggtgctcaggcacacttcctgggctagttgaggagtctggatatttattta
tttatttatttatttatttatttatttattgagacagagtctcattctgtcacccaggctggagtgcagtggtgcaatctcagctca
ctgcaacctccacctcctgggttcaagtgattctcctacctcagcctcctgagtagctgggattacaggtgcccaccacc
atgcctggctaattttcgtgtgtgtatgtattttgttgttgttgttgttgttgttgttgttgttgttgagacggtgtctcgctcttttgccc
aggctggagtgcagtggcgccatctcagcttactgcaagctccgcctcccgggttcacaccattctcctgcctcagcct
cctgagtagctgggtctacaggcgcccaccaccacgcccagctaattttttgtgtttttagtagagacggggtttcaccat
gttggccctgctggtcttgaactcccgacttcaggtgatccacccatgtcggcctcccaaagtgctgggattacaggcat
gagccaccgtgcccaacctggatttttattctgaagactaatagggattctaaggaaggaaccagcctgattgaatttg
catatgtgtccacatctgctggctcacggctgtgtgggaggctgagtgatggggaggaaggattactgagtagggatc
tgaaggtgtggcctcatgctttctttctaaccagctgtgttgtctttgggatggtgcttaaatttgggctagaccagtgggtctt
ggtcaccccccaggggacatcttacaatgtctggaggcgttcttggttgacacagtggggtgagggctgctactggca
gctcgtggggagagaccagggatgctgcttaacatcctacagtacacagggcagcccccaccacaaggaattatc
agctgaaattgtgaacagtgtctacactagacccttgctactcatagtgtggtccgtagaccagcagcattggcatcac
ctgggaccttgttagaaatgctgttagaccccaccccacatccactaaagccagctcttcatttcaacaaactccccga
tgatgtgagtgcacattcaagtctgagaagggcttctttgaggtgagccttagtgcccatccccctttggtggccccggat
accaagggtgtgtgaaagggggggtagggaatatgggtctcacctgccaatctgcttataataacacttgtccacag
gggtgttgtaaccgagtgctggggattccccacagctccatcatgggctacaacttcagcttgctgggtctgcttggaga
gatcatctacattgtgctgctggtgcttgataccgtcggagccggcaatggcatgtgggtcactgggcttaccccccatc
cccttaacactcccctccaactcaggaagaaatgtgtgcagagtccttagctggggcgtgtgcactcggggccaggt
gctcagtaggcttcggtgaatatttgttggctgatttattcagaaattctgtccagcccctaccttggatggatttatcacctct
ccaggccacctcttctttccaaatagggccacctaggtatagaccaaagacacgaaatcttttgtgatcccacaaaca
cagagcaggtcaaataggcccaagccaattgagactgtggttcaggtcgtgatgcagagctttgctgtggacgtgctc
ccactgcgtactagctgggcatgtggcttaacctttctcagcctcagtcgccccattgtaaatggagataatgatactatc
tcccctcacaggactgttgggatgctactggatttaataagctaatgcagggacatgctaagcacaacccatccctga
ggcccagagagggggggccttggctgaggtctcactgcgaggtgggaatgtgggcctccagaccagaggtaggt
cctgtggcccctagacagtggacagcaatggtcagtttgacacaccagagccctagccattacttcctggatgttgtgt
gaatattttctggacatggcttatataaaatgaaaaagtgaattgggcacgatacagggatagatttttagagatgaact
ggtagcatgatgataatcatattcactgataacatttactactgttattgactgctttaaaagtgttgggcattgtgctagaa
accattatatgcattatctccttgaattctcacaaccgcctactgaggtattctcagactctaagaaatgagatttaagag
aagttatctgcccaaggtcactcggctggaacctggctgtaaaaatggctgaagcaggtgatgaggagctgatgcgtt
tggacgtgtctcagagaaatcatggaggcgctgcggttcctaccggttcttggatgccttctacagagacaaccatagc
cccaaattatagggatcacatatcagtgggtgagacatccttgcttgggatgaggaggggatgagctgtgtgaagca
aggcgcctctgtgatgggttccagtgatgtgtctgccactgtcttaataactgtgcaattctaagcagaacctttcctgtctc
tgggcctgagagttcccctctgaaagatgaggacttgacctagcaaggtcctactcacatgcctgtagagaacaggc
aggggaagttagaaaaaaaaaaaagccagtgaaggaagggagctcttcagcttgcacccatcatcacagtgcag
ggacccaggctcagtgttgccagatccaatgacttctcaagagctcaaaatctagagttttgcatgtgctctcccaagta
ctggcagaaaattcaagattgttagtaacactgtgtggctaaattctgcttgtgggctgcctagattcccaattctgtgattc
tgtggttctctggaagcattggttctccacagcacctgcatcacttggaaacttgttagaaatgcaagccctacctacgg
ccccaccccagacctacccagttagaaatctggggggggacctatcagtccatgtttgaacaagccccacaagtgtt
ctcttgcaagctcaagttttagaaccactgacctatagccaaaaaagaaaaagccaatcagtggttttctggtaaagg
attaacttaacaaactggctttccaagaaaataaagccttgattggtagcacttgcaatttctatggtacaaacgcttccc
gcatgactgagttcaagctgtcaaggagacatcactatacatggacttgggaagagatgagaacaatcagcccact
gagcctatgggaactggctccagcacatccctgcaagtcaactctcatcagggtgagtgagttgaggaccaagaag
cagttatcctcttgcctttgcaggacccaggcaaagggaagggcatagtgacagtgatgatctctcttccggaagtcttt
ggtttgctgagagtaaaaggcgtgggcttcaccagtggtgaagccagtcatgcagccttagtcctggtactgaaactct
ctaaatctcagttttctatctgtaaaatgggaaaataagacctatgtcacagggttgctgtgcagatttagcaacagaac
atagccccgttctttatgatgactgatgctgcatccgtatgaggacatctctatgtaatggaaagatggagagaggatta
agcgcaaagtcacaacacttaatgggaactgtggattagctacttggtggcattgggcaagtcagttgactttgcattaa
ttccacaaacaatatttcccaatttcctattcagatgagcatatgtgattgagtcagatgctgtgatcagaaccaggatgg
agcatttcccacaaactgtgggatttttaagtaatgggaaggcacactgaaatggcactgaatcatgcagttgcagata
ctctttttcaattctcagtcctttgattacgtcagggagaaaagaaagtccccacttggcctgagaatctctgcacccttct
agctcttgttaaccactcttttgaatagcagagaaaacctcagactgccatatctgggagagattttagcaacattttgtttt
cattgtatctctttttacagctacctcccatttcccttctatttcaagctagtaactcagttttttttaaattcaattatttaaatgta
aaaataagtctatttggagaaaaaaaattttaatagcatctctggaatgccagtatggctaaattcatgaatgttgtcctc
aaatgctgaaatctgggaagcatctggccaagctttgtggacaggcctgcctagtttgaatcccaagagccacccagt
ccaagccacaaaacattggaattcttggttcacttccctaacctgaacttgccctctgtgaaatagggacactaatagct
cactcacagggctgctgtgaggacatgtgttgagctgagggtctcgccaggggagaccctgtgcagggagactgtta
tcatggtgatggatttctgcttcattcatttctttttccagacagcatcatatagaatgagttgtggggggcagtcagcaggt
ttgggtttatcctctattctgccacttattacttaaaaaaaccccaaaaaacccaacttatatagtataagctatatccaga
aaagtgcaaatatcatacaagtaccatttgatgaatcttctgatatccccacataaccaacacccagaacctcttcttgt
ctcattccaggataaccactaacctgacttctaacagcatcagtcagttttgtctgtttttgtacattatatatgtgatggtttg
aatgtgtcccccaaatttcatgtgctggaaacttaatccttcaattcatatgttgatggtttttggaggaagggcctttggga
agtaattaggattagataaggtcatggggtgaggtatgatggcactggtgacttataagaagagaaagagaaatctg
agctggcatgctcttgccctctcactgtgtgatgacttctccatgtcatgatgcagcaagaaggccctcaccagatggtg
gcaccatgcttttggacttcccagcctctagaactgtgagctaaatcaatttattttctttataatcacccagtttgatattttgt
catagcaacagaatatggacaaagaaagaaaattaatgcaagaagtagagtttttactgtaacagattcctgaaaat
gtggaagtggctttggaactgggtgatgggaataggttggaagagttttgaggagcaggctagaaaaagcctgtattg
tcaagaatggagcattatgccaggcacggtgtctcaggcttataatcccagcactttgggaggccaaagcaggtgga
tcacctgaggtcaggagttcgagaccagcctagctaacatggtgaaacgctgtttctaccaaaaatacaaaaaatta
gctgggcgtggtggcgcacacctgtaatctcagctactcaggaggctgaagcaggagaatcacttgaacccaggag
gcagaggttgcagtgagctgagatcgtgctattgcactccagcttgggcaacaagagcaaaactccatctcaaaaa
aaaaaaaaaagaaagaaaaagaatggagcattaaagacagttctgcagttctggtgagggcttaaaggaagacc
ccagaactagggaaagtctggaacttcttaatggttactgaagtcgttgagatcagagtgctgatagaaatatggctgg
taaaggccattctgatgaggtctcagatagaactgaagaaccacgtgttggaaactggagcaaaggtcatcctttttat
aaagaagcaaagatcttagctgaactttttctgtgccagagtcatttatggaaggcagaaaatctgtaggtcagccatg
ttgtagggaatgaaagaacattttcagctgagaacactgagagtgtgacacaactaccgactgataagaaaactagt
acacataaattagccaggcgtggtggtgggcgcctgtattcccagctacctgggaggctgaggcaggagaatggca
tgaacccgggaggcagagcttgcagtgagccaagatcgcgccactgcactccagcctgggcgacagagcaaaa
ctccgtctcaaaaagaaaaaaaaaaggaagaaagaaaattagtacacatagaacaaagccagaggctgttcatc
aggacaagggagaaaaactccaaagccatttcagagatcttcaagactgcccctcccattactggcccagagctct
aagagggcagaatggtttggaatgaccagctgctgcccagggctgccttgggtctctgctccccacatttctggtgcag
cattcctcagccatcccagctgtggttcaggtggccacaggtgtgatgtggaaggtaaaagtcataaaccttggcagc
atacacatggcactaattttgcaggtgtgcagaatgcaaaagctgagggggcatgccttcttccacctacatttcaaag
ggtgctgtgaacagccaccccagagagcccctagtagagcagggtctagtggagctacaagggtggggccaccg
ccaagaccccagaatggtagagctatcatagtgcaatgccagcttgggagaactgcaggcatgagactccaacctg
tgcgaagtgcaacatgggcagaacccagcaaaaccacaggggcagagctccccgaagcttcgggggtccaaatt
ccatagtgtgtccaggaggtggcacacagagtaaaagatcattctgaaggtttaaggtttaatgttgttttctatgttgggtt
ttgtactttcctggaaccagttaccctttttcccttgcctctttttccttttagaatgggaatgtctgtcctatgcctgttccactgtt
gtattttggaagtcaataacttgttttgactttacaggcttacagccagagggaatctcccatagaatgaattgtaccttaa
gtctcacccacatctgatttagatgagaccatggactttggaattttgagttggtgctggaacaagttaagactttggggg
ttgtctaagtgtggtgtttcatgcctgtaatcccagtgatttgggaggttgaggtgggaggattgcttgagcccaggagct
caagaccagcctgggcaacatagtgagacctgtctctacaaaaaataaaaataaaaaaattagccaggtattgtgg
catatacctgtaattctagctactcaggaggctgaggtgagaggatcacttgagcccaggagtttgaggctgcagtga
gctatggtcgtgccactgcattccagccagggcaacagagtgagactctgtctctacaaataaaattaaataaactta
gctggatatggtggcacacatctgtagtcctagctactcaggaggctgagacaggaggattacttgagccaaggagtt
tgaggctgcagtgagctatgatcatgccactgcattccagcctggatgatagagcaaaatcccatctctaaaaaaaa
aaaaaaaaaaaaaaaaaaaaaaaaaaactttagtgctattggaatgaattttgcatgtaagaaggacatgcattttg
ggggctggggcaggatgctgtggtttgaatgcatccctcaaatttcatgtgttggaaacttaatctccaaattcatatgttg
atgaaattggaggtgaagcctttgggaggtaactaggattagataaagtcatcagggtggggcccctatgatgagact
ggtggcttacaagaggaagagagaactgagctgacatgctcttgccctcttgccatgtgataccctctgccatgtaatg
gcaggcacagcaagaaggtcctcaacagatgccagcagcatgttcttggacttcccagcctccagaaccatgagct
atatatacttattttacaaattacccattctgtggtattctgttatagcaatagaaaatgaactgagataatatacatggaat
catacagtaagtctgtgcttttgtatgcttcttttactcaacattgtagttgtgagattcatccaggttgttaagcattgctgtac
cctttttccactgggatatagtgttctgtcatgcttgggtcttaatttataaaggtgactgagtggcattttcttccagtattattg
gaaggaaagttttgttgttcacagttcccctgtaaacaagaggcagaacacgtcatgcagggccacacaaaactgta
tcatccagggaccaggcagcagaaagagagggggaactgggactatgcctttatgaaaaagagtggtgggagag
taactgggtgagggcatccactaatgggcaggaagtgaaaacacatatgttagaatttgtagctgaggggtttataata
tgagtttcctatgcctgagaaagctgacttgcaagaaaatgagataaacaactttggccattagtgtggccctgtcataa
atgaatgccagataggcaaatagagaatctaagaaaagatagttggaacaagtgttccattgtgtgaatgcagcaga
atttatttatccattattgaggaggatttgggtagtttccagtttggagctattatgaatattctagtattgctcctatgaacattc
tagcacttttatttttggagcacacgaatgcacttctgttgattatatgcctagaagtgaaattgttgaattatacagtattca
cacagtcagctttagtggctactgctaaacaattttctctagtagtttgcgccaatctaatcaccagtagtgtatagaagct
ccttttactccacattttgccaacacttggtgttttccttctttttgattagtcatttagcaatcaaacctattgtttacattttgatat
ctccaataactaactaaatggagcacttttaatatgctttttggacagttgaatatcttttcttgtgaaatgtctattcaagtta
gtttgcccattttctattgtggtgttctgtctttttcttattgatttgtaggaattccttacgtatcctggatatgaatcccactttgtg
cgttacctttttccttctttctttctttttgaaacagagtctccttctgtcacccaggctggaatgcagtggcgctatctcagccc
actacaacctctgcctcccagcttcaagcaattctcatacttcatcctcctgagtagcttagattacaggcgcatgccacc
atgcccagctaacttctgtatagacaaaataatttttggtagagacagggttttgccatgttggacaggctgatcttggact
cctggcctcaactttggcccaccttggcctcccaaagtgccaggattacaggtgtgagccaccatgcccagcccacct
tttactttcttaatggtgtcttttgaacaagagaggttcttaattttaatatagcccaatttatcattgttccctttatgtttagttcttt
tatgtcctttttaagaatttttgcagccagcgcggtggctcacacctgtaatcccagcactttgggaggctgaggctggcg
gatcacaaggtcaagagatcgagatcatcctggccaacatggtgaagccctgtgcctactaaaaatacaaaaaatt
agctgggcgttgtggctcttgcctgtagtctcagctactcgggaggctgagatcacgccactgcactccagcctggtga
cacagcaagactccatctcaaaaaaaaattttttttgcaaggtcatgcatatgtccccctgatttttttcctaaaaatcactt
attattagatcaatgaattgagtaattgactacatttttcagtcattcaacaaatatttccctgaggttttgataacctgaact
gtgtttggagctggggaggaagcaaactattgaagatatacaaagatggcaaagatgagggcctggagcttgccac
acggaaggggggatggctgcctgaatggttgggcaggtagttgttgacatctgcactccctacatgagcagcagggt
ggcaactctttttatctttttaatttatttttcttttctttctttctttttttttttttgagatggagtctcgctgtgttgcccaggctggagtg
cagtggcgtgatctcagctcactgcaaactccacctcccaggttcacgccgttctcctgcctcagcctcctgagtagctg
ggactacaggcgcctgccaccactcccggctaatgttttgtatttttagtagagaaggggtttcactgtgttagccaggat
ggtctccatctcctgacctcatgatctgcccgcctcggcctcccaaagtgtggggattacaggtgtgagccaccacacc
cggccttaatttatttttctagtctgcaggtaattctttttaattctctccactctcctatgatcttatgaggtagggactgtcatta
tttctcccactttataatgaacaatcagtaaagacagggaagataaccaaatgacatacaaggtggggtccacccca
tgaggctgcaggcttggagctttgctttgtcttaaaaatgagaacatgagctgcccacctgttgagacaagaaacagg
aaaggcttaaaaaactggcttgttatgtacaactatccgtggggctgcagtgaacgggctggcagtgcccaggtgca
ggctgaaccctgggacaatcacattcagcatccaagggcccccgtaatagcttaatgtttgaattgaacccctggggtt
gccttgaaggagagaggtcgtggaagtatgttcaaggggtagggatgggcaggggagatgggtctgaaagccaa
gctctaccccacccaccttgccccaagagaaatagaaccttcatctttaattgcctaacgagaaaactggggctggcc
agatgtggtggctcatgtctgtaatcccagcactttgggaggccgaggcgggcagatcacttgaggtcaggagttcga
gatcaccctggtcaacatggtgaaaccccgtctctattaataatacaaaaattatccaggtatggtggcgcatgcctgta
gtcccagctacttgaggcacaagaatcgcttgaacctgggggacagaggttgcagtgagccgaccactgcactcca
gtctggacgacagagtgagactccatctcacaaacaaaaacagaaaaaaaaaaaaaaaaaagagagagaga
gaaaactggaggctctgagaggttgagggacttgcccagggtcttgcagctagtaagtgacagagctgggacttga
gcttgggttttctgactcctggtctggttcattatccatgaggtgctgggaactaaaataagccacaatcttggaatctccg
tcgcctccctccctcccacatgtctgcgtggctttttgggaaaatgccaggggaatgtaccagccagggagaggaccc
ttgttttcctcatggcccttcctggcaatggcactactgacaccgacagtcctttttgtccctgatgacctctgctgcctgatg
cccaagtgaccacctctgctttgtcatttctaggattggcttccaggtcctcctcagcattggggaactcagcttggccatc
gtgatagctctcatgtctggtctcctgacaggtcagtgtgaggccacctttcttccaccattgccaggacacagcaccca
cgtccagagcgcaccctgccgtgtggctggatgtctatgtgccccatctccttccctgaggatcacataatttcagaattg
gaaaggttcttagaggtcacctgctgctaatgtggactgtgaggccagggcagggaagggacatccctgaggttata
agtagggtgagtggcaacgttgcagacttttgaacccagggctggtgatcacactcagttttgcacagaagcccgag
aaaatccttacacccaaaagcctaccttttatttctgaggacacccataatactattttattcaacagatatttattcaatatc
cactatgagccaggcactggggacacagcagtgagcaaaacaaattccctgaccccatggaattgaccttctagtg
ggggaaggtattagcaataaatagacaaataagtgtctactacgccagatgggaagaagtggctgtgaagacaga
gcaaactagagaaacatagagtcaatgtgggatggggtgttcttttaggggggggtcagggaaagcttatctgagta
gttagcttttaagcagagaccccaatgaagaggagggagatatgcgatgcatttagttaggggaagaacattccatg
aaaataggatagcaagtgcaaaggccctgagacagcagcatgctttgtgtgttgagggaacagtaaggagaccag
tgtggttggtgtgaatggagtgagaaggagcagcaggggttgagggcagaatggtagtgaggagcaggcccttata
aaagatgggaagccactggagatctttcaacaaaggggaaaagtatgtttctgttcttgcaataaaatagaacagca
aaaaatctaggggagttgctaattagccagttttacttatatgccaggtgaaaatatgtggctaggtgcagtggctcata
cctgtaattgcagcagtttgggagaccgaagtgggcagatcatctgagatcaggattcaagaccagcatggccaac
atggtgaaaccccatctctactaaaaattaaaaaataagccaggcgtggtgttggatcccagctacttgggaggctga
ggcagtagaattgcttgaacccgggaggcagaggttgcagtgagccgagactctgtctaaaaaaaaagaaaaaa
agaaaatacacattcaggccaggtgcagtggctcacgcctgtaatcccagcactttgggaggctgagacaggtaga
tcacttgaggtcaggagttcgagaccagcctgaccaacatggcaaaaccctgtctctaccagaaatacaaaaattag
ccaggcgtggtggcgtgtgcctgtagtcccagctactggggaggctgaagtaggggaatggcttgaccccaggagg
tggaggttatagtgagtcgaggttgcaccactgccctccagcctaggtgacagagtgagactgtctcaaaaaaaaaa
gaaagaaaatatacattccatccagaactgttcacctttattctacaagcaaacatcttttattggttagacacccatatat
gtgtccctaagcaggaggtgaatgccaaataagagacaaatggcgtaagacactatgagttgtgtgacgttgggcat
gtcactttactccctctgagccttggttagcttctctgtaaaatgaaaggattatggtaactaagctggcttccttccagcttt
aacaaactgtatggaggtactttttggagttacctgggtaatttttgagtgtgagattggctagaattgctttaatataccatg
tctggccttagctttttgcagagtctttgtgaagaagcagaggcggagtagcgttaattccgtaagttaacgttcagttcgt
ggcagctggcaatccaaccctgggaaaggctgccggatttagcaaaaatgcaaggtgtctgtttttaaatttgaaatga
attgggtatcctgcattttatttggcaaccctgtcctgggactcacactattcactgttatcactggtatgttcaaagtggtgct
gacttgccctctgtcttgcaaagtaccaggaggtcttttcttattcttcactggagtcaaaaaagagaatagaggaaaag
acaatcatattgttcctttaagagttaagaccaacaagttttcttctttacatgttgtttttgacatgagcaaactggtgattaa
aaacaacttgggtggctcatacttgtaatcccagcaccttgggaagctgaggtgggagaatagcttgaggccaggag
ttcaagccagggcaacatagtgagaccccatctctacaaaagatacaaaaattagccaggcgtggtggtacacctgt
agtcccagctgctctggaggctgagatgggaggatcagttgagcttgggaggcagaagttgcagtgagctgagatc
atgccactgcactccagcctggacaacagagcaagaccctgtctcaaaaaaggaaacaaaacaacttggacaat
ggaagggggaaaaagttcctcaagcagccaaaattgcaccaaatggactcccagaagacaagcatttaatttgtta
attgagccctctatgggcctgtctgtatttatttaagaaacaatcctatcaagcatagttattgggtttctcagcccaggtag
attagaaatagcagattagaggtgggctaggtttctagaggtaaagtacaccagcagaagttagaagtgaaagcaa
agagcctaacagaggaagagaaattcttttttttttctttttttagacgcagttttgctcttgttgcccaggctggagtgcaatg
gcgctatctcggctcactacaacctcagcctcctgggttcaagtgattctcctgcctcagcctcccgagtagctgggatt
acaggcatgcaccaccacacccggctaattttgtatttttagtagagacagggtttctccatgttggtcatgctggtctcga
actcctgacctcaggtgatccgcccaccttggcctcccaaagtgctgggattacagggataagccactgcgaccggc
cgacaaattcttaaaactggacacaagaacacaaaacgcttgggctgctgagagattagaacaacaaccctccac
agctacacaccttttccacgttatatggcacgttataagtgggtgttcctagtgatggttctgattttttttaaaaaaagtctaa
atatgtttaatgttgtctcagaagacaaaatatattttagacagatattcctcagtgatgagtaagcctcagctatctggaa
aattcatgcaggcgccagagatcgttactgagtaattcaagctaactgcgtcatgctggttgtaccctgcatgccaatat
cagctaaaagcagcaccacgaaagggaaatacgaatctcactaagcactcgcccattcttgttaacgacactggaa
ctgatcatccttaataatacacagataaatctatcaggagcatttccttgcttcctgtgaaaggaagcactcattccatgtg
tcctgtgaaattcatccaacttcaggaagctggaggaatacatatggccaagctatctgggcagagagtagacaggg
aatggaggttgggcacagtggctcacacctgtaatcgcagccatttagaaggcaaaggcgggcagatcacttgagc
tcaggtgttcaagaccagcctgggcaacatggctaagtcctgtctctgcaaaaaataccaaaaactgagctggatat
ggtagcacacacctgtggtcccagctacttgggaggctgaggtgggagggttgcttgaccccgggagtttgaggctg
caatgagctgtgattgtgccactgcactccagcctggataacagaatgagactctgtcccaaaaaaaaaaataaaa
tcaaagacacttaaaaagatggggaaaaggaaggacaggcacttaagcaagttataagctactttcctaactacac
aagtggaatcttaagctgaggttcccaggagttgactggagccagagaagacagacctataggagcacccaattgg
agtcaccctccatagtagcccatatgtcttacatggatcagctttcgtggggcccttttactccatctggggaagggcgtc
agatctgtggctctcatgtactgctcagtacactgccattcccagttctttttttcaaaaaaaaaaaaaaaatgtctacag
aatcggccaggtgtggtggctcatgcctgtaatactagcactttggaaggctgaggtgggtggatcacctgaggtcgg
gagttcgagaccagcctggccaacatggtgaaactccatctctactaaaaaaaaaaaaaaaaaaaaaattagctg
gatgtggtggcaggcgcctataatctcagctacttgggaggctgaggcaggataatcgcttgaacctgggaggcaga
ggctgcagtgagccgagatcacgccattgtactccagcctgggcgatagagtgagactctgtctcaaaataaataaa
ataaaataaaataaaataaaataaaataggctacagaattaagctggtccaggaatgacagggcttccatttatttgtc
tttcaattgtgggagaaaaaggatttctgttgagatactgtcgttttgacacacaatatttcgattaatcttgagattaaaaat
cctgtgctccaaatcttttaacattaaattatgcatttaaacaggtttgctcctaaatcttaaaatatggaaagcacctcatg
aggctaaatattttgatgaccaagttttctggaaggtaagatttttcacctattaacgtgatagattttgagtgcatgaactta
aaaacatacctgagtatatatgttgacttgctgtttatgagtaaaacaaaaacaaaaatggagtaaggagcattgcag
gaggaactagaggagaaacaaatccatgatatgcatgtgtgtgggggagggtggcggggaggtggtaaaggtca
ccatttccctgatacctcaaattcattcagagtcagggatgagacagctttcactggccacacttcccctccccctatctg
cagtcctcagcgtagccaaatagtctgacatggggtgacagaaccccacaatgcaaaagctggaagaaacctca
agccttggagtccaaccccttttttgacagatgctaagagtggagacatgacttatcaagatcttacaactggctgggc
acggtggctcacgcctgtgatcccagcactttgggaggctgaggtggggcgatcacctgaggccaggagttcgaga
ccagcctggccaacgtgtcgaaaccccatctctactaaaaatacaaaagttagctgggtgtggtggcacatgcctgta
atcccagttactcaggaggctgaggcaggagaatcacttgaacctgggaagcgaagtttgcagtgatctgagatcat
gccactgcactccagcctgggtgacagagcgagactttgcctcaaaaacaaaacaaaacaattgtacatatttaaag
tgttgtaaccaagtgagttacagagaaacaccacactttgagcctaattcaggagtcctttattagccggcgacctaga
gacgactagtgctcaaaattctctcggccccaaagaaggggctagattttcttttataccttggtttagaaaggggagcg
ggaattgagctgaagcaatcttacagaagtaaaacaggcaaaaaagttaaaaagacaaatggttacaggaaaac
aaacagttccaggtgcaggagctttaaagccatcacaaggtgacaggtgcgggggctctgggtgctatctgccgga
cacaaacgcaggggcactagagtactatcacccgggcaaattcctgggaactgcggacacagcttgccacagtac
cttatcagctaattgcactctttgatgtgctgggagtcagcttgcacaagttaagtccttgaggaaggggggggtaagg
agcccttaacgtcttgcaaatgaaggagccgaatggaatccctccggctttcttagctaagagagagtcaatcaagtt
aatacaagttagggtatcacaaaagtatataatttgatacattttaacgtatttatacactgaagagaccatcaccaccat
caagacaaggagcacacccatcacttccacacacttcctcctgctcctttgaaattcctccctccctacccacctggtcc
cacccaaaggcaaccactgaactactttctgtcactaaggtttgcattttctgtaatttttttgtttgagacagggtctcactc
cgccacccacaccgtaatgcagtggcaccatcatggctcactgtagcctcaacctccccaggctcaggagatcctcc
cccctcagcctcctgagtagctaggaccacaggtgtaggccaccatggcaggctaatttttgtatttttttgtagagatgg
ggtttcaccgtattacctaggctggtctcgaactcatgggttcaagcaatcctcctgccttggcctctcaaagtgctggga
ttataggcatgagccactgtgcccagccctctgtaatgttacacaaagggaatcatgcagcacgtactgcccttggtct
ggcttcttttgctcagcatgattattctgagaatcatccgtgttgttgcgtgtaactgacttcatcagcttctctctgcagctgtc
agctcttggcttctcccaacagccaatctctctttatcccctgcaagtgttcttgcctatttagcagaatcaaggtactctatc
gaaaagactcggaaaattggtttaatctattcattcattcctcaggtatttatcgaataactattctataccaagtactatgct
aatcaaccaaggacagcacaaacaggagaaatctccagctcagtcacttgagttgcaataaatatttgctggatagg
tcaggtgcagtggctcacacttgtaatcccagcactttggggattactgagacgggaggatctcttgagcccaggagg
ccaaggctgcagagaaccatgatcatgccactgcactccagcctgggtgacagagtgagatcctgtctctgaaaaa
aaatatttgctggataaattaaggaaatctgacgaaccccatcagtagccattgcagcaacaggtaaactagaacga
gtgtgaatttggaatgaggaaacccgatgttggccatcattctgtaatgtcatgtattatgtaatgtattatatattaatgtat
gtattatgtaggcaagttccttgacctctctcactggtaacataagagtagtaatctttgtgctacttcactgggttattttaaa
gatcaagtgaggtaataatgtctgtaacaacattctgtaaaatgcaaaccgccacatgaatgtgaaagtttattactag
ggatttagccaaccacaagggaatgtgtgagcataagagctatcatattgcaagcctacagtttctgattttgtgctaggt
gcttttccacattacctgattttatcctcacaacagccctgcataaaagtaagtatgtcgcccaggtgcggtggctcatgc
ctataatcccagcactttgggagcccgaggtgggcaaatcacttgagatcaggagtttgaaaccagcctggtcaacgt
ggtgcaaccctgtctctactaaaaatacaaaaaaaaattagacaggcgtggtggtggatgcctgtaatcccagctact
tgggaagctgaggcaggagaatggcttgagcccgggagatggagattgcagtgagatgagattgcgccactgcac
tccagcctgggtgacagagcaaggctatgtctcaaaagagaaaaaaaaagtaagtatctcagtcttgaagatgatg
aaatggaggcctagagagattaagtaacttgcccaaaatgacagaactaatgcatagaaaagaagaaatgtgatgt
cttttggctccaaagacaccccacatatgcgttggttacagttactagagaaaagttattccacccccaccccaccccc
agaaatcttctgacttgttttctcgcagttgagtaggaccatttattcggcagtgtaccattctcagcttgcagttgaaagcc
aaatatccattaaagaggcaaggatgcaaacttgctaagctgataaatccaggggtgattttttttttttttgcaaaccatc
caacaagacattttaaatactcattgaatttcatagaactgactgccaggattggaaagacattaaagccagctcagc
cactgcctcgctggttggccagaccacgcctggcacttctgggagggagcactcaccaccccccaagggcacccat
ctcatcctccgaaggtttatgaaaatgcactcatcatttgctaattcattccactacgtgtattacctaatttgtgacacgatg
tgaagtaccagagagataattctaaataaaatatagttatgggtctcaaggagccagatatgctaatctcctatcctcct
gcagtttacagtggtcctcaccagatacttatttacaaaaattcagtttattatttatttttttgagacagagtcttgctctatag
ctcaggctagagtgtaatggtgtgatctcggctcacttcaacctctgcctcccaggttcaagtgattctcctgcctcaacct
cccaagtagctgggactacaggcacctgccaccacggctaatttttggagttttagtagagacagggtttcaccacgtt
ggccaggctggcctcgaactcctgacctcaggtgatctgcccacatcagcctcccaaaatgttgggattacaggcgtg
agccaccatgcccggccaaaacttcagtttataacacaatctttcacgtgtcttctgctttcattaaaagaatagacagtt
cccttctttatttcagtttaataaaccatggattttatttcatgctttgcaaaacacaagggctcactgacatgcacttcttaaa
ctaattctggctggtcgcctgtaattccagcactttgggaggctgaggccgacagatcacttcaagtcaggagttcaag
accagcctggccaatatggtgaaaccacgtctctaccaaaaatataaaaaattagccaggtgtggtggtgcgtgact
ataatcccagctactcaggggcctgaggcagaaaaatcacttgaacccgggaggcggaggttacagtgagctgag
atcgcgccactgcactccagcctgggcgacagagtgagactctgtctcaaaaaataaataaatacaaataatgtaa
aatacgaaacaagcaatcctggcagtagctgctggaatgagaggagggagaggtcatagggaggtcggggaca
atggagcatggagttgtgttggatttggctaagcagcaggaagtgcaaggcattccaagcaagaggaggggggca
ggtggggagcatctgcaagaacagaagcagcatgagcaacctggctcggcagtgtgtgaaaaggctgaaaggtg
gctagagccacttcaatttcatccttcaggcaaatgggaaattcccaaaggtttgagtggggaagcaatgcctacaat
gaaagtttgagagtgaagcagagtgatcgaattaagcatgtaggccgagttctgaaataactgcaatgtgctgaagat
catccattggcttctgaatgagtatttgcagtttattttttaaaatgattttattgccaagaaagataaacactactgttttggta
caaaaacataacaaaatgtgttgagtccctcttgctgttttacgcgaagttttaaaaatctactcttgtcacagtggtatca
cccctacttctgatttcaaataaatgttctagagacacagtaagggcccaacaaacgcttgttcaacaacacaaggag
agccagcttttaaagtaggaaaacaggccgggcgccgtggctcacacctgtaatcccaacactttgggaggctgag
gtgggcagatcacttgaggtcaggagttcaagaacagcttggccaacatggtgaaaccctgtctctactaaaaacac
aaacattagccaggcgtggtggtgcacaccagtagtcccagctattcaggaggctgaggcaggaaaatggcttgaa
ctggggaggcagtggttgcagtgagccgagatcgtgccactgcactccagcctgggggacagagggagactccat
ctcaaaataaaacaaaacaaaaccaaatcatacaaaaacattagctgggtgtggtggtgcatacctgtaatcccag
ctacttgggaagctgaggcagaattacttgaacccctggggggaggttgcagtgagctgagatcttgccactacactc
cagcctgggcaacagagtgaggagactctgtctcaaaaaatatatatattaaaaaaaagaaaaaaaaaagtaaac
taggaaaacacatcagcagcctgccaacagactcccctagcctcggtgagggccagtgttctgggaggcagatctg
aattctagtcctagttcacccactggcaggctggtgcccttgggcaggtcgcttctctggggctcagtttcttcctctataaa
atgagatcaaatcccatgttctaagagtttgtgctctggagtcagacagatctgggttctaccactgccagctctgtgatct
tgtagcttcagtctcgtcatctgacatggagataacagtaactgtctcactgtgttgttagggtttaaaggagataatgtat
gtgaaatgttagcaaacaagtgttagctaccctgatttccggtttcagagttctgtggtcccagtttatgccacatgcagtg
acgttgtatggtaggctgtggtgtggcaccacttcagaactcagcgcatgcacagcttgcagaagagaaggccaga
ggagacctaagaaggctcttcgaacacttgaaagaccggcatgtaggccgggcgcagtgactcacgcctgtaatcc
cagcagttttggaggtcgaggcgggtggatcacctgagtttgggagtttgataccagcctgaccaacaaggtgaaac
cccgtctctactaaaaaatacaaacattagctgggcatggtggcgggtgcctgtaatcccagctactccggtggttgag
gcagaattgcttgaacccgggaggcagaggttgcagtgagctgagattgcatcactgcactccagcctgagacaag
agcgaaactccatctcaaacaaaacaaacaaccaaccaaacaaaaccaaaaaaaaaactggcatgtagaaga
aaaatactttttctctacacttctccaaagaatttaactaggcccaggggaggtgcagtataaatttctaacaatctcaac
tgtctgccaaatggaatgagctacttcatatggcagtagtgagtcctctgtctttggaggcattcaaataaaagccagat
ggccatttatcaacaatccatgtaaaacgttagatgaaataaaacctatatatccaagatctcttccaattcagattttatg
aaagaatttctaaggtctttgtaatgagacatttaggctgtttcaagagatcaagccaaaatcagtatgtgggttcatctg
caataaaaatgtttgttttgcttttacagtttcctcatttggctgttggattttaagcaaaagcatccaagaaaaacaaggcc
tgttcaaaaacaagacaacttcctctcactgttgcctgcatttgtacgtgagaaacgctcatgacagcaaagtctccaat
gttcgcgcaggcactggagtcagagaaaatggagttgaatcctttctctgccactctttgaggagaatctcaccatttatt
atgcactgtagaatacaacaataaaatacagccatgtaccacataacaacatcttggtaaacaacagactgcatata
tgatggtggtcatccagtaagctaaggttaatttattattattccttgtttttttttttttttttttttttttgagatgtagtcttactctgtca
cccaggctagagtgcaatggcaccatcttggctcactgcaacctctacctcctgggttcaagcaaatctcctgcctcag
cctccaaagtagctgggattacaggcacccaccacatctggctaattttttgtatttttagtaaagatggggtttcaccatg
ttggccaggctgatctcaaactcctgacctcaagtgatctgcccgcctcggcctcccaaagtgctggaaccacaggcc
tgagccactgtgcccagccttgtttgcttttttaacagataacagtgtgctcatagaaactgctttgacatgactgcaatcat
gtgcttcatagaaacttaattagattataccactagagtcttcagatttttatactttttttttttgaaacggagtctcactctgtc
accaggctggagtgcagtgccgcaatctcggctcactgcaacctccgcctcccaggttcaagcaattctcctgcctca
gcctcccgagtagctggaattacaagtgcgcactaccacacccagctaatttttgcatttttacttgacagggtttcacca
tgttggctaggatagtttcaccaggatctcttggcctcatgatcagcctgcctcggcctcccaaagtgctgggattacag
gtgtgagccaccgtgcccagcctatacttccctttttgaataccatttggtgttttgaagaattaacagctttgtgaacgtgg
cagtgcttgtgattcaggcttccattgagaccaaggggagaacctggttgcaggacaaacagacggacagcgtgtg
gcagtgtttaaatgctcttctgaaggctgatacgacagctctctgtgcactgattgcatatgcatcccaagattatattattg
ttttctactgctatgtgtcacactttgccaaacaggatgtggaaaatgaataagcggttttcttaggcacttcttaacagac
aattggtcaaaatgaactccattgcttaagaaacacataaacaccatttagtcactgaacatagctatatgtatggttgtt
actatgggaaatcttgttttgccaattttctttgaaaattctggcagaccaaggttctttttgtttacataatacttgaaaaata
aaaatgaacaagctaacaaacta.

I/We claim:

1. A method of genetically modifying one or more genes associated with blood type in a cell, the method comprising introducing into the cell a site-directed nuclease or a nucleotide sequence encoding a site-directed nuclease, wherein the one or more genes associated with blood type are selected from the group consisting of ABO, FUT1, and RHD.

2. The method of claim 1, wherein the site-directed nuclease is selected from the group consisting of a zinc finger nuclease (ZFN), a transcription activator-like effector nuclease (TALEN), a meganuclease, a CRISPR-associated transposase, and a CRISPR/Cas nuclease.

3. The method of claim 2, wherein the site-directed nuclease is a CRISPR/Cas nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

4. The method of claim 3, wherein the method further comprises introducing to the cell a guide RNA (gRNA) targeting the ABO, FUT1, or RHD locus.

5. The method of claim 4, wherein the gRNA comprises a CRISPR RNA (crRNA) and optionally a transactivating CRISPR RNA (tracrRNA).

6. The method of claim 5, wherein the gRNA comprises a crRNA and a tracrRNA as two separate molecules.

7. The method of claim 5, wherein the gRNA comprises a crRNA and a tracrRNA as a single guide RNA (sgRNA).

8. The method of claim 7, wherein the sgRNA comprises a complementary region, a crRNA repeat region, a tetraloop, and a tracrRNA.

9. The method of claim 8, wherein the crRNA repeat region comprises, consists of, or consists essentially of a nucleotide sequence set forth in SEQ ID NO:5, SEQ ID NO:9, SEQ ID NO:13, or SEQ ID NO:18.

10. The method of claim 8 or 9, wherein the tetraloop comprises, consists of, or consists essentially of a nucleotide sequence set forth in SEQ ID NO:6 or SEQ ID NO:17.

11. The method of any one of claims 8-10, wherein the tracrRNA comprises, consists of, or consists essentially of a nucleotide sequence set forth in SEQ ID NO:7, SEQ ID NO:11, SEQ ID NO:15, or SEQ ID NO:16.

12. The method of any one of claims 5-11, wherein the crRNA comprises a complementary region specific to a region of the ABO locus.

13. The method of claim 12, wherein the region of the ABO locus is a coding sequence (CDS), an exon, an intron, a sequence spanning a portion of an exon and a portion of an adjacent intron, or a regulatory region.

14. The method of claim 12 or 13, wherein the complementary region comprises, consists of, or consists essentially of a nucleotide sequence complementary to a nucleotide sequence set forth in any of SEQ ID NOs: 20-203.

15. The method of any one of claims 5-11, wherein the crRNA comprises a complementary region specific to a region of the FUT1 locus.

16. The method of claim 15, wherein the region of the FUT1 locus is a CDS, an exon, an intron, a sequence spanning a portion of an exon and a portion of an adjacent intron, or a regulatory region.

17. The method of claim 15 or 16, wherein the complementary region comprises, consists of, or consists essentially of a nucleotide sequence complementary to a nucleotide sequence set forth in any of SEQ ID NOs: 204-420.

18. The method of any one of claims 5-11, wherein the crRNA comprises a complementary region specific to a region of the RHD locus.

19. The method of claim 18, wherein the region of the RHD locus is a CDS, an exon, an intron, a sequence spanning a portion of an exon and a portion of an adjacent intron, or a regulatory region.

20. The method of claim 18 or 19, wherein the complementary region comprises, consists of, or consists essentially of a nucleotide sequence complementary to a nucleotide sequence set forth in any of SEQ ID NOs: 421-580.

21. The method of any of claims 1-20, wherein the genetic modification is through non-homologous end-joining (NHEJ).

22. The method of any of claims 1-20, wherein the genetic modification is through homology-directed repair (HDR).

23. A guide RNA (gRNA) for use in genetically modifying one or more genes associated with blood type in a cell, wherein the one or more genes associated with blood type is selected from the group consisting of ABO, FUT1, and RHD.

24. The gRNA of claim 23, wherein the genetic modification is through use of a site-directed nuclease selected from the group consisting of a zinc finger nuclease (ZFN), a transcription activator-like effector nuclease (TALEN), a meganuclease, and a CRISPR-associated transposase, and a CRISPR/Cas nuclease.

25. The gRNA of claim 24, wherein the site-directed nuclease is a CRISPR/Cas nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

26. The gRNA of claim 25, wherein the gRNA comprises a crRNA and optionally a tracrRNA.

27. The gRNA of claim 26, wherein the gRNA comprises a crRNA and a tracrRNA as two separate molecules.

28. The gRNA of claim 26, wherein the gRNA comprises a crRNA and a tracrRNA as a single guide RNA (sgRNA).

29. The gRNA of claim 28, wherein the sgRNA comprises a complementary region, a crRNA repeat region, a tetraloop, and a tracrRNA.

30. The gRNA of claim 29, wherein the crRNA repeat region comprises, consists of, or consists essentially of a nucleotide sequence set forth in SEQ ID NO:5, SEQ ID NO:9, SEQ ID NO:13, or SEQ ID NO:18.

31. The gRNA of claim 29 or 30, wherein the tetraloop comprises, consists of, or consists essentially of a nucleotide sequence set forth in SEQ ID NO:6 or SEQ ID NO:17.

32. The gRNA of any one of claims 29-31, wherein the tracrRNA comprises, consists of, or consists essentially of a nucleotide sequence set forth in SEQ ID NO:7, SEQ ID NO:11, SEQ ID NO:15, or SEQ ID NO:16.

33. The gRNA of any one of claims 26-32, wherein the crRNA comprises a complementary region specific to a region of the ABO locus.

34. The gRNA of claim 33, wherein the region of the ABO locus is a coding sequence (CDS), an exon, an intron, a sequence spanning a portion of an exon and a portion of an adjacent intron, or a regulatory region.

35. The gRNA of claim 33 or 34, wherein the complementary region comprises, consists of, or consists essentially of a nucleotide sequence complementary to a nucleotide sequence set forth in any of SEQ ID NOs: 20-203.

36. The gRNA of any one of claims 26-32, wherein the crRNA comprises a complementary region specific to a region of the FUT1 locus.

37. The gRNA of claim 36, wherein the region of the FUT1 locus is a CDS, an exon, an intron, a sequence spanning a portion of an exon and a portion of an adjacent intron, or a regulatory region.

38. The gRNA of claim 36 or 37, wherein the complementary region comprises, consists of, or consists essentially of a nucleotide sequence complementary to a nucleotide sequence set forth in any of SEQ ID NOs: 204-420.

39. The gRNA of any one of claims 26-32, wherein the crRNA comprises a complementary region specific to a region of the RHD locus.

40. The gRNA of claim 39, wherein the region of the RHD locus is a CDS, an exon, an intron, a sequence spanning a portion of an exon and a portion of an adjacent intron, or a regulatory region.

41. The gRNA of claim 39 or 40, wherein the complementary region comprises, consists of, or consists essentially of a nucleotide sequence complementary to a nucleotide sequence set forth in any of SEQ ID NOs: 421-580.

42. A composition comprising the gRNA of any one of claims 23-41.

43. The composition of claim 42, further comprising a site-directed nuclease or a nucleotide sequence encoding a site-directed nuclease protein.

44. The composition of claim 43, wherein the site-directed nuclease is a CRISPR/Cas nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

45. The composition from any one of claims 42-44, wherein the composition is formulated for delivery into a cell.

46. A cell comprising the gRNA of any one of claims 23-41.

47. The cell of claim 46, further comprising a site-directed nuclease or a nucleotide sequence encoding a site-directed nuclease protein.

48. The composition of claim 47, wherein the site-directed nuclease is a CRISPR/Cas nuclease selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7.

49. A method of identifying a new genomic locus for genetically modifying one or more genes associated with blood type in a cell, the method comprising (a) locating a genomic locus based on a known gRNA; and (b) scanning a region of about 500 to 4000 bp on either side of the genomic locus for a PAM sequence, wherein the one or more genes associated with blood type is selected from the group consisting of ABO, FUT1, and RHD.

50. The method of claim 49, wherein the known gRNA targets the ABO, FUT1, or RHD locus.

51. The method of claim 50, wherein the gRNA comprises a complementary region comprising, consisting of, or consisting essentially of a nucleotide sequence complementary to a nucleotide sequence set forth in any of SEQ ID NOs: 20-580.

52. A cell having one or more genes associated with blood type genetically modified according to the method of any one of claims 1-22.

53. The cell of claim 52, wherein the cell is an autologous cell.

54. The cell of claim 52, wherein the cell is an allogeneic cell.

55. The cell of any one of claims 52-54, wherein the cell is a pluripotent stem cell, an embryonic stem cell (ESC), or an induced pluripotent stem cell (iPSC).

56. The cell of any one of claims 52-54, wherein the cell is differentiated from a pluripotent stem cell, an ESC, or an iPSC.

57. The cell of any one of claims 52-54, wherein the cell is a primary cell.

58. The cell of claim 56 or 57, wherein the cell is a mesenchymal stem cell or a hematopoietic stem cell.

59. The cell of claim 56 or 57, wherein the cell is a blood cell.

60. The cell of claim 59, wherein the blood cell is a red blood cell, a platelet cell, a mast cell, a basophil, an eosinophil, a neutrophil, a monocyte, a natural killer (NK) cell, a natural killer T (NKT) cell, a macrophage, a T cell, a B cell, or a plasma cell.

61. The cell of claim 60, wherein the cell is a T cell, an NK cell, or an NKT cell.

62. The cell of claim 56 or 57, wherein the cell is a cardiomyocyte.

63. The cell of claim 56 or 57, wherein the cell is a retinal pigment epithelial cell (RPE).

64. The cell of claim 56 or 57, wherein the cell is an endothelial cell.

65. The cell of claim 56 or 57, wherein the cell is a β islet cell.

66. The cell of claim 56 or 57, wherein the cell is a glial progenitor cell (GPC).

67. The cell of any one of claims 52-66, wherein the cell is modified to have reduced expression of one or more MHC I molecules and/or one or more MHC II molecules, optionally, wherein the one or more MHC I molecules are selected from the group consisting of HLA-A, HLA-B, HLA-C, and optionally, wherein the one or more MHC II molecules are selected from the group consisting of HLA-DR, HLA-DQ, HLA-DP, HLA-DM, and HLA-DO.

68. The cell of claim 67, wherein the modification is by modulation of the B2M, TAP1, CIITA, MIC-A, and/or MIC-B loci.

69. The cell of claim 68, wherein the modulation of the B2M, TAP1, CIITA, MIC-A, and/or MIC-B loci comprises B2M, TAP1, CIITA, MIC-A, and/or MIC-B knockout.

70. The cell of claim 68, wherein the modulation of the B2M, TAP1, CIITA, MIC-A, and/or MIC-B loci comprises knock-in of a transgene at the B2M, TAP1, CIITA, MIC-A, and/or MIC-B loci.

71. The cell of claim 70, wherein the transgene encodes one or more tolerogenic factors selected from the group consisting of A20/TNFAIP3, CD16, CD16 Fc receptor, CD24, CD35, CD39, CD46, CD47, CD52, CD55, CD59, CD200, CCL22, CTLA4-Ig, C1 inhibitor, complement receptor (CR1), DUX4, FASL, H2-M3, IDO1, IL15-RF, HLA-C, HLA-E, HLA-E heavy chain, HLA-G, IL-10, IL-35, MANF, PD-1, PD-L1, SERPINB9, CCL21, and MFGE8.

72. The cell of claim 71, wherein the one or more tolerogenic factors comprise CD47.

73. The cell of claim 72, wherein the CD47 is human CD47.

74. The cell of claim 73, wherein the human CD47 comprises an amino acid sequence that is at least 80% identical to the amino acid sequence set forth in any one of SEQ ID NOs: 583-588.

75. The cell of claim 74, wherein the human CD47 comprises an amino acid sequence that is at least 80% identical to the amino acid sequence set forth in SEQ ID NO:584.

76. The cell of claim 71, wherein the one or more tolerogenic factors comprise HLA-E.

77. The cell of claim 71, wherein the one or more tolerogenic factors comprise CD24.

78. The cell of claim 71, wherein the one or more tolerogenic factors comprise PD-L1.

79. The cell of claim 71, wherein the one or more tolerogenic factors comprise CD24, CD47, and PD-L1.

80. The cell of claim 71, wherein the one or more tolerogenic factors comprise CD46.

81. The cell of claim 71, wherein the one or more tolerogenic factors comprise CD55.

82. The cell of claim 71, wherein the one or more tolerogenic factors comprise CD59.

83. The cell of claim 71, wherein the one or more tolerogenic factors comprise C1 inhibitor.

84. The cell of claim 71, wherein the one or more tolerogenic factors comprise CD46, CD55, CD59, and C1 inhibitor.

85. The cell of claim 71, wherein the one or more tolerogenic factors comprise HLA-E, CD24, CD47, PD-L1, CD46, CD55, CD59, and C1 inhibitor.

86. A pharmaceutical composition comprising the cell of any one of claims 52-85.

87. A method of treating a disease in a subject in need thereof, the method comprising administering the subject the cell of any one of claims 52-85, or the pharmaceutical composition of claim 86.

88. The method of claim 87, wherein the disease is cancer.

89. The method of claim 88, wherein the cancer is a hematologic malignancy.

90. The method of claim 89, wherein the hematologic malignancy is selected from the group consisting of myeloid neoplasm, myelodysplastic syndromes (MDS), myeloproliferative/myelodysplastic syndromes, acute lymphoid leukemia (ALL), chronic lymphocytic leukemia (CLL), acute myeloid leukemia (AML), chronic myelogenous leukemia (CML), B cell acute lymphoid leukemia (B-ALL), T cell acute lymphoid leukemia (T-ALL), T cell lymphoma, and B cell lymphoma.

91. The method of claim 87, wherein the disease is an autoimmune disease.

92. The method of claim 91, wherein the autoimmune disease is selected from the group consisting of lupus, systemic lupus erythematosus, rheumatoid arthritis, psoriasis, psoriatic arthritis, multiple sclerosis, Crohn's disease, ulcerative colitis, Addison's disease, Graves' disease, Sjögren's syndrome, Hashimoto's thyroiditis, and celiac disease.

93. The method of claim 87, wherein the disease is diabetes mellitus.

94. The method of claim 93, wherein the diabetes is selected from the group consisting of Type I diabetes, Type II diabetes, prediabetes, and gestational diabetes.

95. The method of claim 87, wherein the disease is a neurological disease.

96. The method of claim 95, wherein the neurological disease is selected from the group consisting of catalepsy, epilepsy, encephalitis, meningitis, migraine, Huntington's, Alzheimer's, Parkinson's, Pelizaeus-Merzbacher disease, and multiple sclerosis.

97. The method of claim 87, wherein the disease is a cardiac disease.

98. The method of claim 97, wherein the cardiac disease is selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, cardiomegaly, mitral insufficiency, and autoimmune endocarditis.


SEQUENCE LISTING (595 sequences)

SEQ ID NO: 1 - DNA, 42747 bp, Homo sapiens
  misc_feature 1..42747: note = ABO
  source 1..42747: mol_type = genomic DNA
agtgtaaactcctctgagagatatcaggaaaagcaggaagaagcctctgggacccttcgggaggtaactcctcttcgcagcggggcgcgctctcccagtccctgcagccgccgccgccctctcctgagcttcctcgagcggacgccaggcaagggcgggggtcgtagcggggcggagcggggctttgtccacggaccgcgcgaagaggcctcagggcccggcgcgggcgccggagggggactcgctcgcagggggaacgcgaaggttcctcagtctgcgggacgcagagctccgtggggccgcgagccggggccggggaagcgactctgcctagggggacgtcgcgggcgcggggcacagggtcctgcggggctggagggctacaggctgcggcgcgcgcgagccggaaggccggggatcgtgggttctggggccgcagcttcacgggttcgtctcccccgcctccccgggggagcaggatgtcagggggtcgcccccgcccgggagacagggtgtcaaggggcccccggggacggggcttcaggggcacccggagccgctcggccccagggcgggatgcggggacagggccccaaggtaccagggccacgaggggcgcgcgggtcccttggggatgcgcgcgaggaggcgccgtcccttcctagcaggggtccctggggacccgcggccgcctcccgcgcccctctgtcccctcccgtgttcggcctcgggaagtcggggcggcgggcggcgcgggccgggagggggcgcctcgggctcaccccgccccagggccgccgggcggaaggcggaggccgagaccagacgcggagccatggccgaggtgttgcggacgctggccggtgagtgcaggcctcggccccgggtgcccgcgagggagccgctaccgcagggaatgcggggtgcacccgacagccgggccggggtgggggcgctcagggctgcgaggcttcgggccggccgccgccccagcctccgagaccctgcgtcctggggagccggcgggcaggtgggctcggccgcgctgtgggtgcctgggacccgcagggaggatgggcgcggtggcgcggcctggcggggggctcgtctccggggtccccgggtcctggtgagagcggggtccctcgacgccgtggcggtctccagcctctcctcgcccctccacgctccccgccttccatgagctgctattttcagcacctaccgcccgaccctggactaggacaaggctctgggctgccctgcccgccccccagcccttccctcgggcacggcggccaggcgcccgggttgaccgggaacagcctccataccccaaacgcggaggcgcctcgggaaggcgaggtgggcaagttcaatgccaagcgtgacgggggaactgtgccccgggccctcaggtgatataggagttaagaagaaattattgaggcaaccagatgcggtgactcaggcctgtaaccccagcactttgggaggccgagggtggatcacctgtccttaattttcttggcgccagaagatgaattgagtatttacccagacaacaacgtcgcttcagagggagggatgcagaacgcagggccacggggtgcaggctgcaggccagtgaaccccaacgccaaaggccagggagagccgggtggggtacccagagccagcacacagccctttaatttagaggagtgctgtgtacacatctggggagagatgttttactttgatttggaatcaggtggcggataaggcatactgaggcctgacttggtgagggctcctgccccggaggtgcagccctggaggagcgggaggcagaggagtggaaaattcatgaagaaaaccgggtatggtgtaggtcgaggccctgccctcagtaatgctcaccatttgtcagtgtttactgtgaggcagcactgtgttcagtatcgctgagttctcaggaaggaacggtaaatacttcccgggtcattctttcacccacgggaaaacaggtttggagagatctgggacagtgctctggtcctaggcaggaagggctgagtggggcctgggactcaggtctgactgcaaacacctgcctcctccctgtgctgccagcgccttccgggttcttccctgtccctcctttgtggtctttgttttcccttttttgtcttaatatgtttcaacggatgtatacaataaaccgcacatgaaaggtacagctggatacatcttgacccagtcaagatgatgaacacagctgccaccccaggagtctgtcctgccccacgggttatgctgtcttagttggtctcatgtcagggagccttggaggaccagggtcgggcagttggtctctatactcctggggttctggcactggctctggcccatgaccgcacccaagacaaacgtcttgaagactaagaggttaggtctttgagaaacctggcaaatgagtgcccattctcaggttacccacattctgcatgttgatttagtcatccaaccaatgttggttgaacactgatgagaacaagcaggcctgtgctagaaggtgcctgcagccaggagctggtgagctggtgtccttagggacaccaatggcgagggacccagtgtgtggaaatctgggggacaagcatgccagggagaagagctcacatggggaaggcccggctccacaaatcagtcaggcttgttggggcgggcaggagagcagggtagtggagtcagagggagtgatcccccgaaaggcaggaagaggacatgagagagccttggagatgacggtgaggatgtggttggtgggcggtgagctgggtgtcatgtgctggctccttagagaatgctcagctccttcacacccatcataatccctggaggactgagaccacgtgcaggagttttggaagctggcagtgcacccagtcccggctctcctccattctggtgggtctcaccagagattggccaagaagagatcaaactgttcctggaccaaactgagggtggggctgctatctctcgcggcccaataacgagatgcagatgaactggggagaaagagagtttttatttctgtaaccagttacaaggagaagacctggaaattatctccagaccaactcaaaattacaaagttttccagagcttatataccttctaagctatatgtctatgtgtaagtgtgcattcatctcaagacataagtaattgacttatgttaatctataactaaggtctgagtcctgaagaccttcctctggatcctcagtaaatttacttaatctaaaacccttatcttgtctcctaaatcatgggggtttgggaagttccttcagacccccagtaaacttatttgtggagtcctggggaatttcttcagatccccaataaaacttgtttaatcctaaatgggtcctgttaagaattccttcgttattttgtcatgcttgaaggcccaggaaaggtctaggcaaaactcttggtgggattttgttatattccagcctttttataagggcactggcttttaatatttaatttaaccactcagtcagtactgaaacagttgttagggaggcctgcgttagtgagacctgacctgccacaacacatcttactcggaatgctgcccataacttcaaaaaatcagctttgacggagccctactgaacacacctagcatctctcttccttcagcttagggtcaaggggctggggttgatggcaccattgaaagaaacagctttattgccgtgtcattgatatgccataaaattcacctgtttcaaatgaattattttcagttagtttacagagttgtgaaataaattttataacttttccatccccagcccaccaaaactccctggaactcctccgcagtcattccccattcccacctggcctcagacaatcactttctgtctctccagtcttgccttttctggacagttcctatgaatggagtcctgtgttacatggtcttttgcatctgacttccttcacttagaataatgattccgagattcatgtatgttgtagtatgtatcagtatttaattcctttttattactgaataatccattgtacagatagaccacattttgtttatccattcatcagctgaaggacatttcggctgtttctgcttttttagctattttaaactgcacgcagcactgctatgaacatttgtgtacaagactttgtgtgaacatgttttcatttctcttgggttgatacccagcagaggaattgctgggtcatacagaaagtctgatttaacattttaagaaactagcaaactgttttccaaagtgactgccccattttacattcccatcagcagtgtataacggttctaattttcttttcctttttcttttcttgagacaaggttttgctctgtcacccaggctggagtgcagtggcatgatcttggctcactgcagcctcaatctcttgggctcaattggtcctcccacctcagcctcctgagtagctgggactacaggtgagtaccaccacatccagctaatttttgtatttttggtagagatggggttttgccatgttgcccaggctgttctcaaactcctgggctcaagtgatctgcccacctcggcctccccaagtcctgggattataggtgtgagccgctgcgcctggctgagggttccactgtctgtacgtctgcagcaatacataccattcttgtgggtaaaaggtggtatctcattatagttttgatttgcatttccccaaggtcaaatgatggcaagtggcttttcttgtgctttttagccatttgtatatgtttttggtgaaatgcctattgaaatgttttgcctttttaaaaattgagttgtctcctttgttcagttttgagagttctttacatacacagtatcatatatatatatatatatatatatatatatatatatatatacacaccagatatatgatttgcaaatattttcaaccatcaatagtttaacacttttttattggtattttgaagtagaaaagttttacattttgatccattccaatttattaactttttcttttattgtgtatctggtatcatatctaagaaatcttaatccagtgtcacaaagatttaatcttatattttcttctaaccgctttctagtttatgtgtaagaatctgtccattttacctaagttgcataatttgttggcaaacagttgtacatagtatttccttgaaatccttttaatctctgtaagattggaattgctgtctcctcttttagtcctgattttagttatttgtgttctctctctctctggtcaatgtagctaaagctttgtcaattctcttgatcttttcagagaactgccatttgatatttttttactttatctttttctttctgttctctagttcattggtttccactcttctcactttgggtttaattcgttctgtcttttcttattgtagttacttacaatggaagcttacacacttgatttaagatttttttttctaatgtagacatttacagctataaatttcccttgaaacacagctttagttatatctcataaattttggtatgttgtgtttacattttcattcagctcagtgtatcttttgattttcttctttgacccattgcttatttagaattatgttgtttaatttctatgtatttataggtttcccaaatttcttttgttaatttctgatttcattcccctgtggctagagaagaaactctgtggatatcagtcctttcgaatttctcagaattgttttatggtccagcatatgattgaccttggacactgttccatgtgcacttgaggagaacgtgacttcggctcttgccaggtggagtgttctagagatgtcagttggtgtccagtgatgtcaagtcatttgtatctactgattttctttctaattattctatccattactgagagtggggtaggattttggtgtccaattattgtttttttgtttttttttttttgtttgtttttttttttttttgaggtggagtctcactttgtcacccaggctggagtgcagtggtatgatcacagctcactgcagcccccacctcccaaggcttaggtcacctcagcctcctgagtagctgcaactacaggcaagtaccatcatgcctagctgatttttgcattttttatagacagggttttgccatgttacttaggctggtcacgaactcctgggctcaagtgacccacccacctcggcctcccaaagtgctgggattacaagcgtgagtcaccatgcctggcccaaccattactggtgaattgactgtttcacccttcaattctgtgagtttttgcttcatgtattgctgcgggataattaaggaatcagagagaccgatggggttgaggaggaattatttaattatttaggtgcaccgacccaatcagattaacatccaaaggactgggccccaaacaaagagtcaagctaccttttaagcattttgtggggtggggggagatttgtgcagggggaagagtattacagaagcaagaaacaaagacagttattcagttaagacatgcattacattatttcttacttttcaaggaacaacacgttttatgactcaagattatctgtttagtgaccttgcagctgcacagctagagaaacagagtcttcgcaatgcctgggaaagggagagataaggctcactagccacagaaaaacagccagttaatttttaaaggactccagccctttctcttcctcaaggggaattggttttttacatacaactgagtttttgcttacacagtttttaatttcttttaattcctgttctagtattttggggctaggttgtcaggtatgtatatatttctgtttgttatattttcgtgatgtattcactttatatcatggcagaatgtttctctttagtaagatttttgatcttaaaaaagttggccagatggggtggctcatgcctgtgatcccagcactttgggaggccaaggcaggtggatcacctgaggtcaggagttcaagactagcctggcgaacatggtgaaaccccgtctctactgaaaatacaaaaaaattagccagctgtgatggtgtgcgcctgtaatccctgctacttgggaggctgaggcacgagaattgcttgaacccgggaggctgaggttacagtcagccaagatcgtgccactgcactccaacctgggtgatagagtgagactctgtctcaaaaaaacaaaaaaattatttagtctgacgttagcatttctcctcaagctcccttacggttgtttgcatagcaaatctactatccttttgctttgacctatttgtatctttgtttctaaagtatgtatgtctctcacaggcagcatatagtcaaagcttaaaaaaaaaatccagtctaacgatctctgccttttgattggcatgttcattctattcccattcaatgttattattgctgtggttggatttccatctatcagttcacaatttgttttctatgttttatgcctttttgttcttctgttcatcttttactaccttcttttgtattaagtattttctagtgtagcatttaaattccctttttctttttaagtgtatattttaaagttattttgttagtgtttgctccagggattacaatatgcattttaatttatcaggatctacttcagattaatactaattttagtaaaatacaggaacttgactccagtataactccatttcctccctccttgcttgtggtttgtagtattattgtcgtatatgttcatctatatatgttataaactcaacaacatggtgttataattattgtttcacacaatcttatttcttttcaattcagtaagacaagtaaggagaaaaacacttttcaagtcttttatattacactgtatatttatcactgactttactcttgatttcttcctgtatattcaagctattgtctggtgacctttccttgctccagtatatataataacttcattgcctccttgctcctttatgctattattgtcatatatattatatatgtttatgctgtgagcccatcagctaagtcagcttagcaaggtctcaagatacaaagtcaatgaataaatcaggctgggtgtggtggctcacacctgcaatcccagcactttgggaggccaagataggtagatcacttgaggccaggagtttgagagcagcctggcccacatggcaagactccatctttacaaaaatacaaaaaaaaaaaaaaagttgagtgtggtggtgcgcctgtaatcccacctacttgggaggctgaggcatgagaattgcttgaacccaggagggagaggttgcagtgactggagatcacaccactgcactccagactgggcaacagggcaagacacagtctaaaaaaaaaaaaaaaagaaaaaagaaaaacaagaataaatcaattatttttctataatacttgccacaatcaattgaaccatgaaaaattttaaataccatttacagtagcatcataaaacatgaaatatttagagaataatttaccaaattaggagaaatgtctatacaattaaaactgcaatataaaccaggcaccatggctcatgcctgtaatcccagcactttgggaggtcaaggtggccagatcacttgagcccagaagtttgagaccagcctgggtgacatagtgagaccctgtctctacaaaaaaaaaaaaaaattagctgggcatggtggcatgcacctgtagcccctgctactcaggaggctgaggtgagaggatgacttgagcccagtaggcagagattgcagtgcattccagcctgggctacacagcaagaccctgacaaaaaaaaaaaaaaaaagaaaaagaaggtcacaaaaaaacagaaaactgcaaaatattgatgacaaaaattaaagaagacataaataaatggagaaatataccatgctcatggattggaagacttacattgctaaactgtcacttctccccagatggatctacagagtccacataatctcacttaaaactccagaagaaatttttgtaaagttgacagctgattctaaaattttacatagtaatcagaataggttgatattaggatagacagatagttcaatggaatagaatgcagagtttagaaatagacctacacagaaatagtccattgattttttaagagttgctttttgataagggtgctaaggtaatgtgatagagaaaggaaagtattttcaattcaaatggctgaaatgactggatatccattgagggaagaaagggactttagcctttcacacaatacacaaaaattatggaattctgaagaaaataagagaaaatgttcatgaacttggggtaggcaaaaatttaatagatgaggcaaaaaaaaaaaaaggcccaaaccataaaaatggtttcatttttataaggataaattagagtttataaaaattaacacttcccttcaaaagaaaaattaaggaaaaatgaataaataagccaccgactgaaagaaaatatttgttttctaagaagtattttgtttttcattttatgggttcatagtaggtgtatatatttatggggtccctgagatattgtggttcagtcatacaatggaaaattcacatcatggagaactggtatccatcctcttgagcaattatcctttgtgttacaaacaatccaactatactcttttagttatttttattctttttttttttttttttgagatggagtctcgctctgtcacccaggctggagtgtggtggcgccatctcaactcactgcaacctccgcctcccaggtttaagtgattctcctgcctcagtttcctgagtagctgggactacaggcacccaccaccacgcccggctagtttttgtatttttaaactttttttttcttttttctttttcttctttttttttttttgagatggagtctcgctctgtcgcccgggctggagtgcagtggtgcaatctcggctcactgcaacctccgtctcccaggttcaggagattctcctgcctcagcctcccaagtagctgggattacaggcatgcgccaccacacccagctaattttttgtatttttagtagaggcaggatttcaccgtgttggtcaggctagactcgaatgcctgacctcgtgatccacccacctcggcctcccaaagtgctggggttacgggcgtgagccaccacgcctggccttagtttttgtatttttagtagagatgaggtttcaccatgttggccaggctggtcttgaactcctgatctcaggtgatccacctgcctcggcctcccaaagtgctgagattacaggcatgagccactgtgcccgacctctcttttagttatttttaaatgtgcaattaaattattattgactatagtctccctgttatgctatcaaatactgggtcttattcattctttctatttatttgtacacattaaccatccctacataccctctcacccctgccactacccttccaagtctctgcgaaccatccttctattctctatctccatgagtttaattgtttcgatgttttgatactacagataagtgagaacatgcagtgtctgtctctctgtgcctggcgagaaaatatttgtaatatttgtatttggcaaaggacttgtatccaaaatatagaaataaattctgctcaataataaaaagagaaacaacttaatgagacccagtgtctacaaaaagtagaataattagccgggcatgttggtgcatgtctgtagtcccacctaatcaggaggctgagggggaaagatcacttaagctcaggagttcgaggttgcagcgagctgtgatcgtgccactgcactccagcctgggtgacagagtaagatcctgtctaaaaaaaataaaaatagagggccaggtgaggtggcttatgcctgtaatcccagcacattgggaggctgaggcaggtggattgcttaagcccaggagttcaagaccagcctgggcaacatggtgaaaccccatctctaccaaaaccacaaaaaatcagctagatgtggtgcatgcctatagtctcagctactcaaaaggctgaggtgggaggatcacctgagcccaggaagtcgaggctgtagtgagccatgatcatgccaccacactccagcctgggcaactggagtgggactgtgccccccaaaaatatatataagtaaataaataaataaataaataataaaatgagctaaagatctggacaggcttcataagaagcaagcaaatggccaataaatacatgaaaagatgatttacctccttagtcattggacacacttagataccactcctcatccactagcatggctaaaggataaaagagtgaccatcaagtgttggcaaggactgtggttctcgtacattcctggcgggaatggaaaattcagtcaccactttggaacacagtttcttacaaagttaaacatacacttaccatatgacccatcttttccattccttggtatttactcaagggaaatgaaaacacaggtccacaaatacctgcacatgagtgtttacagaagctttgttgctagtggccccgaacagtgaaaaaaccacagtgtctgtcaataggagacagaataaatgaattgtggcggattaacaatggcgtgcttctcagaaatgaaaaagaacgaacgattgatacacgtgacaatataggtgaatcttcaaaacagcatgcggtgtgatgcggccagacataaaagagcatgtatcagaggattccgtgtatgggaaacgccagggaatcagatccatggttgtcagggtcaggagggaccttactggggtgattacatgtgtacatacattgtgtgaaactcatcaaaaccgtatacctaaagtgggcacattttattggatgtaagttatccctcaaattaaattaattttcaaagttttaaaaaatggaaccatttttgccacattgaaggaaaattatttccaccaagatttccctacagccaaacgatctaccaactacaaaaatggaaaaaataatttaggacatgtaaagttcaaatgttttgcctcccacgtttctgtttcaagaagctattcgagataaatcgctccgtggtcacaggacttagaaaggtggaggtaaacacacacaagcattataagataagaagtaacagatgaattagttgaaagggactgatttcgggggaaggaataggaactgggccaggagatagacgcctttcaggcttgcccttgtgaaaacgatagtcccctcttacctgcgggggataggttccgagatccccagtggatgtctgaaaccacagatagtaccaaaccctacagagagtatgttttctcctgtacacacatacctatgatagggtttaatttataaattaggcaccgggtgggattaacaaccataattaatagtaaaatagaacaattataacaatatactgtccgaggcgggtggatcacttgaagtcaggagatcgagaccagcctggccaacatggtgaaacctgtctctactaaaaatacaaaaaattagccaggcgtagtggtgggcgcctgtaaacccagctactcgggaggctgaggcaggagaattgcttgaacccgggaggcggaggctgtagtgagccgaaggtgcccctttgcactccagcctgggcaacgagcaaaactccatctcaaaaacaacaacaaaacaacaacaacaacaaaaacaatatactgtaataaaggttacgtgaatgtggtctctctaaaaaaaaatgtgtatatatcttagtttgtgggttttttgttgttgtagttttgtttattattaagagacagggtcttgctctgttacccaggctggagtgcagtggtgtgatcatggcttactgcagccttcacctcctaggctcaagtgatccttccacctcagcctcctgggaccagaggcatgcaccactatgcccagctaattgttttttttttttttttcttgatagaaatgagttctcactatgttgcccaggctggtctagaactcctggcttccagcaattctgctggctcagcctcccaaagtcctgggattataggcacgagccacgatgcctgtcctcaaaatattttactgtactgtactcacctattattggcctttggttgaccatgggtaactgaaactccagcaagcgaaggtgtggataagggaactactgtacgtgactttaatatacataaaatcaccttaaagacaatgattgtttccagaacatgagcctctgagacagcagggagatactaggggaagttggtggctcctctccctggcaggaactggtctgggctcctggctcagcctggccatgaggctgtcctggcctccctcggtgggttcgacagtgacctcgacgtgctcatttcagtgtggttcattccggtctcccaggggaagggggtgctgagtggaaggaggtcaatgggaagccggggtggctctcagagtctgcaggagcagtcgggctgatgagctgggaggagcagaccgcctccctcttctctgagtgggaggagggccagatctggactgggtttggagatgctcaggtggggctcagagcatcacctgtggggcagagggaccatcttggcagatgaaggcccgtcgcagggtgtgatgcctgaattacaaggcgggacaggtaaagtggggcaggtgagagaaggagggtgagtgatgtgatttttctactcctgttttccaggaaaaccaaaatgccacgcacttcgacctatgatccttttcctaataatgcttgtcttggtcttgtttgggtaagacacatttgaccatcgaggctggcctggtttggggagaagtgaccacagcagccaatcagacccatggggcctccctgagctccccaagtatcacagttatcagggtcctaaggacagttattgcctgcgtccagctctggcggagggtgtgcttacttgctcccttattttagcctcacctgggcaacaggctcatctcactcccatttaaaattttcctaagtgtggagtctggggctgggagagcaagccccttgcccacaattgcgtggctgggggtggggaaggcaattctgggtcccaagctgttagtcgcttccagacacagaaggtcccagaaccaagagtgaagtcacctgtcacctctactggggcatctctggacacggtctggaaacactccctgacgtggcctcagggactgcactgaccaaggcactggtggcggggggtgagggagctggggctctggagctccagcaggtgcccatacgtgagcaatatcccagggaccaccctcctgcccacctcccggtgtgggacgtggcgaggcgcctgagctttgctgagaacttgccctacctgcctcgaggccttgcagcttcaccgggaactcttgtgctcacgctgctggccgcaccatgcactttttggaggaagggaccaacaggcagtcttcgttctgtgtcctgagtcttggcacacttcctttctgcagttacggggtcctaagccccagaagtctaatgccaggaagcctggaacgggggttctggtgagtgcagggaagagcaggtggagcatccatgctggccggggtgctggctgtgggcgggggtcccactctgggaactccccctccccttcctgggcccgctctctatgctctgcccagtgtagacatgctcaactctgtggccttgaagggtcacctggatacctctggagtcagccttgacctcccttctgacctccagtccccaactccaggcttacccagagttcccatgcatggtctctgctcccccatccccacccctcctccaccagccatcctcaactctccctttgctctcacccctacactgggttcccaaatcctgccagccctgctcttcagcagctcgcgtgcccactggcctccctgccttcgacgtcctgctccagggctccggtggggctgccctcatctctgtgacagcctcagacttgtctccccatccccatcagccctgtcccccttcttctcacagcagctggagtgatctttccagaacataaggtaggaggctctccatgatctgatcctgccttcctgacctccccagcctcatctctccctcctctgccctcgccctctgtgctccagccaacgtggcctgtcactcgtccacctgccatactgtcctgactccaggcctttgcctgtgctatggcctctgttgggaccactcttgtctctcccctgctgtgtctgctaattcccactcgtgtcagtgatccatggctgcagaacacaccactccatccatggaaaacaatcacaatcattgattactatctctccctgggcttcctggggtggactgggctcagctgggtggttcactttggggcctcagcagtcatgggcagacagtggctggggtcactgcaaagacttccctgcatctctggcagttgatgctggctgtcatctgagacacctacccagggcctctccctggggcctgggctcctgcttagcttggttgggaggctccaagaccaacatcccaagaaagatgagacagaagccagatcacctttttgggcctggcttcagaagtcacccagcatcacttctgctgcatttatttcttagaaacacagaatatcagaccccatctcttgatgggagggggcctcatggtttgtagacatgttctgaaactccactctgcccggccttggcttcgacgtgtctggtgaatgtgtgggctgtgaggctccccgaacgtagacctcagactgcaacgctggccgttacagggtctggcacacgggcccacgtcaggcccatcgccacagtgatggttgttctgtgactgtttctggtggcctctgctccacactccaggctgacgctgtgccccttccactgggaccctcgggtggcttccatgcacttgtgccctaaatcctgctcctagactaaacttcatctcctgtgttctcattctgcagcatggctgttagggaacctgaccatctgcagcgcgtctcgttgccaaggtataatgtcagtgcctcccttcagtggctcccatgtcacagaattgtcctgcagccctggcacatgtgtgccatgtgggagctggggcaggtcctctttcctcctgtggctccgagggagggggccgctccttccccagtctctaccctgacttggccctcgtcctgcagccactcagagagcacgatggagctggagcttcagttttgaccaaatgcgtgtcgccggcttttgtgtgtgtgtgtgtgtgatggagtctcgctctgtcgcccaggctggagtgcattggcaccatctctgcccactgcaacctctgcctcctgggctcaagtgattctcctgcctcagcctcctgagtagctgggattacaggcgcccaccaccacacctggctaatttttgtatttttagcagagacagcgtttcaccatgttggccaggctagtcttgaactcctgacttcaaatgatccgcccgcctcagcctcccaaagtgctgggattacaggcatgagccccggcacctggccttcaaattggcttttaaagaagatgaccagctggtctgttctgtctggggccttggagggctgtttcccaggatgtgggcctttatcagtgctgacctaggcaatcaaggccaagctggccacactgcttttgattttttttaaattgtgatgaaatacgcttaacataaaatttgccatcttagtcattttggagtgttcagtagctttaagtccattcacattgctgtgcagccatcaccaccatccatctccagaactcttttcatcttgccaacctgaagctctgtccccataaaatagcaactccattttccctcccccagctcaccattccatggtctgttgttatgagtctgctactccaactaccttttataagtggagtcaatggtacctgtgtttttatgtcgggctcaaatcactgagcgtgatgtcctcacggtccatccaggttgtaccaagtgtcagaatttccttcctgttttctgctgactccctgcttttaaaggccatcttggacattcaaattgtggccattactgacagttaaaatggccccggctcatgtctcctgagcctctacaatcctttaggtggggacagtactgcggcccacacgtccccacaggctgcgtgtgacacctgacagtgctttgtgccactgtggagcaggtgtcactctcccacttctcagagaggaagccgggctctggaagccagcagcccacaaggaggcccagcaggcttcgggcccaggtggggaggtctgcacatctcagggcctttccctgggaacaaccagggacagaggccacacacccagcctcctctctgcccacagtgaattgagacacaaccccttacgtccctcccagccttaagtcaagtggaaagcaatggcccctgaggtcacttagtccctcttgccagtttgtaagtgctgaatcagagactctgagctttggccctttcccgagttcccgggattatgagctgacacagcccccaagcaaccacagtgattccagcctgggagtgtggccttggcgggagggtcaatgcccagtcatggttcagcggctgtgcacacccctgcttacctgcatcccacgctttccatgcaaactcacgtggcggcgctttgcgcttgcaggatggtctacccccagccaaaggtgctgacaccgtggtgagtaaagttactgacactgaaactgaacgcagctcaaggggctgttctgaaggtattagagggcggtttccttgatgtaaatctcagttggggctgcttcgttctctcctctcagattctcctgacatctgaattcagaggcccacatggctgtcctctttccctttgctttctctgacttgcgtctcttgtttcctgtccctttgttctccaaagcccctgcaaaggcctgataggtacctcctacctggggaggggcagcgggggttgggtgctggggagggtttgttcctatctctttgccagcaaagctcagcttgctgtgtgttcccacaggtccaatgttgagggagggctgggaatgatttgcccggttggagtcgcatttgcctctggttggtttcccggggaagggcggctgcctctggaagggtggtcagaggaggcagaagctgagtggagtttccaggtgggggcggccgtgtgccagaggcgcatgtgggtggcaccctgccagctccatgtgaccgcacgcctctctccatgtgcagtaggaaggatgtcctcgtggtaccccttggctggctcccattgtctgggagggcacattcaacatcgacatcctcaacgagcagttcaggctccagaacaccaccattgggttaactgtgtttgccatcaagaagtaagtcagtgaggtggccgagggtagagacccaggcagtggcgagtgactgtggacattgaggtctctccttgtgttcaagacagagtggggtggcggccagccttgtcctcccagagggtagatgggaaaggtcattcatgcagcatcttactgagctcatgtgggctcgtgggctcgtgggctcgccaggtcggtaaaacccagctccttctccagaggctgcgtctcacccagggatggtggcttctgctgccccctcctctctgtaactgtggccggccgtcatgctgagccaccccctcaatacaaggctccagatgtttcctgctcactgaccagagatagcaggagggggacacctgtttgctgtccttggaccctagaaagaggatgctggcagagccgtggtcacttctctgtcagatgtaggtggggcaggcaaagcagttggccccagacaccaaaggaagtggctgacccacaaggccctgggactctgggccaggccagagagggagctagccaggcaaccgcagacacatacttgacttctcggcagctgtgggcagctgggccagcgacagtggcggaggccaggaatgacttactcttaggaataggtgcagttcaagcctggagggaggaagctctagggtgcagaggcgggtgtgtggaggcctcgcgtgcagcttataatgagggagcacgtggccggcctggccataagaggggcagctgcgtggggaggcgtggctcaggccaggctgagggggagtgagcagacgccagcctgcggcctgctaccagcctccagccacctgccctcagccctccttagtaagagggggtgctggtggtcccccatcgctgggaagaggatgaagtgaatcgcagcccgaggactcgctcaggacagggcaggagaacgtggtgcatctgctgctctaagccttccaatggccgctggcgggcgggtgcaggacgggcctcctgcagcccaggggtgcacggccggcggctcccccagcccccgtccgcctgccttgcagatacgtggctttcctgaagctgttcctggagacggcggagaagcacttcatggtgggccaccgtgtccactactatgtcttcaccgaccagccggccgcggtgccccgcgtgacgctggggaccggtcggcagctgtcagtgctggaggtgcgcgcctacaagcgctggcaggacgtgtccatgcgccgcatggagatgatcagtgacttctgcgagcggcgcttcctcagcgaggtggattacctggtgtgcgtggacgtggacatggagttccgcgaccacgtgggcgtggagatcctgactccgctgttcggcaccctgcaccccggcttctacggaagcagccgggaggccttcacctacgagcgccggccccagtcccaggcctacatccccaaggacgagggcgatttctactacctgggggggttcttcggggggtcggtgcaagaggtgcagcggctcaccagggcctgccaccaggccatgatggtcgaccaggccaacggcatcgaggccgtgtggcacgacgagagccacctgaacaagtacctgctgcgccacaaacccaccaaggtgctctcccccgagtacttgtgggaccagcagctgctgggctggcccgccgtcctgaggaagctgaggttcactgcggtgcccaagaaccaccaggcggtccggaacccgtgagcggctgccaggggctctgggagggctgccggcagccccgtccccctcccgcccttggttttagcagaacgggtaaactctgtttcctttgtccgtcctgttgtgagtaactgaagcctaggccccgtccccacctcaaatcacacacaccccctccccaccacagagacaccattacatacacagacacacacagaaagacacacacagacacaaaatcacacacacaccctccccgccacagagacaccattacatacacagacacacacagaaagacacagacacaaaatcacacacacaccctccccgccacagagacacaccattacatacacagacacgcaatcgcagatacgcccttccggccacagaaacacaccattacacacacatacacagaaagacacacacagacacacaatcacacgcagcccctccccgccacagagacacaccattacatacacagacacacacagaaagacacacacagacacaaaatcacacacacaccctccccgccacagagacacaccattacatacacagacacacacagacacacaatcacacacagcccctccccgccacagagacacaccattacatacacagacacacacagacacacaatcacagataccccctcccggccatagagacacaccgttacacacacatacacagaaagacacacacagcccctccccgccacagagacacaccattacatacacagacacacacacacacacaatcacacacacaccccccgccacagagacacaccgttacatacacagacatacacacacacacagacatacaccagacacgcaaagacacacagacacagatacacagatacaaagacacagacatatagacacacagacatgcacagagacacatggagacacatgcaaaaatgcacagagaaagacatacagaagtgtacacacagacacatagaccacacagacacacagacatgcatgcaaacacacagacatgcagacatgcacacaaacacagactcacgcacacagacttaggcagcccaaattcagcgcctggggcataagttcctggaggggtggccaccttcagcccccacggtaaggtcctgaggaaccttccccttagacaagggatcatggaggaggtctcttccggagcctggagggaggcctcaagtggtccttccacctcggcatcccaaagtgctaggattataagcatgagccactgcacctggccccaacatcatttattgaacagactgtcgtttccacattgtgtttcttggcatttctgtcaaaaatcagttgaccgtaaatgcatggattcacttccgggctctctattctgtttcattggtctgtttgtttttatgccaataccatgatgttttgattattatagctttatagtatatcttgaagttaggtagtgtattgtctccagctttgttctttttgcttaagattgctttggctatttaggggtctttgtggccattactgacagttaaaatggccctggctcatgtctcctgggcctctacagtactttaggtggggaagtactgtggcccacatgtcccaacaggctgtgtgtggcacctgacagtgctttgtgccactgtggagcaggtgttactccctcatacgaattttaggattattttatttatttcagtgaaaaatgtcaatgaaattttgatagggattgcattgaatctgtagattgtttggggttgtatggacattttaacaatattaattcttccaattcatgaacatgggatgtcttttttttggcacctgatctcagatatgggacagctttctaattatttgtatcttcaatttctttcacaaatgttttatagttttcagcatacagatctttcacttccttggttggatttatttgcaagtattttttgtagctattgtaaatgacattgttttcttgatttctttgttagggtatagaaatgctacagattgttgtatgttaattttatatcctgcaactttactgatttcatttattctaacatcttattggtggaatctttagggttttctataagatcatgtcatctgtaaacagggacaatttaagttcttcctttccaatttggataccttttatttcttcctcttgcctaattattttggctgagggccaacttgttgggtttgttgttgttggtttttgtctgtgtcttttggtgtttcgtgttgctggcttttccagcacacagtcacagatatatgaggcagaaagaaaacccagggaactcctgtcatgtcattcctcaggtcccaaggtccctacatggtctgccttcttctctccaccttttagtgtttgatatacatacgtttatgtatgtagttatatatgtatgtgtaatacccagggaaatatacagagattttaactgtccttagtgtagggcgtggggacaggtgcatctattctaccttgcttggaagtggaaaaatcttcaagaaatcaatttatcaccaactggcatgacagaactcctggtttagtgtctagctgtctccactaaaatcaatcaccatgccatcaggcactctcttccctctggtgtgtactggtacctgaaacagcgtccagcaccctgagagcacagaacgcagcttcttatgtgccccacccaataatcccaaatcgagcacagacctgggagctgaagagacaagctggaggtcaggcagttctattacttaagcctggttcttcctgactccaaaagccaggcgccaccccatgctccttcccagcagaagctggttttcaccgggtcaggaggacaggatggctattcctgaccgttgtacagcagtgccacatctcctagtttccagaacatttaatgtgttgctgtggaggcctggcagctctgaagtagccccttgcgcccaggacaaaccgggcctggaggggtggggtggggtaagctgaagcttccagtatcccacgtgggcattctgttgctgagggcaaccgcccctccctgcaagggaggggaaggagcaggcaggcacacccttcctccctccacatttcactggagttgacctgtgtcctactgtggttgcacctggaccagcccagagggactggagtgactcttcccgcaggtagggcacagccagcctgggcacagggcctcactgcaatccctgcgccccgcctccctgggggatttccaggggttgtcctgctgcttggtgagctgggggtgtgggggaccacagatgaggcaggcgcctaaattgcttgggcggagtccggctgctctctccctccaggccctttcctgtgtcctcgggcccctcgggtccctgcaccaggccagcagcaatcgggtgtcagaggttcaagtccaggctgactcctgggacgcaccgactttgcccaggagggctggggagagggcagccccaaagtgtcatctttgattattctttgtccccaaagccagccttgggcatgtgcccagcacagggagagctgctgcctctgtgggcttgagtcatttttgcttccttgcccctctctgctctgtttggtttaaaaaacaaaaaccaacacccatgggtgaggcgaggcgaggcgaggcttgccttgatccacccgcttcctcccctgcagcctcgggccccatcctcccgcgtggccgcactaggggtgctgggggcggagtggggaccagacctgagtgggttctgggttcagcttcgcagcaggagcagctggggcatcatcagttgggcaggtgacccaggatctgagcaggaatcttccaggtggaggtggggccgcccctctgcttgccacagctgccttctgctctgggggcagcactggctgagaatccagtgaagtaaggcctttgaaatggttttgattgtaaaagtaatgtttatgtttagccattcctttttttattttttattttttgagacgggtctccctctgtcacccaggctggagtgcagtcgtgaaatgtgggctcactgcaacctccacctcccaggttcaagtgattctcctgcctcagcctcctgagtagttgggattacaagtgcctgccaccacacctgactaagttttgtattttttagtagagatggagtttcaccatgttggccaggctggtctcaaactcatatcctcaagtgatccaccctcctcgtcctcccaaagtgctgggattttgccatgagccaccacgctcagccatgtttagccatttttaaaaggtgtgaacagataattaagtctattagcaacattagaaaatttaagttaaaatttaaatgactctagcaagaattagcctttaggtgccctgggtccccctcctcaccgccccccaccccgccgccttctgagaagcgttttgtaatttttcttatttataaaatgggtttgattcaaaactgacttttcctcttcacagtctcacaggtccttccccaggtccaagaggctcttctgtgtcctgatgacaagtagctgcctagccgtggtggcacctcctatcacatgttaagggacccctccccagggccacacctggcagaaggtggcttatgatgttcgcagcttgaaagtagtgtaaaccaaagataaaattctaagcccactcccccagccatcggaatggacccctcctcttggccagggcactccaaagttaacctgaaaaaccggttcaggctgtgaagagaaggtggagtggacatgcctcatttatgtcctcctcccttttggaattcagcaaagctgaccagcatgaacattaacacagaccttaagtctgattagtggcatttacaatctatactctctgaagcgtgctacctggagtcttcctttgcatgataaaactttggtctccacaaccccttatcataacctagacactcctttctagtgataataactctttcaaccaattgccaataaaaaaattttgaatctacctataacctggaacctccccgctccaccttcgagttgtcctacctttctggacagaagcaatgtggatcttgcatgtatttgattgatgtctcatgtctccctaaaatgtatacaattaggctgtgcccagatcaccctgggcacatgttctcaggccctcctgaggtctctgtctcgggccattggtcactcagattcggctcagaataaatctcttcaaatatttttcagagtttgactctttttgtcgacaataagctcatcagattaaagctgttggaactttaaattacttcgagccttgaaagaatgtgattctgacaccctagtcaagcagcaagtcgctgtgaactttgcttctccaattatagattaacgctcttcctttcttgatttgtaaaatgttatgaaagactaaatggcaccagatgtaagaccccttccctttcttagttactgaccttttgttacgaattaacttctttcttttcttgcacccaactcagaccagatggtacaaaagaccccatgcctatcaaattccataagacagtatgttaaatatacttttcccgaagggaacaggatataaccaaacaaattgctgcaactcataagccaatcttatgtggaaaatgtaatcatgctaaacctccttgtattttcccatttaagtgaagccctaatccctccacctcagagcactgagcccattcctttagagtctgtgttttttgagtggtggtcgtcaaacttagagttcgaataaactctataacattggattttgaacctttcaattatttcaggtggcagaagaaacacacagaccccatctcttcctttggagttctgtactgctcatttcaaagggatttcatcctgccctctgtaaccacccaaggggttcctcctgctccctttctaaataaagaccatgcattccagtaaagaaagagtttaatagacatgaggccaaccacatggtagagagagtttgtactcaaatcatctcatcaaaggctcatagctaggggtttctcaaggcagtttggggggtcaggggtggccaagtaacaggtactgattggctggggcagacatgaactcatggagggtcaaagctgtcctcctgcaggctaaatcgcttcttaggggggtgtcacaggagtgaggttgggggttccaggtagagccatgggtatcagacctgcaaaaaacctgaaaagatatctcaaaagaccaatctacagttggggaaattgtgtattttgctagagaggtctacaccttagcttctccctctcgcctcatagcttttcattaggtttacacaggcagaagagttttgggggaggcctgctgttatttaaactataaattaaatgtctcccaaggttagctcagcccaaaagcccaggaatcattaagggaaaggaaagatgaggggcgggttagctcagctcactcttacaatttttctaattgatagagtttttgcaaaggcggtttcactttcagcgtggtcgttctcctaacctctcctgcttccccaggtcctcaccctaaaccagcacccatcctgcactcccagggtgaaccctcccttccctcctgctcacctgggcccaacccagtttccccccaggagctgcggaagggcccagtggtgccaccttggcctccaggcatcactctccaacttcttccctcctccctccacaggacccttgcctcaaatgtcagtcccgctgctctctgctgcactcagcctcttcctggcaagtgtgcccctcccctcttgaagccatcaccagcctctcgtgcccacccccatctaggccccccactcctctccagtcccggtgtcttggaagagcctctggatcacaggggctgggaaattcgacttgtcatccccaccaagcaaacacatgcttccatccagggcccctcagtgggtgaaggctgagccccagtaaaggatagatcatcactgtcagaggcgagtgaactagagcgactccatgttgaatagggactgggtaaaatgaggctgaaccttctgcactgcattctcagggggttaggcattcttagtcacaggataagataggaggttggcaggattagtatcacaagatacaagtcaccaagaccctgctgatcaaatgggatgcagtaaagaagccagccaaaacccaccaaaaccaagatggcgacaaaaggaacctccagttgtcctcactgctcattatgcgctaattataatacattagctgctaaaagacactcccacctgtgccacaacagtttacaaatgccatggcaacgtccagaagttaccccatatggtctaaaaaggagagggaccctcagttcagaaaaatcccctcccctttcttggaaaactcacaaacaatccacctgttgttgagcctataatccagaaatcagtataagtatactcagctgagatccaagaaccctgtcttggggtttggactgggactcctttctggtaacaatactagggtatacggagcccccacatccccgggcaagtggtgcagaagccggagccatcagcagccgcccctggcaggctcctgtgtggactccttgctgcagggcatttggtttaccaggcgtgagagacccacacgtgaagcgctagcccagccccatcaccgcagcacagcctcaccactgcagcccagaaaaaccactgcagcccagacacacgactgcagctgaggcccatcactgcagcccagacacaccactgcacccactgcagctcagcccaccactgcaccccagccccaccactgtagctcagcccgaccactgcagcccagcccaccattgcagcccagacacaccactgcagcccagcccaccactgcagcccagccccaccattgcagcccaggccctgcagttgtccctactaggtctgagtggaggaatcagagagaaaactctggaaagtgctagacacaggggaggccctcaaggaatggtggctgccactgggaactctgtttctgtggtgcttactggacagataggctcctttgcagatttttttattccaaaactaagctcccatcacccagtcctgtcctccctgctggcagagcttgtccctggatccaagctgtctcctctgcaccaaatccggtggcctcaagtcccagctctgctttgcacaggggcttttcgaagccctgtctcccctgctgtgagatgggggtcacccagctgccccccagggggtggtgttgctgacggagcaggtgaagacaatgccaagagctgcgcgtggtctggctttcccaggctggatgtgcccctctgatcctcagtctccccacatggacaatggctgatgccgaccacccacactaggtttgccccagagtcaaagtgagctccttgaggacagggaactgacccccacacagggccccccaggatctgcttgaggaggcaccaggcagttctcccagaaggcagggcgtgtcttcagagctctctgggttgagtgggcctaggccatcgctgcctctgcccaagtctttctagaggcttcccaagtaccagagctgccctggagggccagagttaccagaaagtccaaagggagaaagtcagagagacaccagcacacccaagaagtccagggccttggggacactaagggtgaactctgtgctgtgaagggcaggcctggcccggagagagggagcctgcagaggagacgggatctctcggtgctcttccgaagaagcccctggatctcccactgtgttctgctctcccgggcgggcaggtgtcaggatggcgcctggtacagctggttttgggcagaagccccgcccgtggcctgcacgttgggtgtctgcgtgttgggcccctgctctgcagcctcagcaggtactgtgacgggggatggccgggcagggacctacctggccagctctttgtctcagctgccttcacacggcagggtttgctttgcgcatcccatccctgtgaacatacaggtgcgattagtttgctggtttgtggcagggtggttttttcttcttcctaaaaatggatttagtagatttttagcttcacaggtttcatctgaaccaatgacttggaatttaagacaatggctacacgcttttgactgggtttgggatttgtgatccgaaacggttcatttgttatatggctagaatatgacatgacttttcatgtgttttaaaaaatataatgaggcggcggctgcgtccacgtcgaccaagactggagcgaagtttaaggaagggacagaatcgccggggagtgcccttgttgttgctgggggactcccagccttccctgtggcccgagaaggagaagcagtggctctctgaagcaggcatggagagtagaaaactggagttattagcataccctagtacctcttacaactttcccttccatgttagcactttagtgctggcttctcagttttcttaacattgagacaataaatgtgtgttgtgtcttgtagatggcataaagagtaaataagaagttttagagttgttctggaaaatgtcagaataaatctccacttgagttgtgtattctgctagtccaagtggacagcaacttcctgctaccctcccttgcaaccttgcagacaagtctctcctctgaagaacgaattagatttagctaattagaattaatccttgctttcatcgccatcatctgtaaaatactttgttgggtagaccactttatacctttgcaatacggtctccgtgggtaaaaaacaaatatctgtaatgcagacaggaggatgtgaaaagttctgttgcacgtgttttttttttttgttttttgttttttgttttttgtttttttgagacagtctcgctctgtcccccaggctggagtgcagtggcgcgatctcggctcactgcaagcaagctccgcctcctgggttcacgccattctcctgcctcagcctccggagtagctgggactacggacgcctgccaacatgcccggctaattttttgtatttttagtagagactgggtttcaccggttgcacgtatttttaattctgtggctgtcacatgatagagaatggaattgaatggagatttcccttttgcttcttagggttggaaatattcccatgaaaatgttcaaatttggaatttgaaagccaccaaatgaatctttatgtataaatccttgtaaatgatagattccataggtgagacttttatgtatttcaggtgggagcctactggcatatatttttaaatgttcatattacttagaatctccaataggaagtctttatttgaaatagttgaatccgtgttctagtattttcctttcagcaagatctgttaggtttttaccccttcaaaaataagttttatttcatctgcaaattgtggcaatgttatagcgatcagaaactacgtaaggaatgttatataggcttgtcagttcccatttatcttaacaacaataaatatcacatttcttcttttgaaaatgacacatatttaggccaggcgtggtggctcactcctgtaatcccagccctttgggaggccgaggtgggcggatcacgaggtcaggagatccagacaatctggttaacatggtgaaaccccgtctctattataaatacaaaaaaattagcagggcatggtggcaggtgcctgtagtcccagttactcgggaggctgaggcaggagaatggcgtgaacctgggaggtggagcttgcagtgagcggagatcgcgccactgcactccagcctgggcgacagagcaagactccgcctcataaaacaaaacaaaacatatttaaacacttagaaaataaagttaacacttactgaagtgccggtactacactgtcctagtactaaaaggaagcaggttggaacatacatatggcctatcatttataacagaattaacttgttgaatgtctgtaaatgatttttttttgcaaaggaaaaagttgatcctggaaaagattgttgtgcatagttattagtcatttgtaacctcgcttaagtgtttctcagttgttcaacatagacacttttttctccttaccatgtatttttaaaaatagtctattacttgactttgaacgtaaagctttaatcataatttcccacgtatacatagttcatctgacggtaagctggatttgaaggtaggggtttcagcgttacttaagttggtagctgagggtatcaggcatcagttcatgcaataagacaaaaaaatatatatcctttgcttgccaaggggtagagtgatgtgcatttatctgttttctgttctgtaagtttagactttcaaaccattttgtaaaccaacccttgggaaatttgaaattaccttataacttaagactctgtggtctctggaatcaccctatctgtttcttttctgcgtaggtatttataacattgctgtttgactatagcgtgcactctgaaatgttatcagtggaaatttgtttgagtttcattaatgctatttcactagttagacataattacttctaccagtgtaaatgacactgatgcgcacagagcttccagatctttcagactcaactactaggtcaattagtttgcataataaaacttggcagtttctacaagcctattatgacaaaccaggagctaattctgtaatgaaaaactatccattctgaatgatagggacgtaattatttgctgctgctgtcctttgtaaattttgaacatgacattatactctgtgcctactaaaggtatcctctggagttttttgagaggagggaaactggaaaattaaattgtatttttgccagaagactcttacttgcatgtgtctcagggtcttcagtttttctagaagtttccatatccaaggttcagagttcatgtgaaatacttctttgggagaaaaaaatccttcattcctggtattcattggattggaaatctgcagcaaaatgctgtttaaaattactatgtggtttttctatcttatccttagctctctggctattgaacttttttttcttctttgaagttagcttcaaatttgcttctatgctaaattacctgtaaatattctggataggaactatttgaaatagtatttgttaaaagaaatgataaaatgaaaatgttcaaactacagagattttaaaatgccataacgatcttgcaagactaactttaaaatatactataaatgattattatgattttggtggtaacgatcccccacacacaaccgctgtgaagaaatgacgccacattttcccctattgtaccaaaaagataaagatggtaaacattaatcaaggtattttgtattgtcaacgcgtgcatattctaaagagttaaatgccaactcagcagcactggcttcctggctggtcaaccataggaaacctcgttcatttctcccagtgttgtgatgttcatacttctacaatcttccctgtcatgactttaacttctacgtttcattaaccattcctgatgttagttctcagagcttcttcttttttttttttttttttttttgagatggagtctcactctgtcgcccaggctggagtgcagtggccccatctcggcacactgcaacctccgcctcccgggttcaagcgattctcctgcctcagcctcccaagtagctgggactacaggcgtgcgccaccacgcccagctattttttgtatttttagtagaggtgtggtttcaccctgttggacaggatggtctcaatctcttgacctcgtgatccgcccgcctcggcctcccaaagtgctgggattacacgtgtgagccactgccggttcttagagtttcttaaggcaaaaataaaattattcaattctgaaaaaatataaataaataaataataaaaaatataacgaatctggaggactgcatggtgtcctatagcgtgacggttaaaagcttgggctttggaaccaagtcggcctcgaaatcgcagctttgctgctgcctgggccactggaccttctgagcgtcgttttcctcctgtgtcagctgagggtggtgacttgggctcccgctcaggggttgaaagagattctgcagcctctggagctgcccctcagcgcatcatcactgggtctgctgtcctcggtacctgcctgggtggaggtttctgtgacttctcaacggaagtggctgtctcaggcgaacgctgtaacagtgaccatcccttgaggtgcctgtttcttgctctgtttttatttaaagatctgtggaaatgtagaatatgaaaaatgaactgcaatattcgggatgaaaatggactaggctaagtattcctgatttgaaaaaacttttatcttaggtttaggggatcgtgtgcgggattgttatataggtaaactccgtgtctcgggggtttggtgtacaggttatttagtcacccaggtaataagcatagtacccgataggtatttatttttgatcctttccctactcccacgcttcactctcaagtaggccccattatctgttgttcccttcttcgtgtccaagcactcctgattaatgagcctaaagaaatactgaaagaacaagcgaaacacatctgttctctttcctgctaccctctccgcatgtccccctctctctagaaaacaatggttttaatcttggctgcatgttagagtcacctagggtgcttttaaaattccctttgccaggcgccacacttgaacgttgtgattatttgcctggcccaggtgtcaatattttttttaagagatgggggtctttctctgttgcccaggctagaatgcagtggcgtaatcatagcgcactgtagcgctgaactcctgggttcaagagatcctccgacctcaacctcttgagtagctgggactactgacacgtgccaccatgcctggttaatttttaaaaagtgtttgtagagatggagtcttactatgttgcccaggctggtctcttaacttctaggctcaagggatctcctgcctcaggctcccaaagtgctgggaatataggcgtgagccactgcgcccggctggcatcagtatcttttgaagttccgatgattctacagagcagccacagttgacaaccattgccctagaatcatcgtcttcactcacatttcatttttaaatgaccgtaattgaaaagacacatttggtcacgaggaataggaaatggacaactcacatctcatacaaatcacaagtacctcctggtggaagcagcaggcctctgggaaggttctagaaagaatgggtccctgtgtctgaagctgtgcttctgagcaactgcctggctggggcgcccctgctcactgggtggtcactgaacttcacacactgctccccagctatgctttcctccttcatgcttccttcttgcttgaccggactccaacaaggaagcctctttttcttcatgattaaaagagtaataaatggtcatgggagaaaaataaagtcatatagaagcagacaggctgggtgtggtggctcatgcctgtaatctcagcactttgggaggccaagatgggtggatcactggagctcaggagtttgagaccagcctggggaacataacgaaaccccatctctacaaataatgcaaaaaaaaaaaaattaggcatggtggcgctctcctgtagtcccaattactcgggagggtgaggtgggaggttcacttgagcctgggagttggaggctacaatgagctgtgatcaggccactgcattccagcctgggggacccagccaaaccatgtcccaaaataaaataaaataaaataaaattaacataaacataaaaaaggaaggagatgagcagaaagtaaccacccacccccaaaagctcctctacctgggacagtaggttgggggagtgtggcaccctggagccaaaggtcccaggctcctgtccaactcactacaggccatctctgctctctaaccccaagcctgagtttccttgcctgtaaaatggagaggacagtagagcctgcccctgggcctgggaatgaggagtaaaggagtcggagcattactggcacgtggggcggcagaagtgctgtgtcctgtttttacacacatgcgcagctgtaaaacggtttattttatttttcattttttgacagagtctcgctctgtcgcccagtgcagtggcgcgatctcggctcactgcaagctccgcctcccgggttcacgccattctcctgccttagcctcccgagtagctggaactacagacacccaccaccacgcggggctaattgtgtgtgtgtgtgtgtgtgtctgtgtgtctgtgtgtgtttagtagagacgggatttcaccgtgttagccaggatggtttcagtctcctgatctcatgatccacctgcctcggcctcccaaagtgctgggattacaggcatgagccaccgcgcccggccaaaaggtttttaaaaacatgaaatgagtcattctgtgcacaccattttgttgtcccgactttgtgcccatagctgagtttgccagacgagggacccccagtcagcaccgtgggctcaagagagggcagcctcagaatctcaggtggccggactcagggctagccctggtgtcctggacagtgcccagacctgaaggccgcaggggccccctcccccacaccgcccagagtgaaagctccagaaacagctcagggcctgagtcgggaatccctgatccctaaagtggagttacagaactttcagggctgtggggaggatgatgaaaggggctcggggcggcagccccgttgagtcaagggggagtgcccagctttgacattttatccacaggaggctcggaagggcaaagtcccatttcgcttccatctggctttcccgccgcttgctctgtgtgcctggttttgttttgtttttgttgttgttgttttgagggagtctcgccctgttgccaggctggagtgcagtggtgctatctcggctcactgcaacctccacctcccgggttcaagcgcttctcctgcctcagcctccaatgtagctgggattacaggcgcccgccaccacacccagctacttttttgtgtttttagtagagacgaggtttcaccatgttggtcagggtgatctcgaactcctgacctcaagtgatccgcccacctcagcctccccaaagtgctaggatccgtgtgcctttaactccttgcctttaggagtcgccgtgctgtaagcagcactgccccggggtaagggcgggcgctggagactggcagtgctcaccagcgcccccagctggtaagtatggcagtcatcgagcctggggggaacggaaagcaccactgggatttctccccgctgagctgggcagttctctcctctgcccagggcgctgccgtgcccccagagggctttgggaaatgtgagttggctcttgtggctgttcctatgcctggggaatttggaagagaccaatgtcattgggagggacagaaagtaccacgcactgaagaagtgtgcccacttcccaaccccatgcccagggtgctcaggagaggaccactgggaagaccgcggatgaacattccagatcccctcggggagagaaggcgctcctagccagctggtccttgagcgcacgcgcactccgtgttggagcccatttagaataaacattcaaaccctcagagcccactggagggctcagggcctttcaccttggctaagaaacgatccccacagccttgaagctgggtggaagctcaggtaggtggtcagggtccctgggtcagcctgtctgcccagctccaggcagctttcctgatccttacaaactccgggtgcagctccacaccgccgggtcccgcccaccggctccgctccgaccacgcccactctacccctcaacacagccttgaccctgccccactggccctgccccaaccacgctcatcccactcctctgccccgcccttctcggccccgcctcccccgctcagctcagcccccactgggcctggacctccctggttcctgtctatcctcccctccaccctgccctctctaaccgaatccccgccccatctagagccctaacctctagatcggccgggggttcatagatcataagccaggccctgctggtgacctggaggccgcggacagccgcccttcaccctggtggggccaccctgccaggagctggcctggaggcggggcccggggctgaatttggggtcgctgctcccactgcccacggttcttcccccaaccctgcccagctctgggggcctttctctcatctctctcccttttgttgttgctgaaaaatgctgctgtttttctcgtggctaagttatatatgctcttaaaaagtaatgcccggcatggaggctcacacctttaatcccaacgctttaggaggtggaggcgggaggatcgcttgagcccaggagttcaagaccagcctgggtaacaaagtgagaccccatctccataaacaaacacataaatagtaagaacttgataaaggtctgagaaagaaattagagatcactgtgaatcctttccatgctgacattttggcacattttccccaggctgcgtgaggcccaggctgaatgtgcacattggcatcttgttttctttattaacatcatttcagaagcattttctgagatgagtacaaactgaaaacattcttctgaacacctgcatggagccctcttttcacacatgaccctgtgatgggccagctgggctttggataaaaaaaagaaaaaaaggaaaaaatgattagaggtgatccagtagcgcgatgctcctcccatctgaggcaaaggaagaagaaataatttaaaaatgttcccagagcaaaaacagccgttttctaaagaatggccccacaattggttcaagttgaagagcctccccgaggaataacagcttcgtcatccatctagttcaggcgttgtttctatagtaacctgtgacgtggtccccttcccaaaacacaaaacaaaacaaaaaacagaaacaaaaacaatgaatccctgagaacagtgagaaagaaaaagctcttgcttcctttggcctacggaagggtgtggctgttgagctgagccttggagttcctgggtttgccggtttccccctctggcagtgctgggggctttgagccagcaacctcaaagaatgcccatgccagggaggccagcatgcacgcaggtattgatgttggctttccacgggtgcagtgagccgcataatggtttggggtgaggatttaaacgttttgagacaggaacggcttgcctctgttttaaagatcagctgctgccatttcaaagatacgttctgttactttgccaaggggcctcattccatgttgctgggaatcagcagcagtgctgctctgatgtggctggtggccgtcccaagcccccatgcctccctgacctctcgctgcccagcacttcccgctccaggggtgctctcaccagccatgactccccctcccgatgcctacatcccccgcctgtctgctgtgccctgtccacagcgccctgtccacagcacagctggctcaacacatgtggcatccaagccgggcaccgtgctttgcctcacactgctgagtccacctggccttacatgccttggctggagtccaggcgtggggcaggcttcggggatggtgtcaaggactgggcacttcctgcctctctcctcaaggtccccagggctgcctcatcccaactgggtcccctgctgcagttctcagatgccctctgcatgttccttctctgggctccgtggctcattccagagcaatcacagaccagagggacggcgccccattagatcaatcagacaagccctggagtggggagtgcagtcaccatccatgagggtgcgggtgtgttggggggatgcggggaggggaatggatgccgcgtcagccaccaacagccctgacgggcccctgacttcctgcactaacattgtccctgtgctcacatctgcccagtaccactggggccattctcacctacggctctaccaggtgcccaggaaatgcccccaaacgggcaggatgctcacaaggcaaggccacctgccctgaaggacgcgggggccacagggtaatggaaagcaaagggatgggggaattatccccaaatcagtcacacgcctgatggcagccctgtcaaaatccctagggaggttttttgttttgttttgtttttggagggggatgactttgtagagaaactgcattttttcttaaaaatgtaaaatgatctgcaatgcaaatataccatgttctgtttagggcaagtagcctcttctgattttgagtctacgaataatcagaagcactgaaaattccaaagcaggagttaaagtcctcagcagcgcttgggtgtgcggtggggtccgtgatggatgctgaaaccgtgggctccaaaggccagcacaggaaggaaggagagggcctcaaaggagggggcaggttggacctggccaaccccagcgtggcaccctcagcccttctcagctcccagtgcctcttaacattgctggcaggtgtgagcctggggtcgtgctagcctctgagtgagctcagccccttaccatttccagcctcaccttcctctcccaagaaagtggcatgagggtaacagcaacctcatgggagtgcaaggctggcacaggctttagccgcatctctgagccctttcaccaaaaaggactcactgaaccaaagagctgcacagaacattagcatccacccccaccccagagagggcttcctagtgctcacctgggggagggtgtgtgtgcaggtgggaggacggagatcgtggcctccctccatgtggctcgtggtatgaagaccccacctcagacatctcctgcctcccaggtggcagctgcctgagtggtgggtggtggctggaaccaggagacccagttctgcctggctgggcctcagtttcccaggaggtgacctggacagggtcacgctgccccatctctgtggcacctggcttgggactccctccctccccgctgctcttccttctccttactcctggggtcttccaggaaagtgccgatcccggagtaagatttggggggcagttcctttgctggcctcaagccccctgcgcactccttccccagcggcagagcgagggcatccggtcctccaggagaagagctgggctaggagcttagtgtgttccccatgtcacggggcctcttccaccccctgactcatggaccccatgctggccacgctgcggaggcctgccaggcttccagatgccggcgactcccctcctggggccagccttctggaagggggtgttgagcccatgaggagctccataagtggggaagcaagtggggacggccatgtccacagcatcaccttggcagctgatgggcgggaggacaggtccaggcggcctccaggcacgttcctgtttgtgtgacattcaccgtgacatggtgcatgccgtggcacacagggtcctgtgttcagataagccagggcctcggaggaactcaggcggaggaaagagccggaacacaaacacagcgcgacctttcccgggaagcggcccctccaggaatgcgccctgggcccccctgccaagccctgggcctccatggccaagtacccctcacttccaccatccgcgactcggttcactccagttgagccccaagagcctctgctgagcccaacccggaagggcgagggagcctcggtggccagagagggccgaggcctgttgggatgactgcactccttcaggacaggcaccctctgtgacggggacatgcagggaccactgcggctgccctggcccaagacgccctggaggccaggcgggcagggccccttcatccttcaggaggtgggactgggctgggttttgaaggatggcagcgtccaggtagactgagggcacagacagggaaggccacacaggcctaaaagcacacagacttaggcgggcgggaaatgtgaggtcaaatcctggagccctcggagggggactcaggaaatcagatgctcagtagggggccgagtttggagccttggccttggggcaggcgccgtgtccagcagaggggctgctgcccgccacggctggccttgcccttggcattggccccagggaagtgcagggcgcaggagagatgccacaaggcccttgggtgttcttcccgcagcttccttcggtcgcaggcggatgacgcatctttgtcactgcatccacgtaagatgctctgttagaaaaaagacaggaaggaaactgttccaactcgcgagtgagtcactggggaggtgggattggaagtgattttctcccctcttccagctttctaagatttataacacgtgtgcatcctttaaactcgtgtcaggaagcgacctcactagggcattgtgtctgagcaggaagaatctcaggactccctgggtttactcatttgagcgccagcctccaggagacggggcaaagggctgctcgtcctccgtgaccaggcggcccctctgcctttgtcgcatgtgtggtcaacgtggtggctgaaaggctcacgtgttccttagcccgggggacctgagaaacctgaccgtcaccgtgtctctgtagcctagttggtggcccggcagtggacagaggccctgcaggccaccaaggaggacggaaccccagggacagaggaggagccgcagtgcagggggtccaggcccgcccacagccccaccctcgggacaccccaacccctgcaccctctgtgcttgccttggggcatttgcttttcaactccacagaaaatattgtgacagtaacgtgggagcagcaagaacccaaataaaagaacgtgtcattcacgctgcctccctttgtcctgagctgcaggtaccagcacatctgggcactttctccatggtccgctgtcctaatcctgactatttcagcacccagggagcaagccaagaacaaaaacacctatcatccatgcccggcattagaaaatctcattgttggtgagaatgatggtttccagcttcatccgtgtccctacaaaggacatgaactcatcgttttctatggctgcgtagtattccatggtgtatatgtgccacattttcttaatccagtctatcattgatggacatttgggttggtttcaagtctttgctattgtgaatagtgctgcaataaacatacgtgagcatgtgtctctatgg

SEQ ID NO: 2 - DNA, 7380 bp, Homo sapiens
  misc_feature 1..7380: note = FUT1
  source 1..7380: mol_type = genomic DNA
gaaagtccctgactggagttggcagccaagccaggccctggagtgggcacccagagggaagacaggttggctaatttcctggagcccctaagggtgcaagggtaggccttctgtgtctgagggaggagggctggggctctggactcctgggtctgagggaggaggggtggggggcctggactcctgggtctgagggaggagggtctgggcctgtactcctggatctgagggaggaggggctggggaacttgggctcctgggtctgagggaggagggagctttggtctggactcctgggtctgagggagtaggggctagggatctggactcgtgggtgtgaggaaggaggggctggggtcctggactcctgggtctgaggaaggaggggcagggggcttggactcctgggtctgaggaaggaggggccgggagcctggactcctaagtctgagggaggagggtctgggggcctggactgctgggtgtgagcagaagggtctgggtgctgggagtcccgagcctggggagatgatggttaaacttctgggaatcaagtcaaactcctgagtctttgacattgatgtatcttgaatgggagggtcagtctgtggggaaggattacccaggtgccgaggcaagagactgaaggcacaaactgtttcagtataataaagaaaatagttagaataagaatagttatcatacaaattagatatagagatgatcatggacagtatcaatcattagtgtaaacattattaatcattagctattacttttattctttgttgtataactaatataaccaggaaacaaccggtgggtatagggtcaggtactgaagggacattgtgagaagtgacctagaaggcaagaggtgagccttctgtcacaccggcataagggcctcttgagggctccttggtcaagcgggaacgccagtgtctgggaaggcacccgttactcagcagaccacgaaagggaatctccttttcttggaggagtcagggaacactctgctccaccagcttcttgtgggaggctgggtattatctaggcctgcccgcagtcatcctgctgtgctgtgcttcaatggtcacgctccttgtcctcttgcattttcctcccgtactcctggttcctctttgaagttcgtagtagatagcggtagaagaaatagtgaaagcctttttttttttttttttgaggcggagtctcgctctgtcccccaggctggagtgcagtggcgtgatctcggctcactgcaatctccgcctcctgggttcacaccattctcctgcctcaccctcccaaatagctaggactacaggcgccctccaccacgcgcccggataattttttgtatttttagtagagacagggtttcaccgtgttagccaggatggcctccacctcctgaccttgtgatccgcccgcctcagcctcccaaagtgctgggattacaggcgtgagccaccgcgcccgcccgaaatagtgaaagtcttaaagtctttgatctttcttataagtgcagagaagaaaacgctgacatatgctgccttctctttctgcttcggctgcctaaaagggaagggccccctgtcccatgatcacgtgacttgcttgaccttatcagtcatttggacgactcaccctccttatcctgcccccccttgtcttgtatacaataaatatcagcgcgcccagccattcggggccactaccggtctctgcgtcttgatggtagtggtcccccgggcccagctgttttctctttatctctttgtcttgtgtctttatttcttacaatctctcctctcctcacaggggaagaacacccacccgcaaagccccgtagggctggaccctacgttagcctgccctgctcggggttggcgatgctggaggtgggccttggaccagagaaaatgctttaattaggtgacaagcgggcagaggcctttgtctctggcgccggcagccacggcccccgctgacggcgtgggaaacagaccctgttccactccggtctccagccttggaatggttgccttcgtgcagtgcaggtctggaaagtagcagtttggcacgggaccctagaattccccaaaaggagtgactaggggctgggattctggaatttgagtgtggacggtgaggcggggggtgtgggagatcggagaccctggtgggcgcgggagcacctgcaggctggaggccctcgcgcgctccggcggcagcctggcaaacaggttctccatcccccaggaggacgcggcagagggcggacgatcgctccactcgccgggaccaggtgcgggggccctgcccagccgctggggcgtggccaggctcgaagcacccaggtgtcgggggccgactctaagccctggcaccggaagagagagggcggcggattggacctcccggctccagcattgcaactgggcgctccgtctcctggtccacgcaatgatgctgcggctgctcagaagccaggtagcctgccctgggtgaagccttcgcgcaggtcaatgacggggcggaggggcagggcgcggtcccctgcatccccgatctggggagcggtgggcccaggggccatcgccttagcccctggcgctggggctcggcgccaagtgacgggcggggctccaccttccagccatccgcccggcccgggagggcggacgctgcgagactcccggccgcgccctctccttcctctcctccccaagccctcgctgccagtccggacaggctgcgcggaggggagggctgccgggccggatagccggacgcctggcgttccaggggcggccggatgtggcctgcctttgcggagggtgcgctccggccacgaaaagcggactgtggatctgccacctgcaagcagctcgggtaagtggggactgccccactcagttgttcctgggacccaggaacaactccttcagaaccaggaggtgcacccccaacctcttctccaggtcttcctaaggccctaggaatctccgccacctccccagccattactcctccaggaaccaagatgctccttccgctcctgaccctccagcctctcttgttttacttgaactatcgtttcccatcaccacctctgtggtggattttgcgcctcacagacaggtactcctgagaaacaggctggtggaagagtccagtatcagcggaacttacaggaggggagactcgagattccttcaggaaaggtgtaggaacctggaccactttctttttttttttttttttttttttttaagacagggtccctctctgtcgcgcaagctggagtgcagtcagcggtgctatcgcggctcattgtgagctccggggatcctcccgccttagcatccggtgtagctgagaccacagacatgtgccaccatgccaagctaattttatttatttttttttggagacggagtttcactcttgttgcccaggctggagtgtaatggcatgatctcagctcaccgcaactcccgccccccgggttcaggcgattctcctgcctcagcctcccgagtggctgggattacaggcatgcgccaccatgcccggctaattttgtattttaagtagagacagggtttctccacgttggtcaggctggtctcgaactcccaacctcaggtgatccacccaccttggcctcccaaagtgctggggttacaggtgtgagccaccgcgcctggcccatgccaagctaattttaaaatttttttgtaagagtgctctgttgcccaggctgatcttgaactcctgggctcaagggatcctcccatctcagcctcccaatatgctgggattacaggtgtgagccacagtgcccagccaaaccatggctatcttgaaaaccacttgtcttccagtccccatgccccgaaattccaaggctctcatccctgaaacctaggactcaggctctccctacctcagccccaggagtctaaacctttaacttcctctttccctgggactaaggagtgctgcaccccaggcgcctcccttaccccacatccctcctcagcctcccctcctcagcctcagagcatttgctaattcgcctttcctcccctgcagccatgtggctccggagccatcgtcagctctgcctggccttcctgctagtctgtgtcctctctgtaatcttcttcctccatatccatcaagacagctttccacatggcctaggcctgtcgatcctgtgtccagaccgccgcctggtgacacccccagtggccatcttctgcctgccgggtactgcgatgggccccaacgcctcctcttcctgtccccagcaccctgcttccctctccggcacctggactgtctaccccaatggccggtttggtaatcagatgggacagtatgccacgctgctggctctggcccagctcaacggccgccgggcctttatcctgcctgccatgcatgccgccctggccccggtattccgcatcaccctgcccgtgctggccccagaagtggacagccgcacgccgtggcgggagctgcagcttcacgactggatgtcggaggagtacgcggacttgagagatcctttcctgaagctctctggcttcccctgctcttggactttcttccaccatctccgggaacagatccgcagagagttcaccctgcacgaccaccttcgggaagaggcgcagagtgtgctgggtcagctccgcctgggccgcacaggggaccgcccgcgcacctttgtcggcgtccacgtgcgccgtggggactatctgcaggttatgcctcagcgctggaagggtgtggtgggcgacagcgcctacctccggcaggccatggactggttccgggcacggcacgaagcccccgttttcgtggtcaccagcaacggcatggagtggtgtaaagaaaacatcgacacctcccagggcgatgtgacgtttgctggcgatggacaggaggctacaccgtggaaagactttgccctgctcacacagtgcaaccacaccattatgaccattggcaccttcggcttctgggctgcctacctggctggcggagacactgtctacctggccaacttcaccctgccagactctgagttcctgaagatctttaagccggaggcggccttcctgcccgagtgggtgggcattaatgcagacttgtctccactctggacattggctaagccttgagagccagggagactttctgaagtagcctgatctttctagagccagcagtacgtggcttcagaggcctggcatcttctggagaagcttgtggtgttcctgaagcaaatgggtgcccgtatccagagtgattctagttgggagagttggagagaagggggacgtttctggaactgtctgaatattctagaactagcaaaacatcttttcctgatggctggcaggcagttctagaagccacagtgcccacctgctcttcccagcccatatctacagtacttccagatggctgcccccaggaatggggaactctccctctggtctactctagaagaggggttacttctcccctgggtcctccaaagactgaaggagcatatgattgctccagagcaagcattcaccaagtccccttctgtgtttctggagtgattctagagggagacttgttctagagaggaccaggtttgatgcctgtgaagaaccctgcagggcccttatggacaggatggggttctggaaatccagataactaaggtgaagaatctttttagttttttttttttttttttggagacagggtctcgctctgttgcccaggctggagtgcagtggcgtgatcttggctcactgcaacttccgcctcctgtgttcaagcgattctcctgtctcagcctcctgagtagatgggactacaggcacaggccattatgcctggctaatttttgtatttttagtagagacagggtttcaccatgttggccaggatggtctcgatctcctgaccttgtcatccacctgtcttggcctcccaaagtgctgggattactggcatgagccactgtgcccagcccggatattttttttttaattatttatttatttatttatttattgagacggagtcttgctctgtagcccaggccagagtgcagtggcgcgatctcagctcactgcaagctctgcctcccgggttcatgccattctgcctcagcctcctgagtagctgggactacaggcgcccgccaccacgcccggctaattttttttgtatttttagtagagacggggtttcatcgtgttaaccaggatggtctcgatctcctgacctcgtgatctgcccacctcggcctcccacagtgctgggattaccggcgtgagccaccatgcctggcccggataattttttttaatttttgtagagacgaggtcttgtgatattgcccaggctgttcttcaactcctgggctcaagcagtcctcccaccttggcctcccagaatgctgggtttatagatgtgagccagcacaccgggccaagtgaagaatctaatgaatgtgcaacctaattgtagcatctaatgaatgttccaccattgctggaaaaattgagatggaaaacaaaccatctctagttggccagcgtcttgctctgttcacagtctctggaaaagctggggtagttggtgagcagagcgggactctgtccaacaagccccacagcccctcaaagacttttttttgtttgttttgagcagacaggctaaaatgtgaacgtggggtgagggatcactgccaaaatggtacagcttctggagcagaactttccagggatccagggacacttttttttaaagctcataaactgccaagagctccatatattgggtgtgagttcaggttgcctctcacaatgaaggaagttggtctttgtctgcaggtgggctgctgagggtctgggatctgttttctggaagtgtgcaggtataaacacaccctctgtgcttgtgacaaactggcaggtaccgtgctcattgctaaccactgtctgtccctgaactcccagaaccactacatctggctttgggcaggtctgagataaaacgatctaaaggtaggcagaccctggacccagcctcagatccaggcaggagcacgaggtctggccaaggtggacggggttgtcgagatctcaggagccccttgctgttttttggagggtgaaagaagaaaccttaaacatagtcagctctgatcacatcccctgtctactcatccagaccccatgcctgtaggcttatcagggagttacagttacaattgttacagtactgttcccaactcagctgccacgggtgagagagcaggaggtatgaattaaaagtctacagcactaa

SEQ ID NO: 3 - DNA, 58053 bp, Homo sapiens
  misc_feature 1..58053: note = RHD
  source 1..58053: mol_type = genomic DNA
aactccatagagaggccagcacaaccagccttgcagcctgagataaggcctttggcgggtgtctcccctatcgctccctcaagccctcaagtaggtgttggagagaggggtgatgcctggtgctggtggaacccctgcacagagacggacacaggatgagctctaagtacccgcggtctgtccggcgctgcctgcccctctgggccctaacactggaagcagctctcattctcctcttctatttttttacccactatgacgcttccttagaggatcaaaaggggctcgtggcatcctatcaaggtgagagttcattggaaaagtggtcacaggagcaaatagcaggggcaggggcgggggaggcctgtggttctccaggggcacagatgttcctttctacaaaatcccaaggaaaaagattcccccatcttcttccgtagattgcaccgaaattcagccaacaatgtaagctttcctttagaagcagcctgggcatgccctcttctgtgaagcctgccttgatttttcagcacagtgagaggcatcctctttggtgttcctcaaattccctctaccaaatggtcttcataattctctgcttctctgcttccccttctctctcctcagtggcaaggaatttttttatttttatagatttaggggatacaagtgcagctatcttatgcaagcaatttcatgttgttgggtttttggtttttgtttcctttttgtggcctctcgctcatttcttatttctttttgaggcagggtctcactctgttgcccaggctgaagtgcagtggcatgatcatggttcactgcagccttgacctcctagtctcaagcaatcttcccacctcagcctcccaagaagctgggaccacaggagggcaccaccatgcctggctaattttttttttttttttttttggtagagatgtgggtctccctgtgtttcccagactggtctcaaactcctggacacaagcgatcctccagcctcagtctcccaaagtgctggaattacaggcgtgaagcactgtgcccagctctcttgctcatatctatactagttttcttttggaagcttcagcctgttgctaccccccacccccacccccaccgaccccagctttcttctcacttaggggctgggaagtctgcatgctgtctataaatccagaaccagaaggtatggctgaaggggagggtaggatgatggttattttatattcagctaaaaatattcccagactgtgatgagacaactgtaaataagacagatgtccacaatggtgtgactttgcttttttaaaaatattgaaatgagtttcaggcatctcagtgggctgataggttgttgataatagacagggcctccttgaagaatgtccctgagacaaagttgaagcttgagcctggttgagtccttgcttgttcctaggttgatatgaacggctagttaactggaagcaaagagaagtcatcctgggggccatggcagtgacaagtaggacttagggagggaagcccttataccatttaaggtgctggcccagagaggagccttcagtgacagacaaacaagagctggcacaattttaattcacttcaatttactctaattcatttcaatccaatacaattcaatgcattccattcattcaaccatgtatgacatccaatgtgggatccagactcatgatgattagagctgatatttatgagcacttactatgtaccaggcactattctacatgctttacattgaaccctcacaataacccaatgaggtgggtactattatgatcttcgtttttcatatgaggaaactaggcatatggatgttgagtaatttgcccacggtcgctcagctagcaatagcacagcgtatttaaatttagccaccctggatttagtttccttacacttaaccattatgcatcatggccccattttacagtgggcttgagtctttgtcatataacccagtaggttagcagccactattccaaccctgtagattgactctagggtccatgttctttacccctgcaccgtgctactaacgtaggtacaaaatgtcctcagaaactcactttatacggaagctcagaggagggtccacaacccaggcaggggagacgatggtgtcaggggagggaggtgactgcccagccaggtcttgaaggctcagtaggaattacctgtgggacaaaggagggtcatccaagtgagggcacagtgggtgccatggcgtgcacacacaatagagcagactgagcctgggcttaacattgcattgccctggagcctaaaaggggaaacaaagggccgggcgacgtggctcacgcctgtaatcccggcacattgggaggccaaggctggagaatcacctgaggttaggagttcgagaccagcctggccaacatggcaaaaccgcatctctactaaaattataaaaactggctgggtgtggtggcacacgtctataatccgagctacttgggaggccattacactccagcctgggcgccagagtgagacttcatctcaaaaaaccaaacaacaaaaacaacaacaagaacaacaaaaaaacaaagaggagagcagggactgggtgtggtgactcatgcctgtaatcccaaacactttgggagaccaaggcaggcagatcacctgaggtcaggagttcgagaccagcctggccaacatggtaaaaccctgtctctactaaaaatacaaaaattagccggatgtggtggcacgtgcctgtagtcccagctgcttgggaagctgagggaggagaattgcttgaacccaggaggcagaggttgctgagctgagaacatgccactgcactccaccctgggtgacagagtgggactctgtctgaaaaaaataatagtaataaataaaaataaagagggaagcagcgggtggcagactcactgggctgcatacgaagtttggcttcagtctgaggtccgaatagtaaacagcagcgagacaagtttgggtttgggtcatggaggaagccatgccagggctggtgttgggcacagggaaaggggcatggcttgagacaccagaccagcgtggaggctgtagtgtagtattgacctgaggacttcaacattctgatggtgtacacacgattttttgagcatgtaccatggttatatattacactttaagtattactttaagtattactacattaatatattttgtatgttacaataaatacatacaaattaggaaaattgaaagagatcaaaatgaaatatataatattttcaaattactaatcataatggtgtcaatctccaggcagggtccattgctacagttgacgatagtggatgaaaattcactcctcagagtcttcttgataatttgaaattgtcttgattgacttgtcagatctgattagatcaacatgttttaaatctcgaatgtgactgacagcttgtacgaggagaagtttcactctgccttttcccttttgttcacttgactgccattatttctatgcttccaatctgtgtttttctgcacgagttggttaagccattacttcattttgtgaaagtttgttgagttaaacttaggtaacttaatctgtcaatccacttaattgaattcagtcctggtaaactataatagattattcaaacctgccaattctaaaaagacattttgagacaatcaggaaatctgaatatagcatgaatatcttacgatatacaaggattattgttaattttgttaggtatgataaaagcatggtgggttgtttttgtttttgttttttaagtctccatctgttagagaggcacattgaaatggcatgatatctggggtttgcttttatgccagaaaaaagaaaaagtacagaaggattatagaaacaagattggtctcatgtgacaatcatcagagtttggagatgggcacgtagggtcatcgtgctgttctctctgttttcgtatatgctttaaaagttctgtaatagttaattaaaaaaaaaaaaaaacaccctggctgagcatttagggaggccaagtggggaggatcgcttaaaccaaggagttcaagacgagcctaggaaacatagggagacccccccccatctctaaaaaaaaaaaaaaaaaaaaaaactttaaaatttaacccagtgtggtggcacatgcctatagtcccagctactcagtaggctgaggtgagaggcttgcttgagcctgggagcttgaggctgcagtgggacgggattgtaccacttcactccagcatgggcgacagagcaagaccctgtctcaaaaaaaataaaaatatttgaggtgaagcgaggctgtaataacaaatttaaaaatataaataaaacataaaggctgggtgtagtggctcacgcctgtaatcccagcactttgggaggccaaagcaggcagatcacgaggtctggagatggagaccatcctggctaacacgatgaaaccccatctctaccaaaaatacaaaaaaattagccgggtgtggtggcgggtgcctgtagtcccagctacttgggaggctgaggcaggagaatggcgtgaacccaggaggcggagctttcagtgagctgagattacgccactgcactccagcctgggcaacagagcgagactccgtctaaaaaaaaatgaaaataaaaataaatgaaacataaaaccctgccattagttgcaatatgaagaatatagagaaatgcatatcaaatccttctcattggaccaatattcccttagggcaccttccaaagctaggagactcaaggctgtatgacatcctgagcaagtgaggggtggcttctgggtgaatctgaatattaaatatttgcagaattgaaaacttcacaaagtacctttagagatagaatagcctagatccatgtttctcaaagtgtggtccccagacctgctgcctcagcatctcctggaaatttagtagaaatgcagattctcaggccctaggccagacctactgatcagaagctctgggcctggggcccagcagtctgtgttttcacaagccctcttggtgattcttctgtgcatgaaagttcgagaattcctggagctagactgattcaaatcttgcctctgtatcttagagaccttgggcagattagtcaacctctttctgcctctgtttctacttctgtcagaggatgatagtacttgtttcattaagttgttgaaaggataaatgaattgacacacataaagagtattagcttttattatcaaaagctttttttttgagacagagttttgctcttattgcccaggggagtgcagtggtgcgatcttggctcaccgcaacctccacctcccaggttcaagtaattctcctgcctcagcctcccgagtagctgggattacaggcatgcgccaccacgcccggctaattttgtatttttagtagagatggggtttctccatgttggtgaggctggtctcgaactcccaacctcaggtgatgcacccgccttggcctcccaaagtgctgggattacaggcgtgagccaccgcgcctggcccaaaagctttaatttcttaattttttaaataaaataaataaaactagaattgcttgttttcttccagctaccctggtgattgtattgagcattttctggggtgtgtgttctttgctgtaatgactactggtctggatgacctgtgatgagaccagatgggcaggggcagtggaggagattctagagatatttaggagataagtcagctgtacttgatgaaaagagtggggagttaaggctggctgcagatgtatgatttggcatagagaggtgccagttcctgagatgagagacagaaggggagggacaggttgtgaggatgaatgaacaatgatatgttcattctgggcttggagttaaggggcctatgatatgcttaggggaagcagagagtatcaattacctattgctgcataacagccaccccaaacttagtggcttaaaatagtaaccttttaatttactcatgatcatgattctgtggtgcaacaactgggctgggttcagctgggcagttcttctgttagtttcacccagggtcattcatgcatctgcagtttggggtgggatggcctcagatgacctcattcacgtgtttggcagttggtgattcactgggggccattactgtaacaatcgcctaccaggcagagcttccctaaggcttccaaactaggagactatcctgggtcctgtgctgtggataccactcagtcccccatccccaccccatattcctcaaaggcagagagaggggctactagaagacagaggagttttcccagtgacatgtaaacactccaaaccctggcaccttccacactgcagctttggtctgcccctttgggaaatctctgtttttcttcccaggctgctggaggggtgagagtcgccggtagagtagaggctgtgggcgaggaggtggcggcctcctgaggctgcagtggtctttccaggcagcagtgggagcacagggtggaggtcaaccctagagcctgggagagtgaagctgggtgtgacttcagagctgttggtgctgaagtttctgcaggccagaaggaggggcaagagtgggagggggcgcagatccagaatcacggaggcagctgaccggaggaggcagctgcccaaggggatggactcagaaggccaaagtgctgttatccaaacgaactctttgcaagtggtctctttgcaacaggcctgggggagagcagtcttgcctaaagtcacaccgctaatcagcggccggcacggggtaacagttactaacactcactacgtacccaatgctgggcgaagtgacttgcatgagccagcgagctcaatgctcatggcaatcctctgagcagctggcattgtttcatctcaattttacagctcaggaagctgggacacagaggaagagccaggctctgaacactgacaacctgattgagagacccacactgttcatcaccgttacgctatatatgctgtatagaaaggcaggatggcataatggttaaacctaggtaggtagggtttgaatcctcctgctaccatttactagctctgtgacttggactagttatagcacctctctgtgcctccctttccccatctctaaaatggggataataaatcgtacctcctacctgaggctgttgtgggctaagtctgtaaggcacgtagaacagtgcctggaacgtggggtactgtctatctgtgtgcctgctgttacaacaatggtgagtattgccttatctctcgctgctgaactaccaggttagacttctttctgcaagtcatgaggctttcataaacttttcctgaaggctttccgtagaatgtacaattcccctctgggtccaggcatgggcgcccgggtagcacatccacttcttatcacccctgaacaccttagagcccatcagcttatcaaaccagcagctgatgtgagtgcagagcagactgtgagaggtggaggctgataccagtgaggatgctccaagctgggacccagccctgaagcgggagcccagataatggatgggtggaaatgggcctggagcccaggagaagtgggaggatgagggggcagggggaggagaagcctgaaatcaaatgttatttcctgaccagtttggggtgcatgagctctgtcaacagctcatggaaactgctgccctaatttcatcttgttggctgaggcacaattcctctctcagggacagtgtagagccttggggaggaaggccctgagcgcgtatacctggaatcagggaatcgggatcaggggcagcagctgtgcccaataaagcccccacccaggatcctctgacttcctcatctctttttttttttttttgagctgcagtctcactctgtcatccaggctggagtacagtggtgcgatctcggctcactgcaacctcagccttctgggttcaagcgattctcctgcctcagcctcctgagtagctgggattacaggcatgcgccaccatgccaggctaatttttgtatttttagtagagacggggtttcaccatgttggccaggctggtctcaaactcctgacttcaagtgatctgcccacctcagcctcccaaagtgctaggattacagacataagccactgtgcctggccttttttttttttttttttttgtaaacagggtctccctctgtcacccaggctgctggagtgtagtggtgtgaccgcagctcactgcagccttaaccttctaggcacaagccatcctcctacctcaccctcctgagtagctgggactacaggcactcgccaccacgcccaagtaattttgtattttttgtagagacaaggtcttgctatgttgcctaggctggtcttgaactcctcagctcaagcaatcctccctccttggcctcccaaagtgctgggattgtgctgggattacaggtgtgagccaccatacctggtctgacttcctaatctttagggccccaactctgcccttatccaggcaactctcctctccccatcttccactaacttctttggaatattccagagctgtaaaagccttagagagtatcaagtccaactcctatgtgttacagacagggaaactgaggcctaaagagggtaatggacttgcctaagatcacttagtgaggtgagagaagaaagagctagagacagcctagcctgtgcaaggacatagttccaggcattcagagctgggctctgctgccggcatgtttggggcctggtagttagttcactgctgaactaccaggttagattttctttctccaagttgtggagctttcataaacttttcctgaaggtcttccttacaatgtacaattctcctctgggcccggtcatgagcgcccctcacaggctctctctggtccccttctgtaaaatgagaggaaaatggaagaattgctctactcatggaatcttcaataagtctgggccctatgcatatagcattgctacaaaatggcagatgcactttaacaatcgtgtttaataaaaggttggatttgcatatctgaagtggggcatgcagtctccaactgaacacaagcctcactgctcccgcatgtgcactgcaccttcatatacatatttcctgcttggctcctgagggaatttgagtaatcccaagaggaacccctgtagaaaatgtcccctggccacacacccccattcctaaggatgcaagcaggagatagaaacattccctgcacctccctccttgttgtcagaagaagtgcaaagagttgaatccttcctaatgcccacttctcacccacgccccaaatccccaggtcccatggaggtccttgggggcctcctatatcctggtggtgtcaggttgatttggaaatgtcagtgtcctcccttgtcctctctggcagaccctgggtatgtgtatgtttcaatggaagtgaatttaaatgtactttataaatcaaagactttttctgagactttggagagttccagtaatgagagcttctcattgttatcaaggccagggctggagaccagtggcaggtgagttcctattgctgtgattgtcatgatgatgttgatgaacagtcactatttattgagcgttctccatgtgccagtcactgtactaaacattatttcctttggatttcccagaaacctctcaggtgggtctaattacccttattcagctgataaggaaagtaagcaacttacaagaccacagggctatgaagtggaaacacataaattgatatttcattttatttatttatttattttgagacagagtctcactgtgtcgcccaggctggagtgcagtggtgcggtctcagctcactgcaacctctgcctcccgggttcaagcgattctcctgcctgcctcccgagtagctgggattacaggtgcccaccaccacatccagctaatttttttgtaattttagtagagacggggtttcaccatgttggccaggctagtctcgaactgctgacttcatgatctgcccacctcatcctcctaaattggtatctttatatgtccaaaagagtcaactggtggcaatttagtgaggtttaatctaataggaaatgatagagctgggatcgaacagagccatgtgaactcaaaacctatgcttccccttccacctttttgaaaaacattgtctaggctgggcacgatggctcatgcctgtaatcccagcactttgggagacggaggtgggtggattacatgaggtcaggagttcgagaccagcttggccaaaaattagccaggcgtggtggcgcgcgcctgtggttcccactgaagcacaggaggctgaagcacaagaatcacttgaacccgggaggtggaggttgcagcgagccgagatcgcaccactgcactccaacctgggcaacagagagactctgtctcgaaaaaaaaaaattgtctacatgctggttgcagaaaatttaaacactaaaactaaaaaagtaaaacacctcccaaacttagagacaatattaatgacggaaaaaaaattcttcaagatctctctctctccagtcatttattcatgtgcgaaaacagttggtgattattgataaaatagcttttagagtttggagcaattatgtgcattacatataccatttgattctggcaacctaatgaaggagtatgatcatttcccctatttaacagacaagaacaagaagagggagggcagatggtgtggtagtctaaggcacaggctccagcagattatctaggtgtaaatcttggctgtaggccaggccctgtggctcatgtctgtaatcccatcactttgggaaaccgaggtgggcagatcacttgaggtcaggagttcgagaccagcttggccaacatagcgaaaccccttctctattaaaaatacaaaaattagccgggcacggtggcaggcacctgtaatcccagctacttgggaggctgaggcaggagaatcacttgaacccaggaggcagaggttgcagtgagccaagatcttgccactgtactccagcctgggtgacgagtgaaactctatctcgatattaaaaaaaaaaatcttagctctacccaccggggcaagttacgtaacgcctctgtgccttggttttcatatctgtaaaatggtgacagtaacagcacccacgtcaaagtgtggttgtgagaacgaaacaagatagtctatgtaaagtgattaaaacagcgtaggcacatggtaaacgcttaggaaatgtaggctgttataaagctcagagatgttaagtaactagatcaagatcacacagttagagggtgccagagtcctgatttgaacccaagtttgtctcgttctggagctcaagctgctaaccctttttcaaaactggaattaaaccaaagtgctcaccctccgctttgctgggcccctccctgccctcaggtgcgtctcttccactcacctgccacagcagcctctgctcagggtctgagaccgggaaaggtgagggctacccaggtggccctgatgttttctgccagccagctcaccaggtccctcgcagcaggcggcaaagggagggaggtttgctgtgaagattatgtggttcccaacaacaagagcgctgggcctatctctgccctctcttttctgtgtgtcctgggacaagtcacttggcttctgtggcttcattttctcatgtgcccagccagggggttggccctcatatgcaataacagcagcaatgacctttactgagtgtccatgtgcgtcaagcacgtgtgctttacacttgttcttattattaggtttaataatagaataattgccacatttactgagcactcattatgggccaggccctgccctaagtgcttaattagctttagctcctctaatccttatcttatccccacacggcatgttatgttatccccattattcagttgagaacattgaggctcaaagaggcaaagtaacttgaccaaatacttgtaaacgatcttgcatgccccttccagctgccatttagtaagactctaatttcataccaccctaaatctcgtctgcttccccctcgtccttctcgccatctccccaccgagcagttggccaagatctgaccgtgatggcggccattggcttgggcttcctcacctcgagtttccggagacacagctggagcagtgtggccttcaacctcttcatgctggcgcttggtgtgcagtgggcaatcctgctggacggcttcctgagccagttcccttctgggaaggtggtcatcacactgttcaggtattgggatggtggctggatcacttctgggtcatagagggaatggaccccgaaaggacaggttccagaagatctgggatattgccccctctctgtctagcaccagtgctgtgcaatatttaggacatccttatactaaaagattattcattgtttaaaattcaaattaactgggcatcctgtattttactggacagccctactccgtgtatcacaaggaatccaggcctacattcctcctgcatcctttctttcctgttattgtcgattatgattttgtaaagttacataatcaatataagtttatggaaaacgtaagaaggaaacacgttagacagagagaaatagacatgccacacctagagagacattctatttttttttttttttttgagacggagtttcacttttgttgcccaggctggagtgcaatggcgctatctcggcacaccacaacctcagccttctgggttcaagcgattctcctgcctcagccgcctgagtagctgggattacaggcatgtgccaccgcgcctggctgattttgtatttttagtagagatagggtttctccgtgttggtcaggctagtctcaaactcctgacctcaggtgatccgcccgcctcggcctcccaaagtgctgggattacagacatgagccaccgcgtccagcctgagagacattctcttgaaaagaaaggactttcagccccctaatgctgctagacaataaatagccatgcctttattttcattaaattacctgtgctttgtttacatgcatttgtgtgaaatgctaagaaccatcacaactaatgtatggtgccagaagtcagaatagttgttacctgggcaggaggtggatattgattaggaaggaacacaaaataaccgcatggggtgcagaaaatgttctctatgttcacctgggtgatgattacacatcaagctatacacgttttaaaagggcattggcacttaataggaggaagtaggctaaattttttcctgaaacattgttttgttttgttcaaacctctgaatccctgtgctgcccagatgatggtaaacgtcatcctaggcatcttagggacctctcaaggccattccagcctccccttctaagaccctgctaaacctctgggcactgctgttaaacatttctctatgagccaggaactgtgctgagcactccacaaatattattttgtttaactcttccgggtagggatctaacctggtatacaggtaaggaagtggaagctcagagagggcaaggcacttgcctagggccacacagctaagtggtggagatggctccaactttttattataaccttttccacatgctccagagtgctcagaacatgaaacacagtctagccagctcccgattggccctggagggaaaaaactttatatatttttcttttttaaaaggtttagaggctgggcatggtggttcacacctgtaatcccagtacttttgggaaccgaggtgggcagatcacttgagcccagaagtttaagaccagcctgactaacacagtgagatcctgtctctgcagaaaatagaaaaatcagctaggcgtggtggtgtgcacccacagtcccagctacttgggaggctgaggcaggaggatcacctgaacccagtgaggttgaggctgagtgagccatgatcgtgccacttcactccagcctggacaacagagtgagaccctgtctcaaaaaacagttttaggggccgggcgcagtggttcatgcctgtaatcccagcactttgggaggccaaggcggggggatcatgaggtcaggagatcgagaccatcctggctaactcggagaaaccctgtctctactaaaaatacaaaaaattagccgggcgtggtggtgggcgcctgtagtcccagccactcgggaggctgaggcaggagaatggcgtgaacccgggaggcggagtttgcagtgaaccgagatggtgccactgcactccagcctgggtgacagagcgagactccgtctcaaaaaaaaaaaacaaaaacagttttaggccaggcgcggtggttcatgcctgtaatcctagtactttaggaggcctagcaggtggattacctgaggtcaggagtccgagaccaacctgagcaacatggtgaaatcctgtctctactaaaaacacaaaaattagctgggtgtggcggcaggcacctgtaatcccagctacttgggaggctgaggcaggcgaatcacttgaacccgggaggcggaggctatagtgagccgagatcgcaccattgcactgtagcctgggcgacagagtgaggctctgtctcaaaaacaaaacaaaacaaaaacagtctatgagttaattcccaccagaattcaatacacacacgcacacatgcacgcatacacacactgtgtccacctgggaagtgacaaagggcaccctgggggatttcaaatggtggtggccctggtttggtgttgctgccttagcttaaggtcacaccagccttcagcctcctgccccacagtctagggctgctcccctcatctgatgtccacagggacctgtttgttcttgactcaatctagaaagacgagaagggagagaagtcactcgcagcctgagtgaactcccctgccccacccctgactgcttggatccccctaggggtgacccctgctgaaactggctccttcctgaccggttcccgtcagggctgtgctgatgggtggtgcccaggcctgcccctggggacggggtactctcccttggcaacactccagcttgtgccacttgacttgggactgatttggttctgttttgagtcccttcaggggaggggcctatcttattcaacgttgttgtttgttttcctcacatactgataacttagcaaatggctattggagcaaaaatgaaaataaacggaactctgaagtgggatgttttaaaattttatttatttttttagagacagggtcttgctctgttgcccagtctggagtgcagtggtacaatcatagctcattgcagcctgtgcctcctgggctcaagtgatcctcccacctcagcctcctgagttaaatttttttacaggcgcctgctaccatgccctgctaatttttgtatttttagtagacaaggggtttcaccaggtgggtcaggttggtctggaactcccgacctcaagtgatccacctgcctaggcctcccaaagtactgggattacaggcgtgagccactgtgtccagcctaaaactgtttttgagacagggtctcactctgttgtccaggctggagtgaagtggcatgttcatggctcactcagcctcaacctcactgggttcaggtgatcctcctgcctcagcctcccaagtagctgggactgtgggtgcacaccaccacgcctagctgatttttctattttctgcagagacaggacctcactgtgttgctcaggctggtctcaaactcctgggctcaagtgatctgcccacctcggctctgaaaagtactggaattacagcctcctgagtagctgagaccacaggcacacaccaccacacctagctttttttttttttgctttttgtagagatggagtctcactatgttgcccaggctggtctcaaactccaggccttaagcaatcctcccacctcagcctcccaaagtgcgaagattacaggtgtgagccaccattcctggccttaaaagtgtgatatttttaatgtattttgaaatctgcaggactctccctagaagataatagcaataaccaactcctttattgtgcttgacgtatatcaactcactttgcccttaccgtggctccagaggcattgggtccaccttataaatggaggcaccaaggcacagagtgattaaataaattgcccaggatcacacagccagaaagtgtctgagtcaagattccagcccaggcagcctagacctgagagcacgctcctaaccactgcacatcactgtcttagcacctcctcagcacaaactggcccttgaggaatgaaataccgccgccggcacacacgctcctgagttaagcctttgtcaatgaaatgaacacccacttaaaaggaataacctgtccaggcacgatggaacattgagtaaccccttattctaaattcctggtccctgtaagactccttccccatgcccttgcccttttctgaccttcccctaaagtccttgaggcttaagcgggcatagtctgcagcaaacactggggaagctgagtccagacttcagagcacaggctttggatctaggccagctggatttgaacctcacatttgtgatcagctggcatgactgtttccaaaaagtccattttaatcctctacgtgaccctctgtaaaatgggatactgaatggtgagctagcacgattttacagagagtgaattttttttgtgtgtgtgtgaggcagtcttactctgttgcccaggctggagtgcagtggtgcagtctcggcccactgaaacctctgcctcccgggttcaagcgactgccatgcctcagcctcgagagtggctgggattacaagcatgcaccaccatgcccgggtaatttttgtatttttagttgagacagagtttcaccatgttggccaggccactcttgaacccctggcctcaagtgatccacctgccttggcctcccaaagtgctgggagtacaggcatgagccactgcacccagccttatagggttaaaatttaaaagaggtgatgctgttacaagcctgttttacaaaatgctcttataataaatcattatcatcactgttgctgtggttgtagcatcatcatcattaactcccagagggaggagggagtctcagagcaagctgctcaggggagactggatgtccatggattgtccagctcagtaccacttcctccaggaagtcctccctgataagtccagtcagcatcaccctctccttccaatgaaccccactagccttgtgatatcacagatattcttagttgacaggctcatggtgtagcctgtctagatcataagtacattttttttttttttggatcataagtatcttcaagaccaaaataattttctactcctgagcatgctcattggtcaaaggaaggaaggaatcataatagcgttaataaggctagcgtcttttcagaagttggttctttgtgccagtcttggtgctagacacaccgataggaagaatactccttcacatccccaggacaccaacatgggatacgtttgatcatcattcttaatttgcagaaggagaaataggctcagtgagatgaaatagccactccagtggcaaggctgggactggaagccgggcttgtcctgattccaaatccagtttctttccactgccacggagacggagagaagggacagtggccccagatggggatggggtgactggatgtgggcaggcctgcgggggaagagtgccctctgttgagcatccgaatgatggcagcagaaaagaagactgggcagaatcccagttatcagatcccctgagggaacagtcaccccgatcaccctcagtcagatgagtgtgtgtagatcaatgcctcatagatgaaggcactgaggcacagagtggttaagtcatctgccagaccacatggctcagggtgcagaggccaccttaacgggagaagagatggtcactccactctgcagcatcagcgcccaggtgggtagaaatcttgtcttctattcccacagaaagtaggtgcccaacagtgtttgttgaaagaatgaatgaatgaatgaatgaatgaatgaatgagtgagaggcatccttccttctcagtcgtcctggctctccctctctcccccagtattcggctggccaccatgagtgctttgtcggtgctgatctcagtggatgctgtcttggggaaggtcaacttggcgcagttggtggtgatggtgctggtggaggtgacagctttaggcaacctgaggatggtcatcagtaatatcttcaacgtgagtcatggtgctgggaggagggacctgggagaaaagggccaaaagctccatttggtggggtttccagggttttgaaaaataaagacaacctgtaatcccagctacttgggaggttgaggagggaagatcacttgaggccaggagtttgagaccagcctgggcatcatagcaagatcctcatctctaaaaagtaattttttctaaattatccagttgtggtggcatgcacctgtagtctcagttactcaggaggctgaggtgtgagttggaaggattgtttgagcccaggagttagggaccgagctgggcaacatagcaagacctcatctctaaataaataggtaggtggatagacagatagatagatagacagacagacagacagacagacaggctgggtacagtggctcacacctgtaatcccagcactttgggaggccaaggagggcagatcacctgaggtcaggagttcaagaccagcctggtcaacatgggggaacctcatctctactaaaaatacaaaatttagctgggcatggtggcaggcgcctgtaatcccagctactcaggaggctgaggcaagagaatcgcttgaacccgagaggtggaggttgcagtgaaccgagatcgcgccattgcactgcagcctgggggacaagagcaagacttcatctcaaatttaaaataaagaaaaaagaaaagaaaagattgatagatagatagatatccaaatgagtttacaaaaatgtggtctgtgcaaatgtttaaacacaacaaaccaatgcctttaactactacagtataatcctgtaggattgtgctattcatgatataattatggttatataaaagtaattaattctcagagcctcaccagcagtgggtccagcaagtttgtacagccagcatcttctttcagtcagtgcgtgtcagtaactgcatatgtcctctcattgggagagcctgtcgaaagtctaaatttgaaggcagctgtgaaggtaaggccaatccaaatggctctcccagatcctctgctgtaaccctgaccctgagtgaggacatagccaaccttcccatctcataggtgagaaagctgatgcctggagaggggaagggactgcccaagatcacatagcaagatagtggcagaacccaagcgagaacccacagttccagcctggcttagaagaaagtgcactggacttggagtcaaaggctggggtttgcatcccagctctgccataaatccctgtgtgactctgggcaatttaacctcttagagctttagtttcttcatctgtaatatgagggtagcagtactaccacatagggttttgagggagtaattgaattaatcacatgagatgatgcatgtttacaaaaaaaagcatgaagcccctttactgtgcctcagtgtcccaaaggactttggattttactctgagaaatacagggagaactagggagtgttgggcagaggagagccatgatctgacttatgttttaagatactctggcttctgggttcagaaaagactgaaggggcaagagaggaagcaggtggagaccagagcggcagtgattgccatcatccagactcagactaggacaatagctgtgagagtgatgggaagtggttggatcctgactgtattttaatagcagaattgacaggatttgctgatagactgcacgtggggtgggagagggtcaagatgacttcaaggttctcatctggcacaactcagcggctgctggtgccatttactgagatggggaatgttggggtgggatagatctgggagggaaaacccagagttcagtgtcgaatgtggtagcgttagggttaaggttgggggagggggggtagagatgtgtatgaaacatcccagtggagacactgaatggagatgtacaagtctgaagcttagtggaaaggttagggctagggatataaatttgggagttgttacaatacagatggtgtttaaagccatgagacccaaggagatcactcaggagtgaggataaagagagatgggaagaagtctgaggactgagtcctagaacaccctgcattttagaggggggacatgtgtaagagccagcaaaggagacagaattgtgcttggagaggcaggaggaagcccaggagagcgtgaggtcctggaaggcaaggaaagagagggccccaggtgggctgaatgctgctgagaggtcaagtcggatgagggctgggaagtagccattggatttggccaggagaccttggcatgcatggttgtagaggaggatgaaggcaacagcctggcttgactgattcaagagcaggagatgagaaagtggagacagcatgcaggggcagctctgccaaggactttgctataaaggggaacagagaaatggaggagaagcaggagggcaataatccgatagagaggaaaaatctgatgatacagaagagagatgaactgcaagagtcaagcctttgagttggaaagcaggagtgggattttgagcactgatacctttaggccgatgcagggacagttcatcttttttttttttttatacaacattttatttaaaaaaattattttcatagaatacattttcacattagagattcccattgtgcggaaataacaatttattacttatagttttatatttgtggacagattgttttagaacaagtagaatacatttgagaattaaatctcagtttacaatggataatattttgatatgtctctggggaaacttgcccttaaatggaacttctgtatcttcagaagcactccaagcgtttcttcctaggatttagaaatttataatatgagatagcagcatttcctaattttaaaatttccctagtatatgtaaccatcagtaggtggtatctactgactagagagggaagtttttgaaaattaaacactgtctaattttctgcaaagtttttattcatgaattaagagtatttccctttgtccattattcccaaggcaaatatggaaatttgatcatgtactaatcataataaagctggattctctttaagagattgagaaattaaaaggcaaaagctgatatatcatgtttagttatattgtgagtcttataagaagctgggaggcaaccccattaactcaccagaatacagaactcagtctcacaacttagatataattcctctcaaaccttttcctcaaagattaaattctgaaaataatcttgtgattaagagaagaaggctgtccaccaatgggcttatctgttatttcttccttattgtgagcttaatggcatgacaaagcagaggcaaagaggcatacatcaattcttcaaagtaggaagtcaaaaaggtcagagcttccacagcatggcaacagctttgcagatgcccacatcgtgatagttgaaatagcaaagcccagcaaaggttaaagctgaaaatgccaaaagccctgccttggcagctttctgcgaggcatccccatgaacataatcagtaacaacttgtccaaggccccagtgaccatgaagagtgagggctgcagccagggaatagtccgtcgcagagcaaggattcaaataagcagccggaagcagacccgggagcaaaacactgacaaccctctcgctagtccagtggagagatgcagccttggagccagaatggtggctcggtgacaagtgtatgtgctgcactccacaccattctgggataggtcggtcctgaagaaatgctgagatatgagcaggtctgaccactggagttcgcagcaacagagctcggcctccttgggcaccgcaaacggcactcagcctccagggaaccgccatctcgttcctgaggcggagagttcatcttaacgagagaaatggcagggactgtgaataggccggcagatttggtggcgggtgccacaggttcagtctcctgcagggagaggagaaaatgccttactaattccttgtattttctcagagaaacaagaggcaccgtcatcagcctcatgtgagggtgggaaggagggatggggtttgcggagagggaaagtgtggtatggtcatctgtgggagtggaagagagtgagagggctgcaggggtgcagcgggactgcaggctggcaccagggtccctagggcttgtagttggtggaaagtgcatcagtgaccagggctgtgtgcagctgctccaggcaggtgtggaagaagcagagttgaacttgcccagcctggagtgctgcccagagtgagcccaaagcccaggggagaccagagatggggctgtttgcaaaggaggaagtataacagtagcccacaaaatctgagctggttaagaaaggagagagagtgaaaatggggagcccagcctggcagcctgggtacacatctcagctcaacccacactagctgaatccatttgggccccttcgttgacctctctgtgcctcagtttccctatctatagaatggggataagaataaggctacttcctagggctgttgtgaggattgaacaagtgaccgaacacttgttcaattttgaacactgttctaaagcatttaggacagtgcctggcatggggtaagtgttgcggcagtgctgttattttcatcatcaccattgttctcaggctgcgttgattggagctgctgaagggaggcaatttaaggaagtgagccggacagataggaggtggtggtggttatcaggtgcgatgcttgaaactgaggcttcggaggcaacagttactggtaatgacaaggtctaaggcttgacagtgggtggcagaagtgtaacgcagggaaagagacgagcggtcaaggagccgagagggaaggagttgggtggactaagatcatttgtggaagaatgatggagagaaaggctgaagggcaggggctgacatcatcagtgaccaagaggcggccgggaggctgagaccacagcaagaaagggagagtgtgatggcatcttcttcaagggagctggggatgtttggggtggaaaaaagaacaatggtctgggagggaatatgggaaatttttttttttttttttttttttttttttgagatggagtttcgctgttgtcatccaggctggattgcaatgttgcaatcttggctcactgcaacttctgccttccaggttcaagtgattctcctgtctcagcttcccgagtagctgagattacaggcacacaccaccacgcctggcttacttttgtatttttagtagagacggagttttgccatgttggccaggctggtctcaaactcctgacctcaggtgatccacccgccttggcctcccaaagtgctgggattagaggtgtgagccaccgcgcccagcctggaagtttgtatttattaatttttggttgtcttcatctgtgtatgtgactttaacccctaaatacttcagtgtacatttctttttttttttttctttgagacagagtcttgctccatcaatcacccaggctggagtgcggtggtgtgatctcggctcactgcaacctccgcctcctggattcaagcaattcttgtgcctcaccctcccgagtagctgggattaggggcatgccaccatgcccagttaatttttgtatttttagtagagatggagtttcaccatattggccaggctggtcttgagctcctggcctcagttgatccacctgtctcagcctcccaaattgctgagattacaggcgtgggccaccataaccggcctcagtgtatatttctgatgcagttgggttctgtatccccctccaatctcatctcgaattgtaatccccacgtgttgagggcatgacctcgtgggaggtgattggatcacaggggtggtttcccccatgctgttcttgtgacagtgagtgggttttcaggagagctgatggtttgaaagtgtggcacttcctctctctctttctctctctctctcacctgacaccacgtaagatgtgccttgcttccctttcaccttccaccatgattgtaagtttcctgaggcctccccggccatgccaaactgtgagtcaattcagcctcttttgtttataaattacgcagtctcaggaagtatctttatagcagtgtgaaaacagactaacacaatttcctaaaacaaggggacattctcttacataaccttttttcagttaacaaaaatgagaaattgacattgatatattatgattaccttattctcatttcaccaattttctcaataatatcttttctagaaaaaaatatatattttttgtggtcgaggattacatcttgcatttagttctcatgtcttattaaattccatcaatctggagcagtttcttcatctttctttatctttcatgaccttgacatgttttgaagtttcgagccagttcttttgtagaatgtgggtttgtctgctgttcctcatgattagattgtgggtatgcatttttggtaggaattctccaagagccgtgtgtgcccttcttagtatatcatatcagaagacatgctatcaatttgccccattactgggtgtgttaactgtgatcattgggttaagatggtacctgccaggatcttccactgcaaagttactattttcccctttgtaattaataaacatcttgtgaggagataatttcctatagaaatcctgttgatcatccaactttcacccactgattttagtgttcattgattcttccctgaataaattagtactataataattgccaatggtggttttctaattccatctttccttcagtagttggcattcttctgtaaggaaaagctttcgcttctctgttcatccactcatctatgtacttatttatatcaccatgggctcctggattccggtttacacacttccattttctgccttttctctctgcttaatataaggattaatgagaactccctgattcccaggaagaaaatgtcagcagagctttcttaggcggaatgaagagaattcagtgtaagaaccataaaggtgtatctgtgtagtatggacagttttaaaaaacaaacaaacacaaagaacctccaagggcaggaggtgctgccagactcaggagggcactagaactggctatgagaagccactgagatcccaggtagtctgtgctctccatcttttggctcttattctctccgtacatctaacatctctgtacaccagctttctctttagcgaaaaacgtgtcccctccacccacccatccacctccacttgttcctgcatttctatgtcccagatcctgcagaaaacaactcttttctctcagttagtctcaattctgtagtccagggagagagaatctgatcagtcccctgggtcatttttccactctggtccaagcagctacagctggcatgggaaatagttcacacagtaaaaacatggctgtcaagaagaggagtaaatttcagaggcagaacactccctgtgagcccgaacctcttcctgctttgttgcagtcttcataacgattgctttaaaagactgcattgatataacatcatctctcttctctgcatctttgacttgctagcttaactggtctagaggagggcttagcactgattttgagtattcattttcctcaaaacttcaattcagcctgggtttcttcagcaggagggcccgggggaaccagagccagggaccagagtcatttcagtgcaccagctcaagaaatgaatattccaggccaagaatccccaagtgttcttcctgaactccttcctggtggagttcaaagagatgaaaaacacaagcccgcttttcagttcttatcaggaaactgcatagactttcctctttatgtatgactgagggctttttaccatcatttgttcccttcacaaatatttatttggtatttactatataccagggactcttgtggcagtggaaaatacaactctcatggaacgtctgttccagaaggaaagactgccaataaacaataaaataggcaaaagatatagcatgttagagagtggtaagtaccacagataaaaatgaaatggagaaaagaaacacgaaaagttggggagagaggataactgtttgagagggtggccaggggcagcttcatcttatcaagagggtgattttttgagtacagacctgaaggtaacgagtgcacaagccatatgggtacctgagaacagcggcagaacaatggcagggtgctgggagggctgtttaccagccacgctgtttagaattgtcagcacatggtgataaaaaaaaaaaaaaaaaaaaaaaaacaggctgggagcagtggctcatgcctgtaatcccagcgctttgggaggccaaggcggatggatcacttgaggtcaggagttcgagaccaggctggggaacatggtgaaaccccgtctctactaaaaatacaaaaattagccgggcacggtggtgggtgcctgtaatcccagctacttgggaggctgaagcaggagaatcgcttgaacccaacgggtggaggttgcagtgagccaagatggcaccagtgcactctagcctggcgacagagtgagactccgtctcaaaaataaataaataaataaatacaaataaaaagcagacagactttttagttggctttagaattcttagacaccctctacagacaaggcaccccgattgcttgcacccagggtggactactccctccaccctgcccttgttacaccctggctgggggtcagcatttcaggcagctgaatgacccaaagtgggaacacgctagtgggtttgaggatgagcaagtggaggagggcaataggaggtgacgcccgagaggtcaggtgagagtggatcctgcagggtcgtggcaagaacctggaccttgactttgagtgacatgggagccgctggaggcttctgagcagaggagtaacatgatctgacttgcattttattttatttatttatttgacgcagtgtcactctgtcgctgaagctggagtgcagtggcgacatctcagctcactatagcctccgcctcccaggttccagtgaatctcctgcatcagcctcccaggtagataggattacaagcaagcatcaccacgcctggctaatttttgtatttttagtagagacagggttttgccatgttggccaggctggtatcgaactcctgacctcaggtgatccacccacctcagcctcccaaagtgctgggattacaggcaaaattagaatatatctagaatttcctgaagaccttagtttggtattataagaagtctggttgcttcatgttgcaaaatttatatcactcatcactcccgcagagttaaaattccgctgagaagtaggaatcagtgaggtgcgtgtccatgtgggtttttgccacacctaagtgaaccttggtcaaaagcatataagagctactgataggccgggtgtggtggctcatgcctgtaatctcagcactttgggagggaaggatctcttgagcccaggagttcaagaccagcctgagcaacatagcaagattccatctttacacaaaatttaaaaattggccaggcatggttgtacattcctgtaatcccagctactcaggaggctgaggtgggaggattgcttgagcctgggagttggagactacagtgagctgtggccacaccactgcactccagcttgagcaatggagcaagactctgtctcaaaaaaaaaaaaaaaaggccaggcgcagtggctcatgcctgtaatcccagcactttgggaggccgaggcgggtggatcgcctgaggtcaggagtttgagaccagcctggcaaacacggtgaaaccccatctctactaaaaatacaaaattagcccagcgtagtggcgcatgcctgtaatcccagctactagggaagctgaggcaggagaatcgcgtgaacctgggaggcaaatgttccagtgagccgagatcgtgccattgcactccagcctgggcagagcctgctgggttgggctgggtaagctctgaacaccagtctcatggcttcaagtcacacctcctaagtgaagctctgaactttctccaaggactatcagggcttgccccgggcagaggatgccgacactcactgctcttactgggttttattgcagacagactaccacatgaacatgatgcacatctacgtgttcgcagcctattttgggctgtctgtggcctggtgcctgccaaagcctctacccgagggaacggaggataaagatcagacagcaacgatacccagtttgtctgccatgctgggtaaggacaaggtggggtgagtggtctcctacttgggctgagcagaatggctcagaaaaggctctggctgaaaaaatctccctcctttaccaagttcccctgggtgtctgaagcccttccatcatgattcatttctttgagtagtgtttgctaaattcatacctttgaattaagcacttcacagagcaggttcaggaggcctggggtatgcagatttcaaccctcttggcctttgtttccttgtctgtaaaatgtggttagctggtatcagcttgagagctcggaggggagacgtgacttccccatctaactctaagtgacaaggctgagactctccagccctaggattctcatccaaaacccctcgaggctcagacctttggagcaggagtgtgattctggccaaccaccctctctggcccccaggcgccctcttcttgtggatgttctggccaagtttcaactctgctctgctgagaagtccaatcgaaaggaagaatgccgtgttcaacacctactatgctgtagcagtcagcgtggtgacagccatctcagggtcatccttggctcacccccaagggaagatcagcaaggtgagcagggcgctgcccttgggcagcacttgggtctaacaggactagcacacatatttatgcccctccccaccccagggccagcgtgggttgggagagggcatgccgggtggtggagctgtgcctgcctctacagtggagctctaggtagaatgctgggtggtcacagtgggcctgggactcaggagactgtccagtgatcaaaggctttctgggggtagtgattaaatccatccatgctaacatgaaacagacctcagtttgaaccccatttctgctagttgctaaagtcagtcaccatgagcgagagtcagcagcaacagactagactagaattagccagcctctctcttccccccaacaaatttcaagaatggaaccatcagaatcagaagtagagaagtatgtgacactagccatgtggctctggtcaagccacttcaacgttttgagtctcagtggcctcatctgtaaagtgggaattaagagatggtgcatgtaaagtgcttaacggggagtaaatggtaggcaaacattagctgctgctattagtaaagagagacgatggtgtgtgtgagtcttgtgggcagagatgggtgagaggggagacaaaacaagttctcatgatgatgggggaaggggctccagctggtggtgtcggagggaagtctggacagaccagtggtggggctcgggtgggaggcactgggggggctggagtggaaagaatgtggccacagatgacagcttcacagcagaattcagtgctaagaggaagtgagtggccatgagttccatggtgacagaaagtctaagacacccagcaaggcaggagtgggtgtcaactcagggaagcccagaggctaatcctaggtgagagctgagggtgtcagataagagcaaggcaaggctccggttctggagcagtgaaggacatagcagagctatgacccaggaacaaggcccagcttattgaaactgggcccagtcacacagggtggcacaggcaccaagtagccaataataataataaaaacaataacaatgatttgtgtctactgggcatttattcatgttctatgccagacactgggctaagagctttatatgtggaaactcatttaatccttacaataaccttatgaagaaggtacatccaaaaccccattcttctaggccaggtgcagtggctcacacctgtaatcccaatattttgggaggctgaggcaagaggattggttgaggccaggagttcaagaccagcccaggcaacatagcaagaccctgtctctaaaaaataaaacaaaaacccattcttcccgctgcccagggacacaccactaatgagtgtgatgggtgcctaggatgctgagcacctggacttcccagctcattccctaaatgctgcacaatcagggtaactgtgccctgagcctaagaggcagtagtgagctggcccatcatgtccactgatgaaggacacgtagccccaacacaggggagaagtggtttcaggatcagcaaagcagggaggatgttacagggttgccttgttcccagcgtgctggtcacttgcagcaagatggtgttctctctctaccttgcttcctttacccacacgctatttctttgcagacttatgtgcacagtgcggtgttggcaggaggcgtggctgtgggtacctcgtgtcacctgatcccttctccgtggcttgccatggtgctgggtcttgtggctgggctgatctccgtcgggggagccaagtacctgccggtaagaaactagacaactaacctcctctgctttggctgaaggccagcaggacgctgggacctgatgggccactgtgcagtgcacagctgcattaggcaggtgtcggcgcattctcttattggcttcaacgcctagtgagggatccatcctggctcggtggcgcatttgttaagatgctcgggagcaggtggcagaacccatttgagcttgcttgggcattggggagaatttgttatcaggctactggggtgtcacagaactcaaggacagggactggagtgttgtggggagccccgaagcccctgttttacttctttctttgcttttcctgaatatctgctttattcttactctatagacatgcttcctcctctttcaccccacattgtggggtgtagtcttttgcttcaagaaagcagcctggtggatggaatctcttggccccaatcccaaattctctggagaaggggctctttggtttaacttggataatgttgtcttcagctgggggtgggcacatcgtgcatatgtggctgctgccggggaaccacgtggatgatgtgagaggagcagcacccagaagagggagtgctgggctgatggtccaggtcgtgtccacttctgattgtttaattcttcttctaagtggatggatctttctccaatactcagcaaatcctgatcgttccagaatacttcattatagccaattggttataatgtgcttctctaagagaaatatttagggacaacaaatcttcatgggtttgaagacttgatggaggaaaaaggagtagattttcgaaggctggatttggatgaacaggggctattcagggagtgcattccaacctaaaattaggaaaaactggctgggcgcagtggctcacgcgctttgggaggccgaggcgggcagatggcctgaggtcaggagttcaagaccagcctggccaacatggtgaaacccatctctactaaaagtacaaaaattagccaggcatggtggcgggcacctgtcatcttagcgactcaggaggctgagacacgagaatcacttgaacctgggagacagagcttgcagtgagctgaaatcgtgccatggcactccagcctgggcgacagaacaagactctgtcttaaaaaaaaaaaaagtggtttatatacagagtggaatattatttagccataaaaagaatgaaatcctgtcatttgcagcaacatggatggaactggaggtaattaaaaaataaaattaaataaggaaaaacgtatcaatacttcgattaaccaaaaccagggcaaatctgattttcatctttgcaaggggaacaaatttcttttatctcctctggctttgaaaccctgaaatgaaaggaggaagggcagaaaaaagaacacatagcaagttatcatcagtctcagcgcccatcgcattccctgagcttgtttccttgacttcatcactggcaggactattcaaaaatgattcgctcattcattcatatattcattcattcatcattccttcattcaacacatacgttttaacactcatcttgcttttcaagctatagtttagtgagcgaaatggatacacacaatacagtgtgagaacagcaagagggcacatctgagctagcctgggatgggtctggaaatgcttcctggagcagaggaaacggttgacagccaagtgttgacagagaagtagtattagccaggcagagacatggggaatgtattccaggcagaaggcacagtgtgtatgaaagcttattgttaagaagagtgtgtggcccaaccaggaaacagacattctaaaggcatagggtccacccaggagcatggtggacccagatccctgaaagatgggaggtgctcaggcacacttcctgggctagttgaggagtctggatatttatttatttatttatttatttatttatttatttattgagacagagtctcattctgtcacccaggctggagtgcagtggtgcaatctcagctcactgcaacctccacctcctgggttcaagtgattctcctacctcagcctcctgagtagctgggattacaggtgcccaccaccatgcctggctaattttcgtgtgtgtatgtattttgttgttgttgttgttgttgttgttgttgttgttgagacggtgtctcgctcttttgcccaggctggagtgcagtggcgccatctcagcttactgcaagctccgcctcccgggttcacaccattctcctgcctcagcctcctgagtagctgggtctacaggcgcccaccaccacgcccagctaattttttgtgtttttagtagagacggggtttcaccatgttggccctgctggtcttgaactcccgacttcaggtgatccacccatgtcggcctcccaaagtgctgggattacaggcatgagccaccgtgcccaacctggatttttattctgaagactaatagggattctaaggaaggaaccagcctgattgaatttgcatatgtgtccacatctgctggctcacggctgtgtgggaggctgagtgatggggaggaaggattactgagtagggatctgaaggtgtggcctcatgctttctttctaaccagctgtgttgtctttgggatggtgcttaaatttgggctagaccagtgggtcttggtcaccccccaggggacatcttacaatgtctggaggcgttcttggttgacacagtggggtgagggctgctactggcagctcgtggggagagaccagggatgctgcttaacatcctacagtacacagggcagcccccaccacaaggaattatcagctgaaattgtgaacagtgtctacactagacccttgctactcatagtgtggtccgtagaccagcagcattggcatcacctgggaccttgttagaaatgctgttagaccccaccccacatccactaaagccagctcttcatttcaacaaactccccgatgatgtgagtgcacattcaagtctgagaagggcttctttgaggtgagccttagtgcccatccccctttggtggccccggataccaagggtgtgtgaaaggggtgggtagggaatatgggtctcacctgccaatctgcttataataacacttgtccacaggggtgttgtaaccgagtgctggggattccccacagctccatcatgggctacaacttcagcttgctgggtctgcttggagagatcatctacattgtgctgctggtgcttgataccgtcggagccggcaatggcatgtgggtcactgggcttaccccccatccccttaacactcccctccaactcaggaagaaatgtgtgcagagtccttagctggggcgtgtgcactcggggccaggtgctcagtaggcttcggtgaatatttgttggctgatttattcagaaattctgtccagcccctaccttggatggatttatcacctctccaggccacctcttctttccaaatagggccacctaggtatagaccaaagacacgaaatcttttgtgatcccacaaacacagagcaggtcaaataggcccaagccaattgagactgtggttcaggtcgtgatgcagagctttgctgtggacgtgctcccactgcgtactagctgggcatgtggcttaacctttctcagcctcagtcgccccattgtaaatggagataatgatactatctcccctcacaggactgttgggatgctactggatttaataagctaatgcagggacatgctaagcacaacccatccctgaggcccagagaggggtgggccttggctgaggtctcactgcgaggtgggaatgtgggcctccagaccagaggtaggtcctgtggcccctagacagtggacagcaatggtcagtttgacacaccagagccctagccattacttcctggatgttgtgtgaatattttctggacatggcttatataaaatgaaaaagtgaattgggcacgatacagggatagatttttagagatgaactggtagcatgatgataatcatattcactgataacatttactactgttattgactgctttaaaagtgttgggcattgtgctagaaaccattatatgcattatctccttgaattctcacaaccgcctactgaggtattctcagactctaagaaatgagatttaagagaagttatctgcccaaggtcactcggctggaacctggctgtaaaaatggctgaagcaggtgatgaggagctgatgcgtttggacgtgtctcagagaaatcatggaggcgctgcggttcctaccggttcttggatgccttctacagagacaaccatagccccaaattatagggatcacatatcagtgggtgagacatccttgcttgggatgaggaggggatgagctgtgtgaagcaaggcgcctctgtgatgggttccagtgatgtgtctgccactgtcttaataactgtgcaattctaagcagaacctttcctgtctctgggcctgagagttcccctctgaaagatgaggacttgacctagcaaggtcctactcacatgcctgtagagaacaggcaggggaagttagaaaaaaaaaaaagccagtgaaggaagggagctcttcagcttgcacccatcatcacagtgcagggacccaggctcagtgttgccagatccaatgacttctcaagagctcaaaatctagagttttgcatgtgctctcccaagtactggcagaaaattcaagattgttagtaacactgtgtggctaaattctgcttgtgggctgcctagattcccaattctgtgattctgtggttctctggaagcattggttctccacagcacctgcatcacttggaaacttgttagaaatgcaagccctacctacggccccaccccagacctacccagttagaaatctgggggtgggacctatcagtccatgtttgaacaagccccacaagtgttctcttgcaagctcaagttttagaaccactgacctatagccaaaaaagaaaaagccaatcagtggttttctggtaaaggattaacttaacaaactggctttccaagaaaataaagccttgattggtagcacttgcaatttctatggtacaaacgcttcccgcatgactgagttcaagctgtcaaggagacatcactatacatggacttgggaagagatgagaacaatcagcccactgagcctatgggaactggctccagcacatccctgcaagtcaactctcatcagggtgagtgagttgaggaccaagaagcagttatcctcttgcctttgcaggacccaggcaaagggaagggcatagtgacagtgatgatctctcttccggaagtctttggtttgctgagagtaaaaggcgtgggcttcaccagtggtgaagccagtcatgcagccttagtcctggtactgaaactctctaaatctcagttttctatctgtaaaatgggaaaataagacctatgtcacagggttgctgtgcagatttagcaacagaacatagccccgttctttatgatgactgatgctgcatccgtatgaggacatctctatgtaatggaaagatggagagaggattaagcgcaaagtcacaacacttaatgggaactgtggattagctacttggtggcattgggcaagtcagttgactttgcattaattccacaaacaatatttcccaatttcctattcagatgagcatatgtgattgagtcagatgctgtgatcagaaccaggatggagcatttcccacaaactgtgggatttttaagtaatgggaaggcacactgaaatggcactgaatcatgcagttgcagatactctttttcaattctcagtcctttgattacgtcagggagaaaagaaagtccccacttggcctgagaatctctgcacccttctagctcttgttaaccactcttttgaatagcagagaaaacctcagactgccatatctgggagagattttagcaacattttgttttcattgtatctctttttacagctacctcccatttcccttctatttcaagctagtaactcagttttcttttaaattcaattatttaaatgtaaaaataagtctatttggagaaaaaaaattttaatagcatctctggaatgccagtatggctaaattcatgaatgttgtcctcaaatgctgaaatctgggaagcatctggccaagctttgtggacaggcctgcctagtttgaatcccaagagccacccagtccaagccacaaaacattggaattcttggttcacttccctaacctgaacttgccctctgtgaaatagggacactaatagctcactcacagggctgctgtgaggacatgtgttgagctgagggtctcgccaggggagaccctgtgcagggagactgttatcatggtgatggatttctgcttcattcatttctttttccagacagcatcatatagaatgagttgtggggtggcagtcagcaggtttgggtttatcctctattctgccacttattacttaaaaaaaccccaaaaaacccaacttatatagtataagctatatccagaaaagtgcaaatatcatacaagtaccatttgatgaatcttctgatatccccacataaccaacacccagaacctcttcttgtctcattccaggataaccactaacctgacttctaacagcatcagtcagttttgtctgtttttgtacattatatatgtgatggtttgaatgtgtcccccaaatttcatgtgctggaaacttaatccttcaattcatatgttgatggtttttggaggaagggcctttgggaagtaattaggattagataaggtcatggggtgaggtatgatggcactggtgacttataagaagagaaagagaaatctgagctggcatgctcttgccctctcactgtgtgatgacttctccatgtcatgatgcagcaagaaggccctcaccagatggtggcaccatgcttttggacttcccagcctctagaactgtgagctaaatcaatttattttctttataatcacccagtttgatattttgtcatagcaacagaatatggacaaagaaagaaaattaatgcaagaagtagagtttttactgtaacagattcctgaaaatgtggaagtggctttggaactgggtgatgggaataggttggaagagttttgaggagcaggctagaaaaagcctgtattgtcaagaatggagcattatgccaggcacggtgtctcaggcttataatcccagcactttgggaggccaaagcaggtggatcacctgaggtcaggagttcgagaccagcctagctaacatggtgaaacgctgtttctaccaaaaatacaaaaaattagctgggcgtggtggcgcacacctgtaatctcagctactcaggaggctgaagcaggagaatcacttgaacccaggaggcagaggttgcagtgagctgagatcgtgctattgcactccagcttgggcaacaagagcaaaactccatctcaaaaaaaaaaaaaaagaaagaaaaagaatggagcattaaagacagttctgcagttctggtgagggcttaaaggaagaccccagaactagggaaagtctggaacttcttaatggttactgaagtcgttgagatcagagtgctgatagaaatatggctggtaaaggccattctgatgaggtctcagatagaactgaagaaccacgtgttggaaactggagcaaaggtcatcctttttataaagaagcaaagatcttagctgaactttttctgtgccagagtcatttatggaaggcagaaaatctgtaggtcagccatgttgtagggaatgaaagaacattttcagctgagaacactgagagtgtgacacaactaccgactgataagaaaactagtacacataaattagccaggcgtggtggtgggcgcctgtattcccagctacctgggaggctgaggcaggagaatggcatgaacccgggaggcagagcttgcagtgagccaagatcgcgccactgcactccagcctgggcgacagagcaaaactccgtctcaaaaagaaaaaaaaaaggaagaaagaaaattagtacacatagaacaaagccagaggctgttcatcaggacaagggagaaaaactccaaagccatttcagagatcttcaagactgcccctcccattactggcccagagctctaagagggcagaatggtttggaatgaccagctgctgcccagggctgccttgggtctctgctccccacatttctggtgcagcattcctcagccatcccagctgtggttcaggtggccacaggtgtgatgtggaaggtaaaagtcataaaccttggcagcatacacatggcactaattttgcaggtgtgcagaatgcaaaagctgagggggcatgccttcttccacctacatttcaaagggtgctgtgaacagccaccccagagagcccctagtagagcagggtctagtggagctacaagggtggggccaccgccaagaccccagaatggtagagctatcatagtgcaatgccagcttgggagaactgcaggcatgagactccaacctgtgcgaagtgcaacatgggcagaacccagcaaaaccacaggggcagagctccccgaagcttcgggggtccaaattccatagtgtgtccaggaggtggcacacagagtaaaagatcattctgaaggtttaaggtttaatgttgttttctatgttgggttttgtactttcctggaaccagttaccctttttcccttgcctctttttccttttagaatgggaatgtctgtcctatgcctgttccactgttgtattttggaagtcaataacttgttttgactttacaggcttacagccagagggaatctcccatagaatgaattgtaccttaagtctcacccacatctgatttagatgagaccatggactttggaattttgagttggtgctggaacaagttaagactttgggggttgtctaagtgtggtgtttcatgcctgtaatcccagtgatttgggaggttgaggtgggaggattgcttgagcccaggagctcaagaccagcctgggcaacatagtgagacctgtctctacaaaaaataaaaataaaaaaattagccaggtattgtggcatatacctgtaattctagctactcaggaggctgaggtgagaggatcacttgagcccaggagtttgaggctgcagtgagctatggtcgtgccactgcattccagccagggcaacagagtgagactctgtctctacaaataaaattaaataaacttagctggatatggtggcacacatctgtagtcctagctactcaggaggctgagacaggaggattacttgagccaaggagtttgaggctgcagtgagctatgatcatgccactgcattccagcctggatgatagagcaaaatcccatctctaaaaaaaaaaaaaaaaaaaaaaaaaaaaaaaaaaactttagtgctattggaatgaattttgcatgtaagaaggacatgcattttgggggctggggcaggatgctgtggtttgaatgcatccctcaaatttcatgtgttggaaacttaatctccaaattcatatgttgatgaaattggaggtgaagcctttgggaggtaactaggattagataaagtcatcagggtggggcccctatgatgagactggtggcttacaagaggaagagagaactgagctgacatgctcttgccctcttgccatgtgataccctctgccatgtaatggcaggcacagcaagaaggtcctcaacagatgccagcagcatgttcttggacttcccagcctccagaaccatgagctatatatacttattttacaaattacccattctgtggtattctgttatagcaatagaaaatgaactgagataatatacatggaatcatacagtaagtctgtgcttttgtatgcttcttttactcaacattgtagttgtgagattcatccaggttgttaagcattgctgtaccctttttccactgggatatagtgttctgtcatgcttgggtcttaatttataaaggtgactgagtggcattttcttccagtattattggaaggaaagttttgttgttcacagttcccctgtaaacaagaggcagaacacgtcatgcagggccacacaaaactgtatcatccagggaccaggcagcagaaagagagggggaactgggactatgcctttatgaaaaagagtggtgggagagtaactgggtgagggcatccactaatgggcaggaagtgaaaacacatatgttagaatttgtagctgaggggtttataatatgagtttcctatgcctgagaaagctgacttgcaagaaaatgagataaacaactttggccattagtgtggccctgtcataaatgaatgccagataggcaaatagagaatctaagaaaagatagttggaacaagtgttccattgtgtgaatgcagcagaatttatttatccattattgaggaggatttgggtagtttccagtttggagctattatgaatattctagtattgctcctatgaacattctagcacttttatttttggagcacacgaatgcacttctgttgattatatgcctagaagtgaaattgttgaattatacagtattcacacagtcagctttagtggctactgctaaacaattttctctagtagtttgcgccaatctaatcaccagtagtgtatagaagctccttttactccacattttgccaacacttggtgttttccttctttttgattagtcatttagcaatcaaacctattgtttacattttgatatctccaataactaactaaatggagcacttttaatatgctttttggacagttgaatatcttttcttgtgaaatgtctattcaagttagtttgcccattttctattgtggtgttctgtctttttcttattgatttgtaggaattccttacgtatcctggatatgaatcccactttgtgcgttacctttttccttctttctttctttttgaaacagagtctccttctgtcacccaggctggaatgcagtggcgctatctcagcccactacaacctctgcctcccagcttcaagcaattctcatacttcatcctcctgagtagcttagattacaggcgcatgccaccatgcccagctaacttctgtatagacaaaataatttttggtagagacagggttttgccatgttggacaggctgatcttggactcctggcctcaactttggcccaccttggcctcccaaagtgccaggattacaggtgtgagccaccatgcccagcccaccttttactttcttaatggtgtcttttgaacaagagaggttcttaattttaatatagcccaatttatcattgttccctttatgtttagttcttttatgtcctttttaagaatttttgcagccagcgcggtggctcacacctgtaatcccagcactttgggaggctgaggctggcggatcacaaggtcaagagatcgagatcatcctggccaacatggtgaagccctgtgcctactaaaaatacaaaaaattagctgggcgttgtggctcttgcctgtagtctcagctactcgggaggctgagatcacgccactgcactccagcctggtgacacagcaagactccatctcaaaaaaaaattttttttgcaaggtcatgcatatgtccccctgatttttttcctaaaaatcacttattattagatcaatgaattgagtaattgactacatttttcagtcattcaacaaatatttccctgaggttttgataacctgaactgtgtttggagctggggaggaagcaaactattgaagatatacaaagatggcaaagatgagggcctggagcttgccacacggaaggggggatggctgcctgaatggttgggcaggtagttgttgacatctgcactccctacatgagcagcagggtggcaactctttttatctttttaatttatttttcttttctttctttctttttttttttttgagatggagtctcgctgtgttgcccaggctggagtgcagtggcgtgatctcagctcactgcaaactccacctcccaggttcacgccgttctcctgcctcagcctcctgagtagctgggactacaggcgcctgccaccactcccggctaatgttttgtatttttagtagagaaggggtttcactgtgttagccaggatggtctccatctcctgacctcatgatctgcccgcctcggcctcccaaagtgtggggattacaggtgtgagccaccacacccggccttaatttatttttctagtctgcaggtaattctttttaattctctccactctcctatgatcttatgaggtagggactgtcattatttctcccactttataatgaacaatcagtaaagacagggaagataaccaaatgacatacaaggtggggtccaccccatgaggctgcaggcttggagctttgctttgtcttaaaaatgagaacatgagctgcccacctgttgagacaagaaacaggaaaggcttaaaaaactggcttgttatgtacaactatccgtggggctgcagtgaacgggctggcagtgcccaggtgcaggctgaaccctgggacaatcacattcagcatccaagggcccccgtaatagcttaatgtttgaattgaacccctggggttgccttgaaggagagaggtcgtggaagtatgttcaaggggtagggatgggcaggggagatgggtctgaaagccaagctctaccccacccaccttgccccaagagaaatagaaccttcatctttaattgcctaacgagaaaactggggctggccagatgtggtggctcatgtctgtaatcccagcactttgggaggccgaggcgggcagatcacttgaggtcaggagttcgagatcaccctggtcaacatggtgaaaccccgtctctattaataatacaaaaattatccaggtatggtggcgcatgcctgtagtcccagctacttgaggcacaagaatcgcttgaacctgggggacagaggttgcagtgagccgaccactgcactccagtctggacgacagagtgagactccatctcacaaacaaaaacagaaaaaaaaaaaaaaaaaagagagagagagaaaactggaggctctgagaggttgagggacttgcccagggtcttgcagctagtaagtgacagagctgggacttgagcttgggttttctgactcctggtctggttcattatccatgaggtgctgggaactaaaataagccacaatcttggaatctccgtcgcctccctccctcccacatgtctgcgtggctttttgggaaaatgccaggggaatgtaccagccagggagaggacccttgttttcctcatggcccttcctggcaatggcactactgacaccgacagtcctttttgtccctgatgacctctgctgcctgatgcccaagtgaccacctctgctttgtcatttctaggattggcttccaggtcctcctcagcattggggaactcagcttggccatcgtgatagctctcatgtctggtctcctgacaggtcagtgtgaggccacctttcttccaccattgccaggacacagcacccacgtccagagcgcaccctgccgtgtggctggatgtctatgtgccccatctccttccctgaggatcacataatttcagaattggaaaggttcttagaggtcacctgctgctaatgtggactgtgaggccagggcagggaagggacatccctgaggttataagtagggtgagtggcaacgttgcagacttttgaacccagggctggtgatcacactcagttttgcacagaagcccgagaaaatccttacacccaaaagcctaccttttatttctgaggacacccataatactattttattcaacagatatttattcaatatccactatgagccaggcactggggacacagcagtgagcaaaacaaattccctgaccccatggaattgaccttctagtgggggaaggtattagcaataaatagacaaataagtgtctactacgccagatgggaagaagtggctgtgaagacagagcaaactagagaaacatagagtcaatgtgggatggggtgttcttttaggggggtggtcagggaaagcttatctgagtagttagcttttaagcagagaccccaatgaagaggagggagatatgcgatgcatttagttaggggaagaacattccatgaaaataggatagcaagtgcaaaggccctgagacagcagcatgctttgtgtgttgagggaacagtaaggagaccagtgtggttggtgtgaatggagtgagaaggagcagcaggggttgagggcagaatggtagtgaggagcaggcccttataaaagatgggaagccactggagatctttcaacaaaggggaaaagtatgtttctgttcttgcaataaaatagaacagcaaaaaatctaggggagttgctaattagccagttttacttatatgccaggtgaaaatatgtggctaggtgcagtggctcatacctgtaattgcagcagtttgggagaccgaagtgggcagatcatctgagatcaggattcaagaccagcatggccaacatggtgaaaccccatctctactaaaaattaaaaaataagccaggcgtggtgttggatcccagctacttgggaggctgaggcagtagaattgcttgaacccgggaggcagaggttgcagtgagccgagactctgtctaaaaaaaaagaaaaaaagaaaatacacattcaggccaggtgcagtggctcacgcctgtaatcccagcactttgggaggctgagacaggtagatcacttgaggtcaggagttcgagaccagcctgaccaacatggcaaaaccctgtctctaccagaaatacaaaaattagccaggcgtggtggcgtgtgcctgtagtcccagctactggggaggctgaagtaggggaatggcttgaccccaggaggtggaggttatagtgagtcgaggttgcaccactgccctccagcctaggtgacagagtgagactgtctcaaaaaaaaaagaaagaaaatatacattccatccagaactgttcacctttattctacaagcaaacatcttttattggttagacacccatatatgtgtccctaagcaggaggtgaatgccaaataagagacaaatggcgtaagacactatgagttgtgtgacgttgggcatgtcactttactccctctgagccttggttagcttctctgtaaaatgaaaggattatggtaactaagctggcttccttccagctttaacaaactgtatggaggtactttttggagttacctgggtaatttttgagtgtgagattggctagaattgctttaatataccatgtctggccttagctttttgcagagtctttgtgaagaagcagaggcggagtagcgttaattccgtaagttaacgttcagttcgtggcagctggcaatccaaccctgggaaaggctgccggatttagcaaaaatgcaaggtgtctgtttttaaatttgaaatgaattgggtatcctgcattttatttggcaaccctgtcctgggactcacactattcactgttatcactggtatgttcaaagtggtgctgacttgccctctgtcttgcaaagtaccaggaggtcttttcttattcttcactggagtcaaaaaagagaatagaggaaaagacaatcatattgttcctttaagagttaagaccaacaagttttcttctttacatgttgtttttgacatgagcaaactggtgattaaaaacaacttgggtggctcatacttgtaatcccagcaccttgggaagctgaggtgggagaatagcttgaggccaggagttcaagccagggcaacatagtgagaccccatctctacaaaagatacaaaaattagccaggcgtggtggtacacctgtagtcccagctgctctggaggctgagatgggaggatcagttgagcttgggaggcagaagttgcagtgagctgagatcatgccactgcactccagcctggacaacagagcaagaccctgtctcaaaaaaggaaacaaaacaacttggacaatggaagggggaaaaagttcctcaagcagccaaaattgcaccaaatggactcccagaagacaagcatttaatttgttaattgagccctctatgggcctgtctgtatttatttaagaaacaatcctatcaagcatagttattgggtttctcagcccaggtagattagaaatagcagattagaggtgggctaggtttctagaggtaaagtacaccagcagaagttagaagtgaaagcaaagagcctaacagaggaagagaaattcttttttttttctttttttagacgcagttttgctcttgttgcccaggctggagtgcaatggcgctatctcggctcactacaacctcagcctcctgggttcaagtgattctcctgcctcagcctcccgagtagctgggattacaggcatgcaccaccacacccggctaattttgtatttttagtagagacagggtttctccatgttggtcatgctggtctcgaactcctgacctcaggtgatccgcccaccttggcctcccaaagtgctgggattacagggataagccactgcgaccggccgacaaattcttaaaactggacacaagaacacaaaacgcttgggctgctgagagattagaacaacaaccctccacagctacacaccttttccacgttatatggcacgttataagtgggtgttcctagtgatggttctgattttttttaaaaaaagtctaaatatgtttaatgttgtctcagaagacaaaatatattttagacagatattcctcagtgatgagtaagcctcagctatctggaaaattcatgcaggcgccagagatcgttactgagtaattcaagctaactgcgtcatgctggttgtaccctgcatgccaatatcagctaaaagcagcaccacgaaagggaaatacgaatctcactaagcactcgcccattcttgttaacgacactggaactgatcatccttaataatacacagataaatctatcaggagcatttccttgcttcctgtgaaaggaagcactcattccatgtgtcctgtgaaattcatccaacttcaggaagctggaggaatacatatggccaagctatctgggcagagagtagacagggaatggaggttgggcacagtggctcacacctgtaatcgcagccatttagaaggcaaaggcgggcagatcacttgagctcaggtgttcaagaccagcctgggcaacatggctaagtcctgtctctgcaaaaaataccaaaaactgagctggatatggtagcacacacctgtggtcccagctacttgggaggctgaggtgggagggttgcttgaccccgggagtttgaggctgcaatgagctgtgattgtgccactgcactccagcctggataacagaatgagactctgtcccaaaaataaaaaataaaatcaaagacacttaaaaagatggggaaaaggaaggacaggcacttaagcaagttataagctactttcctaactacacaagtggaatcttaagctgaggttcccaggagttgactggagccagagaagacagacctataggagcacccaattggagtcaccctccatagtagcccatatgtcttacatggatcagctttcgtggggcccttttactccatctggggaagggcgtcagatctgtggctctcatgtactgctcagtacactgccattcccagttctttttttcaaaaaaaaaaaaaaaatgtctacagaatcggccaggtgtggtggctcatgcctgtaatactagcactttggaaggctgaggtgggtggatcacctgaggtcgggagttcgagaccagcctggccaacatggtgaaactccatctctactaaaaaaaaaaaaaaaaaaaaaattagctggatgtggtggcaggcgcctataatctcagctacttgggaggctgaggcaggataatcgcttgaacctgggaggcagaggctgcagtgagccgagatcacgccattgtactccagcctgggcgatagagtgagactctgtctcaaaataaataaaataaaataaaataaaataaaataaaataggctacagaattaagctggtccaggaatgacagggcttccatttatttgtctttcaattgtgggagaaaaaggatttctgttgagatactgtcgttttgacacacaatatttcgattaatcttgagattaaaaatcctgtgctccaaatcttttaacattaaattatgcatttaaacaggtttgctcctaaatcttaaaatatggaaagcacctcatgaggctaaatattttgatgaccaagttttctggaaggtaagatttttcacctattaacgtgatagattttgagtgcatgaacttaaaaacatacctgagtatatatgttgacttgctgtttatgagtaaaacaaaaacaaaaatggagtaaggagcattgcaggaggaactagaggagaaacaaatccatgatatgcatgtgtgtgggggagggtggcggggaggtggtaaaggtcaccatttccctgatacctcaaattcattcagagtcagggatgagacagctttcactggccacacttcccctccccctatctgcagtcctcagcgtagccaaatagtctgacatgcgggtgacagaaccccacaatgcaaaagctggaagaaacctcaagccttggagtccaaccccttttttgacagatgctaagagtggagacatgacttatcaagatcttacaactggctgggcacggtggctcacgcctgtgatcccagcactttgggaggctgaggtggggcgatcacctgaggccaggagttcgagaccagcctggccaacgtgtcgaaaccccatctctactaaaaatacaaaagttagctgggtgtggtggcacatgcctgtaatcccagttactcaggaggctgaggcaggagaatcacttgaacctgggaagcgaagtttgcagtgatctgagatcatgccactgcactccagcctgggtgacagagcgagactttgcctcaaaaacaaaacaaaacaattgtacatatttaaagtgttgtaaccaagtgagttacagagaaacaccacactttgagcctaattcaggagtcctttattagccggcgacctagagacgactagtgctcaaaattctctcggccccaaagaaggggctagattttcttttataccttggtttagaaaggggagcgggaattgagctgaagcaatcttacagaagtaaaacaggcaaaaaagttaaaaagacaaatggttacaggaaaacaaacagttccaggtgcaggagctttaaagccatcacaaggtgacaggtgcgggggctctgggtgctatctgccggacacaaacgcaggggcactagagtactatcacccgggcaaattcctgggaactgcggacacagcttgccacagtaccttatcagctaattgcactctttgatgtgctgggagtcagcttgcacaagttaagtccttgaggaagggggtgggtaaggagcccttaacgtcttgcaaatgaaggagccgaatggaatccctccggctttcttagctaagagagagtcaatcaagttaatacaagttagggtatcacaaaagtatataatttgatacattttaacgtatttatacactgaagagaccatcaccaccatcaagacaaggagcacacccatcacttccacacacttcctcctgctcctttgaaattcctccctccctacccacctggtcccacccaaaggcaaccactgaactactttctgtcactaaggtttgcattttctgtaatttttttgtttgagacagggtctcactccgccacccacaccgtaatgcagtggcaccatcatggctcactgtagcctcaacctccccaggctcaggagatcctcccccctcagcctcctgagtagctaggaccacaggtgtaggccaccatggcaggctaatttttgtatttttttgtagagatggggtttcaccgtattacctaggctggtctcgaactcatgggttcaagcaatcctcctgccttggcctctcaaagtgctgggattataggcatgagccactgtgcccagccctctgtaatgttacacaaagggaatcatgcagcacgtactgcccttggtctggcttcttttgctcagcatgattattctgagaatcatccgtgttgttgcgtgtaactgacttcatcagcttctctctgcagctgtcagctcttggcttctcccaacagccaatctctctttatcccctgcaagtgttcttgcctatttagcagaatcaaggtactctatcgaaaagactcggaaaattggtttaatctattcattcattcctcaggtatttatcgaataactattctataccaagtactatgctaatcaaccaaggacagcacaaacaggagaaatctccagctcagtcacttgagttgcaataaatatttgctggataggtcaggtgcagtggctcacacttgtaatcccagcactttggggattactgagacgggaggatctcttgagcccaggaggccaaggctgcagagaaccatgatcatgccactgcactccagcctgggtgacagagtgagatcctgtctctgaaaaaaaatatttgctggataaattaaggaaatctgacgaaccccatcagtagccattgcagcaacaggtaaactagaacgagtgtgaatttggaatgaggaaacccgatgttggccatcattctgtaatgtcatgtattatgtaatgtattatatattaatgtatgtattatgtaggcaagttccttgacctctctcactggtaacataagagtagtaatctttgtgctacttcactgggttattttaaagatcaagtgaggtaataatgtctgtaacaacattctgtaaaatgcaaaccgccacatgaatgtgaaagtttattactagggatttagccaaccacaagggaatgtgtgagcataagagctatcatattgcaagcctacagtttctgattttgtgctaggtgcttttccacattacctgattttatcctcacaacagccctgcataaaagtaagtatgtcgcccaggtgcggtggctcatgcctataatcccagcactttgggagcccgaggtgggcaaatcacttgagatcaggagtttgaaaccagcctggtcaacgtggtgcaaccctgtctctactaaaaatacaaaaaaaaattagacaggcgtggtggtggatgcctgtaatcccagctacttgggaagctgaggcaggagaatggcttgagcccgggagatggagattgcagtgagatgagattgcgccactgcactccagcctgggtgacagagcaaggctatgtctcaaaagagaaaaaaaaagtaagtatctcagtcttgaagatgatgaaatggaggcctagagagattaagtaacttgcccaaaatgacagaactaatgcatagaaaagaagaaatgtgatgtcttttggctccaaagacaccccacatatgcgttggttacagttactagagaaaagttattccacccccaccccacccccagaaatcttctgacttgttttctcgcagttgagtaggaccatttattcggcagtgtaccattctcagcttgcagttgaaagccaaatatccattaaagaggcaaggatgcaaacttgctaagctgataaatccaggggtgattttttttttttttgcaaaccatccaacaagacattttaaatactcattgaatttcatagaactgactgccaggattggaaagacattaaagccagctcagccactgcctcgctggttggccagaccacgcctggcacttctgggagggagcactcaccaccccccaagggcacccatctcatcctccgaaggtttatgaaaatgcactcatcatttgctaattcattccactacgtgtattacctaatttgtgacacgatgtgaagtaccagagagataattctaaataaaatatagttatgggtctcaaggagccagatatgctaatctcctatcctcctgcagtttacagtggtcctcaccagatacttatttacaaaaattcagtttattatttatttttttgagacagagtcttgctctatagctcaggctagagtgtaatggtgtgatctcggctcacttcaacctctgcctcccaggttcaagtgattctcctgcctcaacctcccaagtagctgggactacaggcacctgccaccacggctaatttttggagttttagtagagacagggtttcaccacgttggccaggctggcctcgaactcctgacctcaggtgatctgcccacatcagcctcccaaaatgttgggattacaggcgtgagccaccatgcccggccaaaacttcagtttataacacaatctttcacgtgtcttctgctttcattaaaagaatagacagttcccttctttatttcagtttaataaaccatggattttatttcatgctttgcaaaacacaagggctcactgacatgcacttcttaaactaattctggctggtcgcctgtaattccagcactttgggaggctgaggccgacagatcacttcaagtcaggagttcaagaccagcctggccaatatggtgaaaccacgtctctaccaaaaatataaaaaattagccaggtgtggtggtgcgtgactataatcccagctactcaggggcctgaggcagaaaaatcacttgaacccgggaggcggaggttacagtgagctgagatcgcgccactgcactccagcctgggcgacagagtgagactctgtctcaaaaaataaataaatacaaataatgtaaaatacgaaacaagcaatcctggcagtagctgctggaatgagaggagggagaggtcatagggaggtcggggacaatggagcatggagttgtgttggatttggctaagcagcaggaagtgcaaggcattccaagcaagaggaggggggcaggtggggagcatctgcaagaacagaagcagcatgagcaacctggctcggcagtgtgtgaaaaggctgaaaggtggctagagccacttcaatttcatccttcaggcaaatgggaaattcccaaaggtttgagtggggaagcaatgcctacaatgaaagtttgagagtgaagcagagtgatcgaattaagcatgtaggccgagttctgaaataactgcaatgtgctgaagatcatccattggcttctgaatgagtatttgcagtttattttttaaaatgattttattgccaagaaagataaacactactgttttggtacaaaaacataacaaaatgtgttgagtccctcttgctgttttacgcgaagttttaaaaatctactcttgtcacagtggtatcacccctacttctgatttcaaataaatgttctagagacacagtaagggcccaacaaacgcttgttcaacaacacaaggagagccagcttttaaagtaggaaaacaggccgggcgccgtggctcacacctgtaatcccaacactttgggaggctgaggtgggcagatcacttgaggtcaggagttcaagaacagcttggccaacatggtgaaaccctgtctctactaaaaacacaaacattagccaggcgtggtggtgcacaccagtagtcccagctattcaggaggctgaggcaggaaaatggcttgaactggggaggcagtggttgcagtgagccgagatcgtgccactgcactccagcctgggggacagagggagactccatctcaaaataaaacaaaacaaaaccaaatcatacaaaaacattagctgggtgtggtggtgcatacctgtaatcccagctacttgggaagctgaggcagaattacttgaacccctggggggaggttgcagtgagctgagatcttgccactacactccagcctgggcaacagagtgaggagactctgtctcaaaaaatatatatattaaaaaaaagaaaaaaaaaagtaaactaggaaaacacatcagcagcctgccaacagactcccctagcctcggtgagggccagtgttctgggaggcagatctgaattctagtcctagttcacccactggcaggctggtgcccttgggcaggtcgcttctctggggctcagtttcttcctctataaaatgagatcaaatcccatgttctaagagtttgtgctctggagtcagacagatctgggttctaccactgccagctctgtgatcttgtagcttcagtctcgtcatctgacatggagataacagtaactgtctcactgtgttgttagggtttaaaggagataatgtatgtgaaatgttagcaaacaagtgttagctaccctgatttccggtttcagagttctgtggtcccagtttatgccacatgcagtgacgttgtatggtaggctgtggtgtggcaccacttcagaactcagcgcatgcacagcttgcagaagagaaggccagaggagacctaagaaggctcttcgaacacttgaaagaccggcatgtaggccgggcgcagtgactcacgcctgtaatcccagcagttttggaggtcgaggcgggtggatcacctgagtttgggagtttgataccagcctgaccaacaaggtgaaaccccgtctctactaaaaaatacaaacattagctgggcatggtggcgggtgcctgtaatcccagctactccggtggttgaggcagaattgcttgaacccgggaggcagaggttgcagtgagctgagattgcatcactgcactccagcctgagacaagagcgaaactccatctcaaacaaaacaaacaaccaaccaaacaaaaccaaaaaaaaaactggcatgtagaagaaaaatactttttctctacacttctccaaagaatttaactaggcccaggggaggtgcagtataaatttctaacaatctcaactgtctgccaaatggaatgagctacttcatatggcagtagtgagtcctctgtctttggaggcattcaaataaaagccagatggccatttatcaacaatccatgtaaaacgttagatgaaataaaacctatatatccaagatctcttccaattcagattttatgaaagaatttctaaggtctttgtaatgagacatttaggctgtttcaagagatcaagccaaaatcagtatgtgggttcatctgcaataaaaatgtttgttttgcttttacagtttcctcatttggctgttggattttaagcaaaagcatccaagaaaaacaaggcctgttcaaaaacaagacaacttcctctcactgttgcctgcatttgtacgtgagaaacgctcatgacagcaaagtctccaatgttcgcgcaggcactggagtcagagaaaatggagttgaatcctttctctgccactctttgaggagaatctcaccatttattatgcactgtagaatacaacaataaaatacagccatgtaccacataacaacatcttggtaaacaacagactgcatatatgatggtggtcatccagtaagctaaggttaatttattattattccttgtttttttttttttttttttttttttgagatgtagtcttactctgtcacccaggctagagtgcaatggcaccatcttggctcactgcaacctctacctcctgggttcaagcaaatctcctgcctcagcctccaaagtagctgggattacaggcacccaccacatctggctaattttttgtatttttagtaaagatggggtttcaccatgttggccaggctgatctcaaactcctgacctcaagtgatctgcccgcctcggcctcccaaagtgctggaaccacaggcctgagccactgtgcccagccttgtttgcttttttaacagataacagtgtgctcatagaaactgctttgacatgactgcaatcatgtgcttcatagaaacttaattagattataccactagagtcttcagatttttatactttttttttttgaaacggagtctcactctgtcaccaggctggagtgcagtgccgcaatctcggctcactgcaacctccgcctcccaggttcaagcaattctcctgcctcagcctcccgagtagctggaattacaagtgcgcactaccacacccagctaatttttgcatttttacttgacagggtttcaccatgttggctaggatagtttcaccaggatctcttggcctcatgatcagcctgcctcggcctcccaaagtgctgggattacaggtgtgagccaccgtgcccagcctatacttccctttttgaataccatttggtgttttgaagaattaacagctttgtgaacgtggcagtgcttgtgattcaggcttccattgagaccaaggggagaacctggttgcaggacaaacagacggacagcgtgtggcagtgtttaaatgctcttctgaaggctgatacgacagctctctgtgcactgattgcatatgcatcccaagattatattattgttttctactgctatgtgtcacactttgccaaacaggatgtggaaaatgaataagcggttttcttaggcacttcttaacagacaattggtcaaaatgaactccattgcttaagaaacacataaacaccatttagtcactgaacatagctatatgtatggttgttactatgggaaatcttgttttgccaattttctttgaaaattctggcagaccaaggttctttttgtttacataatacttgaaaaataaaaatgaacaagctaacaaacta

SEQ ID NO: 4 - skipped

SEQ ID NO: 5 - RNA, 12 nt, Streptococcus pyogenes
  misc_feature 1..12: note = Exemplary spCas9 1 crRNA repeat region
  source 1..12: mol_type = genomic RNA
gttttagagcta

SEQ ID NO: 6 - skipped

SEQ ID NO: 7 - RNA, 66 nt, Streptococcus pyogenes
  misc_feature 1..66: note = Exemplary spCas9 1 tracrRNA
  source 1..66: mol_type = genomic RNA
tagcaagttaaaataaggctagtccgttatcaacttgaaaaagtggcaccgagtcggtgctttttt

SEQ ID NO: 8 - skipped

SEQ ID NO: 9 - RNA, 17 nt, Streptococcus pyogenes
  misc_feature 1..17: note = Exemplary spCas9 2 crRNA repeat region
  source 1..17: mol_type = genomic RNA
gtttsagagctatgctg

SEQ ID NO: 10 - skipped

SEQ ID NO: 11 - RNA, 71 nt, Streptococcus pyogenes
  misc_feature 1..71: note = Exemplary spCas9 2 tracrRNA
  source 1..71: mol_type = genomic RNA
cagcatagcaagttsaaataaggctagtccgttatcaacttgaaaaagtggcaccgagtcggtgctttttt

SEQ ID NO: 12 - skipped

SEQ ID NO: 13 - RNA, 14 nt, Streptococcus pyogenes
  misc_feature 1..14: note = Exemplary saCas9 crRNA repeat region
  source 1..14: mol_type = genomic RNA
gttttagtactctg

SEQ ID NO: 14 - skipped

SEQ ID NO: 15 - RNA, 64 nt, Streptococcus pyogenes
  misc_feature 1..64: note = Exemplary saCas9 tracrRNA
  source 1..64: mol_type = genomic RNA
cagaatctactaaaacaaggcaaaatgccgtgtttatctcgtcaacttgttggcgagatttttt

SEQ ID NO: 16 - RNA, 88 nt, synthetic construct
  misc_feature 1..88: note = Synthetic: Exemplary AkCas12b tracrRNA
  source 1..88: mol_type = other RNA
gtcgtctataggacggcgaggacaacgggaagtgccaatgtgctctttccaagagcaaacaccccgttggcttcaagatgaccgctcg

SEQ ID NO: 17 - skipped

SEQ ID NO: 18 - RNA, 23 nt, synthetic construct
  misc_feature 1..23: note = Synthetic: Exemplary AkCas12b crRNA repeat region
  source 1..23: mol_type = other RNA
cgagcggtctgagaagtggcact

SEQ ID NO: 19 - skipped

SEQ ID NO: 20 - DNA, 20 bp, Homo sapiens
  misc_feature 1..20: note = ABO
  source 1..20: mol_type = genomic DNA
ggccagcgtccgcaacacct

SEQ ID NO: 21 - DNA, 20 bp, Homo sapiens
  misc_feature 1..20: note = ABO
  source 1..20: mol_type = genomic DNA
ggccgaggtgttgcggacgc

SEQ ID NO: 22 - DNA, 20 bp, Homo sapiens
  misc_feature 1..20: note = ABO
  source 1..20: mol_type = genomic DNA
ggtcgaagtgcgtggcattt

SEQ ID NO: 23 - DNA, 20 bp, Homo sapiens
  misc_feature 1..20: note = ABO
  source 1..20: mol_type = genomic DNA
ggatcataggtcgaagtgcg

SEQ ID NO: 24 - DNA, 20 bp, Homo sapiens
  misc_feature 1..20: note = ABO
  source 1..20: mol_type = genomic DNA
attattaggaaaaggatcat

SEQ ID NO: 25 - DNA, 20 bp, Homo sapiens
  misc_feature 1..20: note = ABO
  source 1..20: mol_type = genomic DNA
agacaagcattattaggaaa

SEQ ID NO: 26 - DNA, 20 bp, Homo sapiens
  misc_feature 1..20: note = ABO
  source 1..20: mol_type = genomic DNA
agaccaagacaagcattatt

SEQ ID NO: 27 - DNA, 20 bp, Homo sapiens
  misc_feature 1..20: note = ABO
  source 1..20: mol_type = genomic DNA
tttcctaataatgcttgtct

SEQ ID NO: 28 - DNA, 20 bp, Homo sapiens
  misc_feature 1..20: note = ABO
  source 1..20: mol_type = genomic DNA
atgcttgtcttggtcttgtt

SEQ ID NO: 29 - DNA, 20 bp, Homo sapiens
  misc_feature 1..20: note = ABO
  source 1..20: mol_type = genomic DNA
gcattagacttctggggctt

SEQ ID NO: 30 - DNA, 20 bp, Homo sapiens
  misc_feature 1..20: note = ABO
  source 1..20: mol_type = genomic DNA
ttcctggcattagacttctg

SEQ ID NO: 31 - DNA, 20 bp, Homo sapiens
  misc_feature 1..20: note = ABO
  source 1..20: mol_type = genomic DNA
cttcctggcattagacttct

SEQ ID NO: 32 - DNA, 20 bp, Homo sapiens
  misc_feature 1..20: note = ABO
  source 1..20: mol_type = genomic DNA
gcttcctggcattagacttc

SEQ ID NO: 33 - DNA, 20 bp, Homo sapiens
  misc_feature 1..20: note = ABO
  source 1..20: mol_type = genomic DNA
agccccagaagtctaatgcc

SEQ ID NO: 34 - DNA, 20 bp, Homo sapiens
  misc_feature 1..20: note = ABO
  source 1..20: mol_type = genomic DNA
aacccccgttccaggcttcc

SEQ ID NO: 35 - DNA, 20 bp, Homo sapiens
  misc_feature 1..20: note = ABO
  source 1..20: mol_type = genomic DNA
aagtctaatgccaggaagcc

SEQ ID NO: 36 - DNA, 20 bp, Homo sapiens
  misc_feature 1..20: note = ABO
  source 1..20: mol_type = genomic DNA
taatgccaggaagcctggaa

SEQ ID NO: 37 - DNA, 20 bp, Homo sapiens
  misc_feature 1..20: note = ABO
  source 1..20: mol_type = genomic DNA
aatgccaggaagcctggaac

SEQ ID NO: 38 - DNA, 20 bp, Homo sapiens
  misc_feature 1..20: note = ABO
  source 1..20: mol_type = genomic DNA
atgccaggaagcctggaacg

SEQ ID NO: 39 - DNA, 20 bp, Homo sapiens
  misc_feature 1..20: note = ABO
  source 1..20: mol_type = genomic DNA
tgccaggaagcctggaacgg

SEQ ID NO: 40 - DNA, 20 bp, Homo sapiens
  misc_feature 1..20: note = ABO
  source 1..20: mol_type = genomic DNA
gagacgcgctgcagatggtc

SEQ ID NO: 41 - DNA, 20 bp, Homo sapiens
  misc_feature 1..20: note = ABO
  source 1..20: mol_type = genomic DNA
gcaacgagacgcgctgcaga

SEQ ID NO: 42 - DNA, 20 bp, Homo sapiens
  misc_feature 1..20: note = ABO
  source 1..20: mol_type = genomic DNA
gtgtcagcacctttggctgg

SEQ ID NO: 43 - DNA, 20 bp, Homo sapiens
  misc_feature 1..20: note = ABO
  source 1..20: mol_type = genomic DNA
ggtgtcagcacctttggctg

SEQ ID NO: 44 - DNA, 20 bp, Homo sapiens
  misc_feature 1..20: note = ABO
  source 1..20: mol_type = genomic DNA
gatggtctacccccagccaa

SEQ ID NO: 45 - DNA, 20 bp, Homo sapiens
  misc_feature 1..20: note = ABO
  source 1..20: mol_type = genomic DNA
cggtgtcagcacctttggct

SEQ ID NO: 46 - DNA, 20 bp, Homo sapiens
  misc_feature 1..20: note = ABO
  source 1..20: mol_type = genomic DNA
acggtgtcagcacctttggc

SEQ ID NO: 47 - DNA, 20 bp, Homo sapiens
  misc_feature 1..20: note = ABO
  source 1..20: mol_type = genomic DNA
gacaatgggagccagccaag

SEQ ID NO: 48 - DNA, 20 bp, Homo sapiens
  misc_feature 1..20: note = ABO
  source 1..20: mol_type = genomic DNA
agacaatgggagccagccaa

SEQ ID NO: 49 - DNA, 20 bp, Homo sapiens
  misc_feature 1..20: note = ABO
  source 1..20: mol_type = genomic DNA
cagacaatgggagccagcca

SEQ ID NO: 50 - DNA, 20 bp, Homo sapiens
  misc_feature 1..20: note = ABO
  source 1..20: mol_type = genomic DNA
cttggctggctcccattgtc

SEQ ID NO: 51 - DNA, 20 bp, Homo sapiens
  misc_feature 1..20: note = ABO
  source 1..20: mol_type = genomic DNA
aatgtgccctcccagacaat

SEQ ID NO: 52 - DNA, 20 bp, Homo sapiens
  misc_feature 1..20: note = ABO
  source 1..20: mol_type = genomic DNA
ttggctggctcccattgtct

SEQ ID NO: 53 - DNA, 20 bp, Homo sapiens
  misc_feature 1..20: note = ABO
  source 1..20: mol_type = genomic DNA
gaatgtgccctcccagacaa

SEQ ID NO: 54 - DNA, 20 bp, Homo sapiens
  misc_feature 1..20: note = ABO
  source 1..20: mol_type = genomic DNA
gctggctcccattgtctggg

SEQ ID NO: 55 - DNA, 20 bp, Homo sapiens
  misc_feature 1..20: note = ABO
  source 1..20: mol_type = genomic DNA
ctggctcccattgtctggga

SEQ ID NO: 56 - DNA, 20 bp, Homo sapiens
  misc_feature 1..20: note = ABO
  source 1..20: mol_type = genomic DNA
ggagcctgaactgctcgttg

SEQ ID NO: 57 - DNA, 20 bp, Homo sapiens
  misc_feature 1..20: note = ABO
  source 1..20: mol_type = genomic DNA
acatcctcaacgagcagttc

SEQ ID NO: 58 - DNA, 20 bp, Homo sapiens
  misc_feature 1..20: note = ABO
  source 1..20: mol_type = genomic DNA
ttaacccaatggtggtgttc

SEQ ID NO: 59 - DNA, 20 bp, Homo sapiens
  misc_feature 1..20: note = ABO
  source 1..20: mol_type = genomic DNA
aggctccagaacaccaccat

SEQ ID NO: 60 - DNA, 20 bp, Homo sapiens
  misc_feature 1..20: note = ABO
  source 1..20: mol_type = genomic DNA
ggctccagaacaccaccatt

SEQ ID NO: 61 - DNA, 20 bp, Homo sapiens
  misc_feature 1..20: note = ABO
  source 1..20: mol_type = genomic DNA
aaacacagttaacccaatgg

SEQ ID NO: 62 - DNA, 20 bp, Homo sapiens
  misc_feature 1..20: note = ABO
  source 1..20: mol_type = genomic DNA
ggcaaacacagttaacccaa

SEQ ID NO: 63 - DNA, 20 bp, Homo sapiens
  misc_feature 1..20: note = ABO
  source 1..20: mol_type = genomic DNA
ccgtctccaggaacagcttc

SEQ ID NO: 64 - DNA, 20 bp, Homo sapiens
  misc_feature 1..20: note = ABO
  source 1..20: mol_type = genomic DNA
ggctttcctgaagctgttcc

SEQ ID NO: 65 - DNA, 20 bp, Homo sapiens
  misc_feature 1..20: note = ABO
  source 1..20: mol_type = genomic DNA
cctgaagctgttcctggaga

SEQ ID NO: 66 - DNA, 20 bp, Homo sapiens
  misc_feature 1..20: note = ABO
  source 1..20: mol_type = genomic DNA
agtgcttctccgccgtctcc

SEQ ID NO: 67 - DNA, 20 bp, Homo sapiens
  misc_feature 1..20: note = ABO
  source 1..20: mol_type = genomic DNA
gaagctgttcctggagacgg

SEQ ID NO: 68 - DNA, 20 bp, Homo sapiens
  misc_feature 1..20: note = ABO
  source 1..20: mol_type = genomic DNA
gacggcggagaagcacttca

SEQ ID NO: 69 - DNA, 20 bp, Homo sapiens
  misc_feature 1..20: note = ABO
  source 1..20: mol_type = genomic DNA
ggcggagaagcacttcatgg

SEQ ID NO: 70 - DNA, 20 bp, Homo sapiens
  misc_feature 1..20: note = ABO
  source 1..20: mol_type = genomic DNA
gcggagaagcacttcatggt

SEQ ID NO: 71 - DNA, 20 bp, Homo sapiens
  misc_feature 1..20: note = ABO
  source 1..20: mol_type = genomic DNA
agacatagtagtggacacgg

SEQ ID NO: 72 - DNA, 20 bp, Homo sapiens
  misc_feature 1..20: note = ABO
  source 1..20: mol_type = genomic DNA
tgaagacatagtagtggaca

SEQ ID NO: 73 - DNA, 20 bp, Homo sapiens
  misc_feature 1..20: note = ABO
  source 1..20: mol_type = genomic DNA
ggtcggtgaagacatagtag

SEQ ID NO: 74 - DNA, 20 bp, Homo sapiens
  misc_feature 1..20: note = ABO
  source 1..20: mol_type = genomic DNA
ctatgtcttcaccgaccagc

SEQ ID NO: 75 - DNA, 20 bp, Homo sapiens
  misc_feature 1..20: note = ABO
  source 1..20: mol_type = genomic DNA
gggcaccgcggccggctggt

SEQ ID NO: 76 - DNA, 20 bp, Homo sapiens
  misc_feature 1..20: note = ABO
  source 1..20: mol_type = genomic DNA
cgcggggcaccgcggccggc

SEQ ID NO: 77 - DNA, 20 bp, Homo sapiens
  misc_feature 1..20: note = ABO
  source 1..20: mol_type = genomic DNA
cttcaccgaccagccggccg

SEQ ID NO: 78 - DNA, 20 bp, Homo sapiens
  misc_feature 1..20: note = ABO
  source 1..20: mol_type = genomic DNA
gtcacgcggggcaccgcggc

SEQ ID NO: 79 - DNA, 20 bp, Homo sapiens
  misc_feature 1..20: note = ABO
  source 1..20: mol_type = genomic DNA
cagcgtcacgcggggcaccg

SEQ ID NO: 80 - DNA, 20 bp, Homo sapiens
  misc_feature 1..20: note = ABO
  source 1..20: mol_type = genomic DNA
ccggtccccagcgtcacgcg

SEQ ID NO: 81 - DNA, 20 bp, Homo sapiens
  misc_feature 1..20: note = ABO
  source 1..20: mol_type = genomic DNA
accggtccccagcgtcacgc

SEQ ID NO: 82 - DNA, 20 bp, Homo sapiens
  misc_feature 1..20: note = ABO
  source 1..20: mol_type = genomic DNA
gaccggtccccagcgtcacg

SEQ ID NO: 83 - DNA, 20 bp, Homo sapiens
  misc_feature 1..20: note = ABO
  source 1..20: mol_type = genomic DNA
cgcggtgccccgcgtgacgc

SEQ ID NO: 84 - DNA, 20 bp, Homo sapiens
  misc_feature 1..20: note = ABO
  source 1..20: mol_type = genomic DNA
gcggtgccccgcgtgacgct

SEQ ID NO: 85 - DNA, 20 bp, Homo sapiens
  misc_feature 1..20: note = ABO
  source 1..20: mol_type = genomic DNA
cggtgccccgcgtgacgctg

SEQ ID NO: 86 - DNA, 20 bp, Homo sapiens
  misc_feature 1..20: note = ABO
  source 1..20: mol_type = genomic DNA
ccccgcgtgacgctggggac

SEQ ID NO: 87 - DNA, 20 bp, Homo sapiens
  misc_feature 1..20: note = ABO
  source 1..20: mol_type = genomic DNA
gcgtgacgctggggaccggt

SEQ ID NO: 88 - DNA, 20 bp, Homo sapiens
  misc_feature 1..20: note = ABO
  source 1..20: mol_type = genomic DNA
cagcactgacagctgccgac

SEQ ID NO: 89 - DNA, 20 bp, Homo sapiens
  misc_feature 1..20: note = ABO
  source 1..20: mol_type = genomic DNA
cggtcggcagctgtcagtgc

SEQ ID NO: 90 - DNA, 20 bp, Homo sapiens
  misc_feature 1..20: note = ABO
  source 1..20: mol_type = genomic DNA
tcggcagctgtcagtgctgg

SEQ ID NO: 91 - DNA, 20 bp, Homo sapiens
  misc_feature 1..20: note = ABO
  source 1..20: mol_type = genomic DNA
cacgtcctgccagcgcttgt

SEQ ID NO: 92 - DNA, 20 bp, Homo sapiens
  misc_feature 1..20: note = ABO
  source 1..20: mol_type = genomic DNA
aggtgcgcgcctacaagcgc

SEQ ID NO: 93 - DNA, 20 bp, Homo sapiens
  misc_feature 1..20: note = ABO
  source 1..20: mol_type = genomic DNA
gcgcgcctacaagcgctggc

SEQ ID NO: 94 - DNA, 20 bp, Homo sapiens
  misc_feature 1..20: note = ABO
  source 1..20: mol_type = genomic DNA
gatcatctccatgcggcgca

SEQ ID NO: 95 - DNA, 20 bp, Homo sapiens
  misc_feature 1..20: note = ABO
  source 1..20: mol_type = genomic DNA
ggacgtgtccatgcgccgca

SEQ ID NO: 96 - DNA, 20 bp, Homo sapiens
  misc_feature 1..20: note = ABO
  source 1..20: mol_type = genomic DNA
agtcactgatcatctccatg

SEQ ID NO: 97 - DNA, 20 bp, Homo sapiens
  misc_feature 1..20: note = ABO
  source 1..20: mol_type = genomic DNA
tgatcagtgacttctgcgag

SEQ ID NO: 98 - DNA, 20 bp, Homo sapiens
  misc_feature 1..20: note = ABO
  source 1..20: mol_type = genomic DNA
cgagcggcgcttcctcagcg

SEQ ID NO: 99 - DNA, 20 bp, Homo sapiens
  misc_feature 1..20: note = ABO
  source 1..20: mol_type = genomic DNA
ccaggtaatccacctcgctg

SEQ ID NO: 100 - DNA, 20 bp, Homo sapiens
  misc_feature 1..20: note = ABO
  source 1..20: mol_type = genomic DNA
gcggcgcttcctcagcgagg

SEQ ID NO: 101 - DNA, 20 bp, Homo sapiens
  misc_feature 1..20: note = ABO
  source 1..20: mol_type = genomic DNA
cctcagcgaggtggattacc

SEQ ID NO: 102 - DNA, 20 bp, Homo sapiens
  misc_feature 1..20: note = ABO
  source 1..20: mol_type = genomic DNA
tgtccacgtccacgcacacc

SEQ ID NO: 103 - DNA, 20 bp, Homo sapiens
  misc_feature 1..20: note = ABO
  source 1..20: mol_type = genomic DNA
ggtggattacctggtgtgcg

SEQ ID NO: 104 - DNA, 20 bp, Homo sapiens
  misc_feature 1..20: note = ABO
  source 1..20: mol_type = genomic DNA
ttacctggtgtgcgtggacg

SEQ ID NO: 105 - DNA, 20 bp, Homo sapiens
  misc_feature 1..20: note = ABO
  source 1..20: mol_type = genomic DNA
ggtgtgcgtggacgtggaca

SEQ ID NO: 106 - DNA, 20 bp, Homo sapiens
  misc_feature 1..20: note = ABO
  source 1..20: mol_type = genomic DNA
tctccacgcccacgtggtcg

SEQ ID NO: 107 - DNA, 20 bp, Homo sapiens
  misc_feature 1..20: note = ABO
  source 1..20: mol_type = genomic DNA
catggagttccgcgaccacg

SEQ ID NO: 108 - DNA, 20 bp, Homo sapiens
  misc_feature 1..20: note = ABO
  source 1..20: mol_type = genomic DNA
atggagttccgcgaccacgt

SEQ ID NO: 109 - DNA, 20 bp, Homo sapiens
  misc_feature 1..20: note = ABO
  source 1..20: mol_type = genomic DNA
tcaggatctccacgcccacg

SEQ ID NO: 110 - DNA, 20 bp, Homo sapiens
  misc_feature 1..20: note = ABO
  source 1..20: mol_type = genomic DNA
gttccgcgaccacgtgggcg

SEQ ID NO: 111 - DNA, 20 bp, Homo sapiens
  misc_feature 1..20: note = ABO
  source 1..20: mol_type = genomic DNA
gggtgccgaacagcggagtc

SEQ ID NO: 112 - DNA, 20 bp, Homo sapiens
  misc_feature 1..20: note = ABO
  source 1..20: mol_type = genomic DNA
gagatcctgactccgctgtt

SEQ ID NO: 113 - DNA, 20 bp, Homo sapiens
  misc_feature 1..20: note = ABO
  source 1..20: mol_type = genomic DNA
gggtgcagggtgccgaacag

SEQ ID NO: 114 - DNA, 20 bp, Homo sapiens
  misc_feature 1..20: note = ABO
  source 1..20: mol_type = genomic DNA
tccgtagaagccggggtgca

SEQ ID NO: 115 - DNA, 20 bp, Homo sapiens
  misc_feature 1..20: note = ABO
  source 1..20: mol_type = genomic DNA
ctgttcggcaccctgcaccc

SEQ ID NO: 116 - DNA, 20 bp, Homo sapiens
  misc_feature 1..20: note = ABO
  source 1..20: mol_type = genomic DNA
ttccgtagaagccggggtgc

SEQ ID NO: 117 - DNA, 20 bp, Homo sapiens
  misc_feature 1..20: note = ABO
  source 1..20: mol_type = genomic DNA
ggctgcttccgtagaagccg

SEQ ID NO: 118 - DNA, 20 bp, Homo sapiens
  misc_feature 1..20: note = ABO
  source 1..20: mol_type = genomic DNA
cggctgcttccgtagaagcc

SEQ ID NO: 119 - DNA, 20 bp, Homo sapiens
  misc_feature 1..20: note = ABO
  source 1..20: mol_type = genomic DNA
ccggctgcttccgtagaagc

SEQ ID NO: 120 - DNA, 20 bp, Homo sapiens
  misc_feature 1..20: note = ABO
  source 1..20: mol_type = genomic DNA
accctgcaccccggcttcta

SEQ ID NO: 121 - DNA, 20 bp, Homo sapiens
  misc_feature 1..20: note = ABO
  source 1..20: mol_type = genomic DNA
ccggcttctacggaagcagc

SEQ ID NO: 122 - DNA, 20 bp, Homo sapiens
  misc_feature 1..20: note = ABO
  source 1..20: mol_type = genomic DNA
cggcttctacggaagcagcc

SEQ ID NO: 123 - DNA, 20 bp, Homo sapiens
  misc_feature 1..20: note = ABO
  source 1..20: mol_type = genomic DNA
cttctacggaagcagccggg

SEQ ID NO: 124 - DNA, 20 bp, Homo sapiens
  misc_feature 1..20: note = ABO
  source 1..20: mol_type = genomic DNA
gctcgtaggtgaaggcctcc

SEQ ID NO: 125 - DNA, 20 bp, Homo sapiens
  misc_feature 1..20: note = ABO
  source 1..20: mol_type = genomic DNA
gggccggcgctcgtaggtga

SEQ ID NO: 126 - DNA, 20 bp, Homo sapiens
  misc_feature 1..20: note = ABO
  source 1..20: mol_type = genomic DNA
ggactggggccggcgctcgt

SEQ ID NO: 127 - DNA, 20 bp, Homo sapiens
  misc_feature 1..20: note = ABO
  source 1..20: mol_type = genomic DNA
aggccttcacctacgagcgc

SEQ ID NO: 128 - DNA, 20 bp, Homo sapiens
  misc_feature 1..20: note = ABO
  source 1..20: mol_type = genomic DNA
tgtaggcctgggactggggc

SEQ ID NO: 129 - DNA, 20 bp, Homo sapiens
  misc_feature 1..20: note = ABO
  source 1..20: mol_type = genomic DNA
gggatgtaggcctgggactg

SEQ ID NO: 130 - DNA, 20 bp, Homo sapiens
  misc_feature 1..20: note = ABO
  source 1..20: mol_type = genomic DNA
cgagcgccggccccagtccc

SEQ ID NO: 131 - DNA, 20 bp, Homo sapiens
  misc_feature 1..20: note = ABO
  source 1..20: mol_type = genomic DNA
ggggatgtaggcctgggact

SEQ ID NO: 132 - DNA, 20 bp, Homo sapiens
  misc_feature 1..20: note = ABO
  source 1..20: mol_type = genomic DNA
tggggatgtaggcctgggac

SEQ ID NO: 133 - DNA, 20 bp, Homo sapiens
  misc_feature 1..20: note = ABO
  source 1..20: mol_type = genomic DNA
gtccttggggatgtaggcct

SEQ ID NO: 134 - DNA, 20 bp, Homo sapiens
  misc_feature 1..20: note = ABO
  source 1..20: mol_type = genomic DNA
cgtccttggggatgtaggcc

SEQ ID NO: 135 - DNA, 20 bp, Homo sapiens
  misc_feature 1..20: note = ABO
  source 1..20: mol_type = genomic DNA
gccctcgtccttggggatgt

SEQ ID NO: 136 - DNA, 20 bp, Homo sapiens
  misc_feature 1..20: note = ABO
  source 1..20: mol_type = genomic DNA
gtcccaggcctacatcccca

SEQ ID NO: 137 - DNA, 20 bp, Homo sapiens
  misc_feature 1..20: note = ABO
  source 1..20: mol_type = genomic DNA
agaaatcgccctcgtccttg

SEQ ID NO: 138 - DNA, 20 bp, Homo sapiens
  misc_feature 1..20: note = ABO
  source 1..20: mol_type = genomic DNA
tagaaatcgccctcgtcctt

SEQ ID NO: 139 - DNA, 20 bp, Homo sapiens
  misc_feature 1..20: note = ABO
  source 1..20: mol_type = genomic DNA
ggcctacatccccaaggacg

SEQ ID NO: 140 - DNA, 20 bp, Homo sapiens
  misc_feature 1..20: note = ABO
  source 1..20: mol_type = genomic DNA
gtagaaatcgccctcgtcct

SEQ ID NO: 141 - DNA, 20 bp, Homo sapiens
  misc_feature 1..20: note = ABO
  source 1..20: mol_type = genomic DNA
gcctacatccccaaggacga

SEQ ID NO: 142 - DNA, 20 bp, Homo sapiens
  misc_feature 1..20: note = ABO
  source 1..20: mol_type = genomic DNA
cgagggcgatttctactacc

SEQ ID NO: 143 - DNA, 20 bp, Homo sapiens
  misc_feature 1..20: note = ABO
  source 1..20: mol_type = genomic DNA
gagggcgatttctactacct

SEQ ID NO: 144 - DNA, 20 bp, Homo sapiens
  misc_feature 1..20: note = ABO
  source 1..20: mol_type = genomic DNA
agggcgatttctactacctg

SEQ ID NO: 145 - DNA, 20 bp, Homo sapiens
  misc_feature 1..20: note = ABO
  source 1..20: mol_type = genomic DNA
gggcgatttctactacctgg

SEQ ID NO: 146 - DNA, 20 bp, Homo sapiens
  misc_feature 1..20: note = ABO
  source 1..20: mol_type = genomic DNA
ggcgatttctactacctggg

SEQ ID NO: 147 - DNA, 20 bp, Homo sapiens
  misc_feature 1..20: note = ABO
  source 1..20: mol_type = genomic DNA
gcgatttctactacctgggg

SEQ ID NO: 148 - DNA, 20 bp, Homo sapiens
  misc_feature 1..20: note = ABO
  source 1..20: mol_type = genomic DNA
accccccgaagaaccccccc

SEQ ID NO: 149 - DNA, 20 bp, Homo sapiens
  misc_feature 1..20: note = ABO
  source 1..20: mol_type = genomic DNA
tactacctgggggggttctt

SEQ ID NO: 150 - DNA, 20 bp, Homo sapiens
  misc_feature 1..20: note = ABO
  source 1..20: mol_type = genomic DNA
actacctgggggggttcttc

SEQ ID NO: 151 - DNA, 20 bp, Homo sapiens
  misc_feature 1..20: note = ABO
  source 1..20: mol_type = genomic DNA
ctacctgggggggttcttcg

SEQ ID NO: 152 - DNA, 20 bp, Homo sapiens
  misc_feature 1..20: note = ABO
  source 1..20: mol_type = genomic DNA
tacctgggggggttcttcgg

SEQ ID NO: 153 - DNA, 20 bp, Homo sapiens
  misc_feature 1..20: note = ABO
  source 1..20: mol_type = genomic DNA
acctgggggggttcttcggg

SEQ ID NO: 154 - DNA, 20 bp, Homo sapiens
  misc_feature 1..20: note = ABO
  source 1..20: mol_type = genomic DNA
gggggggttcttcggggggt

SEQ ID NO: 155 - DNA, 20 bp, Homo sapiens
  misc_feature 1..20: note = ABO
  source 1..20: mol_type = genomic DNA
cttcggggggtcggtgcaag

SEQ ID NO: 156 - DNA, 20 bp, Homo sapiens
  misc_feature 1..20: note = ABO
  source 1..20: mol_type = genomic DNA
ggtcggtgcaagaggtgcag

SEQ ID NO: 157 - DNA, 20 bp, Homo sapiens
  misc_feature 1..20: note = ABO
  source 1..20: mol_type = genomic DNA
aagaggtgcagcggctcacc

SEQ ID NO: 158 - DNA, 20 bp, Homo sapiens
  misc_feature 1..20: note = ABO
  source 1..20: mol_type = genomic DNA
agaggtgcagcggctcacca

SEQ ID NO: 159 - DNA, 20 bp, Homo sapiens
  misc_feature 1..20: note = ABO
  source 1..20: mol_type = genomic DNA
catggcctggtggcaggccc

SEQ ID NO: 160 - DNA, 20 bp, Homo sapiens
  misc_feature 1..20: note = ABO
  source 1..20: mol_type = genomic DNA
gctcaccagggcctgccacc

SEQ ID NO: 161 - DNA, 20 bp, Homo sapiens
  misc_feature 1..20: note = ABO
  source 1..20: mol_type = genomic DNA
gaccatcatggcctggtggc

SEQ ID NO: 162 - DNA, 20 bp, Homo sapiens
  misc_feature 1..20: note = ABO
  source 1..20: mol_type = genomic DNA
ggtcgaccatcatggcctgg

SEQ ID NO: 163 - DNA, 20 bp, Homo sapiens
  misc_feature 1..20: note = ABO
  source 1..20: mol_type = genomic DNA
cctggtcgaccatcatggcc

SEQ ID NO: 164 - DNA, 20 bp, Homo sapiens
  misc_feature 1..20: note = ABO
  source 1..20: mol_type = genomic DNA
ggcctgccaccaggccatga

SEQ ID NO: 165 - DNA, 20 bp, Homo sapiens
  misc_feature 1..20: note = ABO
  source 1..20: mol_type = genomic DNA
gttggcctggtcgaccatca

SEQ ID NO: 166 - DNA, 20 bp, Homo sapiens
  misc_feature 1..20: note = ABO
  source 1..20: mol_type = genomic DNA
ccaggccatgatggtcgacc

SEQ ID NO: 167 - DNA, 20 bp, Homo sapiens
  misc_feature 1..20: note = ABO
  source 1..20: mol_type = genomic DNA
atgatggtcgaccaggccaa

SEQ ID NO: 168 - DNA, 20 bp, Homo sapiens
  misc_feature 1..20: note = ABO
  source 1..20: mol_type = genomic DNA
cggcctcgatgccgttggcc

SEQ ID NO: 169 - DNA, 20 bp, Homo sapiens
  misc_feature 1..20: note = ABO
  source 1..20: mol_type = genomic DNA
ccacacggcctcgatgccgt

SEQ ID NO: 170 - DNA, 20 bp, Homo sapiens
  misc_feature 1..20: note = ABO
  source 1..20: mol_type = genomic DNA
cgaccaggccaacggcatcg

SEQ ID NO: 171 - DNA, 20 bp, Homo sapiens
  misc_feature 1..20: note = ABO
  source 1..20: mol_type = genomic DNA
ccaacggcatcgaggccgtg

SEQ ID NO: 172 - DNA, 20 bp, Homo sapiens
  misc_feature 1..20: note = ABO
  source 1..20: mol_type = genomic DNA
gtggctctcgtcgtgccaca

SEQ ID NO: 173 - DNA, 20 bp, Homo sapiens
  misc_feature 1..20: note = ABO
  source 1..20: mol_type = genomic DNA
gcagcaggtacttgttcagg

SEQ ID NO: 174 - DNA, 20 bp, Homo sapiens
  misc_feature 1..20: note = ABO
  source 1..20: mol_type = genomic DNA
ggcgcagcaggtacttgttc

SEQ ID NO: 175 - DNA, 20 bp, Homo sapiens
  misc_feature 1..20: note = ABO
  source 1..20: mol_type = genomic DNA
tggtgggtttgtggcgcagc

SEQ ID NO: 176 - DNA, 20 bp, Homo sapiens
  misc_feature 1..20: note = ABO
  source 1..20: mol_type = genomic DNA
agagcaccttggtgggtttg

SEQ ID NO: 177 - DNA, 20 bp, Homo sapiens
  misc_feature 1..20: note = ABO
  source 1..20: mol_type = genomic DNA
gctgcgccacaaacccacca

SEQ ID NO: 178 - DNA, 20 bp, Homo sapiens
  misc_feature 1..20: note = ABO
  source 1..20: mol_type = genomic DNA
tcgggggagagcaccttggt

SEQ ID NO: 179 - DNA, 20 bp, Homo sapiens
  misc_feature 1..20: note = ABO
  source 1..20: mol_type = genomic DNA
ctcgggggagagcaccttgg

SEQ ID NO: 180 - DNA, 20 bp, Homo sapiens
  misc_feature 1..20: note = ABO
  source 1..20: mol_type = genomic DNA
gtactcgggggagagcacct

SEQ ID NO: 181 - DNA, 20 bp, Homo sapiens
  misc_feature 1..20: note = ABO
  source 1..20: mol_type = genomic DNA
ctggtcccacaagtactcgg

SEQ ID NO: 182 - DNA, 20 bp, Homo sapiens
  misc_feature 1..20: note = ABO
  source 1..20: mol_type = genomic DNA
gctggtcccacaagtactcg

SEQ ID NO: 183 - DNA, 20 bp, Homo sapiens
  misc_feature 1..20: note = ABO
  source 1..20: mol_type = genomic DNA
tgctggtcccacaagtactc

SEQ ID NO: 184 - DNA, 20 bp, Homo sapiens
  misc_feature 1..20: note = ABO
  source 1..20: mol_type = genomic DNA
ctgctggtcccacaagtact

SEQ ID NO: 185 - DNA, 20 bp, Homo sapiens
  misc_feature 1..20: note = ABO
  source 1..20: mol_type = genomic DNA
tgctctcccccgagtacttg

SEQ ID NO: 186 - DNA, 20 bp, Homo sapiens
  misc_feature 1..20: note = ABO
  source 1..20: mol_type = genomic DNA
gctctcccccgagtacttgt

SEQ ID NO: 187 - DNA, 20 bp, Homo sapiens
  misc_feature 1..20: note = ABO
  source 1..20: mol_type = genomic DNA
cgggccagcccagcagctgc

SEQ ID NO: 188 - DNA, 20 bp, Homo sapiens
  misc_feature 1..20: note = ABO
  source 1..20: mol_type = genomic DNA
cttgtgggaccagcagctgc

SEQ ID NO: 189 - DNA, 20 bp, Homo sapiens
  misc_feature 1..20: note = ABO
  source 1..20: mol_type = genomic DNA
ttgtgggaccagcagctgct

SEQ ID NO: 190 - DNA, 20 bp, Homo sapiens
  misc_feature 1..20: note = ABO
  source 1..20: mol_type = genomic DNA
gggaccagcagctgctgggc

SEQ ID NO: 191 - DNA, 20 bp, Homo sapiens
  misc_feature 1..20: note = ABO
  source 1..20: mol_type = genomic DNA
ctcagcttcctcaggacggc

SEQ ID NO: 192 - DNA, 20 bp, Homo sapiens
  misc_feature 1..20: note = ABO
  source 1..20: mol_type = genomic DNA
cctcagcttcctcaggacgg

SEQ ID NO: 193 - DNA, 20 bp, Homo sapiens
  misc_feature 1..20: note = ABO
  source 1..20: mol_type = genomic DNA
tgggctggcccgccgtcctg

SEQ ID NO: 194 - DNA, 20 bp, Homo sapiens
  misc_feature 1..20: note = ABO
  source 1..20: mol_type = genomic DNA
gaacctcagcttcctcagga

SEQ ID NO: 195 - DNA, 20 bp, Homo sapiens
  misc_feature 1..20: note = ABO
  source 1..20: mol_type = genomic DNA
cagtgaacctcagcttcctc

SEQ ID NO: 196 - DNA, 20 bp, Homo sapiens
  misc_feature 1..20: note = ABO
  source 1..20: mol_type = genomic DNA
ccgccgtcctgaggaagctg

SEQ ID NO: 197 - DNA, 20 bp, Homo sapiens
  misc_feature 1..20: note = ABO
  source 1..20: mol_type = genomic DNA
gaggaagctgaggttcactg

SEQ ID NO: 198 - DNA, 20 bp, Homo sapiens
  misc_feature 1..20: note = ABO
  source 1..20: mol_type = genomic DNA
cggaccgcctggtggttctt

SEQ ID NO: 199 - DNA, 20 bp, Homo sapiens
  misc_feature 1..20: note = ABO
  source 1..20: mol_type = genomic DNA
ccggaccgcctggtggttct

SEQ ID NO: 200 - DNA, 20 bp, Homo sapiens
  misc_feature 1..20: note = ABO
  source 1..20: mol_type = genomic DNA
tgcggtgcccaagaaccacc

SEQ ID NO: 201 - DNA, 20 bp, Homo sapiens
  misc_feature 1..20: note = ABO
  source 1..20: mol_type = genomic DNA
ggtgcccaagaaccaccagg

SEQ ID NO: 202 - DNA, 20 bp, Homo sapiens
  misc_feature 1..20: note = ABO
  source 1..20: mol_type = genomic DNA
acgggttccggaccgcctgg

SEQ ID NO: 203 - DNA, 20 bp, Homo sapiens
  misc_feature 1..20: note = ABO
  source 1..20: mol_type = genomic DNA
ccaagaaccaccaggcggtc

SEQ ID NO: 204 - DNA, 20 bp, Homo sapiens
  misc_feature 1..20: note = FUT1
  source 1..20: mol_type = genomic DNA
ggcagagctgacgatggctc

SEQ ID NO: 205 - DNA, 20 bp, Homo sapiens
  misc_feature 1..20: note = FUT1
  source 1..20: mol_type = genomic DNA
aggccaggcagagctgacga

SEQ ID NO: 206 - DNA, 20 bp, Homo sapiens
  misc_feature 1..20: note = FUT1
  source 1..20: mol_type = genomic DNA
gagccatcgtcagctctgcc

SEQ ID NO: 207 - DNA, 20 bp, Homo sapiens
  misc_feature 1..20: note = FUT1
  source 1..20: mol_type = genomic DNA
cacagactagcaggaaggcc

SEQ ID NO: 208 - DNA, 20 bp, Homo sapiens
  misc_feature 1..20: note = FUT1
  source 1..20: mol_type = genomic DNA
gaggacacagactagcagga

SEQ ID NO: 209 - DNA, 20 bp, Homo sapiens
  misc_feature 1..20: note = FUT1
  source 1..20: mol_type = genomic DNA
cagagaggacacagactagc

SEQ ID NO: 210 - DNA, 20 bp, Homo sapiens
  misc_feature 1..20: note = FUT1
  source 1..20: mol_type = genomic DNA
ggaggaagaagattacagag

SEQ ID NO: 211 - DNA, 20 bp, Homo sapiens
  misc_feature 1..20: note = FUT1
  source 1..20: mol_type = genomic DNA
agctgtcttgatggatatgg

SEQ ID NO: 212 - DNA, 20 bp, Homo sapiens
  misc_feature 1..20: note = FUT1
  source 1..20: mol_type = genomic DNA
gaaagctgtcttgatggata

SEQ ID NO: 213 - DNA, 20 bp, Homo sapiens
  misc_feature 1..20: note = FUT1
  source 1..20: mol_type = genomic DNA
catgtggaaagctgtcttga

SEQ ID NO: 214 - DNA, 20 bp, Homo sapiens
  misc_feature 1..20: note = FUT1
  source 1..20: mol_type = genomic DNA
catcaagacagctttccaca

SEQ ID NO: 215 - DNA, 20 bp, Homo sapiens
  misc_feature 1..20: note = FUT1
  source 1..20: mol_type = genomic DNA
atcgacaggcctaggccatg

SEQ ID NO: 216 - DNA, 20 bp, Homo sapiens
  misc_feature 1..20: note = FUT1
  source 1..20: mol_type = genomic DNA
gacagctttccacatggcct

SEQ ID NO: 217 - DNA, 20 bp, Homo sapiens
  misc_feature 1..20: note = FUT1
  source 1..20: mol_type = genomic DNA
gacacaggatcgacaggcct

SEQ ID NO: 218 - DNA, 20 bp, Homo sapiens
  misc_feature 1..20: note = FUT1
  source 1..20: mol_type = genomic DNA
ggtctggacacaggatcgac

SEQ ID NO: 219 - DNA, 20 bp, Homo sapiens
  misc_feature 1..20: note = FUT1
  source 1..20: mol_type = genomic DNA
ccaggcggcggtctggacac

SEQ ID NO: 220 - DNA, 20 bp, Homo sapiens
  misc_feature 1..20: note = FUT1
  source 1..20: mol_type = genomic DNA
ggtgtcaccaggcggcggtc

SEQ ID NO: 221 - DNA, 20 bp, Homo sapiens
  misc_feature 1..20: note = FUT1
  source 1..20: mol_type = genomic DNA
cctgtgtccagaccgccgcc

SEQ ID NO: 222 - DNA, 20 bp, Homo sapiens
  misc_feature 1..20: note = FUT1
  source 1..20: mol_type = genomic DNA
ctgggggtgtcaccaggcgg

SEQ ID NO: 223 - DNA, 20 bp, Homo sapiens
  misc_feature 1..20: note = FUT1
  source 1..20: mol_type = genomic DNA
ccactgggggtgtcaccagg

SEQ ID NO: 224 - DNA, 20 bp, Homo sapiens
  misc_feature 1..20: note = FUT1
  source 1..20: mol_type = genomic DNA
tggccactgggggtgtcacc

SEQ ID NO: 225 - DNA, 20 bp, Homo sapiens
  misc_feature 1..20: note = FUT1
  source 1..20: mol_type = genomic DNA
ccgcctggtgacacccccag

SEQ ID NO: 226 - DNA, 20 bp, Homo sapiens
  misc_feature 1..20: note = FUT1
  source 1..20: mol_type = genomic DNA
aggcagaagatggccactgg

SEQ ID NO: 227 - DNA, 20 bp, Homo sapiens
  misc_feature 1..20: note = FUT1
  source 1..20: mol_type = genomic DNA
caggcagaagatggccactg

SEQ ID NO: 228 - DNA, 20 bp, Homo sapiens
  misc_feature 1..20: note = FUT1
  source 1..20: mol_type = genomic DNA
gcaggcagaagatggccact

SEQ ID NO: 229 - DNA, 20 bp, Homo sapiens
  misc_feature 1..20: note = FUT1
  source 1..20: mol_type = genomic DNA
ggcaggcagaagatggccac

SEQ ID NO: 230 - DNA, 20 bp, Homo sapiens
  misc_feature 1..20: note = FUT1
  source 1..20: mol_type = genomic DNA
agtacccggcaggcagaaga

SEQ ID NO: 231 - DNA, 20 bp, Homo sapiens
  misc_feature 1..20: note = FUT1
  source 1..20: mol_type = genomic DNA
agtggccatcttctgcctgc

SEQ ID NO: 232 - DNA, 20 bp, Homo sapiens
  misc_feature 1..20: note = FUT1
  source 1..20: mol_type = genomic DNA
gtggccatcttctgcctgcc

SEQ ID NO: 233 - DNA, 20 bp, Homo sapiens
  misc_feature 1..20: note = FUT1
  source 1..20: mol_type = genomic DNA
ggcccatcgcagtacccggc

SEQ ID NO: 234 - DNA, 20 bp, Homo sapiens
  misc_feature 1..20: note = FUT1
  source 1..20: mol_type = genomic DNA
ttggggcccatcgcagtacc

SEQ ID NO: 235 - DNA, 20 bp, Homo sapiens
  misc_feature 1..20: note = FUT1
  source 1..20: mol_type = genomic DNA
ctgcctgccgggtactgcga

SEQ ID NO: 236 - DNA, 20 bp, Homo sapiens
  misc_feature 1..20: note = FUT1
  source 1..20: mol_type = genomic DNA
tgcctgccgggtactgcgat

SEQ ID NO: 237 - DNA, 20 bp, Homo sapiens
  misc_feature 1..20: note = FUT1
  source 1..20: mol_type = genomic DNA
gacaggaagaggaggcgttg

SEQ ID NO: 238 - DNA, 20 bp, Homo sapiens
  misc_feature 1..20: note = FUT1
  source 1..20: mol_type = genomic DNA
ggacaggaagaggaggcgtt

SEQ ID NO: 239 - DNA, 20 bp, Homo sapiens
  misc_feature 1..20: note = FUT1
  source 1..20: mol_type = genomic DNA
gggacaggaagaggaggcgt

SEQ ID NO: 240 - DNA, 20 bp, Homo sapiens
  misc_feature 1..20: note = FUT1
  source 1..20: mol_type = genomic DNA
gtgctggggacaggaagagg

SEQ ID NO: 241 - DNA, 20 bp, Homo sapiens
  misc_feature 1..20: note = FUT1
  source 1..20: mol_type = genomic DNA
agggtgctggggacaggaag

SEQ ID NO: 242 - DNA, 20 bp, Homo sapiens
  misc_feature 1..20: note = FUT1
  source 1..20: mol_type = genomic DNA
ggaagcagggtgctggggac

SEQ ID NO: 243 - DNA, 20 bp, Homo sapiens
  misc_feature 1..20: note = FUT1
  source 1..20: mol_type = genomic DNA
gagagggaagcagggtgctg

SEQ ID NO: 244 - DNA, 20 bp, Homo sapiens
  misc_feature 1..20: note = FUT1
  source 1..20: mol_type = genomic DNA
ggagagggaagcagggtgct

SEQ ID NO: 245 - DNA, 20 bp, Homo sapiens
  misc_feature 1..20: note = FUT1
  source 1..20: mol_type = genomic DNA
cggagagggaagcagggtgc

SEQ ID NO: 246 - DNA, 20 bp, Homo sapiens
  misc_feature 1..20: note = FUT1
  source 1..20: mol_type = genomic DNA
aggtgccggagagggaagca

SEQ ID NO: 247 - DNA, 20 bp, Homo sapiens
  misc_feature 1..20: note = FUT1
  source 1..20: mol_type = genomic DNA
caggtgccggagagggaagc

SEQ ID NO: 248 - DNA, 20 bp, Homo sapiens
  misc_feature 1..20: note = FUT1
  source 1..20: mol_type = genomic DNA
cagcaccctgcttccctctc

SEQ ID NO: 249 - DNA, 20 bp, Homo sapiens
  misc_feature 1..20: note = FUT1
  source 1..20: mol_type = genomic DNA
gacagtccaggtgccggaga

SEQ ID NO: 250 - DNA, 20 bp, Homo sapiens
  misc_feature 1..20: note = FUT1
  source 1..20: mol_type = genomic DNA
agacagtccaggtgccggag

SEQ ID NO: 251 - DNA, 20 bp, Homo sapiens
  misc_feature 1..20: note = FUT1
  source 1..20: mol_type = genomic DNA
ctgcttccctctccggcacc

SEQ ID NO: 252 - DNA, 20 bp, Homo sapiens
  misc_feature 1..20: note = FUT1
  source 1..20: mol_type = genomic DNA
ggggtagacagtccaggtgc

SEQ ID NO: 253 - DNA, 20 bp, Homo sapiens
  misc_feature 1..20: note = FUT1
  source 1..20: mol_type = genomic DNA
gccattggggtagacagtcc

SEQ ID NO: 254 - DNA, 20 bp, Homo sapiens
  misc_feature 1..20: note = FUT1
  source 1..20: mol_type = genomic DNA
acctggactgtctaccccaa

SEQ ID NO: 255 - DNA, 20 bp, Homo sapiens
  misc_feature 1..20: note = FUT1
  source 1..20: mol_type = genomic DNA
gattaccaaaccggccattg

SEQ ID NO: 256 - DNA, 20 bp, Homo sapiens
  misc_feature 1..20: note = FUT1
  source 1..20: mol_type = genomic DNA
ggactgtctaccccaatggc

SEQ ID NO: 257 - DNA, 20 bp, Homo sapiens
  misc_feature 1..20: note = FUT1
  source 1..20: mol_type = genomic DNA
tgattaccaaaccggccatt

SEQ ID NO: 258 - DNA, 20 bp, Homo sapiens
  misc_feature 1..20: note = FUT1
  source 1..20: mol_type = genomic DNA
ctgattaccaaaccggccat

SEQ ID NO: 259 - DNA, 20 bp, Homo sapiens
  misc_feature 1..20: note = FUT1
  source 1..20: mol_type = genomic DNA
gtctaccccaatggccggtt

SEQ ID NO: 260 - DNA, 20 bp, Homo sapiens
  misc_feature 1..20: note = FUT1
  source 1..20: mol_type = genomic DNA
gtcccatctgattaccaaac

SEQ ID NO: 261 - DNA, 20 bp, Homo sapiens
  misc_feature 1..20: note = FUT1
  source 1..20: mol_type = genomic DNA
tggccggtttggtaatcaga

SEQ ID NO: 262 - DNA, 20 bp, Homo sapiens
  misc_feature 1..20: note = FUT1
  source 1..20: mol_type = genomic DNA
ggccggtttggtaatcagat

SEQ ID NO: 263 - DNA, 20 bp, Homo sapiens
  misc_feature 1..20: note = FUT1
  source 1..20: mol_type = genomic DNA
gggacagtatgccacgctgc

SEQ ID NO: 264 - DNA, 20 bp, Homo sapiens
  misc_feature 1..20: note = FUT1
  source 1..20: mol_type = genomic DNA
ctgggccagagccagcagcg

SEQ ID NO: 265 - DNA, 20 bp, Homo sapiens
  misc_feature 1..20: note = FUT1
  source 1..20: mol_type = genomic DNA
gtatgccacgctgctggctc

SEQ ID NO: 266 - DNA, 20 bp, Homo sapiens
  misc_feature 1..20: note = FUT1
  source 1..20: mol_type = genomic DNA
ggcccggcggccgttgagct

SEQ ID NO: 267 - DNA, 20 bp, Homo sapiens
  misc_feature 1..20: note = FUT1
  source 1..20: mol_type = genomic DNA
ctggctctggcccagctcaa

SEQ ID NO: 268 - DNA, 20 bp, Homo sapiens
  misc_feature 1..20: note = FUT1
  source 1..20: mol_type = genomic DNA
aggcccggcggccgttgagc

SEQ ID NO: 269 - DNA, 20 bp, Homo sapiens
  misc_feature 1..20: note = FUT1
  source 1..20: mol_type = genomic DNA
tggcccagctcaacggccgc

SEQ ID NO: 270 - DNA, 20 bp, Homo sapiens
  misc_feature 1..20: note = FUT1
  source 1..20: mol_type = genomic DNA
ggcccagctcaacggccgcc

SEQ ID NO: 271 - DNA, 20 bp, Homo sapiens
  misc_feature 1..20: note = FUT1
  source 1..20: mol_type = genomic DNA
caggcaggataaaggcccgg

SEQ ID NO: 272 - DNA, 20 bp, Homo sapiens
  misc_feature 1..20: note = FUT1
  source 1..20: mol_type = genomic DNA
tggcaggcaggataaaggcc

SEQ ID NO: 273 - DNA, 20 bp, Homo sapiens
  misc_feature 1..20: note = FUT1
  source 1..20: mol_type = genomic DNA
atgcatggcaggcaggataa

SEQ ID NO: 274 - DNA, 20 bp, Homo sapiens
  misc_feature 1..20: note = FUT1
  source 1..20: mol_type = genomic DNA
gggcggcatgcatggcaggc

SEQ ID NO: 275 - DNA, 20 bp, Homo sapiens
  misc_feature 1..20: note = FUT1
  source 1..20: mol_type = genomic DNA
gccagggcggcatgcatggc

SEQ ID NO: 276 - DNA, 20 bp, Homo sapiens
  misc_feature 1..20: note = FUT1
  source 1..20: mol_type = genomic DNA
cggggccagggcggcatgca

SEQ ID NO: 277 - DNA, 20 bp, Homo sapiens
  misc_feature 1..20: note = FUT1
  source 1..20: mol_type = genomic DNA
gcctgccatgcatgccgccc

SEQ ID NO: 278 - DNA, 20 bp, Homo sapiens
  misc_feature 1..20: note = FUT1
  source 1..20: mol_type = genomic DNA
gcggaataccggggccaggg

SEQ ID NO: 279 - DNA, 20 bp, Homo sapiens
  misc_feature 1..20: note = FUT1
  source 1..20: mol_type = genomic DNA
catgcatgccgccctggccc

SEQ ID NO: 280 - DNA, 20 bp, Homo sapiens
  misc_feature 1..20: note = FUT1
  source 1..20: mol_type = genomic DNA
gatgcggaataccggggcca

SEQ ID NO: 281 - DNA, 20 bp, Homo sapiens
  misc_feature 1..20: note = FUT1
  source 1..20: mol_type = genomic DNA
tgatgcggaataccggggcc

SEQ ID NO: 282 - DNA, 20 bp, Homo sapiens
  misc_feature 1..20: note = FUT1
  source 1..20: mol_type = genomic DNA
cagggtgatgcggaataccg

SEQ ID NO: 283 - DNA, 20 bp, Homo sapiens
  misc_feature 1..20: note = FUT1
  source 1..20: mol_type = genomic DNA
gcagggtgatgcggaatacc

SEQ ID NO: 284 - DNA, 20 bp, Homo sapiens
  misc_feature 1..20: note = FUT1
  source 1..20: mol_type = genomic DNA
ggcagggtgatgcggaatac

SEQ ID NO: 285 - DNA, 20 bp, Homo sapiens
  misc_feature 1..20: note = FUT1
  source 1..20: mol_type = genomic DNA
ccagcacgggcagggtgatg

SEQ ID NO: 286 - DNA, 20 bp, Homo sapiens
  misc_feature 1..20: note = FUT1
  source 1..20: mol_type = genomic DNA
ttctggggccagcacgggca

SEQ ID NO: 287 - DNA, 20 bp, Homo sapiens
  misc_feature 1..20: note = FUT1
  source 1..20: mol_type = genomic DNA
cttctggggccagcacgggc

SEQ ID NO: 288 - DNA, 20 bp, Homo sapiens
  misc_feature 1..20: note = FUT1
  source 1..20: mol_type = genomic DNA
ccgcatcaccctgcccgtgc

SEQ ID NO: 289 - DNA, 20 bp, Homo sapiens
  misc_feature 1..20: note = FUT1
  source 1..20: mol_type = genomic DNA
tccacttctggggccagcac

SEQ ID NO: 290 - DNA, 20 bp, Homo sapiens
  misc_feature 1..20: note = FUT1
  source 1..20: mol_type = genomic DNA
gtccacttctggggccagca

SEQ ID NO: 291 - DNA, 20 bp, Homo sapiens
  misc_feature 1..20: note = FUT1
  source 1..20: mol_type = genomic DNA
gcccgtgctggccccagaag

SEQ ID NO: 292 - DNA, 20 bp, Homo sapiens
  misc_feature 1..20: note = FUT1
  source 1..20: mol_type = genomic DNA
cgtgcggctgtccacttctg

SEQ ID NO: 293 - DNA, 20 bp, Homo sapiens
  misc_feature 1..20: note = FUT1
  source 1..20: mol_type = genomic DNA
gcgtgcggctgtccacttct

SEQ ID NO: 294 - DNA, 20 bp, Homo sapiens
  misc_feature 1..20: note = FUT1
  source 1..20: mol_type = genomic DNA
ggcgtgcggctgtccacttc

SEQ ID NO: 295 - DNA, 20 bp, Homo sapiens
  misc_feature 1..20: note = FUT1
  source 1..20: mol_type = genomic DNA
gcagctcccgccacggcgtg

SEQ ID NO: 296 - DNA, 20 bp, Homo sapiens
  misc_feature 1..20: note = FUT1
  source 1..20: mol_type = genomic DNA
aagtggacagccgcacgccg

SEQ ID NO: 297 - DNA, 20 bp, Homo sapiens
  misc_feature 1..20: note = FUT1
  source 1..20: mol_type = genomic DNA
tggacagccgcacgccgtgg

SEQ ID NO: 298 - DNA, 20 bp, Homo sapiens
  misc_feature 1..20: note = FUT1
  source 1..20: mol_type = genomic DNA
ggacagccgcacgccgtggc

SEQ ID NO: 299 - DNA, 20 bp, Homo sapiens
  misc_feature 1..20: note = FUT1
  source 1..20: mol_type = genomic DNA
tgaagctgcagctcccgcca

SEQ ID NO: 300 - DNA, 20 bp, Homo sapiens
  misc_feature 1..20: note = FUT1
  source 1..20: mol_type = genomic DNA
gggagctgcagcttcacgac

SEQ ID NO: 301 - DNA, 20 bp, Homo sapiens
  misc_feature 1..20: note = FUT1
  source 1..20: mol_type = genomic DNA
gcagcttcacgactggatgt

SEQ ID NO: 302 - DNA, 20 bp, Homo sapiens
  misc_feature 1..20: note = FUT1
  source 1..20: mol_type = genomic DNA
gcttcacgactggatgtcgg

SEQ ID NO: 303 - DNA, 20 bp, Homo sapiens
  misc_feature 1..20: note = FUT1
  source 1..20: mol_type = genomic DNA
ctggatgtcggaggagtacg

SEQ ID NO: 304 - DNA, 20 bp, Homo sapiens
  misc_feature 1..20: note = FUT1
  source 1..20: mol_type = genomic DNA
aagccagagagcttcaggaa

SEQ ID NO: 305 - DNA, 20 bp, Homo sapiens
  misc_feature 1..20: note = FUT1
  source 1..20: mol_type = genomic DNA
aggggaagccagagagcttc

SEQ ID NO: 306 - DNA, 20 bp, Homo sapiens
  misc_feature 1..20: note = FUT1
  source 1..20: mol_type = genomic DNA
gatcctttcctgaagctctc

SEQ ID NO: 307 - DNA, 20 bp, Homo sapiens
  misc_feature 1..20: note = FUT1
  source 1..20: mol_type = genomic DNA
ggaagaaagtccaagagcag

SEQ ID NO: 308 - DNA, 20 bp, Homo sapiens
  misc_feature 1..20: note = FUT1
  source 1..20: mol_type = genomic DNA
tctctggcttcccctgctct

SEQ ID NO: 309 - DNA, 20 bp, Homo sapiens
  misc_feature 1..20: note = FUT1
  source 1..20: mol_type = genomic DNA
tggaagaaagtccaagagca

SEQ ID NO: 310 - DNA, 20 bp, Homo sapiens
  misc_feature 1..20: note = FUT1
  source 1..20: mol_type = genomic DNA
gtggaagaaagtccaagagc

SEQ ID NO: 311 - DNA, 20 bp, Homo sapiens
  misc_feature 1..20: note = FUT1
  source 1..20: mol_type = genomic DNA
ggatctgttcccggagatgg

SEQ ID NO: 312 - DNA, 20 bp, Homo sapiens
  misc_feature 1..20: note = FUT1
  source 1..20: mol_type = genomic DNA
ggactttcttccaccatctc

SEQ ID NO: 313 - DNA, 20 bp, Homo sapiens
  misc_feature 1..20: note = FUT1
  source 1..20: mol_type = genomic DNA
gactttcttccaccatctcc

SEQ ID NO: 314 - DNA, 20 bp, Homo sapiens
  misc_feature 1..20: note = FUT1
  source 1..20: mol_type = genomic DNA
tgcggatctgttcccggaga

SEQ ID NO: 315 - DNA, 20 bp, Homo sapiens
  misc_feature 1..20: note = FUT1
  source 1..20: mol_type = genomic DNA
actctctgcggatctgttcc

SEQ ID NO: 316 - DNA, 20 bp, Homo sapiens
  misc_feature 1..20: note = FUT1
  source 1..20: mol_type = genomic DNA
cgtgcagggtgaactctctg

SEQ ID NO: 317 - DNA, 20 bp, Homo sapiens
  misc_feature 1..20: note = FUT1
  source 1..20: mol_type = genomic DNA
ttcccgaaggtggtcgtgca

SEQ ID NO: 318 - DNA, 20 bp, Homo sapiens
  misc_feature 1..20: note = FUT1
  source 1..20: mol_type = genomic DNA
cttcccgaaggtggtcgtgc

SEQ ID NO: 319 - DNA, 20 bp, Homo sapiens
  misc_feature 1..20: note = FUT1
  source 1..20: mol_type = genomic DNA
tcaccctgcacgaccacctt

SEQ ID NO: 320 - DNA, 20 bp, Homo sapiens
  misc_feature 1..20: note = FUT1
  source 1..20: mol_type = genomic DNA
caccctgcacgaccaccttc

SEQ ID NO: 321 - DNA, 20 bp, Homo sapiens
  misc_feature 1..20: note = FUT1
  source 1..20: mol_type = genomic DNA
tctgcgcctcttcccgaagg

SEQ ID NO: 322 - DNA, 20 bp, Homo sapiens
  misc_feature 1..20: note = FUT1
  source 1..20: mol_type = genomic DNA
cactctgcgcctcttcccga

SEQ ID NO: 323 - DNA, 20 bp, Homo sapiens
  misc_feature 1..20: note = FUT1
  source 1..20: mol_type = genomic DNA
gcacgaccaccttcgggaag

SEQ ID NO: 324 - DNA, 20 bp, Homo sapiens
  misc_feature 1..20: note = FUT1
  source 1..20: mol_type = genomic DNA
ggaagaggcgcagagtgtgc

SEQ ID NO: 325 - DNA, 20 bp, Homo sapiens
  misc_feature 1..20: note = FUT1
  source 1..20: mol_type = genomic DNA
gaagaggcgcagagtgtgct

SEQ ID NO: 326 - DNA, 20 bp, Homo sapiens
  misc_feature 1..20: note = FUT1
  source 1..20: mol_type = genomic DNA
tgtgctgggtcagctccgcc

SEQ ID NO: 327 - DNA, 20 bp, Homo sapiens
  misc_feature 1..20: note = FUT1
  source 1..20: mol_type = genomic DNA
gtgctgggtcagctccgcct

SEQ ID NO: 328 - DNA, 20 bp, Homo sapiens
  misc_feature 1..20: note = FUT1
  source 1..20: mol_type = genomic DNA
ggtcccctgtgcggcccagg

SEQ ID NO: 329 - DNA, 20 bp, Homo sapiens
  misc_feature 1..20: note = FUT1
  source 1..20: mol_type = genomic DNA
ggcggtcccctgtgcggccc

SEQ ID NO: 330 - DNA, 20 bp, Homo sapiens
  misc_feature 1..20: note = FUT1
  source 1..20: mol_type = genomic DNA
cagctccgcctgggccgcac

SEQ ID NO: 331 - DNA, 20 bp, Homo sapiens
  misc_feature 1..20: note = FUT1
  source 1..20: mol_type = genomic DNA
agctccgcctgggccgcaca

SEQ ID NO: 332 - DNA, 20 bp, Homo sapiens
  misc_feature 1..20: note = FUT1
  source 1..20: mol_type = genomic DNA
gctccgcctgggccgcacag

SEQ ID NO: 333 - DNA, 20 bp, Homo sapiens
  misc_feature 1..20: note = FUT1
  source 1..20: mol_type = genomic DNA
tgcgcgggcggtcccctgtg

SEQ ID NO: 334 - DNA, 20 bp, Homo sapiens
  misc_feature 1..20: note = FUT1
  source 1..20: mol_type = genomic DNA
cgccgacaaaggtgcgcggg

SEQ ID NO: 335 - DNA, 20 bp, Homo sapiens
  misc_feature 1..20: note = FUT1
  source 1..20: mol_type = genomic DNA
ggacgccgacaaaggtgcgc

SEQ ID NO: 336 - DNA, 20 bp, Homo sapiens
  misc_feature 1..20: note = FUT1
  source 1..20: mol_type = genomic DNA
tggacgccgacaaaggtgcg

SEQ ID NO: 337 - DNA, 20 bp, Homo sapiens
  misc_feature 1..20: note = FUT1
  source 1..20: mol_type = genomic DNA
gaccgcccgcgcacctttgt

SEQ ID NO: 338 - DNA, 20 bp, Homo sapiens
  misc_feature 1..20: note = FUT1
  source 1..20: mol_type = genomic DNA
gcgcacgtggacgccgacaa

SEQ ID NO: 339 - DNA, 20 bp, Homo sapiens
  misc_feature 1..20: note = FUT1
  source 1..20: mol_type = genomic DNA
gatagtccccacggcgcacg

SEQ ID NO: 340 - DNA, 20 bp, Homo sapiens
  misc_feature 1..20: note = FUT1
  source 1..20: mol_type = genomic DNA
gtcggcgtccacgtgcgccg

SEQ ID NO: 341 - DNA, 20 bp, Homo sapiens
  misc_feature 1..20: note = FUT1
  source 1..20: mol_type = genomic DNA
tcggcgtccacgtgcgccgt

SEQ ID NO: 342 - DNA, 20 bp, Homo sapiens
  misc_feature 1..20: note = FUT1
  source 1..20: mol_type = genomic DNA
cggcgtccacgtgcgccgtg

SEQ ID NO: 343 - DNA, 20 bp, Homo sapiens
  misc_feature 1..20: note = FUT1
  source 1..20: mol_type = genomic DNA
taacctgcagatagtcccca

SEQ ID NO: 344 - DNA, 20 bp, Homo sapiens
  misc_feature 1..20: note = FUT1
  source 1..20: mol_type = genomic DNA
gcgccgtggggactatctgc

SEQ ID NO: 345 - DNA, 20 bp, Homo sapiens
  misc_feature 1..20: note = FUT1
  source 1..20: mol_type = genomic DNA
tgcaggttatgcctcagcgc

SEQ ID NO: 346 - DNA, 20 bp, Homo sapiens
  misc_feature 1..20: note = FUT1
  source 1..20: mol_type = genomic DNA
accacacccttccagcgctg

SEQ ID NO: 347 - DNA, 20 bp, Homo sapiens
  misc_feature 1..20: note = FUT1
  source 1..20: mol_type = genomic DNA
ggttatgcctcagcgctgga

SEQ ID NO: 348 - DNA, 20 bp, Homo sapiens
  misc_feature 1..20: note = FUT1
  source 1..20: mol_type = genomic DNA
gttatgcctcagcgctggaa

SEQ ID NO: 349 - DNA, 20 bp, Homo sapiens
  misc_feature 1..20: note = FUT1
  source 1..20: mol_type = genomic DNA
gcctcagcgctggaagggtg

SEQ ID NO: 350 - DNA, 20 bp, Homo sapiens
  misc_feature 1..20: note = FUT1
  source 1..20: mol_type = genomic DNA
tcagcgctggaagggtgtgg

SEQ ID NO: 351 - DNA, 20 bp, Homo sapiens
  misc_feature 1..20: note = FUT1
  source 1..20: mol_type = genomic DNA
cagcgctggaagggtgtggt

SEQ ID NO: 352 - DNA, 20 bp, Homo sapiens
  misc_feature 1..20: note = FUT1
  source 1..20: mol_type = genomic DNA
tgggcgacagcgcctacctc

SEQ ID NO: 353 - DNA, 20 bp, Homo sapiens
  misc_feature 1..20: note = FUT1
  source 1..20: mol_type = genomic DNA
gtccatggcctgccggaggt

SEQ ID NO: 354 - DNA, 20 bp, Homo sapiens
  misc_feature 1..20: note = FUT1
  source 1..20: mol_type = genomic DNA
cgacagcgcctacctccggc

SEQ ID NO: 355 - DNA, 20 bp, Homo sapiens
  misc_feature 1..20: note = FUT1
  source 1..20: mol_type = genomic DNA
accagtccatggcctgccgg

SEQ ID NO: 356 - DNA, 20 bp, Homo sapiens
  misc_feature 1..20: note = FUT1
  source 1..20: mol_type = genomic DNA
ggaaccagtccatggcctgc

SEQ ID NO: 357 - DNA, 20 bp, Homo sapiens
  misc_feature 1..20: note = FUT1
  source 1..20: mol_type = genomic DNA
cgcctacctccggcaggcca

SEQ ID NO: 358 - DNA, 20 bp, Homo sapiens
  misc_feature 1..20: note = FUT1
  source 1..20: mol_type = genomic DNA
acctccggcaggccatggac

SEQ ID NO: 359 - DNA, 20 bp, Homo sapiens
  misc_feature 1..20: note = FUT1
  source 1..20: mol_type = genomic DNA
ccgtgcccggaaccagtcca

SEQ ID NO: 360 - DNA, 20 bp, Homo sapiens
  misc_feature 1..20: note = FUT1
  source 1..20: mol_type = genomic DNA
ggcaggccatggactggttc

SEQ ID NO: 361 - DNA, 20 bp, Homo sapiens
  misc_feature 1..20: note = FUT1
  source 1..20: mol_type = genomic DNA
gcaggccatggactggttcc

SEQ ID NO: 362 - DNA, 20 bp, Homo sapiens
  misc_feature 1..20: note = FUT1
  source 1..20: mol_type = genomic DNA
ccatggactggttccgggca

SEQ ID NO: 363 - DNA, 20 bp, Homo sapiens
  misc_feature 1..20: note = FUT1
  source 1..20: mol_type = genomic DNA
cgggggcttcgtgccgtgcc

SEQ ID NO: 364 - DNA, 20 bp, Homo sapiens
  misc_feature 1..20: note = FUT1
  source 1..20: mol_type = genomic DNA
gctggtgaccacgaaaacgg

SEQ ID NO: 365 - DNA, 20 bp, Homo sapiens
  misc_feature 1..20: note = FUT1
  source 1..20: mol_type = genomic DNA
tgctggtgaccacgaaaacg

SEQ ID NO: 366 - DNA, 20 bp, Homo sapiens
  misc_feature 1..20: note = FUT1
  source 1..20: mol_type = genomic DNA
ttgctggtgaccacgaaaac

SEQ ID NO: 367 - DNA, 20 bp, Homo sapiens
  misc_feature 1..20: note = FUT1
  source 1..20: mol_type = genomic DNA
gcacgaagcccccgttttcg

SEQ ID NO: 368 - DNA, 20 bp, Homo sapiens
  misc_feature 1..20: note = FUT1
  source 1..20: mol_type = genomic DNA
gttgctggtgaccacgaaaa

SEQ ID NO: 369 - DNA, 20 bp, Homo sapiens
  misc_feature 1..20: note = FUT1
  source 1..20: mol_type = genomic DNA
gttttcgtggtcaccagcaa

SEQ ID NO: 370 - DNA, 20 bp, Homo sapiens
  misc_feature 1..20: note = FUT1
  source 1..20: mol_type = genomic DNA
acaccactccatgccgttgc

SEQ ID NO: 371 - DNA, 20 bp, Homo sapiens
  misc_feature 1..20: note = FUT1
  source 1..20: mol_type = genomic DNA
cgtggtcaccagcaacggca

SEQ ID NO: 372 - DNA, 20 bp, Homo sapiens
  misc_feature 1..20: note = FUT1
  source 1..20: mol_type = genomic DNA
tcaccagcaacggcatggag

SEQ ID NO: 373 - DNA, 20 bp, Homo sapiens
  misc_feature 1..20: note = FUT1
  source 1..20: mol_type = genomic DNA
agaaaacatcgacacctccc

SEQ ID NO: 374 - DNA, 20 bp, Homo sapiens
  misc_feature 1..20: note = FUT1
  source 1..20: mol_type = genomic DNA
gaaaacatcgacacctccca

SEQ ID NO: 375 - DNA, 20 bp, Homo sapiens
  misc_feature 1..20: note = FUT1
  source 1..20: mol_type = genomic DNA
aaacgtcacatcgccctggg

SEQ ID NO: 376 - DNA, 20 bp, Homo sapiens
  misc_feature 1..20: note = FUT1
  source 1..20: mol_type = genomic DNA
agcaaacgtcacatcgccct

SEQ ID NO: 377 - DNA, 20 bp, Homo sapiens
  misc_feature 1..20: note = FUT1
  source 1..20: mol_type = genomic DNA
cagcaaacgtcacatcgccc

SEQ ID NO: 378 - DNA, 20 bp, Homo sapiens
  misc_feature 1..20: note = FUT1
  source 1..20: mol_type = genomic DNA
cagggcgatgtgacgtttgc

SEQ ID NO: 379 - DNA, 20 bp, Homo sapiens
  misc_feature 1..20: note = FUT1
  source 1..20: mol_type = genomic DNA
gatgtgacgtttgctggcga

SEQ ID NO: 380 - DNA, 20 bp, Homo sapiens
  misc_feature 1..20: note = FUT1
  source 1..20: mol_type = genomic DNA
gacgtttgctggcgatggac

SEQ ID NO: 381 - DNA, 20 bp, Homo sapiens
  misc_feature 1..20: note = FUT1
  source 1..20: mol_type = genomic DNA
gtttgctggcgatggacagg

SEQ ID NO: 382 - DNA, 20 bp, Homo sapiens
  misc_feature 1..20: note = FUT1
  source 1..20: mol_type = genomic DNA
atggacaggaggctacaccg

SEQ ID NO: 383 - DNA, 20 bp, Homo sapiens
  misc_feature 1..20: note = FUT1
  source 1..20: mol_type = genomic DNA
agcagggcaaagtctttcca

SEQ ID NO: 384 - DNA, 20 bp, Homo sapiens
  misc_feature 1..20: note = FUT1
  source 1..20: mol_type = genomic DNA
gtggttgcactgtgtgagca

SEQ ID NO: 385 - DNA, 20 bp, Homo sapiens
  misc_feature 1..20: note = FUT1
  source 1..20: mol_type = genomic DNA
tgtggttgcactgtgtgagc

SEQ ID NO: 386 - DNA, 20 bp, Homo sapiens
  misc_feature 1..20: note = FUT1
  source 1..20: mol_type = genomic DNA
tgccaatggtcataatggtg

SEQ ID NO: 387 - DNA, 20 bp, Homo sapiens
  misc_feature 1..20: note = FUT1
  source 1..20: mol_type = genomic DNA
gaaggtgccaatggtcataa

SEQ ID NO: 388 - DNA, 20 bp, Homo sapiens
  misc_feature 1..20: note = FUT1
  source 1..20: mol_type = genomic DNA
aaccacaccattatgaccat

SEQ ID NO: 389 - DNA, 20 bp, Homo sapiens
  misc_feature 1..20: note = FUT1
  source 1..20: mol_type = genomic DNA
ccagaagccgaaggtgccaa

SEQ ID NO: 390 - DNA, 20 bp, Homo sapiens
  misc_feature 1..20: note = FUT1
  source 1..20: mol_type = genomic DNA
attatgaccattggcacctt

SEQ ID NO: 391 - DNA, 20 bp, Homo sapiens
  misc_feature 1..20: note = FUT1
  source 1..20: mol_type = genomic DNA
gtaggcagcccagaagccga

SEQ ID NO: 392 - DNA, 20 bp, Homo sapiens
  misc_feature 1..20: note = FUT1
  source 1..20: mol_type = genomic DNA
ccattggcaccttcggcttc

SEQ ID NO: 393 - DNA, 20 bp, Homo sapiens
  misc_feature 1..20: note = FUT1
  source 1..20: mol_type = genomic DNA
cattggcaccttcggcttct

SEQ ID NO: 394 - DNA, 20 bp, Homo sapiens
  misc_feature 1..20: note = FUT1
  source 1..20: mol_type = genomic DNA
cggcttctgggctgcctacc

SEQ ID NO: 395 - DNA, 20 bp, Homo sapiens
  misc_feature 1..20: note = FUT1
  source 1..20: mol_type = genomic DNA
agtgtctccgccagccaggt

SEQ ID NO: 396 - DNA, 20 bp, Homo sapiens
  misc_feature 1..20: note = FUT1
  source 1..20: mol_type = genomic DNA
ttctgggctgcctacctggc

SEQ ID NO: 397 - DNA, 20 bp, Homo sapiens
  misc_feature 1..20: note = FUT1
  source 1..20: mol_type = genomic DNA
tgggctgcctacctggctgg

SEQ ID NO: 398 - DNA, 20 bp, Homo sapiens
  misc_feature 1..20: note = FUT1
  source 1..20: mol_type = genomic DNA
agacagtgtctccgccagcc

SEQ ID NO: 399 - DNA, 20 bp, Homo sapiens
  misc_feature 1..20: note = FUT1
  source 1..20: mol_type = genomic DNA
tggcggagacactgtctacc

SEQ ID NO: 400 - DNA, 20 bp, Homo sapiens
  misc_feature 1..20: note = FUT1
  source 1..20: mol_type = genomic DNA
ctggcagggtgaagttggcc

SEQ ID NO: 401 - DNA, 20 bp, Homo sapiens
  misc_feature 1..20: note = FUT1
  source 1..20: mol_type = genomic DNA
agagtctggcagggtgaagt

SEQ ID NO: 402 - DNA, 20 bp, Homo sapiens
  misc_feature 1..20: note = FUT1
  source 1..20: mol_type = genomic DNA
caggaactcagagtctggca

SEQ ID NO: 403 - DNA, 20 bp, Homo sapiens
  misc_feature 1..20: note = FUT1
  source 1..20: mol_type = genomic DNA
tcaggaactcagagtctggc

SEQ ID NO: 404 - DNA, 20 bp, Homo sapiens
  misc_feature 1..20: note = FUT1
  source 1..20: mol_type = genomic DNA
atcttcaggaactcagagtc

SEQ ID NO: 405 - DNA, 20 bp, Homo sapiens
  misc_feature 1..20: note = FUT1
  source 1..20: mol_type = genomic DNA
cctccggcttaaagatcttc

SEQ ID NO: 406 - DNA, 20 bp, Homo sapiens
  misc_feature 1..20: note = FUT1
  source 1..20: mol_type = genomic DNA
gttcctgaagatctttaagc

SEQ ID NO: 407 - DNA, 20 bp, Homo sapiens
  misc_feature 1..20: note = FUT1
  source 1..20: mol_type = genomic DNA
cctgaagatctttaagccgg

SEQ ID NO: 408 - DNA, 20 bp, Homo sapiens
  misc_feature 1..20: note = FUT1
  source 1..20: mol_type = genomic DNA
gaagatctttaagccggagg

SEQ ID NO: 409 - DNA, 20 bp, Homo sapiens
  misc_feature 1..20: note = FUT1
  source 1..20: mol_type = genomic DNA
tcgggcaggaaggccgcctc

SEQ ID NO: 410 - DNA, 20 bp, Homo sapiens
  misc_feature 1..20: note = FUT1
  source 1..20: mol_type = genomic DNA
gcccacccactcgggcagga

SEQ ID NO: 411 - DNA, 20 bp, Homo sapiens
  misc_feature 1..20: note = FUT1
  source 1..20: mol_type = genomic DNA
taatgcccacccactcgggc

SEQ ID NO: 412 - DNA, 20 bp, Homo sapiens
  misc_feature 1..20: note = FUT1
  source 1..20: mol_type = genomic DNA
aggcggccttcctgcccgag

SEQ ID NO: 413 - DNA, 20 bp, Homo sapiens
  misc_feature 1..20: note = FUT1
  source 1..20: mol_type = genomic DNA
ggcggccttcctgcccgagt

SEQ ID NO: 414 - DNA, 20 bp, Homo sapiens
  misc_feature 1..20: note = FUT1
  source 1..20: mol_type = genomic DNA
gcattaatgcccacccactc

SEQ ID NO: 415 - DNA, 20 bp, Homo sapiens
  misc_feature 1..20: note = FUT1
  source 1..20: mol_type = genomic DNA
ggccttcctgcccgagtggg

SEQ ID NO: 416 - DNA, 20 bp, Homo sapiens
  misc_feature 1..20: note = FUT1
  source 1..20: mol_type = genomic DNA
tgcattaatgcccacccact

SEQ ID NO: 417 - DNA, 20 bp, Homo sapiens
  misc_feature 1..20: note = FUT1
  source 1..20: mol_type = genomic DNA
gccttcctgcccgagtgggt

SEQ ID NO: 418 - DNA, 20 bp, Homo sapiens
  misc_feature 1..20: note = FUT1
  source 1..20: mol_type = genomic DNA
atgcagacttgtctccactc

SEQ ID NO: 419 - DNA, 20 bp, Homo sapiens
  misc_feature 1..20: note = FUT1
  source 1..20: mol_type = genomic DNA
ggcttagccaatgtccagag

SEQ ID NO: 420 - DNA, 20 bp, Homo sapiens
  misc_feature 1..20: note = FUT1
  source 1..20: mol_type = genomic DNA
cttgtctccactctggacat

SEQ ID NO: 421 - DNA, 20 bp, Homo sapiens
  misc_feature 1..20: note = RHD
  source 1..20: mol_type = genomic DNA
ggcagcgccggacagaccgc

SEQ ID NO: 422 - DNA, 20 bp, Homo sapiens
  misc_feature 1..20: note = RHD
  source 1..20: mol_type = genomic DNA
aggcagcgccggacagaccg

SEQ ID NO: 423 - DNA, 20 bp, Homo sapiens
  misc_feature 1..20: note = RHD
  source 1..20: mol_type = genomic DNA
ctaagtacccgcggtctgtc

SEQ ID NO: 424 - DNA, 20 bp, Homo sapiens
  misc_feature 1..20: note = RHD
  source 1..20: mol_type = genomic DNA
cccagaggggcaggcagcgc

SEQ ID NO: 425 - DNA, 20 bp, Homo sapiens
  misc_feature 1..20: note = RHD
  source 1..20: mol_type = genomic DNA
gtgttagggcccagaggggc

SEQ ID NO: 426 - DNA, 20 bp, Homo sapiens
  misc_feature 1..20: note = RHD
  source 1..20: mol_type = genomic DNA
tccggcgctgcctgcccctc

SEQ ID NO: 427 - DNA, 20 bp, Homo sapiens
  misc_feature 1..20: note = RHD
  source 1..20: mol_type = genomic DNA
ccggcgctgcctgcccctct

SEQ ID NO: 428 - DNA, 20 bp, Homo sapiens
  misc_feature 1..20: note = RHD
  source 1..20: mol_type = genomic DNA
tccagtgttagggcccagag

SEQ ID NO: 429 - DNA, 20 bp, Homo sapiens
  misc_feature 1..20: note = RHD
  source 1..20: mol_type = genomic DNA
ttccagtgttagggcccaga

SEQ ID NO: 430 - DNA, 20 bp, Homo sapiens
  misc_feature 1..20: note = RHD
  source 1..20: mol_type = genomic DNA
cttccagtgttagggcccag

SEQ ID NO: 431 - DNA, 20 bp, Homo sapiens
  misc_feature 1..20: note = RHD
  source 1..20: mol_type = genomic DNA
gcccctctgggccctaacac

SEQ ID NO: 432 - DNA, 20 bp, Homo sapiens
  misc_feature 1..20: note = RHD
  source 1..20: mol_type = genomic DNA
gagagctgcttccagtgtta

SEQ ID NO: 433 - DNA, 20 bp, Homo sapiens
  misc_feature 1..20: note = RHD
  source 1..20: mol_type = genomic DNA
tgagagctgcttccagtgtt

SEQ ID NO: 434 - DNA, 20 bp, Homo sapiens
  misc_feature 1..20: note = RHD
  source 1..20: mol_type = genomic DNA
agtgggtaaaaaaatagaag

SEQ ID NO: 435 - DNA, 20 bp, Homo sapiens
  misc_feature 1..20: note = RHD
  source 1..20: mol_type = genomic DNA
ctctaaggaagcgtcatagt

SEQ ID NO: 436 - DNA, 20 bp, Homo sapiens
  misc_feature 1..20: note = RHD
  source 1..20: mol_type = genomic DNA
cctctaaggaagcgtcatag

SEQ ID NO: 437 - DNA, 20 bp, Homo sapiens
  misc_feature 1..20: note = RHD
  source 1..20: mol_type = genomic DNA
ccactatgacgcttccttag

SEQ ID NO: 438 - DNA, 20 bp, Homo sapiens
  misc_feature 1..20: note = RHD
  source 1..20: mol_type = genomic DNA
gagccccttttgatcctcta

SEQ ID NO: 439 - DNA, 20 bp, Homo sapiens
  misc_feature 1..20: note = RHD
  source 1..20: mol_type = genomic DNA
cgcttccttagaggatcaaa

SEQ ID NO: 440 - DNA, 20 bp, Homo sapiens
  misc_feature 1..20: note = RHD
  source 1..20: mol_type = genomic DNA
gcttccttagaggatcaaaa

SEQ ID NO: 441 - DNA, 20 bp, Homo sapiens
  misc_feature 1..20: note = RHD
  source 1..20: mol_type = genomic DNA
cttccttagaggatcaaaag

SEQ ID NO: 442 - DNA, 20 bp, Homo sapiens
  misc_feature 1..20: note = RHD
  source 1..20: mol_type = genomic DNA
agaggatcaaaaggggctcg

SEQ ID NO: 443 - DNA, 20 bp, Homo sapiens
  misc_feature 1..20: note = RHD
  source 1..20: mol_type = genomic DNA
ccgccatcacggtcagatct

SEQ ID NO: 444 - DNA, 20 bp, Homo sapiens
  misc_feature 1..20: note = RHD
  source 1..20: mol_type = genomic DNA
tggccaagatctgaccgtga

SEQ ID NO: 445 - DNA, 20 bp, Homo sapiens
  misc_feature 1..20: note = RHD
  source 1..20: mol_type = genomic DNA
ccaagatctgaccgtgatgg

SEQ ID NO: 446 - DNA, 20 bp, Homo sapiens
  misc_feature 1..20: note = RHD
  source 1..20: mol_type = genomic DNA
caagccaatggccgccatca

SEQ ID NO: 447 - DNA, 20 bp, Homo sapiens
  misc_feature 1..20: note = RHD
  source 1..20: mol_type = genomic DNA
ctgaccgtgatggcggccat

SEQ ID NO: 448 - DNA, 20 bp, Homo sapiens
  misc_feature 1..20: note = RHD
  source 1..20: mol_type = genomic DNA
cgtgatggcggccattggct

SEQ ID NO: 449 - DNA, 20 bp, Homo sapiens
  misc_feature 1..20: note = RHD
  source 1..20: mol_type = genomic DNA
ggtgaggaagcccaagccaa

SEQ ID NO: 450 - DNA, 20 bp, Homo sapiens
  misc_feature 1..20: note = RHD
  source 1..20: mol_type = genomic DNA
gtgatggcggccattggctt

SEQ ID NO: 451 - DNA, 20 bp, Homo sapiens
  misc_feature 1..20: note = RHD
  source 1..20: mol_type = genomic DNA
gtctccggaaactcgaggtg

SEQ ID NO: 452 - DNA, 20 bp, Homo sapiens
  misc_feature 1..20: note = RHD
  source 1..20: mol_type = genomic DNA
gctgtgtctccggaaactcg

SEQ ID NO: 453 - DNA, 20 bp, Homo sapiens
  misc_feature 1..20: note = RHD
  source 1..20: mol_type = genomic DNA
gcttcctcacctcgagtttc

SEQ ID NO: 454 - DNA, 20 bp, Homo sapiens
  misc_feature 1..20: note = RHD
  source 1..20: mol_type = genomic DNA
cactgctccagctgtgtctc

SEQ ID NO: 455 - DNA, 20 bp, Homo sapiens
  misc_feature 1..20: note = RHD
  source 1..20: mol_type = genomic DNA
cgagtttccggagacacagc

SEQ ID NO: 456 - DNA, 20 bp, Homo sapiens
  misc_feature 1..20: note = RHD
  source 1..20: mol_type = genomic DNA
gagacacagctggagcagtg

SEQ ID NO: 457 - DNA, 20 bp, Homo sapiens
  misc_feature 1..20: note = RHD
  source 1..20: mol_type = genomic DNA
cgccagcatgaagaggttga

SEQ ID NO: 458 - DNA, 20 bp, Homo sapiens
  misc_feature 1..20: note = RHD
  source 1..20: mol_type = genomic DNA
caccaagcgccagcatgaag

SEQ ID NO: 459 - DNA, 20 bp, Homo sapiens
  misc_feature 1..20: note = RHD
  source 1..20: mol_type = genomic DNA
ggccttcaacctcttcatgc

SEQ ID NO: 460 - DNA, 20 bp, Homo sapiens
  misc_feature 1..20: note = RHD
  source 1..20: mol_type = genomic DNA
aacctcttcatgctggcgct

SEQ ID NO: 461 - DNA, 20 bp, Homo sapiens
  misc_feature 1..20: note = RHD
  source 1..20: mol_type = genomic DNA
tgctggcgcttggtgtgcag

SEQ ID NO: 462 - DNA, 20 bp, Homo sapiens
  misc_feature 1..20: note = RHD
  source 1..20: mol_type = genomic DNA
gctggcgcttggtgtgcagt

SEQ ID NO: 463 - DNA, 20 bp, Homo sapiens
  misc_feature 1..20: note = RHD
  source 1..20: mol_type = genomic DNA
tgtgcagtgggcaatcctgc

SEQ ID NO: 464 - DNA, 20 bp, Homo sapiens
  misc_feature 1..20: note = RHD
  source 1..20: mol_type = genomic DNA
cagtgggcaatcctgctgga

SEQ ID NO: 465 - DNA, 20 bp, Homo sapiens
  misc_feature 1..20: note = RHD
  source 1..20: mol_type = genomic DNA
ggctcaggaagccgtccagc

SEQ ID NO: 466 - DNA, 20 bp, Homo sapiens
  misc_feature 1..20: note = RHD
  source 1..20: mol_type = genomic DNA
tcccagaagggaactggctc

SEQ ID NO: 467 - DNA, 20 bp, Homo sapiens
  misc_feature 1..20: note = RHD
  source 1..20: mol_type = genomic DNA
ccaccttcccagaagggaac

SEQ ID NO: 468 - DNA, 20 bp, Homo sapiens
  misc_feature 1..20: note = RHD
  source 1..20: mol_type = genomic DNA
ttcctgagccagttcccttc

SEQ ID NO: 469 - DNA, 20 bp, Homo sapiens
  misc_feature 1..20: note = RHD
  source 1..20: mol_type = genomic DNA
tcctgagccagttcccttct

SEQ ID NO: 470 - DNA, 20 bp, Homo sapiens
  misc_feature 1..20: note = RHD
  source 1..20: mol_type = genomic DNA
tgatgaccaccttcccagaa

SEQ ID NO: 471 - DNA, 20 bp, Homo sapiens
  misc_feature 1..20: note = RHD
  source 1..20: mol_type = genomic DNA
gtgatgaccaccttcccaga

SEQ ID NO: 472 - DNA, 20 bp, Homo sapiens
  misc_feature 1..20: note = RHD
  source 1..20: mol_type = genomic DNA
gagccagttcccttctggga

SEQ ID NO: 473 - DNA, 20 bp, Homo sapiens
  misc_feature 1..20: note = RHD
  source 1..20: mol_type = genomic DNA
ccagttcccttctgggaagg

SEQ ID NO: 474 - DNA, 20 bp, Homo sapiens
  misc_feature 1..20: note = RHD
  source 1..20: mol_type = genomic DNA
caccgacaaagcactcatgg

SEQ ID NO: 475 - DNA, 20 bp, Homo sapiens
  misc_feature 1..20: note = RHD
  source 1..20: mol_type = genomic DNA
cagcaccgacaaagcactca

SEQ ID NO: 476 - DNA, 20 bp, Homo sapiens
  misc_feature 1..20: note = RHD
  source 1..20: mol_type = genomic DNA
ggccaccatgagtgctttgt

SEQ ID NO: 477 - DNA, 20 bp, Homo sapiens
  misc_feature 1..20: note = RHD
  source 1..20: mol_type = genomic DNA
tttgtcggtgctgatctcag

SEQ ID NO: 478 - DNA, 20 bp, Homo sapiens
  misc_feature 1..20: note = RHD
  source 1..20: mol_type = genomic DNA
gatctcagtggatgctgtct

SEQ ID NO: 479 - DNA, 20 bp, Homo sapiens
  misc_feature 1..20: note = RHD
  source 1..20: mol_type = genomic DNA
atctcagtggatgctgtctt

SEQ ID NO: 480 - DNA, 20 bp, Homo sapiens
  misc_feature 1..20: note = RHD
  source 1..20: mol_type = genomic DNA
tctcagtggatgctgtcttg

SEQ ID NO: 481 - DNA, 20 bp, Homo sapiens
  misc_feature 1..20: note = RHD
  source 1..20: mol_type = genomic DNA
agtggatgctgtcttgggga

SEQ ID NO: 482 - DNA, 20 bp, Homo sapiens
  misc_feature 1..20: note = RHD
  source 1..20: mol_type = genomic DNA
tgtcttggggaaggtcaact

SEQ ID NO: 483 - DNA, 20 bp, Homo sapiens
  misc_feature 1..20: note = RHD
  source 1..20: mol_type = genomic DNA
gaaggtcaacttggcgcagt

SEQ ID NO: 484 - DNA, 20 bp, Homo sapiens
  misc_feature 1..20: note = RHD
  source 1..20: mol_type = genomic DNA
ggtcaacttggcgcagttgg

SEQ ID NO: 485 - DNA, 20 bp, Homo sapiens
  misc_feature 1..20: note = RHD
  source 1..20: mol_type = genomic DNA
cttggcgcagttggtggtga

SEQ ID NO: 486 - DNA, 20 bp, Homo sapiens
  misc_feature 1..20: note = RHD
  source 1..20: mol_type = genomic DNA
gcagttggtggtgatggtgc

SEQ ID NO: 487 - DNA, 20 bp, Homo sapiens
  misc_feature 1..20: note = RHD
  source 1..20: mol_type = genomic DNA
gttggtggtgatggtgctgg

SEQ ID NO: 488 - DNA, 20 bp, Homo sapiens
  misc_feature 1..20: note = RHD
  source 1..20: mol_type = genomic DNA
ggtggtgatggtgctggtgg

SEQ ID NO: 489 - DNA, 20 bp, Homo sapiens
  misc_feature 1..20: note = RHD
  source 1..20: mol_type = genomic DNA
ctggtggaggtgacagcttt

SEQ ID NO: 490 - DNA, 20 bp, Homo sapiens
  misc_feature 1..20: note = RHD
  source 1..20: mol_type = genomic DNA
tgacagctttaggcaacctg

SEQ ID NO: 491 - DNA, 20 bp, Homo sapiens
  misc_feature 1..20: note = RHD
  source 1..20: mol_type = genomic DNA
agctttaggcaacctgagga

SEQ ID NO: 492 - DNA, 20 bp, Homo sapiens
  misc_feature 1..20: note = RHD
  source 1..20: mol_type = genomic DNA
tattactgatgaccatcctc

SEQ ID NO: 493 - DNA, 20 bp, Homo sapiens
  misc_feature 1..20: note = RHD
  source 1..20: mol_type = genomic DNA
agatgtgcatcatgttcatg

SEQ ID NO: 494 - DNA, 20 bp, Homo sapiens
  misc_feature 1..20: note = RHD
  source 1..20: mol_type = genomic DNA
tacgtgttcgcagcctattt

SEQ ID NO: 495 - DNA, 20 bp, Homo sapiens
  misc_feature 1..20: note = RHD
  source 1..20: mol_type = genomic DNA
acgtgttcgcagcctatttt

SEQ ID NO: 496 - DNA, 20 bp, Homo sapiens
  misc_feature 1..20: note = RHD
  source 1..20: mol_type = genomic DNA
ggccacagacagcccaaaat

SEQ ID NO: 497 - DNA, 20 bp, Homo sapiens
  misc_feature 1..20: note = RHD
  source 1..20: mol_type = genomic DNA
agcctattttgggctgtctg

SEQ ID NO: 498 - DNA, 20 bp, Homo sapiens
  misc_feature 1..20: note = RHD
  source 1..20: mol_type = genomic DNA
attttgggctgtctgtggcc

SEQ ID NO: 499 - DNA, 20 bp, Homo sapiens
  misc_feature 1..20: note = RHD
  source 1..20: mol_type = genomic DNA
tagaggctttggcaggcacc

SEQ ID NO: 500 - DNA, 20 bp, Homo sapiens
  misc_feature 1..20: note = RHD
  source 1..20: mol_type = genomic DNA
cctcgggtagaggctttggc

SEQ ID NO: 501 - DNA, 20 bp, Homo sapiens
  misc_feature 1..20: note = RHD
  source 1..20: mol_type = genomic DNA
gttccctcgggtagaggctt

SEQ ID NO: 502 - DNA, 20 bp, Homo sapiens
  misc_feature 1..20: note = RHD
  source 1..20: mol_type = genomic DNA
tcctccgttccctcgggtag

SEQ ID NO: 503 - DNA, 20 bp, Homo sapiens
  misc_feature 1..20: note = RHD
  source 1..20: mol_type = genomic DNA
cctgccaaagcctctacccg

SEQ ID NO: 504 - DNA, 20 bp, Homo sapiens
  misc_feature 1..20: note = RHD
  source 1..20: mol_type = genomic DNA
ctgccaaagcctctacccga

SEQ ID NO: 505 - DNA, 20 bp, Homo sapiens
  misc_feature 1..20: note = RHD
  source 1..20: mol_type = genomic DNA
tctttatcctccgttccctc

SEQ ID NO: 506 - DNA, 20 bp, Homo sapiens
  misc_feature 1..20: note = RHD
  source 1..20: mol_type = genomic DNA
aaagcctctacccgagggaa

SEQ ID NO: 507 - DNA, 20 bp, Homo sapiens
  misc_feature 1..20: note = RHD
  source 1..20: mol_type = genomic DNA
atctttatcctccgttccct

SEQ ID NO: 508 - DNA, 20 bp, Homo sapiens
  misc_feature 1..20: note = RHD
  source 1..20: mol_type = genomic DNA
gcctctacccgagggaacgg

SEQ ID NO: 509 - DNA, 20 bp, Homo sapiens
  misc_feature 1..20: note = RHD
  source 1..20: mol_type = genomic DNA
acccagtttgtctgccatgc

SEQ ID NO: 510 - DNA, 20 bp, Homo sapiens
  misc_feature 1..20: note = RHD
  source 1..20: mol_type = genomic DNA
ccagaacatccacaagaaga

SEQ ID NO: 511 - DNA, 20 bp, Homo sapiens
  misc_feature 1..20: note = RHD
  source 1..20: mol_type = genomic DNA
gccagaacatccacaagaag

SEQ ID NO: 512 - DNA, 20 bp, Homo sapiens
  misc_feature 1..20: note = RHD
  source 1..20: mol_type = genomic DNA
ccctcttcttgtggatgttc

SEQ ID NO: 513 - DNA, 20 bp, Homo sapiens
  misc_feature 1..20: note = RHD
  source 1..20: mol_type = genomic DNA
agcagagcagagttgaaact

SEQ ID NO: 514 - DNA, 20 bp, Homo sapiens
  misc_feature 1..20: note = RHD
  source 1..20: mol_type = genomic DNA
tgctgagaagtccaatcgaa

SEQ ID NO: 515 - DNA, 20 bp, Homo sapiens
  misc_feature 1..20: note = RHD
  source 1..20: mol_type = genomic DNA
acggcattcttcctttcgat

SEQ ID NO: 516 - DNA, 20 bp, Homo sapiens
  misc_feature 1..20: note = RHD
  source 1..20: mol_type = genomic DNA
agcatagtaggtgttgaaca

SEQ ID NO: 517 - DNA, 20 bp, Homo sapiens
  misc_feature 1..20: note = RHD
  source 1..20: mol_type = genomic DNA
gctgactgctacagcatagt

SEQ ID NO: 518 - DNA, 20 bp, Homo sapiens
  misc_feature 1..20: note = RHD
  source 1..20: mol_type = genomic DNA
ctatgctgtagcagtcagcg

SEQ ID NO: 519 - DNA, 20 bp, Homo sapiens
  misc_feature 1..20: note = RHD
  source 1..20: mol_type = genomic DNA
agcgtggtgacagccatctc

SEQ ID NO: 520 - DNA, 20 bp, Homo sapiens
  misc_feature 1..20: note = RHD
  source 1..20: mol_type = genomic DNA
gcgtggtgacagccatctca

SEQ ID NO: 521 - DNA, 20 bp, Homo sapiens
  misc_feature 1..20: note = RHD
  source 1..20: mol_type = genomic DNA
agccaaggatgaccctgaga

SEQ ID NO: 522 - DNA, 20 bp, Homo sapiens
  misc_feature 1..20: note = RHD
  source 1..20: mol_type = genomic DNA
agccatctcagggtcatcct

SEQ ID NO: 523 - DNA, 20 bp, Homo sapiens
  misc_feature 1..20: note = RHD
  source 1..20: mol_type = genomic DNA
cttcccttgggggtgagcca

SEQ ID NO: 524 - DNA, 20 bp, Homo sapiens
  misc_feature 1..20: note = RHD
  source 1..20: mol_type = genomic DNA
tcatccttggctcaccccca

SEQ ID NO: 525 - DNA, 20 bp, Homo sapiens
  misc_feature 1..20: note = RHD
  source 1..20: mol_type = genomic DNA
catccttggctcacccccaa

SEQ ID NO: 526 - DNA, 20 bp, Homo sapiens
  misc_feature 1..20: note = RHD
  source 1..20: mol_type = genomic DNA
ttatgtgcacagtgcggtgt

SEQ ID NO: 527 - DNA, 20 bp, Homo sapiens
  misc_feature 1..20: note = RHD
  source 1..20: mol_type = genomic DNA
gtgcacagtgcggtgttggc

SEQ ID NO: 528 - DNA, 20 bp, Homo sapiens
  misc_feature 1..20: note = RHD
  source 1..20: mol_type = genomic DNA
cacagtgcggtgttggcagg

SEQ ID NO: 529 - DNA, 20 bp, Homo sapiens
  misc_feature 1..20: note = RHD
  source 1..20: mol_type = genomic DNA
tgcggtgttggcaggaggcg

SEQ ID NO: 530 - DNA, 20 bp, Homo sapiens
  misc_feature 1..20: note = RHD
  source 1..20: mol_type = genomic DNA
gttggcaggaggcgtggctg

SEQ ID NO: 531 - DNA, 20 bp, Homo sapiens
  misc_feature 1..20: note = RHD
  source 1..20: mol_type = genomic DNA
ttggcaggaggcgtggctgt

SEQ ID NO: 532 - DNA, 20 bp, Homo sapiens
  misc_feature 1..20: note = RHD
  source 1..20: mol_type = genomic DNA
agaagggatcaggtgacacg

SEQ ID NO: 533 - DNA, 20 bp, Homo sapiens
  misc_feature 1..20: note = RHD
  source 1..20: mol_type = genomic DNA
caagccacggagaagggatc

SEQ ID NO: 534 - DNA, 20 bp, Homo sapiens
  misc_feature 1..20: note = RHD
  source 1..20: mol_type = genomic DNA
ccatggcaagccacggagaa

SEQ ID NO: 535 - DNA, 20 bp, Homo sapiens
  misc_feature 1..20: note = RHD
  source 1..20: mol_type = genomic DNA
gtcacctgatcccttctccg

SEQ ID NO: 536 - DNA, 20 bp, Homo sapiens
  misc_feature 1..20: note = RHD
  source 1..20: mol_type = genomic DNA
accatggcaagccacggaga

SEQ ID NO: 537 - DNA, 20 bp, Homo sapiens
  misc_feature 1..20: note = RHD
  source 1..20: mol_type = genomic DNA
cccagcaccatggcaagcca

SEQ ID NO: 538 - DNA, 20 bp, Homo sapiens
  misc_feature 1..20: note = RHD
  source 1..20: mol_type = genomic DNA
cccttctccgtggcttgcca

SEQ ID NO: 539 - DNA, 20 bp, Homo sapiens
  misc_feature 1..20: note = RHD
  source 1..20: mol_type = genomic DNA
tccgtggcttgccatggtgc

SEQ ID NO: 540 - DNA, 20 bp, Homo sapiens
  misc_feature 1..20: note = RHD
  source 1..20: mol_type = genomic DNA
agccacaagacccagcacca

SEQ ID NO: 541 - DNA, 20 bp, Homo sapiens
  misc_feature 1..20: note = RHD
  source 1..20: mol_type = genomic DNA
ccgtggcttgccatggtgct

SEQ ID NO: 542 - DNA, 20 bp, Homo sapiens
  misc_feature 1..20: note = RHD
  source 1..20: mol_type = genomic DNA
tgccatggtgctgggtcttg

SEQ ID NO: 543 - DNA, 20 bp, Homo sapiens
  misc_feature 1..20: note = RHD
  source 1..20: mol_type = genomic DNA
atggtgctgggtcttgtggc

SEQ ID NO: 544 - DNA, 20 bp, Homo sapiens
  misc_feature 1..20: note = RHD
  source 1..20: mol_type = genomic DNA
tggtgctgggtcttgtggct

SEQ ID NO: 545 - DNA, 20 bp, Homo sapiens
  misc_feature 1..20: note = RHD
  source 1..20: mol_type = genomic DNA
gtggctgggctgatctccgt

SEQ ID NO: 546 - DNA, 20 bp, Homo sapiens
  misc_feature 1..20: note = RHD
  source 1..20: mol_type = genomic DNA
tggctgggctgatctccgtc

SEQ ID NO: 547 - DNA, 20 bp, Homo sapiens
  misc_feature 1..20: note = RHD
  source 1..20: mol_type = genomic DNA
ggctgggctgatctccgtcg

SEQ ID NO: 548 - DNA, 20 bp, Homo sapiens
  misc_feature 1..20: note = RHD
  source 1..20: mol_type = genomic DNA
gctgggctgatctccgtcgg

SEQ ID NO: 549 - DNA, 20 bp, Homo sapiens
  misc_feature 1..20: note = RHD
  source 1..20: mol_type = genomic DNA
caggtacttggctcccccga

SEQ ID NO: 550 - DNA, 20 bp, Homo sapiens
  misc_feature 1..20: note = RHD
  source 1..20: mol_type = genomic DNA
gggtgttgtaaccgagtgct

SEQ ID NO: 551 - DNA, 20 bp, Homo sapiens
  misc_feature 1..20: note = RHD
  source 1..20: mol_type = genomic DNA
tgtggggaatccccagcact

SEQ ID NO: 552 - DNA, 20 bp, Homo sapiens
  misc_feature 1..20: note = RHD
  source 1..20: mol_type = genomic DNA
ggtgttgtaaccgagtgctg

SEQ ID NO: 553 - DNA, 20 bp, Homo sapiens
  misc_feature 1..20: note = RHD
  source 1..20: mol_type = genomic DNA
tagcccatgatggagctgtg

SEQ ID NO: 554 - DNA, 20 bp, Homo sapiens
  misc_feature 1..20: note = RHD
  source 1..20: mol_type = genomic DNA
gtagcccatgatggagctgt

SEQ ID NO: 555 - DNA, 20 bp, Homo sapiens
  misc_feature 1..20: note = RHD
  source 1..20: mol_type = genomic DNA
tgtagcccatgatggagctg

SEQ ID NO: 556 - DNA, 20 bp, Homo sapiens
  misc_feature 1..20: note = RHD
  source 1..20: mol_type = genomic DNA
gattccccacagctccatca

SEQ ID NO: 557 - DNA, 20 bp, Homo sapiens
  misc_feature 1..20: note = RHD
  source 1..20: mol_type = genomic DNA
attccccacagctccatcat

SEQ ID NO: 558 - DNA, 20 bp, Homo sapiens
  misc_feature 1..20: note = RHD
  source 1..20: mol_type = genomic DNA
gctgaagttgtagcccatga

SEQ ID NO: 559 - DNA, 20 bp, Homo sapiens
  misc_feature 1..20: note = RHD
  source 1..20: mol_type = genomic DNA
gggctacaacttcagcttgc

SEQ ID NO: 560 - DNA, 20 bp, Homo sapiens
  misc_feature 1..20: note = RHD
  source 1..20: mol_type = genomic DNA
ggctacaacttcagcttgct

SEQ ID NO: 561 - DNA, 20 bp, Homo sapiens
  misc_feature 1..20: note = RHD
  source 1..20: mol_type = genomic DNA
ttcagcttgctgggtctgct

SEQ ID NO: 562 - DNA, 20 bp, Homo sapiens
  misc_feature 1..20: note = RHD
  source 1..20: mol_type = genomic DNA
gatcatctacattgtgctgc

SEQ ID NO: 563 - DNA, 20 bp, Homo sapiens
  misc_feature 1..20: note = RHD
  source 1..20: mol_type = genomic DNA
ctgctggtgcttgataccgt

SEQ ID NO: 564 - DNA, 20 bp, Homo sapiens
  misc_feature 1..20: note = RHD
  source 1..20: mol_type = genomic DNA
gtgcttgataccgtcggagc

SEQ ID NO: 565 - DNA, 20 bp, Homo sapiens
  misc_feature 1..20: note = RHD
  source 1..20: mol_type = genomic DNA
gataccgtcggagccggcaa

SEQ ID NO: 566 - DNA, 20 bp, Homo sapiens
  misc_feature 1..20: note = RHD
  source 1..20: mol_type = genomic DNA
ccccaatgctgaggaggacc

SEQ ID NO: 567 - DNA, 20 bp, Homo sapiens
  misc_feature 1..20: note = RHD
  source 1..20: mol_type = genomic DNA
tgagttccccaatgctgagg

SEQ ID NO: 568 - DNA, 20 bp, Homo sapiens
  misc_feature 1..20: note = RHD
  source 1..20: mol_type = genomic DNA
ttccaggtcctcctcagcat

SEQ ID NO: 569 - DNA, 20 bp, Homo sapiens
  misc_feature 1..20: note = RHD
  source 1..20: mol_type = genomic DNA
agctgagttccccaatgctg

SEQ ID NO: 570 - DNA, 20 bp, Homo sapiens
  misc_feature 1..20: note = RHD
  source 1..20: mol_type = genomic DNA
tccaggtcctcctcagcatt

SEQ ID NO: 571 - DNA, 20 bp, Homo sapiens
  misc_feature 1..20: note = RHD
  source 1..20: mol_type = genomic DNA
ccaggtcctcctcagcattg

SEQ ID NO: 572 - DNA, 20 bp, Homo sapiens
  misc_feature 1..20: note = RHD
  source 1..20: mol_type = genomic DNA
cagcattggggaactcagct

SEQ ID NO: 573 - DNA, 20 bp, Homo sapiens
  misc_feature 1..20: note = RHD
  source 1..20: mol_type = genomic DNA
agacatgagagctatcacga

SEQ ID NO: 574 - DNA, 20 bp, Homo sapiens
  misc_feature 1..20: note = RHD
  source 1..20: mol_type = genomic DNA
atcgtgatagctctcatgtc

SEQ ID NO: 575 - DNA, 20 bp, Homo sapiens
  misc_feature 1..20: note = RHD
  source 1..20: mol_type = genomic DNA
ctttccatattttaagattt

SEQ ID NO: 576 - DNA, 20 bp, Homo sapiens
  misc_feature 1..20: note = RHD
  source 1..20: mol_type = genomic DNA
tgctcctaaatcttaaaata

SEQ ID NO: 577 - DNA, 20 bp, Homo sapiens
  misc_feature 1..20: note = RHD
  source 1..20: mol_type = genomic DNA
aatatggaaagcacctcatg

SEQ ID NO: 578 - DNA, 20 bp, Homo sapiens
  misc_feature 1..20: note = RHD
  source 1..20: mol_type = genomic DNA
tcaaaatatttagcctcatg

SEQ ID NO: 579 - DNA, 20 bp, Homo sapiens
  misc_feature 1..20: note = RHD
  source 1..20: mol_type = genomic DNA
attttgatgaccaagttttc

SEQ ID NO: 580 - DNA, 20 bp, Homo sapiens
  misc_feature 1..20: note = RHD
  source 1..20: mol_type = genomic DNA
taaaatccaacagccaaatg

SEQ ID NO: 581 - DNA, 20 bp, synthetic construct
  misc_feature 1..20: note = Synthetic: gRNA targeting sequence
  source 1..20: mol_type = other DNA
gatgaccctggctcatatca

SEQ ID NO: 582 - DNA, 20 bp, synthetic construct
  misc_feature 1..20: note = Synthetic: gRNA targeting sequence
  source 1..20: mol_type = other DNA
gtttctgcctgtcatagcgc

SEQ ID NO: 583 - protein, 305 aa, synthetic construct
  REGION 1..305: note = Synthetic: CD47-201 amino acid sequence
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 584 - protein, 323 aa, synthetic construct
  REGION 1..323: note = Synthetic: CD47-202 amino acid sequence
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 585 - protein, 305 aa, synthetic construct
  REGION 1..305: note = Synthetic: CD47-202 amino acid sequence without signal peptide
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 586 - protein, 86 aa, synthetic construct
  REGION 1..86: note = Synthetic: CD47-203 amino acid sequence
XWTESLYCGVYTNAWPSSDFRFEYLSSSTITWTSLYEICGVQRDTYSQLKGKKRQASNQKTIQPPRKAVEEPLNAFKESKGMMNDE

SEQ ID NO: 587 - protein, 109 aa, synthetic construct
  REGION 1..109: note = Synthetic: CD47-205 amino acid sequence
XKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNE

SEQ ID NO: 588 - protein, 183 aa, synthetic construct
  REGION 1..183: note = Synthetic: CD47-206 amino acid sequence
MEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVF

SEQ ID NO: 589 - DNA, 918 bp, synthetic construct
  misc_feature 1..918: note = Synthetic: CD47-201 CDS nucleotide sequence
  source 1..918: mol_type = other DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 590 - DNA, 972 bp, synthetic construct
  misc_feature 1..972: note = Synthetic: CD47-202 CDS nucleotide sequence
  source 1..972: mol_type = other DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 591 - DNA, 258 bp, synthetic construct
  misc_feature 1..258: note = Synthetic: CD47-203 CDS nucleotide sequence
  source 1..258: mol_type = other DNA
tggactgagtctctgtattgcggcgtgtataccaatgcatggccctcttctgatttcaggtttgagtatcttagctctagcacaattacttggactagtttatatgaaatttgtggggttcagagggacacctacagtcagttgaaaggcaagaagagacaagcttccaatcagaagactatacaacctcctaggaaagctgtagaggaaccccttaatgcattcaaagaatcaaaaggaatgatgaatgatgaataa

SEQ ID NO: 592 - DNA, 6796 bp, synthetic construct
  misc_feature 1..6796: note = Synthetic: CD47-204 CDS nucleotide sequence
  source 1..6796: mol_type = other DNA
agatattttcttgttcaatttaaggagaggtaaatttggtatcaatagaaaaaatgtttctgaaaaatttaaaccctggaaatgtatttatggcatggagtcagatgtttcagggagagaagaacaaatcaagaagcattgcaagtatgctcatatggaatgcttaaggcttgtggttaaaaaatatatatatatggctgtcaatgtcttaggctcatggtagcagcagaaatcgtaataattcttttgtcacatgggttatatccatattggagagaattaactcaggtgaaattaacttgtacactgtttggttttataatatttagagggatcacaactgactgatgtccctttgaagtaccattcttcataaatctttttttttcagaatgggccagccaactgtgacatcccttggatcggagatttagaactagaaagtattctttctacattattagggaagaaaaggagttacttggcggttagcaatattctattttgttttgttttgtttttagagacagggtctcattatgttgaccaggctggcctcgagctcctgggctcaagcaatgctcccacctcagcctcccaagtagctgggactacaggcatgtgccactacacctggcagtgtttattctgataaatacatttatgagctcaaaaatgtaactctaaaaccttatctctgaacttccatattaccatcagaaatttagatagttgtttagttctctttttctttgtagaacatagatataaggcatggtttcattgaagtcagttgtatatacatgtaactatcctgatgttcccaaataaagctctgtatttctgcttagtttattggggaggctgctaaatgtagtgcatcccaacccattttaccctgttctactttaaaaagaggttggcttcttgtttggatacaaggaccaagtcactcccccaggttcctccacagtaagggaggcctatttaaagccgcccatggcactaacagaaactggactcctatgagctcagatacataactgggcctcacaggggtgggacagtatgtagtctaggaattggaaggatccattccatatcaaagaactgaagcatcgtgttgccctctcagcagcaagagtaaggtgatgcccctgtcagttatagttcctgagttcctctgtctttgattctttgcctattagccagctagctcaccctcttgtttatgccactgttttttatcctattcatgccttctcacagacaacttttcttacctacagctttggactcatccttgtctcctttctgtttctttttcactttcccttcccatcaccaactttctgggtttttttctgtttcttcttagagtccagtggcagggagaaacttgtcagtccagtctgttgccatttttcctgtttgagaaagactcaccagcttttggctggctcacagattggctttccttgggtcaggacccacccttttccctgccagctttggaagcttgacagaattcgagtgtgcagtggtggtaaataaatagtaaggaacacagagcagtcctggaggcgtgcctccatctgctgatgagaaaatccagtgctgtcatccagcccaggtcccagcggaatgggcctctctgttcagtaggatccccctcctgctgagtggttcatggcatgtttctgttcaacgcttttccatctgtaggattcttattctgtatttatttgtttttttgggtttttttattttttgagatggagtctcgctctgtcgcccaggctggagtgcagtggcacgaccccagctcgctgcagcctctgcctcccaggacgagggagatcctcccacctcagccttccacgtagctgggactacaggcatgcaccacaggcatgcaccaccacgccagctaatttttgtatttttggtagagacagggttgcatcatgttgcccaggctggtcttgaatgcctgagctcaagcaatctatttgccttggcctcccaaagtgctgggattacaggcatgagccaccacggccagccttctcatttgttttttttataaggaagctatctcttcttccctccccaactagggtattctttttccctttcgtcactttgctcatgtactgtattccttcaacttcattaatgaatccatttggaagcagtgaaaaaggcaactcagaaagctaagaagaaatagatagaggaatactcagagctatctgagtattttctttagtttgttagctctttggagctttgaaactggaaagacccagggagtgatgtggagaaagagactgagcttgtaagacacaggagcagtgagctaagggagatggagtagtggggacaaattctggcacattctgtctacactctgggtagatagaggagggaggatggagcacccatggtgggggtatgttggtgacagcattttcccaccagccagtgtaacaagtggctgatttgggggaaagatggcataaacaaatgagagaatgtgtttactatttgatgtagatgggttatttgcttcatttttcaaatcagtgtatataatcaagaatattcagcatgtttgaatagactgtcagagctggaactctttcattaacatctctggcacctttagttttagccctgaacattttatcttaaaattaaacattaccaaatgccttagtttatttcatttattaaatttatattcttatttgttatttatatcagcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctat

SEQ ID NO: 593 - DNA, 327 bp, synthetic construct
  misc_feature 1..327: note = Synthetic: CD47-205 CDS nucleotide sequence
  source 1..327: mol_type = other DNA
aagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataa

SEQ ID NO: 594 - DNA, 549 bp, synthetic construct
  misc_feature 1..549: note = Synthetic: CD47-206 CDS nucleotide sequence
  source 1..549: mol_type = other DNA
atggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgttt

SEQ ID NO: 595 - DNA, 969 bp, synthetic construct
  misc_feature 1..969: note = Synthetic: Codon-optimized nucleotide sequence encoding CD47
  source 1..969: mol_type = other DNA
atgtggcccctggtcgccgccctgttgctgggctcggcatgctgcggatcagctcagctactgtttaataaaacaaaatctgtagaattcacgttttgtaacgacactgtcgtgatcccatgctttgttactaatatggaggcacaaaacaccactgaagtgtacgtgaagtggaaattcaaaggcagagacatttacacctttgacggcgccctcaacaagtccaccgtgcccactgactttagtagcgcaaaaattgaggtcagccaattactaaaaggagatgcctctttgaagatggacaagagcgatgctgtcagccacacagggaactacacttgtgaagtaacagagttaacccgcgaaggtgaaacgatcatcgagctgaagtatcgagtggtgtcctggttttctccgaacgagaatatccttatcgtaattttcccaattttcgctatcctcctgttctggggccagtttggtatcaagacactcaaatatcggtccggtgggatggatgagaagacaattgccctgcttgttgctggactcgtgatcaccgtcatcgtgattgttggggccatccttttcgtcccaggggagtacagcctgaagaatgctacgggcctgggattaattgtgacctctacagggatactcatcctgcttcactactatgtgttcagtaccgcgattggactgacctccttcgtcattgccatattggtgattcaggtgatagcctacatcctcgccgtggttggcctgagtctctgtatcgcggcgtgcatacccatgcatggccctcttctgatttcagggttgagtatcctcgcactagcacagttgctgggactggtttatatgaaatttgtggcctccaaccagaagactatacagcctcctaggaaggctgtagaggagcccctgaatgcattcaaggaatcaaaaggcatgatgaatgatgaa